(19) United States
(12) Patent Application Publication
(10) Pub. No.: US 2025/0386663 A1
(43) Pub. Date: Dec. 18, 2025

(54) LIGHT-EMITTING ELEMENT, AMINE COMPOUND FOR THE SAME, AND ELECTRONIC DEVICE INCLUDING THE SAME

(71) Applicant: Samsung Display Co., Ltd., Yongin-si (KR)

(72) Inventor: Ichinori TAKADA, Yokohama (JP)

(21) Appl. No.: 19/188,985

(22) Filed: Apr. 24, 2025

(30) Foreign Application Priority Data
Jun. 14, 2024 (KR) 10-2024-0077483

(51) Int. Cl. H10K 50/15; H10K 59/38; H10K 59/80; H10K 85/30; H10K 85/40; H10K 85/60

(52) U.S. Cl. CPC H10K 50/15; H10K 59/38; H10K 59/879; H10K 85/342; H10K 85/346; H10K 85/40; H10K 85/622; H10K 85/623; H10K 85/626; H10K 85/633; H10K 85/636; H10K 85/654; H10K 85/6572; H10K 85/6574; H10K 85/6576; H10K 85/658

(57) ABSTRACT

A light-emitting element including a first electrode, and a second electrode arranged on the first electrode is provided. At least one functional layer is arranged between the first electrode and the second electrode and includes an amine compound represented by Formula 1.

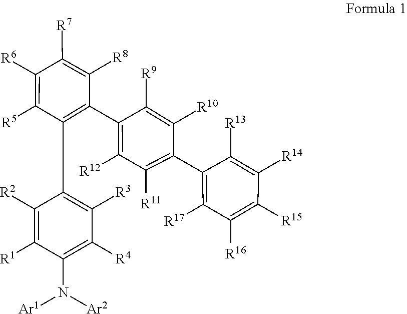

### CROSS-REFERENCE TO RELATED APPLICATION

This application claims priority to and the benefit of Korean Patent Application No. 10-2024-0077483, filed on Jun. 14, 2024, in the Korean Intellectual Property Office, the entire content of which is incorporated herein by reference.

### BACKGROUND

### 1. Field

The present disclosure herein relates to a light-emitting element, an amine compound for the light-emitting element, and an electronic device including the light-emitting element.

### 2. Description of the Related Art

Recently, the development of organic electroluminescence display devices as image display devices has been actively conducted. These devices recombine holes and electrons in a light-emitting layer, injected from a first electrode and a second electrode, respectively. This recombination causes an emission material in the light-emitting layer to emit light, thereby implementing image display. In the application of the organic light-emitting elements to display devices, there is a consistent demand or desire for increased emission efficiency and longer lifespan. Consequently, there is ongoing development of materials that can stably achieve these requirements or desires.

For example, to implement the light-emitting element with high efficiency and long lifespan, there is active development of materials for the hole transport region that exhibit enhanced (e.g., excellent or suitable) hole transporting properties and stability.

### SUMMARY

One or more aspects of embodiments of the present disclosure are directed toward a light-emitting element with (having) enhanced (improved) emission efficiency and element lifespan.

One or more aspects of embodiments of the present disclosure are directed toward an amine compound capable of enhancing (improving) emission efficiency and element lifespan of a light-emitting element.

One or more aspects of embodiments of the present disclosure are directed toward a display device including a light-emitting element having enhanced (improved) emission efficiency and lifespan, thereby having excellent or suitable display quality. One or more aspects of embodiments of the present disclosure are directed toward an electronic device including a display device with a light-emitting element having enhanced (improved) emission efficiency and lifespan, thereby having excellent or suitable display quality. However, aspects of the present disclosure are not restricted to those set forth herein.

Additional aspects will be set forth in part in the description which follows and, in part, will be apparent from the description, or may be learned by practice of the presented embodiments of the disclosure.

One or more embodiments of the disclosure provides a light-emitting element including a first electrode, a second electrode arranged on the first electrode, and at least one functional layer arranged between the first electrode and the second electrode and including an amine compound represented by Formula 1.

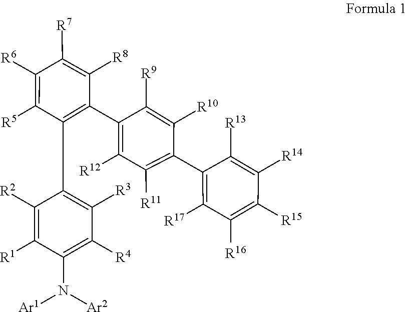

In Formula 1, R¹ to R¹⁷ may each independently be a hydrogen atom, a deuterium atom, a substituted or unsubstituted oxy group, a substituted or unsubstituted thio group, a substituted or unsubstituted amine group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 25 ring-forming carbons, and/or may be bonded to an adjacent group to form a ring; a case where R⁵ is a substituted or unsubstituted carbazolyl group is excluded; Ar¹ and Ar² may each independently be a substituted or unsubstituted aryl group having 6 to 30 ring-forming carbons, or a substituted or unsubstituted heteroaryl group having 2 to 30 ring-forming carbons, and at least one selected from among Ar¹ and Ar² includes a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group, and if (e.g., when) each of Ar¹ and Ar² includes (e.g., a substituted group) a substituted naphthyl group, a substituted naphthylphenyl group, a substituted dibenzofuranyl group, a substituted dibenzothiophenyl group, or a substituted carbazolyl group, the substituent (e.g., a substituent of the substituted group) may be (e.g., is optionally or is configured to be) bonded to an adjacent group to form a ring; a case where at least one selected from among Ar¹ and Ar² is a 4-phenyl-dibenzofuran-1-yl group is excluded; a case where at least one selected from among (e.g., or both) Ar¹ and Ar² include a heteroaryl group substituted with a heteroaryl group is excluded; a case where the amine compound represented by Formula 1 includes at least one among a chlorine atom and acenaphthene is excluded; and the amine compound represented by Formula 1 includes a structure in which at least one hydrogen atom is substituted with a deuterium atom.

In one or more embodiments, the at least one functional layer may include a light-emitting layer, a hole transport region arranged between the first electrode and the light-emitting layer, and an electron transport region arranged between the light-emitting layer and the second electrode, and the hole transport region may include the amine compound represented by Formula 1.

In one or more embodiments, the hole transport region may include a hole injection layer arranged on the first electrode, and a hole transport layer arranged on the hole injection layer, and the hole transport layer may include the amine compound represented by Formula 1.

In one or more embodiments, at least one selected from among Ar¹ and Ar² may be represented by any one among Formula 2-1 to Formula 2-3.

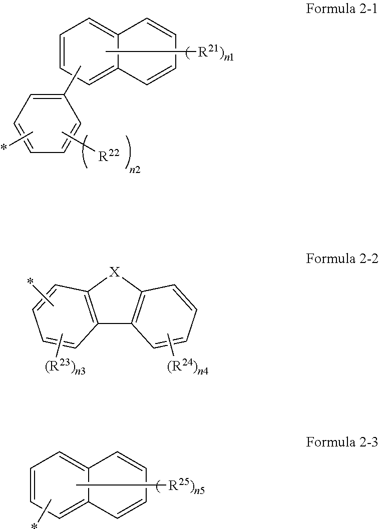

In Formula 2-1 to Formula 2-3, X may be O, S, or NR²⁶, R²¹, R²², R²⁵, and R²⁶ may each independently be a hydrogen atom, a deuterium atom, a halogen atom, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or may be bonded to an adjacent group to form a ring, R²³ and R²⁴ may each independently be a hydrogen atom, a deuterium atom, a halogen atom, a substituted or unsubstituted alkyl group having 1 to 20 carbons, a substituted or unsubstituted alkenyl group having 2 to 20 carbons, or a substituted or unsubstituted aryl group having 6 to 30 ring-forming carbons, and/or may be is bonded to an adjacent group to form a ring, n1 may be an integer of 0 to 7, n2 and n4 may each independently be an integer of 0 to 4, n3 may be an integer of 0 to 3, n5 may be an integer of 0 to 7, and [symbol] is a position to which Formula 1 may be is connected.

In one or more embodiments, one selected from among Ar¹ and Ar² may be represented by any one selected from among Formula 2-1 to Formula 2-3, and the (e.g., other) one remaining Ar¹ or Ar² may be a substituted or unsubstituted phenyl group, a substituted or unsubstituted biphenylyl group, a substituted or unsubstituted terphenylyl group, a substituted or unsubstituted quarterphenylyl group, a substituted or unsubstituted naphthyl group, a substituted or unsubstituted naphthylphenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a substituted or unsubstituted phenanthrenyl group.

In one or more embodiments, the amine compound represented by Formula 1 may be represented by Formula 3.

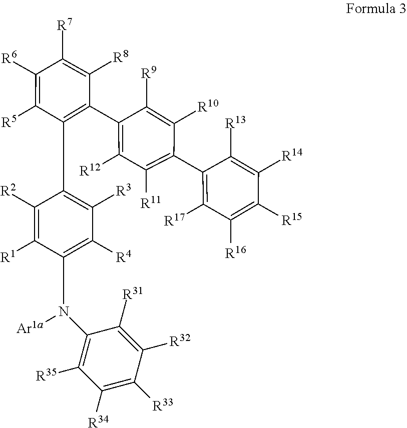

In Formula 3, R³¹ to R³⁵ may each independently be a hydrogen atom, a deuterium atom, a halogen atom, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, a substituted or unsubstituted heteroaryl group having 2 to 30 ring-forming carbons, and/or may bonded to an adjacent group to form a ring; a case where at least one selected from among R³¹ to R³⁵ is a heteroaryl group substituted with a heteroaryl group may be excluded; Ar^{1a} includes a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group, if (e.g., when) Ar^{1a} includes a substituted naphthyl group, a substituted naphthylphenyl group, a substituted dibenzofuranyl group, a substituted dibenzothiophenyl group, or a substituted carbazolyl group, then the substituent may be bonded to an adjacent group to form a ring; a case where Ar^{1a} includes a heteroaryl group substituted with a heteroaryl group is excluded; and a case where Ar^{1a} is represented by Formula 3-a is excluded.

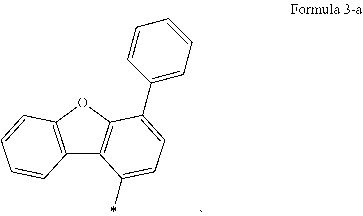 [SMILES: *c1ccc(-c2ccccc2)c2oc3ccccc3c12]

The descriptions R¹ to R¹⁷ defined in Formula 1 may be similarly applied to Formula 3.

In one or more embodiments, R³¹ to R³⁵ may each independently be a hydrogen atom, a deuterium atom, a substituted or unsubstituted phenyl group, a substituted or unsubstituted naphthyl group, or a substituted or unsubstituted phenanthrenyl group; or at least one pair among pairs including two selected from among R³¹ to R³⁵ may be bonded to each other to form a ring.

In one or more embodiments, the amine compound represented by Formula 1 may be represented by any one selected from among Formula 4-1 to Formula 4-3.

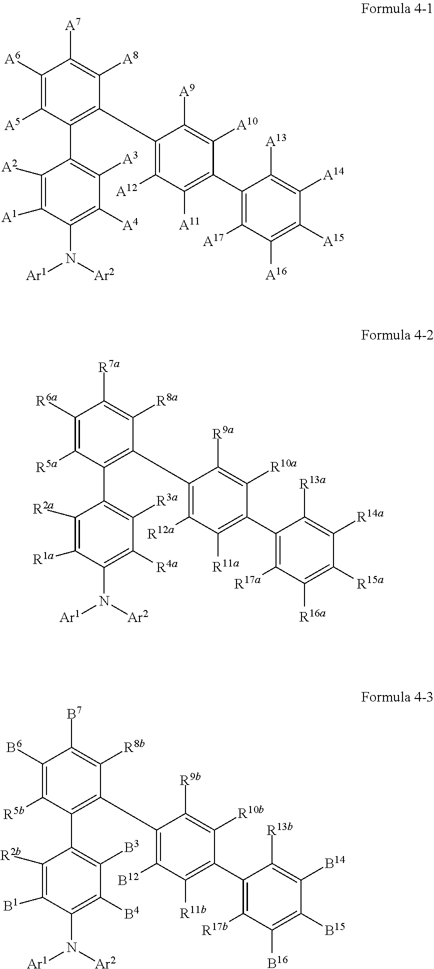

In Formula 4-1, A¹ to A¹⁷ may each independently be a hydrogen atom, a deuterium atom, or a substituted or unsubstituted aryl group having 6 to 30 ring-forming carbons; in Formula 4-2, R^{1a} to R^{17a} may each independently be a hydrogen atom, a deuterium atom, a substituted or unsubstituted oxy group, a substituted or unsubstituted thio group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or may be bonded to an adjacent group to form a ring; at least one pair among pairs including (e.g., composed of) two selected from among R^{1a} to R^{17a} may be bonded to each other to form an aromatic hydrocarbon ring; in Formula 4-3, B¹, B³, B⁴, B⁶, B⁷, B¹², B¹⁴, B¹⁵, and B¹⁶ may each independently be a hydrogen atom or a deuterium atom; R^{2b}, R^{5b}, R^{8b}, R^{9b}, R^{10b}, R^{11b}, R^{13b}, and R^{17b} may each independently be a hydrogen atom, a deuterium atom, a substituted or unsubstituted oxy group, a substituted or unsubstituted thio group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or may be bonded to an adjacent group to form a ring, and at least one pair selected from among R^{2b} and R^{5b}, R^{8b} and R^{9b}, R^{10b} and R^{13b}, and R^{11b} and R^{17b} may be bonded to each other to form a ring.

The descriptions of Ar¹ and Ar² defined in Formula 1 may be similarly applied to Formula 4-1 to 4-3.

In one or more embodiments, the amine compound represented by Formula 1 may be represented by Formula 5, and the amine compound may satisfy any one selected from among combinations present in Compound Combination Table 1.

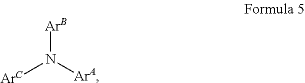

In Formula 5, Ar^{A} may be selected from among the group consisting of Substituent 1 to Substituent 12 in Substituent Group A, Ar^{B} may be selected from among the group consisting of Substituent 1 to Substituent 12, and Substituent 21 to Substituent 52 in Substituent Group A, and Ar^{C} may be selected from among the group consisting of Substituent 2 to Substituent 7, Substituent 9 to Substituent 11, and Substituent 31 to Substituent 52 in Substituent Group A.

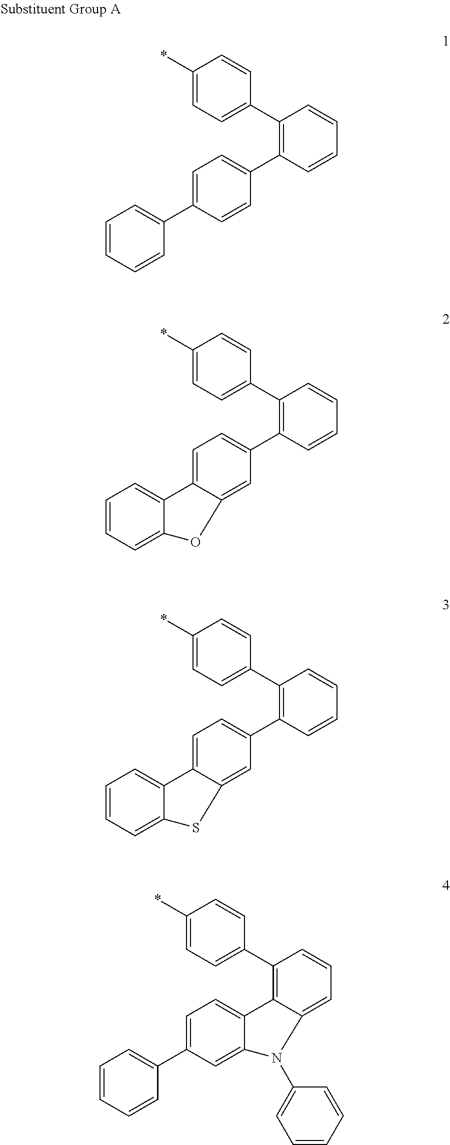 [SMILES: *c1ccc(-c2ccccc2-c2ccc(-c3ccccc3)cc2)cc1 ; *c1ccc(-c2ccccc2-c2ccc3c(c2)oc2ccccc23)cc1 ; *c1ccc(-c2ccccc2-c2ccc3c(c2)sc2ccccc23)cc1 ; *c1ccc(-c2cccc3c2c2ccc(-c4ccccc4)cc2n3-c2ccccc2)cc1]

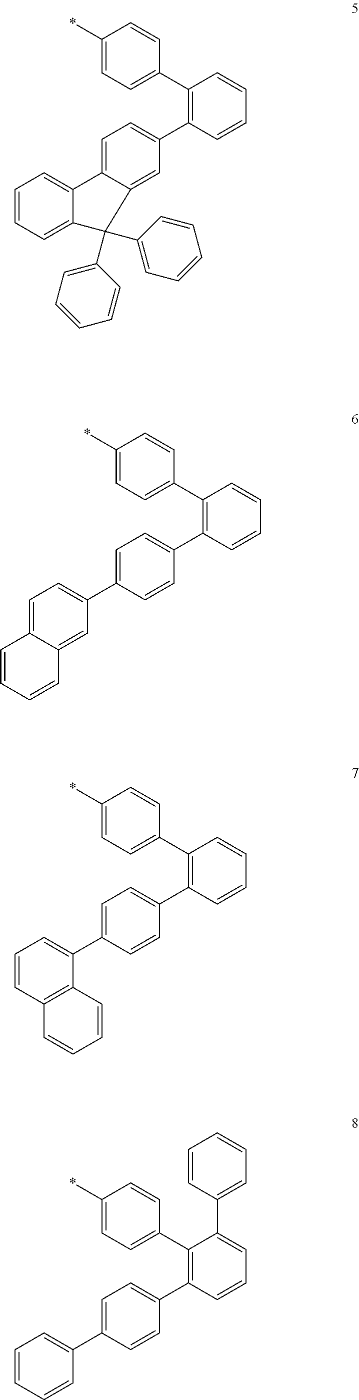 [SMILES: *c1ccc(-c2ccccc2-c2ccc3c(c2)C(c2ccccc2)(c2ccccc2)c2ccccc2-3)cc1 ; *c1ccc(-c2ccccc2-c2ccc(-c3ccc4ccccc4c3)cc2)cc1 ; *c1ccc(-c2ccccc2-c2ccc(-c3cccc4ccccc34)cc2)cc1 ; *c1ccc(-c2c(-c3ccccc3)cccc2-c2ccc(-c3ccccc3)cc2)cc1]

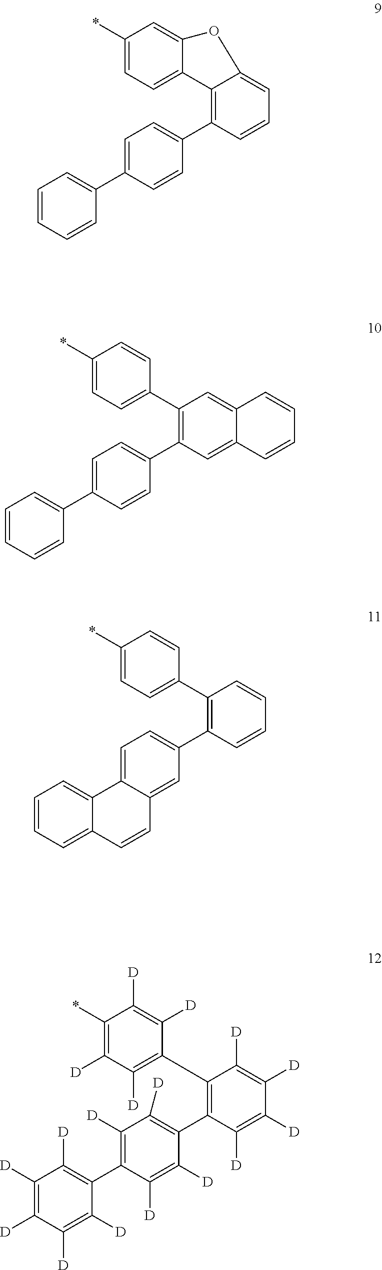 [SMILES: *c1ccc2c(c1)oc1cccc(-c3ccc(-c4ccccc4)cc3)c12 ; *c1ccc(-c2cc3ccccc3cc2-c2ccc(-c3ccccc3)cc2)cc1 ; *c1ccc(-c2ccccc2-c2ccc3c(ccc4ccccc43)c2)cc1 ; *c1c([2H])c([2H])c(-c2c([2H])c([2H])c([2H])c([2H])c2-c2c([2H])c([2H])c(-c3c([2H])c([2H])c([2H])c([2H])c3[2H])c([2H])c2[2H])c([2H])c1[2H]]

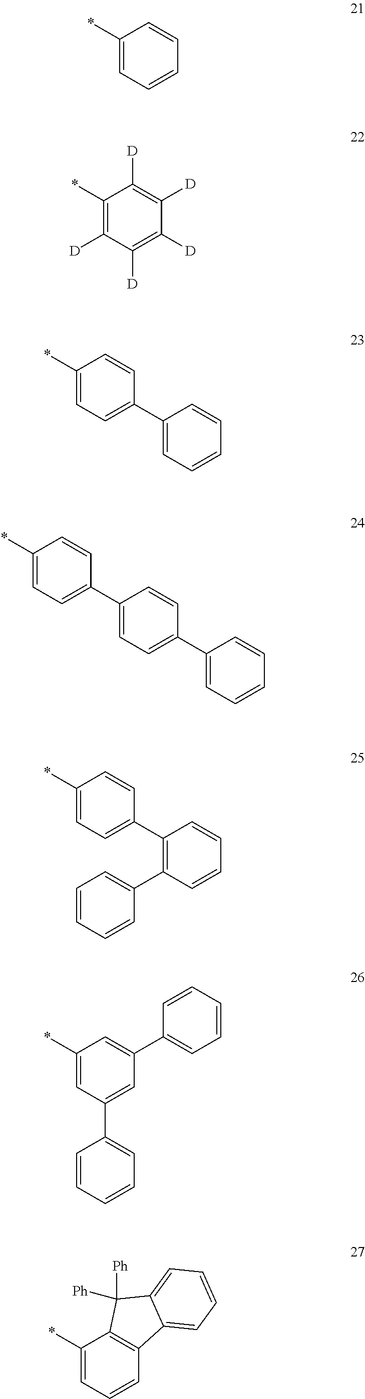

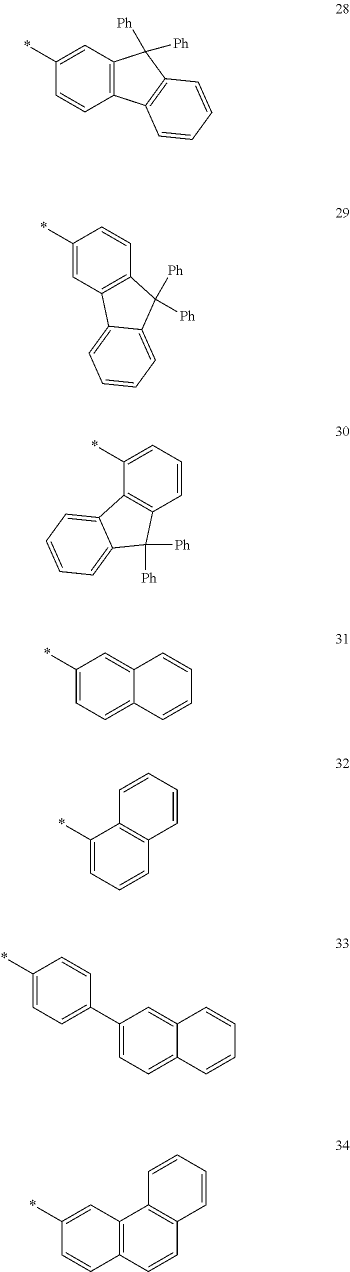

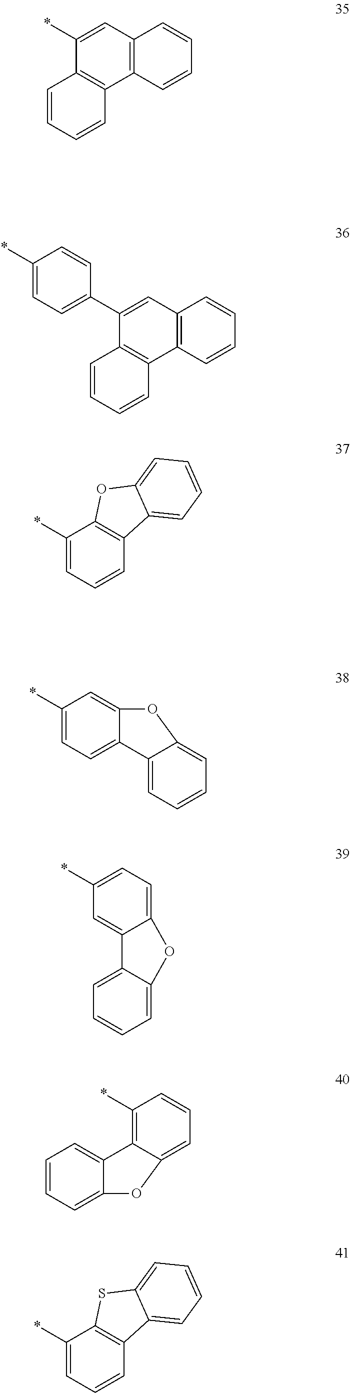

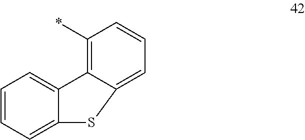 [SMILES: *c1cccc2sc3ccccc3c12]

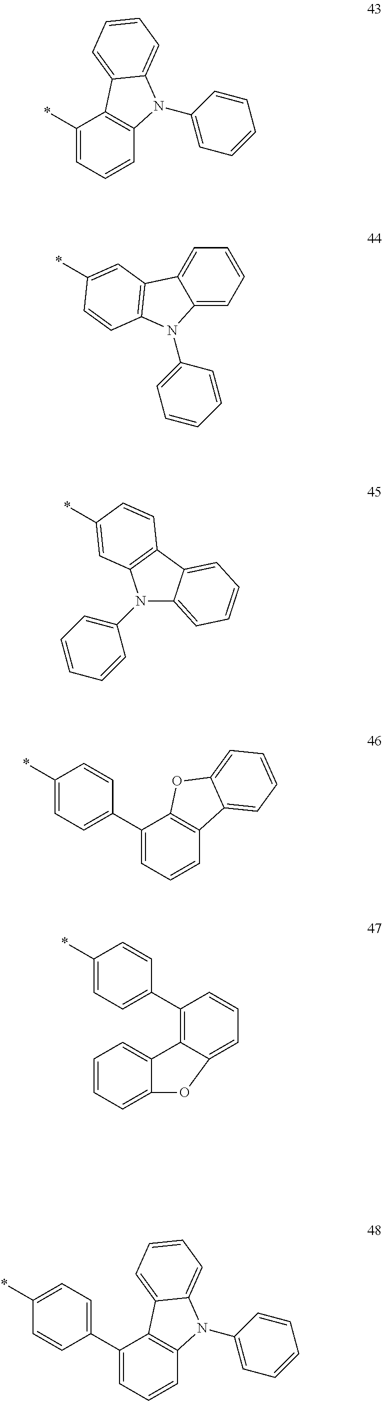 [SMILES: *c1cccc2c1c1ccccc1n2-c1ccccc1 ; *c1ccc2c(c1)c1ccccc1n2-c1ccccc1 ; *c1ccc2c3ccccc3n(-c3ccccc3)c2c1 ; *c1ccc(-c2cccc3c2oc2ccccc23)cc1 ; *c1ccc(-c2cccc3oc4ccccc4c23)cc1 ; *c1ccc(-c2cccc3c2c2ccccc2n3-c2ccccc2)cc1]

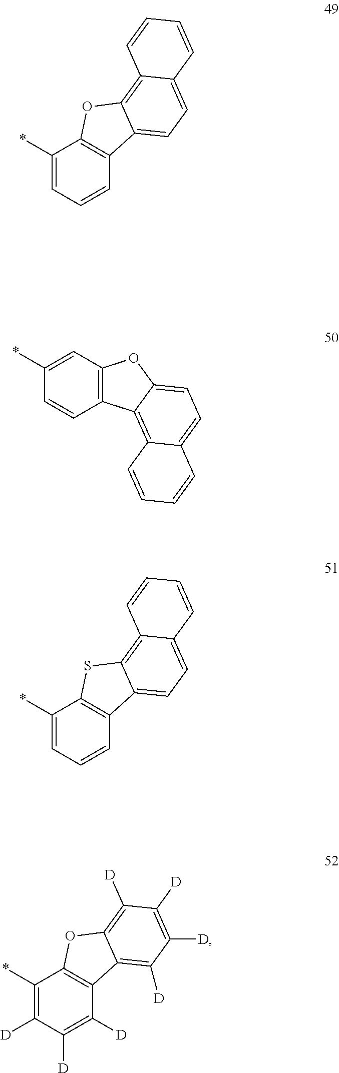

and [symbol]
is a position to which Formula 1 may be connected.

In one or more embodiments of the present disclosure, a display device includes a base layer, a circuit layer arranged on the base layer, and a display element layer arranged on the circuit layer and including a light-emitting element, and the light-emitting element includes a first electrode, a second electrode arranged on the first electrode, and at least one functional layer arranged between the first electrode and the second electrode and including the amine compound represented by Formula 1.

In one or more embodiments, the light-emitting element may further include a capping layer arranged on the second electrode, and the capping layer may have a refractive index of at least about 1.6 (e.g., or greater) for light in a wavelength range of about 550 nanometer (nm) to about 660 nm.

In one or more embodiments, the display device may further include an optical control layer arranged on the display element layer and including (e.g., containing) a quantum dot, the light-emitting element may be to emit first color light, and the optical control layer may include a first optical control part that includes a first quantum dot configured to convert (e.g., converting) the first color light into a second color light in a longer wavelength region than the first color light (e.g., the second color light having a wavelength region that is longer than a wavelength region of the first color light), a second optical control part that includes a second quantum dot configured to convert (e.g., converting) the first color light into a third color light in a longer wavelength region than the first color light and the second color light (e.g., the third color light having a wavelength region that is longer than a wavelength region of the first color light and a wavelength region of the second color light), and a third optical control part configured to transmit (e.g., transmitting) the first color light.

In one or more embodiments, the display device may further include a color filter layer arranged on the optical control layer, and the color filter layer may include a first filter transmitting the second color light, a second filter transmitting the third color light, and a third filter transmitting the first color light.

In one or more embodiments of the disclosure, an amine compound is represented by Formula 1.

### BRIEF DESCRIPTION OF THE DRAWINGS

The accompanying drawings are included to provide a further understanding of the preceding and other aspects, features, and advantages of certain embodiments of the present disclosure, and are incorporated in and constitute a part of this specification. The drawings illustrate example embodiments of the present disclosure and, together in conjunction with the description, serve to explain principles of the present disclosure. In the drawings:

FIG. 1 is a plan view illustrating a display device according to one or more embodiments;

FIG. 2 is a cross-sectional view of a display device according to one or more embodiments;

FIG. 3 is a cross-sectional view schematically illustrating a light-emitting element according to one or more embodiments;

FIG. 4 is a cross-sectional view schematically illustrating a light-emitting element according to one or more embodiments;

FIG. 5 is a cross-sectional view schematically illustrating a light-emitting element according to one or more embodiments;

FIG. 6 is a cross-sectional view schematically illustrating a light-emitting element according to one or more embodiments;

FIG. 7 is a cross-sectional view of a display device according to one or more embodiments;

FIG. 8 is a cross-sectional view of a display device according to one or more embodiments;

FIG. 9 is a cross-sectional view illustrating a display device according to one or more embodiments;

FIG. 10 is a cross-sectional view illustrating a display device according to one or more embodiments; and

FIG. 11 is a view illustrating an inside of a vehicle in which a display device according to one or more embodiments is arranged.

### DETAILED DESCRIPTION

Reference will now be made in more detail to one or more embodiments of the present disclosure that may be modified in one or more suitable manners and have many forms. Thus, specific embodiments are illustrated and will be exemplified in the drawings and described in more detail in the detailed description of present disclosure. It should be understood, however, that it is not intended to limit the disclosure to the particular forms disclosed, but rather, is intended to cover all modifications, equivalents, and alternatives falling within the spirit and scope of the disclosure.

When explaining each of drawings, like reference numbers are used for referring to like elements and duplicative descriptions thereof may not be provided. In the accompanying drawings, the dimensions of each structure may be exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the disclosure. As used herein, the singular forms, “a,” “an,” and “the” are intended to include the plural forms as well, unless the context clearly indicates otherwise.

In the present application, it will be understood that the terms “comprises,” “comprising,” “comprise,” “includes,” “including,” “include,” “have” “has,” “having,” and/or the like specify the presence of features, numbers, steps, operations, component, parts, and/or one or more (e.g., any suitable) combinations thereof disclosed in the specification, but do not exclude the possibility of presence or addition of one or more other features, numbers, steps, operations, component, parts, and/or one or more (e.g., any suitable) combinations thereof. Additionally, the terms “comprise(s)/comprising,” “include(s)/including,” “have/has/having” or similar terms include or support the terms “consisting of” and “consisting essentially of,” indicating the presence of stated features, integers, steps, operations, elements, and/or components, without or essentially without the presence of other features, integers, steps, operations, elements, components, and/or groups thereof. In this context, “consisting essentially of” indicates that any additional components will not materially affect the chemical, physical, optical or electrical properties of the semiconductor film. For example, throughout the disclosure, the expression “at least one of a, b or c” indicates only a, only b, only c, both (e.g., simultaneously) a and b, both (e.g., simultaneously) a and c, both (e.g., simultaneously) b and c, all of a, b, and c, or variations thereof.

In the present application, if (e.g., when) a layer, a film, a region, or a plate is referred to as being “connected to,” “on,” or “in an upper portion of” another layer, film, region, or plate, it may be not only “directly on” the layer, film, region, or plate, but intervening layers, films, regions, or plates may also be present. On the contrary to this, if (e.g., when) a layer, a film, a region, or a plate is referred to as being “below”, “in a lower portion of” another layer, film, region, or plate, it can be not only directly under the layer, film, region, or plate, but intervening layers, films, regions, or plates may also be present. In some embodiments, it will be understood that if (e.g., when) a part is referred to as being “on” another part, it can be arranged above the other part, or arranged under the other part as well.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

As used herein, the term “and/or” includes any and all combinations of one or more of the associated listed items. Expressions such as “at least one of,” “one of,” “selected from,” and “selected from among,” when preceding a list of elements, modify the entire list of elements and do not modify the individual elements of the list.

Unless otherwise defined, all chemical names, technical and scientific terms, and terms defined in common dictionaries should be interpreted as having meanings consistent with the context of the related art, and should not be interpreted in an ideal or overly formal sense. The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

Further, in this specification, the phrase “on a plane,” or “plan view,” indicates viewing a target portion from the top, and the phrase “on a cross-section” indicates viewing a cross-section formed by vertically cutting a target portion from the side.

### Definitions

In the specification, the term “substituted or unsubstituted” may refer to substituted or unsubstituted with at least one substituent selected from among the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In some embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but one or more embodiments of the disclosure is not limited thereto.

In the specification, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but one or more embodiments of the disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl aryl group, a styrenyl group, a styryl vinyl group, and/or the like, but one or more embodiments of the disclosure is not limited thereto.

In the present specification, an aryl group refers to any functional group or substituent, derived from an aromatic hydrocarbon ring. The aryl group may be a monocyclic aryl group or a polycyclic aryl group. The ring-forming carbon number of the aryl group may be 6 to 40, 6 to 30, 6 to 20, or 6 to 15. Examples of the aryl group may include a phenyl group, a naphthyl group, a fluorenyl group, an anthracenyl group, a phenanthrenyl group, a biphenylyl group, a terphenylyl group, a quarterphenylyl group, a quinquephenylyl group, a sexiphenylyl group, triphenylenyl group, a pyrenyl group, a benzo fluoranthenyl group, a chrysenyl group, and/or the like, but are not limited thereto.

In the specification, the fluorenyl group may be substituted, and two substituents may be bonded to each other to form a spiro structure. Examples of the substituted fluorenyl group are as follows. However, one or more embodiments of the disclosure is not limited thereto.

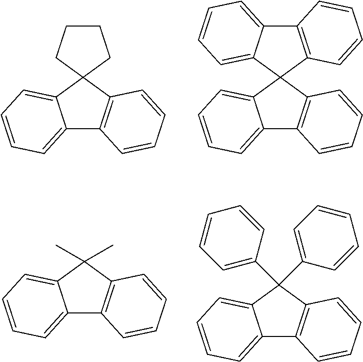

The heterocyclic group herein refers to any functional group or substituent derived from a ring containing at least one of B, O, N, P, Si, or Se as a heteroatom. The heterocyclic group includes an aliphatic heterocyclic group and an aromatic heterocyclic group. The aromatic heterocyclic group may be a heteroaryl group. The aliphatic heterocycle and the aromatic heterocycle may be monocyclic or polycyclic.

As used herein, the heteroaryl group may contain one or more among B, O, N, P, Si and S as hetero atoms. When the heteroaryl group contains two or more hetero atoms, the two or more hetero atoms may be the same as, or may be different from each other. The heteroaryl group may be a monocyclic heterocyclic group, or a polycyclic heterocyclic group. The heteroaryl group may have 2 to 40, 2 to 30, 2 to 20, or 2 to 10 ring-forming carbons. Examples of heteroaryl group may include a thienyl group, a furyl group, a pyrrolyl group, a imidazolyl group, a pyridyl group, a bipyridinyl group, a triazolyl group, a acridinyl group, a pyridazinyl group, a pyazinyl group, a quinolyl group, a quinazolinyl group, a quinoxalinyl group, a phenoxazinyl group, a phthalazinyl group, a pyridopyrimidinyl group, a pyridopyrazinyl group, a pyrazinopyrazinyl group, a isoquinolinyl group, a indolyl group, a carbazolyl group, N-arylcarbazolyl group, N-heteroarylcarbazolyl group, N-alkylcarbazolyl group, a benzoxazolyl group, a benzimidazolyl group, a benzothiazolyl group, a benzocarbazolyl group, a benzothiophenyl group, a dibenzothiophenyl group, a thienothiophenyl group, a benzofuranyl group, a phenanthrolinyl group, a thiazolyl group, a isoxazolyl group, a oxazolyl group, a oxadiazolyl group, a thiadiazolyl group, a phenothiazinyl group, a dibenzosilolyl group, a dibenzofuranyl group, and/or the like, but are not limited thereto.

In the specification, the preceding description of the aryl group may be applied to an arylene group except that the arylene group is a divalent group. The preceding description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but one or more embodiments of the disclosure is not limited thereto.

In the specification, the thio group may include an alkylthio group and an arylthio group. The thio group may refer to that a sulfur atom is bonded to the alkyl group or the aryl group as defined herein. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but one or more embodiments of the disclosure is not limited thereto.

In the specification, an oxy group may refer to that an oxygen atom is bonded to the alkyl group or the aryl group as defined herein. The oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but one or more embodiments of the disclosure is not limited thereto.

The boron group herein may refer to that a boron atom is bonded to the alkyl group or the aryl group as defined herein. The boron group includes an alkyl boron group and an aryl boron group. Examples of the boron group may include a dimethylboron group, a trimethylboron group, a t-butyldimethylboron group, a diphenylboron group, a phenylboron group, and/or the like, but one or more embodiments of the disclosure is not limited thereto.

In the specification, the number of carbon atoms in an amine group is not specifically limited, but may be 1 to 30. The amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, and/or the like, but one or more embodiments of the disclosure is not limited thereto.

In the specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described herein.

In the specification, the aryl group among an aryloxy group, an arylthio group, an arylsulfoxy group, an arylamino group, an arylboron group, an arylsilyl group, an arylamine group is the same as the examples of the aryl group described herein.

In the specification, a direct linkage may refer to a single bond.

In one or more embodiments, in the specification,

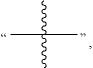

and “ [symbol] ” refer to a position to be connected.

Hereinafter, embodiments of the disclosure will be described with reference to the accompanying drawings.

### Display Apparatus

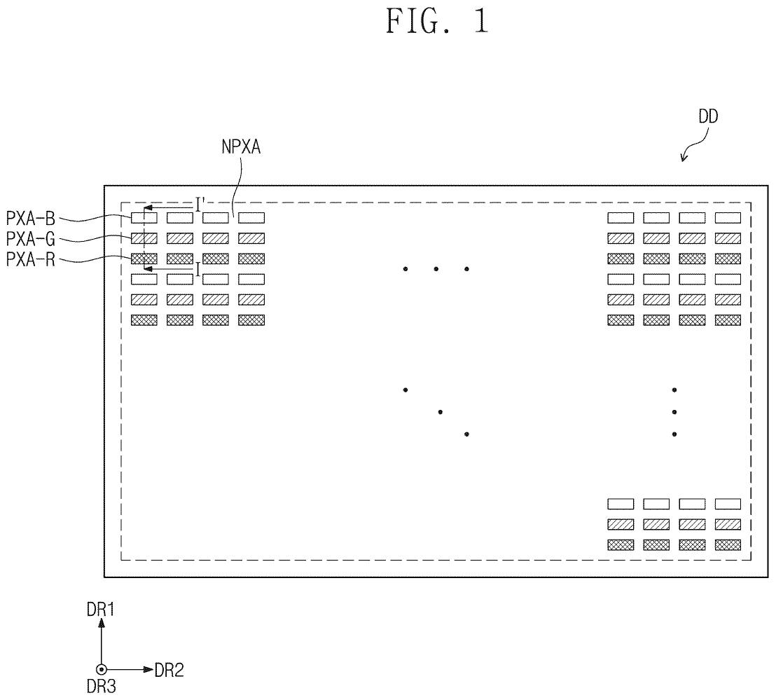

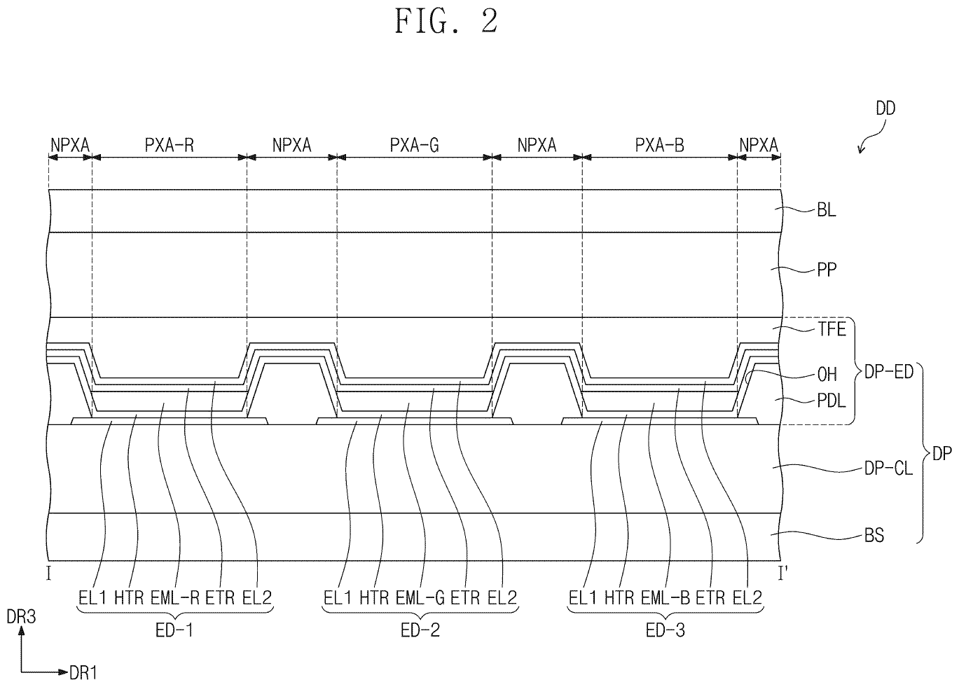

FIG. 1 is a plan view illustrating one or more embodiments of a display apparatus DD. FIG. 2 is a cross-sectional view of the display apparatus DD of the embodiment. FIG. 2 is a cross-sectional view illustrating a part taken along the line I-I′ of FIG. 1.

The display apparatus DD may include a display panel DP and an optical layer PP arranged on the display panel DP. The display panel DP includes light-emitting elements ED-1, ED-2, and ED-3. The display apparatus DD may include a plurality of light-emitting elements ED-1, ED-2, and ED-3. The optical layer PP may be arranged on the display panel DP to control reflected light in the display panel DP due to external light. The optical layer PP may include, for example, a polarization layer or a color filter layer. In one or more embodiments, unlike the configuration illustrated in the drawing, the optical layer PP may not be provided from the display apparatus DD of one or more embodiments.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, one or more embodiments of the disclosure is not limited thereto, and the base substrate BL may be an inorganic layer, an organic layer, or a composite material layer. In some embodiments, unlike the configuration illustrated, in one or more embodiments, the base substrate BL may not be provided.

The display apparatus DD according to one or more embodiments may further include a filling layer. The filling layer may be arranged between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light-emitting elements ED-1, ED-2, and ED-3 arranged between portions of the pixel defining film PDL, and an encapsulation layer TFE arranged on the light-emitting elements ED-1, ED-2, and ED-3.

The base layer BS may be a member which provides a base surface on which the display device layer DP-ED is arranged. The base layer BS may be a glass substrate, a metal substrate, a plastic substrate, and/or the like. However, one or more embodiments is not limited thereto, and the base layer BS may be an inorganic layer, an organic layer, or a composite material layer.

In one or more embodiments, the circuit layer DP-CL is arranged on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light-emitting elements ED-1, ED-2, and ED-3 of the display device layer DP-ED.

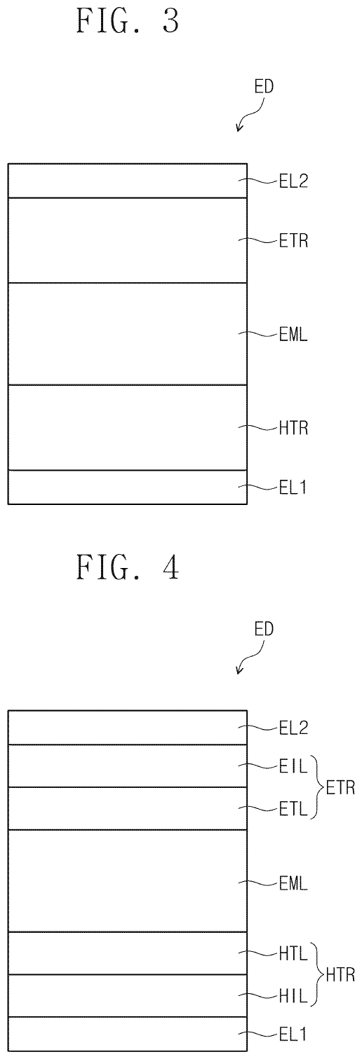

Each of the light-emitting elements ED-1, ED-2, and ED-3 may have a structure of each light-emitting element ED of embodiments according to FIGS. 3 to 6, which will be described in more detail later. Each of the light-emitting elements ED-1, ED-2, and ED-3 may include a first electrode EL1, a hole transport region HTR, light-emitting layers EML-R, EML-G, and EML-B, an electron transport region ETR, and a second electrode EL2.

FIG. 2 illustrates one or more embodiments in which the light-emitting layers EML-R, EML-G, and EML-B of the light-emitting elements ED-1, ED-2, and ED-3 are arranged in openings OH defined in the pixel defining film PDL, and the hole transport region HTR, the electron transport region ETR, and the second electrode EL2 are provided as a common layer in the entire light-emitting elements ED-1, ED-2, and ED-3. However, one or more embodiments of the disclosure is not limited thereto, and unlike the configuration illustrated in FIG. 2, the hole transport region HTR and the electron transport region ETR in one or more embodiments may be provided by being patterned inside the openings OH defined in the pixel defining film PDL. For example, the hole transport region HTR, the light-emitting layers EML-R, EML-G, and EML-B, and the electron transport region ETR of the light-emitting elements ED-1, ED-2, and ED-3 in one or more embodiments may be provided by being patterned in an inkjet printing method.

The encapsulation layer TFE may cover the light-emitting elements ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to one or more embodiments may include at least one inorganic film (hereinafter, an encapsulation-inorganic film). The encapsulation layer TFE according to one or more embodiments may also include at least one organic film (hereinafter, an encapsulation-organic film) and at least one encapsulation-inorganic film.

The encapsulation-inorganic film protects the display device layer DP-ED from moisture and/or oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but one or more embodiments of the disclosure is not particularly limited thereto. The encapsulation-organic film may include an acrylic-based compound, an epoxy-based compound, and/or the like. The encapsulation-organic film may include a photopolymerizable organic material, but one or more embodiments of the disclosure is not particularly limited thereto.

The encapsulation layer TFE may be arranged on the second electrode EL2 and may be arranged filling the opening OH.

Referring to FIGS. 1 and 2, the display apparatus DD may include a non-light emitting region NPXA and light emitting regions PXA-R, PXA-G, and PXA-B. The light emitting regions PXA-R, PXA-G, and PXA-B may be regions in which light generated by the respective light-emitting elements ED-1, ED-2, and ED-3 is emitted. The light emitting regions PXA-R, PXA-G, and PXA-B may be spaced and/or apart (e.g., spaced apart or separated) from each other on a plane.

Each of the light emitting regions PXA-R, PXA-G, and PXA-B may be a region divided by the pixel defining film PDL. The non-light emitting areas NPXA may be areas between the adjacent light emitting areas PXA-R, PXA-G, and PXA-B, which correspond to the pixel defining film PDL. In one or more embodiments, in the specification, the light emitting regions PXA-R, PXA-G, and PXA-B may respectively correspond to pixels. The pixel defining film PDL may divide the light-emitting elements ED-1, ED-2, and ED-3. The light-emitting layers EML-R, EML-G, and EML-B of the light-emitting elements ED-1, ED-2, and ED-3 may be arranged in openings OH defined in the pixel defining film PDL and separated from each other.

The light emitting regions PXA-R, PXA-G, and PXA-B may be divided into a plurality of groups according to the color of light generated from the light-emitting elements ED-1, ED-2, and ED-3. In the display apparatus DD of one or more embodiments illustrated in FIGS. 1 and 2, three light emitting regions PXA-R, PXA-G, and PXA-B, which emit red light, green light, and blue light, respectively, are exemplarily illustrated. For example, the display device DD of one or more embodiments may include the red light emitting region PXA-R, the green light emitting region PXA-G, and the blue light emitting region PXA-B that are separated from each other.

In the display apparatus DD according to one or more embodiments, the plurality of light-emitting elements ED-1, ED-2 and ED-3 may be to emit light beams having wavelengths different from each other. For example, in one or more embodiments, the display apparatus DD may include a first light-emitting element ED-1 that emits red light, a second light-emitting element ED-2 that emits green light, and a third light-emitting element ED-3 that emits blue light. For example, the red light emitting region PXA-R, the green light emitting region PXA-G, and the blue light emitting region PXA-B of the display apparatus DD may correspond to the first light-emitting element ED-1, the second light-emitting element ED-2, and the third light-emitting element ED-3, respectively.

However, one or more embodiments of the disclosure is not limited thereto, and the first to third light-emitting elements ED-1, ED-2, and ED-3 may be to emit light beams in substantially the same wavelength range or at least one light-emitting element may be to emit a light beam in a wavelength range different from the others. For example, the first to third light-emitting elements ED-1, ED-2, and ED-3 may all emit blue light.

The light emitting regions PXA-R, PXA-G, and PXA-B in the display apparatus DD according to one or more embodiments may be arranged in a stripe form. Referring to FIG. 1, the plurality of red light emitting regions PXA-R, the plurality of green light emitting regions PXA-G, and the plurality of blue light emitting regions PXA-B each may be arranged along a second directional axis DR2. In some embodiments, the red light emitting region PXA-R, the green light emitting region PXA-G, and the blue light emitting region PXA-B may be alternately arranged in this order along a first directional axis DR1.

FIGS. 1 and 2 illustrate that all the light emitting regions PXA-R, PXA-G, and PXA-B have similar area, but one or more embodiments of the disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas if (e.g., when) viewed on a plane defined by the first directional axis DR1 and the second directional axis DR2.

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display apparatus DD. For example, the arrangement form of the light emitting regions PXA-R, PXA-G, and PXA-B may be a pentile (PENTILE™) arrangement form or a diamond (Diamond Pixel™) arrangement form, (PENTILE™ and Diamond Pixel™ are registered trademarks owned by Samsung Display Co., Ltd.).

In some embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but one or more embodiments of the disclosure is not limited thereto.

Hereinafter, FIG. 3 to FIG. 6 are cross-sectional views schematically showing light-emitting elements according to one or more embodiments. The light-emitting element ED according to one or more embodiments may include a first electrode EL1, a second electrode EL2 oppositely arranged to the first electrode EL1, and at least one functional layer arranged between the first electrode EL1 and the second electrode EL2. The light-emitting element ED of one or more embodiments may include an amine compound of one or more embodiments, which will be explained in more detail later, in the at least one functional layer.

The light-emitting element ED may include a hole transport region HTR, a light-emitting layer EML, an electron transport region ETR, and/or the like, stacked in order, as the at least one functional layer. Referring to FIG. 3, the light-emitting element ED of one or more embodiments may include a first electrode EL1, a hole transport region HTR, a light-emitting layer EML, an electron transport region ETR, and a second electrode EL2, stacked in order.

The light-emitting element ED according to one or more embodiments contains, in a light-emitting layer EML arranged between the first electrode EL1 and the second electrode EL2, an amine compound according to one or more embodiments to be described in more detail later. However, one or more embodiments of the disclosure is not limited thereto, and the light-emitting element ED according to one or more embodiments contains the amine compound according to one or more embodiments to be described in more detail later, in addition to in the light-emitting layer EML, in a hole transport region HTR or an electron transport region ETR, which are a plurality of functional layers arranged between the first electrode EL1 and the second electrode EL2. In one or more embodiments, the amine compound according to one or more embodiments to be described in more detail later may be contained in a capping layer CPL arranged on the second electrode EL2.

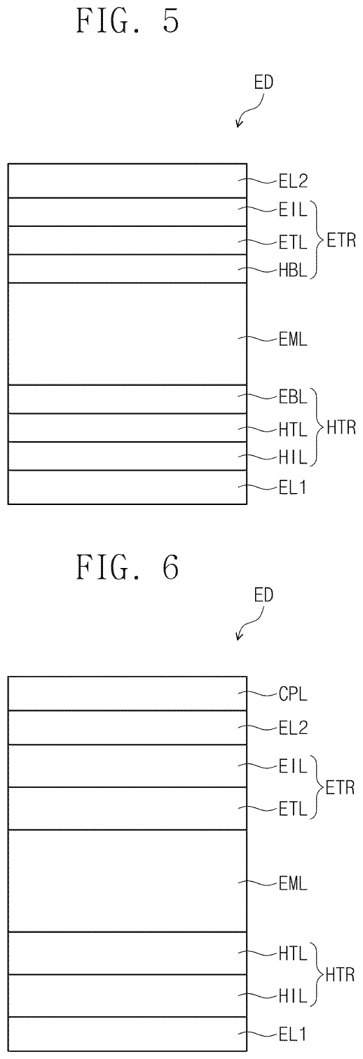

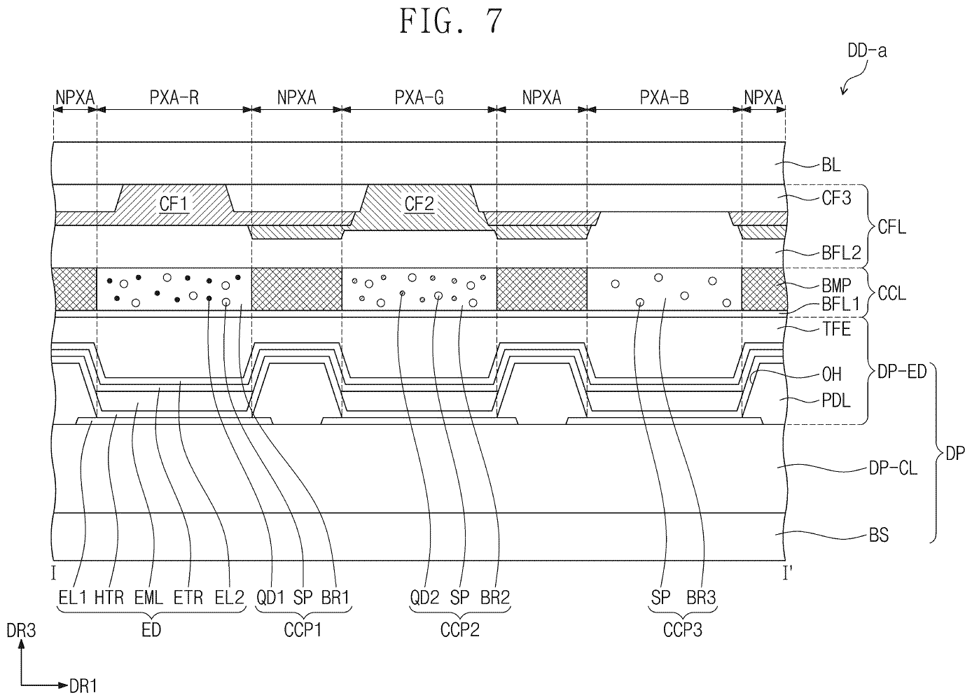

Compared with FIG. 3, FIG. 4 illustrates a cross-sectional view of a light-emitting element ED of one or more embodiments, in which a hole transport region HTR includes a hole injection layer HIL and a hole transport layer HTL, and an electron transport region ETR includes an electron injection layer EIL and an electron transport layer ETL. In some embodiments, compared with FIG. 3, FIG. 5 illustrates a cross-sectional view of a light-emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 illustrates a cross-sectional view of a light-emitting element ED of one or more embodiments including a capping layer CPL arranged on a second electrode EL2.

The light-emitting element ED according to one or more embodiments may contain the amine compound according to one or more embodiments, to be described in more detail later, in the hole transport region HTR. The light-emitting element ED according to one or more embodiments contain the amine compound according to one or more embodiments in at least one among a hole injection layer HIL, a hole transport layer HTL, and an electron blocking layer EBL of the hole transport region HTR. For example, in the light-emitting element ED, the hole transport layer HTL may contain the amine compound according to one or more embodiments.

The first electrode EL1 has conductivity (e.g., is a conductor). The first electrode EL1 may be formed of a metal material, a metal alloy, or a conductive compound. The first electrode EL1 may be an anode or a cathode. However, one or more embodiments of the disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these, a mixture of two or more selected from among these, or an oxide thereof.

If the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In one or more embodiments, the first electrode EL1 may have a multilayer structure including a reflective film or a transflective film formed of the herein-described materials, and a transparent conductive film formed of ITO, IZO, ZnO, ITZO, and/or the like. For example, the first electrode EL1 may have a three-layer structure of ITO/Ag/ITO, but one or more embodiments of the disclosure is not limited thereto. In some embodiments, one or more embodiments of the disclosure is not limited thereto, and the first electrode EL1 may include the herein-described metal materials, combinations of at least two metal materials of the herein-described metal materials, oxides of the herein-described metal materials, and/or the like. The thickness of the first electrode EL1 may be from about 700 angstrom (Å) to about 10,000 Å. For example, the thickness of the first electrode EL1 may be from about 1,000 Å to about 3,000 Å.

The hole transport region HTR is provided on the first electrode EL1. The hole transport region HTR may have a single layer formed of a single material, a single layer formed of a plurality of different materials, or a multilayer structure including a plurality of layers formed of a plurality of different materials.

The hole transport region HTR may include at least one of a hole injection layer HIL, a hole transport layer HTL, or an electron blocking layer EBL. In some embodiments, though not shown, the hole transport region HTR may include multiple hole transport layers stacked.

For example, the hole transport region HTR may have a single layer structure of the hole injection layer HIL or the hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1, but one or more embodiments of the disclosure is not limited thereto.

The thickness of the hole transport region HTR may be, for example, about 50 Å to about 15,000 Å. The hole transport region HTR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The light-emitting element ED according to one or more embodiments may contain the amine compound according to one or more embodiments in the hole transport region HTR. In the light-emitting element ED according to one or more embodiments, the hole transport region HTR includes an hole injection layer HIL, and a hole transport layer HTL, and hole transport layer HTL may contain the amine compound according to one or more embodiments. The amine compound according to one or more embodiments may be contained in a layer, among layers included in the hole transport region HTR, adjacent to the light-emitting layer EML.

### Amine Compound

The amine compound according to one or more embodiments includes an amine group, and a first substituent, a second substituent, and a third substituent, which are linked to the amine group. For example, the amine compound according to one or more embodiments includes an amine group, that is a core nitrogen atom, and includes a structure in which the first substituent, the second substituent, and the third substituent are bonded to the core nitrogen atom.

The first substituent includes a quarterphenyl moiety in which substituted or unsubstituted four benzene rings are connected to each other in a specific linking structure. For example, in the amine compound according to one or more embodiments, the quarterphenyl moiety may refer to 1,1′:2′,1″:4″,1′″-quarterphenyl moiety represented by Formula S. In one or more embodiments, the quarterphenyl moiety may be directly bonded to a nitrogen atom of amine group. In the amine compound according to one or more embodiments, the quarterphenyl moiety may be directly bonded to the nitrogen atom at a “a1” position as represented in Formula S. For example, the quarterphenyl may be directly bonded to the nitrogen atom of amine group via a 4th carbon. In one or more embodiments, for convenience of explanation, in Formula S, a substituent substituted for the quarterphenyl moiety is not included in a drawing.

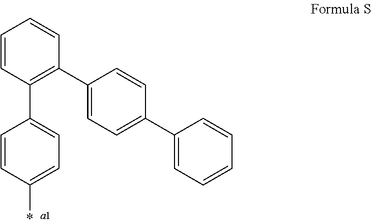 [SMILES: *c1ccc(-c2ccccc2-c2ccc(-c3ccccc3)cc2)cc1]

Because the amine compound according to one or more embodiments includes the first substituent, a charge balance is adjusted by a steric effect, and thus the amine compound may exhibit excellent or suitable charge transporting properties and high thermal stability. For example, the amine compound according to one or more embodiments has a structure in which 1,1′:2′,1″:4″,1′″-quarterphenyl moiety is bonded to the nitrogen atom of amine group at a specific position, thereby having excellent or suitable charge transporting properties and material stability, which may contribute to improvements in high efficiency and long lifespan of the light-emitting element.

In the amine compound according to one or more embodiments, the second and third substituents may each be directly bonded to the nitrogen atom of the amine group. For example, the second and third substituents may each be directly bonded to the nitrogen atom without an additional linker. In one or more embodiments, the second and third substituents may each independently be a substituted or unsubstituted aryl group having 6 to 30 ring-forming carbons, or a substituted or unsubstituted heteroaryl group having 2 to 30 ring-forming carbons. In one or more embodiments, as used herein, the second substituent may refer to a substituent represented by Ar¹ in Formula 1 to be described in more detail later, and the third substituent may refer to a substituent represented by Ar² in Formula 1 to be described in more detail later.

In one or more embodiments, at least one among the second and third substituents may be a substituted or unsubstituted naphthylphenyl group, a substituted or unsubstituted dibenzofuranyl group, a substituted or unsubstituted dibenzothiophenyl group, or a substituted or unsubstituted carbazolyl group. In some embodiments, if (e.g., when) the second and third substituents are each a substituted or unsubstituted naphthylphenyl group, a substituted or unsubstituted dibenzofuranyl group, a substituted or unsubstituted dibenzothiophenyl group, or a substituted or unsubstituted carbazolyl group, the substituent may be bonded to an adjacent group to form a ring.

As used herein, a dibenzofuranyl group may be substituted, and adjacent substituents may be bonded to each other to form a fused ring structure. For example, the dibenzofuranyl group may be substituted and two adjacent substituents may be bonded to each other to form a fused ring structure c. However, one or more embodiments of the disclosure is not limited thereto.

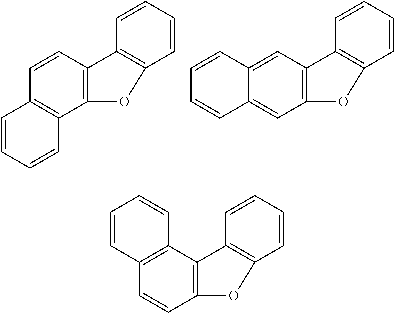

As used herein, a dibenzothiophenyl group may be substituted, and adjacent substituents may be bonded to each other to form a fused ring structure. For example, the dibenzothiophenyl group is substituted and two adjacent substituents may be bonded to each other to form a fused ring structure as a structure. However, one or more embodiments of the disclosure is not limited thereto.

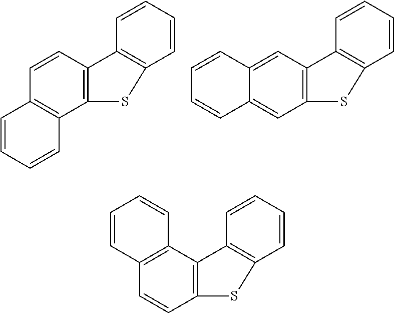

As used herein, a carbazolyl group may be substituted, and adjacent substituents may be bonded to each other to form a fused ring structure. An example of a case where the cabazolyl group is substituted is as follows. However, one or more embodiments of the disclosure is not limited thereto.

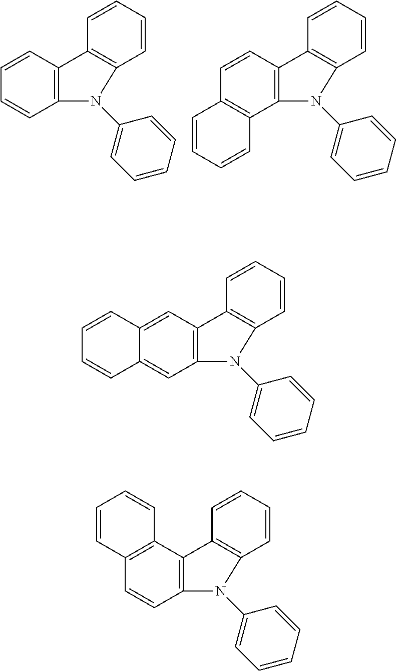

Hole transport materials used in the light-emitting element are desired or required to have characteristics of thermal and chemical stability of a molecule and molecular orientation. When the thermal and chemical stability of a molecule decreases, the materials tend to be decomposed easily during deposition, and if (e.g., when) the molecular orientation characteristics are not unique, there is a limitation in that light emission efficiency is reduced because the intermolecular interactions become weaker, making it difficult for carriers to move.

In the amine compound according to the disclosure, at least one among (e.g., selected from among) the first and third substituents includes a substituted or unsubstituted naphthylphenyl group, a substituted or unsubstituted dibenzofuranyl group, a substituted or unsubstituted dibenzothiophenyl group, or a substituted or unsubstituted carbazolyl group, thereby exhibiting high molecular stability and improved molecular orientation characteristics. Therefore, materials that are not easily decomposed during the deposition process and maintain high purity may be used in the light-emitting element. In some embodiments, the amine compound according to one or more embodiments may exhibit relatively uniformly (e.g., substantially uniformly)-aligned characteristics of molecular orientation by introducing the substituent, which improves characteristics of charge transporting, and thus may contribute to implementation of the light-emitting element having high luminous efficiency.

The amine compound according to one or more embodiments may be a monoamine compound including one amine group. The amine compound according to one or more embodiments may be a monoamine compound having only one amine group that exists without forming a ring within a molecular structure thereof.

In one or more embodiments, the amine compound may be represented by Formula 1.

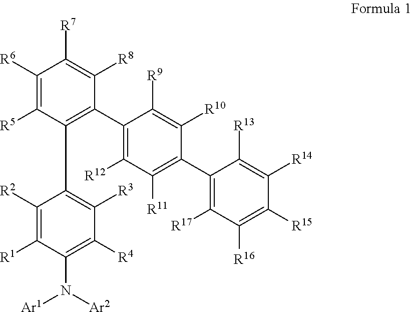

In Formula 1, R¹ to R¹⁷ may each independently be a hydrogen atom, a deuterium atom, a substituted or unsubstituted oxy group, a substituted or unsubstituted thio group, a substituted or unsubstituted amine group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 25 ring-forming carbons. In one or more embodiments, each of R¹ to R¹⁷ may combine with an adjacent group to each other to form a ring. For example, R¹ to R¹⁷ may each independently be a hydrogen atom, a deuterium atom, a substituted or unsubstituted phenyl group.

In one or more embodiments, at least one pair among pairs composed of two selected from among R¹ to R¹⁷ combines each other to form a ring. For example, at least one pair among R¹ and R², R³ and R⁴, R² and R⁵, R⁵ and R⁶, R⁶ and R⁷, R⁷ and R⁸, R⁸ and R⁹, R⁹ and R¹⁰, R¹¹ and R¹², R¹⁰ and R¹³, R¹³ and R¹⁴, R¹⁴ and R¹⁵, R¹⁵ and R¹⁶, R¹⁶ and R¹⁷, and R¹¹ and R¹⁷ combines each other to form an aromatic hydrocarbon ring or an aromatic heterocycle.

In one or more embodiments, at least one pair among R¹ and R², R³ and R⁴, R⁵ and R⁶, R⁶ and R⁷, R⁷ and R⁸, R⁹ and R¹⁰, R¹¹ and R¹², R¹³ and R¹⁴, R¹⁴ and R¹⁵, R¹⁵ and R¹⁶, and R¹⁶ and R¹⁷ may combine each other to form an aromatic hydrocarbon ring. For example, R¹⁵ and R¹⁶ may combine each other to form an aromatic hydrocarbon ring such as a naphthalene ring.

In one or more embodiments, at least one pair among R² and R⁵, R⁸ and R⁹, R¹⁰ and R¹³, and R¹¹ and R¹⁷ may combine each other to form an aromatic hydrocarbon ring or an aromatic heterocycle. For example, R¹⁰ and R¹³ combine each other to form an aromatic hydrocarbon ring such as a fluorene ring, or an aromatic heterocycle such as a dibenzofuran ring and a dibenzothiophene ring.

In Formula 1, a case where R⁵ is a substituted or unsubstituted carbazolyl group is excluded. For example, in Formula 1, a case where R⁵ is represented by Formula 1-a may be excluded.

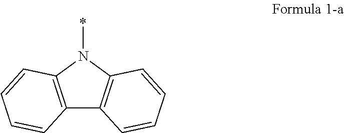 [SMILES: *n1c2ccccc2c2ccccc21]

In one or more embodiments, cases where R¹ to R¹⁷ are substituted or unsubstituted heteroaryl groups having total ring-forming carbons of 26 or more may be excluded. For example, a case where at least one among R¹ to R¹⁷ is a substituted or unsubstituted heteroaryl group, and the total number of ring-forming carbons of whole rings configuring the heteroaryl group is 26 or more may be excluded. When the amine compound according to one or more embodiments includes a heteroaryl group having 26 or more ring-forming carbons as a substituent linked to a quaterphenyl moiety, a deposition temperature increases, and thus, the compound may decompose during deposition, which may reduce a lifespan. According to the present disclosure, because the cases where R¹ to R¹⁷ substituted for the quarterphenyl moiety are heteroaryl groups having 26 or more ring-forming carbons are excluded, decomposition of the compound may be inhibited or reduced. Therefore, if (e.g., when) the amine compound according to one or more embodiments is applied to the light-emitting element ED, characteristics of high efficiency and long lifespan may be achieved.

In Formula 1, Ar¹ and Ar² may each independently be a substituted or unsubstituted aryl group having 6 to 30 ring-forming carbons, or a substituted or unsubstituted heteroaryl group having 2 to 30 ring-forming carbons.

In Formula 1, at least one among Ar¹ and Ar² includes a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group. In some embodiments, if (e.g., when) each of Ar¹ and Ar² includes a substituted naphthyl group, a substituted naphthylphenyl group, a substituted dibenzofuranyl group, a substituted dibenzothiophenyl group, or a substituted carbazolyl group, the substituent may combine an adjacent group to form a ring. In one or more embodiments, as used herein, “Substituent a includes Substituent b” refers to a case where the Substituent a is the Substituent b or a case where the Substituent a includes the Substituent b as a substituent. For example, at least one among Ar¹ and Ar² may be a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group, or at least one among Ar¹ and Ar² may be an aryl group or a heteroaryl group, substituted with a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group.

In one or more embodiments, one among Ar¹ and Ar² may be a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group, and the other may be a substituted or unsubstituted phenyl group, a substituted or unsubstituted biphenylyl group, a substituted or unsubstituted naphthylphenyl group, or a substituted or unsubstituted phenanthrenyl group.

In one or more embodiments, Ar¹ and Ar² may each independently be a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group.

In Formula 1, a case where at least one among Ar¹ and Ar² is a 4-phenyl-dibenzofuran-1-yl group is excluded. For example, a case where at least one among Ar¹ and Ar² is represented by Formula 3-a may be excluded. When the 4-phenyl-dibenzofuran-1-yl group represented by Formula 3-a is linked to a core nitrogen atom of the amine group, the dibenzofuran ring has relatively small resonance stabilization, and a first carbon and a fourth carbon of the dibenzofuran ring are bonded to the nitrogen atom of amine group and the phenyl group, respectively, which causes a decrease in thermal stability of the whole molecule due to distortion of the molecule. In the present disclosure, because the case where at least one among Ar¹ and Ar² is the 4-phenyl-dibenzofuran-1-yl group is excluded, thermal stability of materials may be improved, and thus characteristics of high efficiency and long lifespan may be achieved if (e.g., when) the amine compound according to one or more embodiments is applied to the light-emitting element ED.

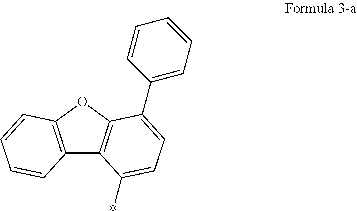 [SMILES: *c1ccc(-c2ccccc2)c2oc3ccccc3c12]

In Formula 1, cases where each of Ar¹ and Ar² includes a heteroaryl group substituted with a heteroaryl group is excluded. For example, cases where each of Ar¹ and Ar² includes a dibenzofuranyl group substituted with a heteroaryl group such as a dibenzofuranyl group, and a dibenzothiophenyl group may be excluded. Two consecutively bonded heteroaryl groups may be a factor that reduces the stability of the whole molecule due to steric reasons. In the disclosure, because the cases where each of Ar¹ and Ar² includes a heteroaryl group substituted with a heteroaryl group is excluded, the stability of molecule may be improved, and thus characteristics of high efficiency and long lifespan may be achieved if (e.g., when) the amine compound according to one or more embodiments is applied to the light-emitting element. In one or more embodiments, as used herein, “Substituent a includes Substituent b” refers that the Substituent a is the Substituent b, or that the Substituent a includes the Substituent b as a substituent.

In one or more embodiments, cases where each of Ar¹ and Ar² is a heteroaryl group represented by Formula 1-c may be excluded.

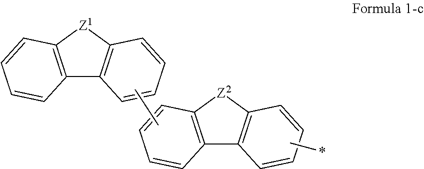

In Formula 1-c, Z¹ and Z² may each independently be O or S.

In the amine compound represented by Formula 1, a case where at least one among a chlorine atom and acenaphthene is included (e.g., contained) is excluded.

In one or more embodiments, a case where the amine compound represented by Formula 1 includes a halogen atom such as a chlorine atom may be excluded. In a compound including a chlorine atom in a molecule structure, because the reactivity is high by the chlorine atom, stability of the compound decreases, and thus element lifespan may be reduced if (e.g., when) the compound is applied to the light-emitting element. According to the disclosure, because the case where the amine compound according to one or more embodiments includes a chlorine atom is excluded, chemical stability improves, which may exhibit effect of the improved element lifespan of the light-emitting element ED.

In one or more embodiments, a case where the amine compound represented by Formula 1 includes acenaphthene is excluded. For example, the amine compound represented by Formula 1 may not include (e.g., may exclude) acenaphthene represented by Formula 1-d in a molecule. The acenaphthene having a fused structure of a cycloalkyl ring on an aryl ring is thermally and chemically unstable due to a cycloalkyl skeleton containing two sp³ carbons, which may cause a decrease in the element lifespan. According to the disclosure, because the case where the amine compound represented by Formula 1 includes acenaphthene is excluded, the thermal and chemical stability may be improved, and thus characteristics of the improved element lifespan may be exhibited.

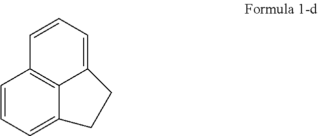 [SMILES: c1cc2c3c(cccc3c1)CC2]

In one or more embodiments, in Formula 1, at least one among Ar¹ and Ar² may be represented by any one among Formula 2-1 to Formula 2-3.

In one or more embodiments, in Formula 1, one among Ar¹ and Ar² may be represented by any one among Formula 2-1 to Formula 2-3, and the other among Ar¹ and Ar² may be a substituted or unsubstituted phenyl group, a substituted or unsubstituted biphenylyl group, a substituted or unsubstituted terphenylyl group, a substituted or unsubstituted quarterphenylyl group, a substituted or unsubstituted naphthyl group, a substituted or unsubstituted naphthylphenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a substituted or unsubstituted phenanthrenyl group.

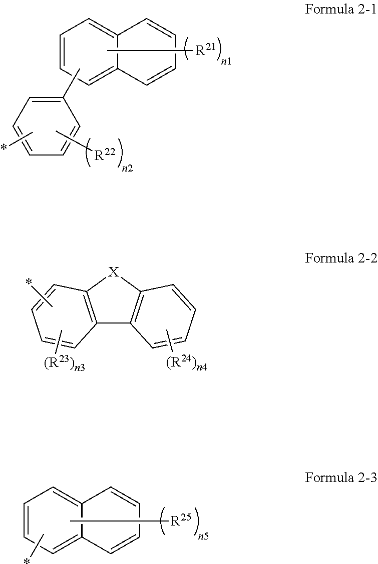

In Formula 2-2, X may be O, S, or NR²⁶.

In Formula 2-1 to Formula 2-3, R²¹, R²², R²⁵, and R²⁶ may each independently be a hydrogen atom, a deuterium atom, a halogen atom, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons. In one or more embodiments, R²¹, R²², R²⁵, and R²⁶ may be each bonded to an adjacent group to form a ring. For example, R²¹, R²², and R²⁵ may each independently be a hydrogen atom, a deuterium atom, and R²⁶ may be a substituted or unsubstituted phenyl group.

In Formula 2-2, R²³ and R²⁴ may each independently be a hydrogen atom, a deuterium atom, a halogen atom, a substituted or unsubstituted alkyl group having 1 to 20 carbons, a substituted or unsubstituted alkenyl group having 2 to 20 carbons, or a substituted or unsubstituted aryl group having 6 to 30 ring-forming carbons. In one or more embodiments, R²³ and R²⁴ may be each bonded to an adjacent group to form a ring. For example, R²³ and R²⁴ may each independently be a hydrogen atom, or a deuterium atom. In some embodiments, in Formula 2-2, R²³ may be provided in plural, and the plurality of R²³, which are adjacent, may be bonded to each other to form a ring. When, R²³ is bonded to adjacent group to form a ring, R²³ may be bonded to the adjacent group to each other to form an aromatic hydrocarbon ring or an aromatic heterocycle. In some embodiments, in Formula 2-2, R²⁴ may be provided in plural, and the plurality of R²⁴, which are adjacent, are bonded to each other to form a ring. When, R²⁴ is bonded to the adjacent group to form a ring, R²⁴ may be bonded to the adjacent group to each other to form an aromatic hydrocarbon ring or an aromatic heterocycle.

In Formula 2-1 and Formula 2-3, n1 and n5 may each independently be an integer of 0 to 7. If n1 and n5 are each 0, the amine compound according to one or more embodiments may be respectively unsubstituted with of R²¹ and R²⁵. Cases where n1 and n5 are each 7 and R²¹ and R²⁵ are all hydrogen atoms in Formula 2-1 and Formula 2-3, may be the same as cases where n1 and n5 are each 0 in Formula 2-1 and Formula 2-3. If each of n1 and n5 is an integer of 2 or more, R²¹ and R²⁵, provided in plural, may be all the same or at least one among the plurality of R²¹ and R²⁵ may be different.

n2 and n4 may each independently be an integer of 0 to 4 in Formula 2-1 and Formula 2-2. If n2 and n4 are each 0, the amine compound according to one or more embodiments may be unsubstituted with each of R²² and R²⁴. Cases where each of n2 and n4 is a hydrogen atom may be the same as cases where each of n2 and n4 is 0. If n2 and n4 are each an integer of 2 or greater, each of R²² and R²⁴ provided in the plurality may be the same or at least one among the plurality of R²² and R²⁴ may be different.

In Formula 2-2, n3 may be an integer of 0 to 3. If n3 is 0, the amine compound according to one or more embodiments may be unsubstituted with R²³. A case where n3 is 3 and all R²³ are hydrogen atoms in Formula 2-2 may be the same as the case where n3 is 0 in Formula 2-2. If n3 is an integer of 2 or more, all R²³ provided in plural may be the same, or at least one among the plurality of R²³ may be different.

In Formula 2-1 to Formula 2-3, [symbol] is a position to which Formula 1 is connected.

In one or more embodiments, the amine compound represented by Formula 1 may be represented by Formula 3.

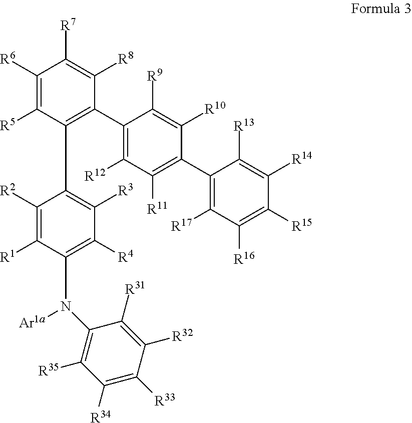

Formula 3 represents a case where types (kinds) of Ar¹ and Ar² in Formula 1 are specified.

In Formula 3, R³¹ to R³⁵ may each independently be a hydrogen atom, a deuterium atom, a halogen atom, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons. In one or more embodiments, R³¹ to R³⁵ may be each bonded to an adjacent group to form a ring.

In one or more embodiments, R³¹ to R³⁵ may each independently be a hydrogen atom, a deuterium atom, a substituted or unsubstituted phenyl group, a substituted or unsubstituted naphthyl group, or a substituted or unsubstituted phenanthrenyl group, or at least one pair among pairs composed of two selected from among R³¹ to R³⁵ may be bonded to an adjacent group to form a ring. For example, R³¹ and R³², and R³³ and R³⁴ may each be bonded to each other to form an aromatic hydrocarbon ring. In one or more embodiments, R³² and R³³, and R³⁴ and R³⁵ may be each bonded to each other to form an aromatic hydrocarbon ring.

In Formula 3, a case where at least one among R³¹ to R³⁵ is a heteroaryl group substituted with a heteroaryl group may be excluded. For example, a case where at least one among R³¹ to R³⁵ is a dibenzofuranyl group substituted with a dibenzofuranyl group or a dibenzothiophenyl group may be excluded.

In Formula 3, Ar^{1a} may be a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group. In some embodiments, if (e.g., when) Ar^{1a} is a substituted naphthyl group, a substituted naphthylphenyl group, a substituted dibenzofuranyl group, a substituted dibenzothiophenyl group, or a substituted carbazolyl group, the substituent may be bonded to an adjacent group to form a ring.

In Formula 3, a case where Ar^{1a} includes a heteroaryl group substituted with a heteroaryl group may be excluded. For example, a case where Ar^{1a} includes a dibenzofuranyl group substituted with a dibenzofuranyl group or a dibenzothiophenyl group may be excluded.

In Formula 3, a case where Arla is represented by Formula 3-a may be excluded.

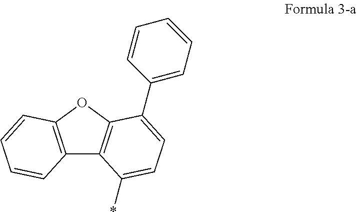 [SMILES: *c1ccc(-c2ccccc2)c2oc3ccccc3c12]

The content (e.g., definitions) of R¹ to R¹⁷ described in Formula 1 may be similarly applied to Formula 3.

In one or more embodiments, the amine compound represented by Formula 1 may be represented by any one selected from among Formula 4-1 to Formula 4-3.

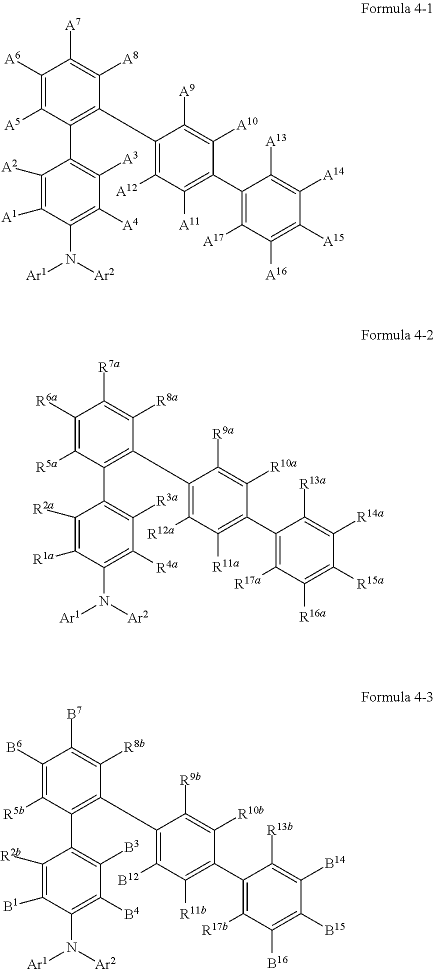

In Formula 4-1, A¹ to A¹⁷ may each independently be a hydrogen atom, a deuterium atom, or a substituted or unsubstituted aryl group having 6 to 30 ring-forming carbons. A¹ to A¹⁷ may each independently be a hydrogen atom, a deuterium atom, or a substituted or unsubstituted phenyl group.

In Formula 4-2, R^{1a} to R^{17a} may each independently be a hydrogen atom, a deuterium atom, a substituted or unsubstituted oxy group, a substituted or unsubstituted thio group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons. In one or more embodiments, R^{1a} to R^{17a} may be each bonded to an adjacent group to form a ring.

In Formula 4-2, at least one pair among pairs including (e.g., composed of) two selected from among R^{1a} to R^{17a} may be bonded to each other to form an aromatic hydrocarbon ring. In one or more embodiments, at least one pair among R^{1a} and R^{2a}, R^{3a} and R^{4a}, R^{5a} and R^{6a}, R^{6a} and R^{7a}, R^{7a} and R^{8a}, R^{9a} and R^{10a}, R^{11a} and R^{12a}, R^{13a} and R^{14a}, R^{14a} and R^{15a}, R^{15a} and R^{16a}, and R^{16a} and R^{17a} may be bonded to each other to form an aromatic hydrocarbon ring. For example, R^{15a} and R^{16a} may be bonded to each other to form an aromatic hydrocarbon ring such as naphthalene.

In one or more embodiments, at least one pair among pairs including (e.g., composed of) two selected from among R^{1a} to R^{17a} may be bonded to each other to form an aromatic hydrocarbon ring, and the others that do not form a ring among R^{1a} to R^{17a} may each independently be a hydrogen atom, or a deuterium atom.

In Formula 4-3, B¹, B³, B⁴, B⁶, B⁷, B¹², B¹⁴, B¹⁵, and B¹⁶ may each independently be a hydrogen atom, or a deuterium atom.

In Formula 4-3, R^{2b}, R^{5b}, R^{8b}, R^{9b}, R^{10b}, R^{11b}, R^{13b}, and R^{17b} may each independently be a hydrogen atom, a substituted or unsubstituted oxy group, a substituted or unsubstituted thio group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons.

In one or more embodiments, R^{2b}, R^{5b}, R^{8b}, R^{9b}, R^{10b}, R^{11b}, R^{13b}, and R^{17b} may be each bonded to an adjacent group to form a ring.

In Formula 4-3, at least one pair among R^{2b} and R^{5b}, R^{8b} and R^{9b}, R^{10b} and R^{13b}, and R^{11b} and R^{17b} may be bonded to each other to form a ring. In one or more embodiments, at least one pair among R^{2b} and R^{5b}, R^{8b} and R^{9b}, R^{10b} and R^{13b}, and R^{11b} and R^{17b} may be bonded to each other to form an aromatic hydrocarbon ring or an aromatic heterocycle. For example, R^{10b} and R^{13b} may be bonded to each other to form an aromatic hydrocarbon ring such as a fluorene ring, or an aromatic heterocycle such as a dibenzofuran ring, and dibenzothiophene ring.

The definitions (e.g., contents) of Ar¹ and Ar² described in Formula 1 may be similarly applied to Formula 4-1 to 4-3.

In one or more embodiments, the amine compound according to one or more embodiments represented by Formula 1 may include (e.g., contain) at least one deuterium atom as a substituent. The amine compound according to one or more embodiments represented by Formula 1 may include a structure in which at least one hydrogen atom is substituted with a deuterium atom.

In one or more embodiments, the amine compound represented by Formula 1 may be represented Formula 5, and the amine compound may be a compound satisfying any one among combinations present in Compound Combination Table 1.

The hole transport region HTR of the light-emitting element ED may include (e.g., contain) at least one among the amine compounds satisfying combinations present in Compound Combination Table 1. For example, the hole transport layer HTL of the light-emitting element ED may include (e.g., contain) at least one among the amine compounds satisfying combinations present in Compound Combination Table 1.

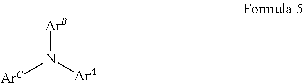

In Formula 5, Ar^{A} may be selected from among the group consisting of Substituent 1 to Substituent 12 in Substituent Group A, Ar^{B} may be selected from among the group consisting of Substituent 1 to Substituent 12, and Substituent 21 to Substituent 52 in Substituent Group A, and Ar^{C} may be selected from among the group consisting of Substituent 2 to Substituent 7, Substituent 9 to Substituent 11 and Substituent 31 to Substituent 52 in Substituent Group A.

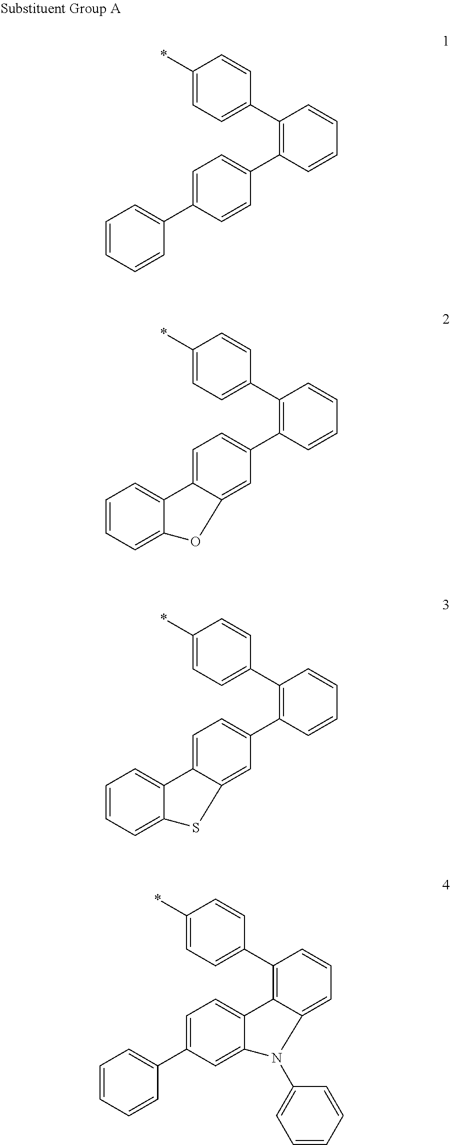 [SMILES: *c1ccc(-c2ccccc2-c2ccc(-c3ccccc3)cc2)cc1 ; *c1ccc(-c2ccccc2-c2ccc3c(c2)oc2ccccc23)cc1 ; *c1ccc(-c2ccccc2-c2ccc3c(c2)sc2ccccc23)cc1 ; *c1ccc(-c2cccc3c2c2ccc(-c4ccccc4)cc2n3-c2ccccc2)cc1]

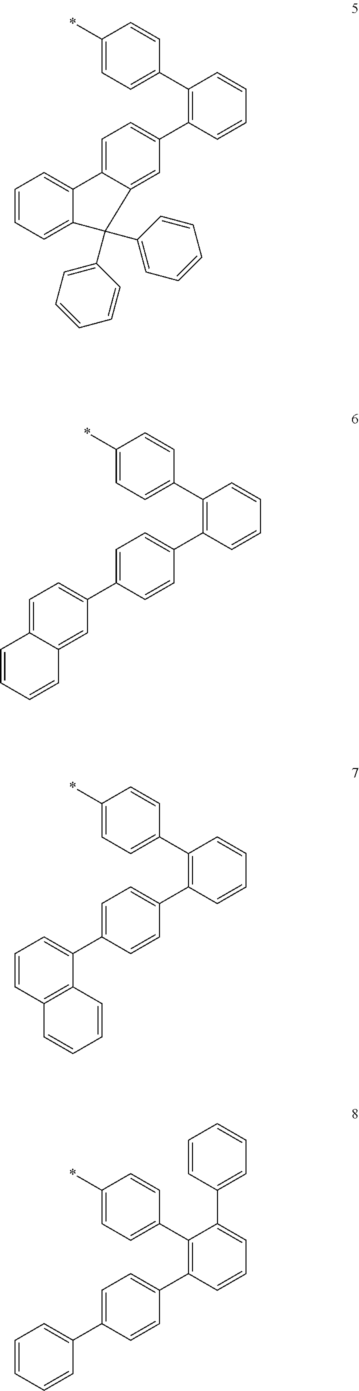 [SMILES: *c1ccc(-c2ccccc2-c2ccc3c(c2)C(c2ccccc2)(c2ccccc2)c2ccccc2-3)cc1 ; *c1ccc(-c2ccccc2-c2ccc(-c3ccc4ccccc4c3)cc2)cc1 ; *c1ccc(-c2ccccc2-c2ccc(-c3cccc4ccccc34)cc2)cc1 ; *c1ccc(-c2c(-c3ccccc3)cccc2-c2ccc(-c3ccccc3)cc2)cc1]

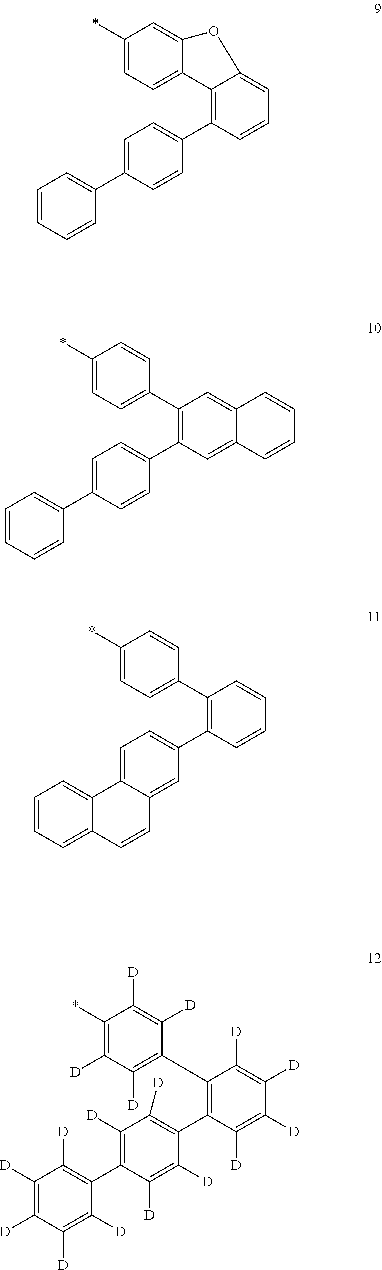 [SMILES: *c1ccc2c(c1)oc1cccc(-c3ccc(-c4ccccc4)cc3)c12 ; *c1ccc(-c2cc3ccccc3cc2-c2ccc(-c3ccccc3)cc2)cc1 ; *c1ccc(-c2ccccc2-c2ccc3c(ccc4ccccc43)c2)cc1 ; *c1c([2H])c([2H])c(-c2c([2H])c([2H])c([2H])c([2H])c2-c2c([2H])c([2H])c(-c3c([2H])c([2H])c([2H])c([2H])c3[2H])c([2H])c2[2H])c([2H])c1[2H]]

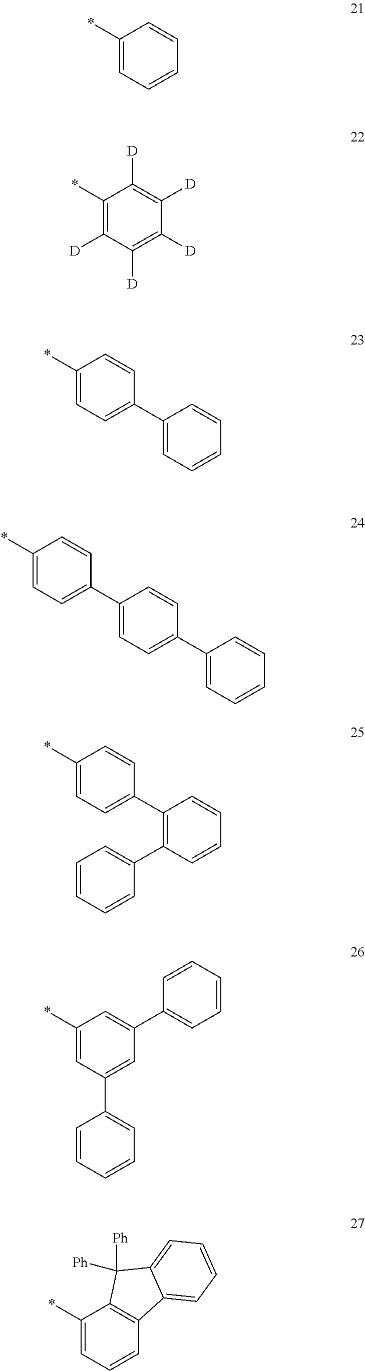

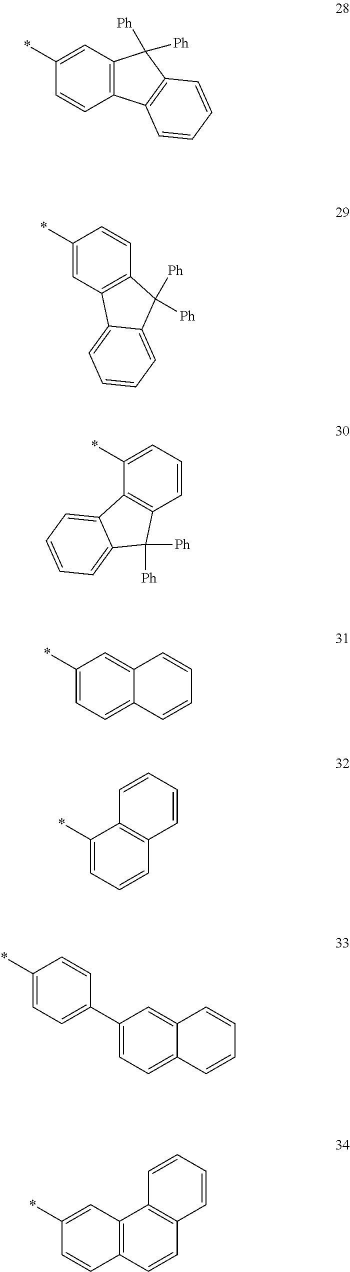

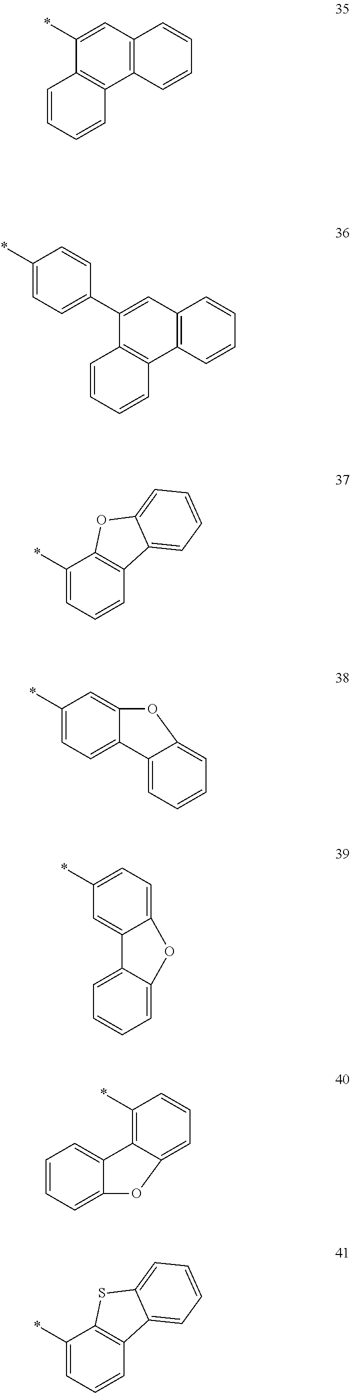

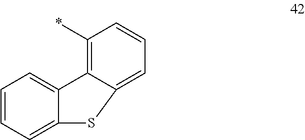 [SMILES: *c1cccc2sc3ccccc3c12]

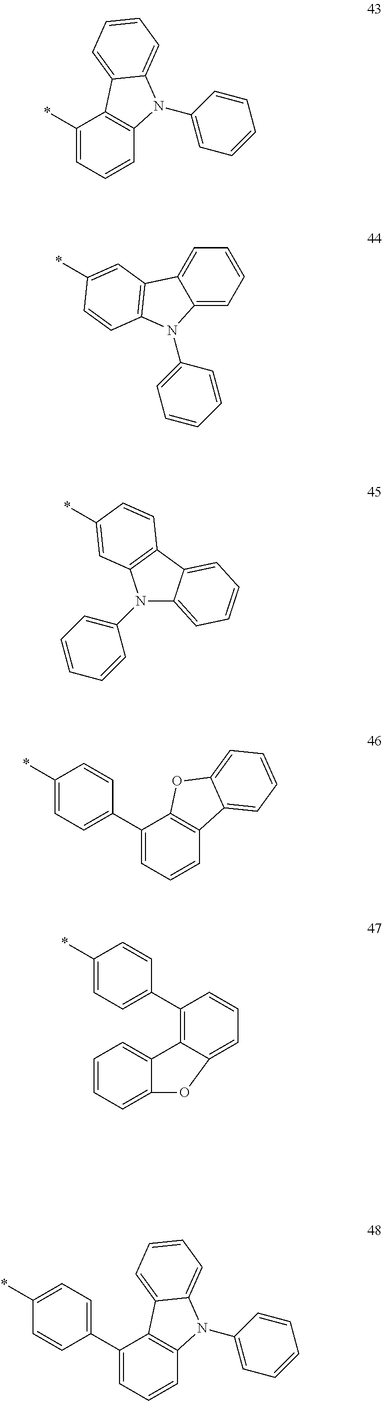 [SMILES: *c1cccc2c1c1ccccc1n2-c1ccccc1 ; *c1ccc2c(c1)c1ccccc1n2-c1ccccc1 ; *c1ccc2c3ccccc3n(-c3ccccc3)c2c1 ; *c1ccc(-c2cccc3c2oc2ccccc23)cc1 ; *c1ccc(-c2cccc3oc4ccccc4c23)cc1 ; *c1ccc(-c2cccc3c2c2ccccc2n3-c2ccccc2)cc1]

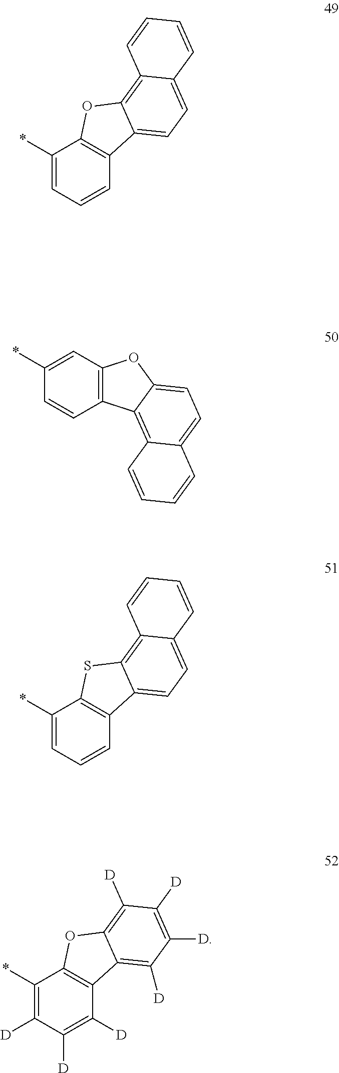

Compound Combination Table 1

| No. | Ar^{A} | Ar^{B} | Ar^{C} |
|---|---|---|---|
| 1 | 1 | 21 | 33 |
| 2 | 2 | 21 | 33 |
| 3 | 3 | 21 | 33 |
| 4 | 4 | 21 | 33 |
| 5 | 5 | 21 | 33 |
| 6 | 6 | 21 | 33 |
| 7 | 7 | 21 | 33 |
| 8 | 8 | 21 | 33 |
| 9 | 9 | 21 | 33 |
| 10 | 10 | 21 | 33 |
| 11 | 11 | 21 | 33 |
| 12 | 1 | 21 | 34 |
| 13 | 2 | 21 | 34 |
| 14 | 3 | 21 | 34 |
| 15 | 4 | 21 | 34 |
| 16 | 5 | 21 | 34 |
| 17 | 6 | 21 | 34 |
| 18 | 7 | 21 | 34 |
| 19 | 8 | 21 | 34 |
| 20 | 9 | 21 | 34 |
| 21 | 10 | 21 | 34 |
| 22 | 11 | 21 | 34 |
| 23 | 1 | 21 | 37 |
| 24 | 2 | 21 | 37 |
| 25 | 3 | 21 | 37 |
| 26 | 4 | 21 | 37 |
| 27 | 5 | 21 | 37 |
| 28 | 6 | 21 | 37 |
| 29 | 7 | 21 | 37 |
| 30 | 8 | 21 | 37 |
| 31 | 9 | 21 | 37 |
| 32 | 10 | 21 | 37 |
| 33 | 11 | 21 | 37 |
| 34 | 1 | 22 | 33 |
| 35 | 2 | 22 | 33 |
| 36 | 3 | 22 | 33 |
| 37 | 4 | 22 | 33 |
| 38 | 5 | 22 | 33 |
| 39 | 6 | 22 | 33 |
| 40 | 7 | 22 | 33 |
| 41 | 8 | 22 | 33 |
| 42 | 9 | 22 | 33 |
| 43 | 10 | 22 | 33 |
| 44 | 11 | 22 | 33 |
| 45 | 1 | 23 | 33 |
| 46 | 2 | 23 | 33 |
| 47 | 3 | 23 | 33 |
| 48 | 4 | 23 | 33 |
| 49 | 5 | 23 | 33 |
| 50 | 6 | 23 | 33 |
| 51 | 7 | 23 | 33 |
| 52 | 8 | 23 | 33 |
| 53 | 9 | 23 | 33 |
| 54 | 10 | 23 | 33 |
| 55 | 11 | 23 | 33 |
| 56 | 1 | 23 | 34 |
| 57 | 2 | 23 | 34 |
| 58 | 3 | 23 | 34 |
| 59 | 4 | 23 | 34 |
| 60 | 5 | 23 | 34 |
| 61 | 6 | 23 | 34 |
| 62 | 7 | 23 | 34 |
| 63 | 8 | 23 | 34 |
| 64 | 9 | 23 | 34 |
| 65 | 10 | 23 | 34 |
| 66 | 11 | 23 | 34 |
| 67 | 1 | 23 | 37 |
| 68 | 2 | 23 | 37 |
| 69 | 3 | 23 | 37 |
| 70 | 4 | 23 | 37 |
| 71 | 5 | 23 | 37 |
| 72 | 6 | 23 | 37 |
| 73 | 7 | 23 | 37 |
| 74 | 8 | 23 | 37 |
| 75 | 9 | 23 | 37 |
| 76 | 10 | 23 | 37 |
| 77 | 11 | 23 | 37 |
| 78 | 1 | 33 | 34 |
| 79 | 2 | 33 | 34 |
| 80 | 3 | 33 | 34 |
| 81 | 4 | 33 | 34 |
| 82 | 5 | 33 | 34 |
| 83 | 6 | 33 | 34 |
| 84 | 7 | 33 | 34 |
| 85 | 8 | 33 | 34 |
| 86 | 9 | 33 | 34 |
| 87 | 10 | 33 | 34 |
| 88 | 11 | 33 | 34 |
| 89 | 1 | 38 | 41 |
| 90 | 2 | 38 | 41 |
| 91 | 3 | 38 | 41 |
| 92 | 4 | 38 | 41 |
| 93 | 5 | 38 | 41 |
| 94 | 6 | 38 | 41 |
| 95 | 7 | 38 | 41 |
| 96 | 8 | 38 | 41 |
| 97 | 9 | 38 | 41 |
| 98 | 10 | 38 | 41 |
| 99 | 11 | 38 | 41 |
| 100 | 1 | 1 | 31 |
| 101 | 1 | 1 | 33 |
| 102 | 1 | 1 | 38 |
| 103 | 1 | 2 | 21 |
| 104 | 1 | 6 | 21 |
| 105 | 6 | 6 | 21 |
| 106 | 6 | 6 | 6 |
| 107 | 1 | 21 | 31 |
| 108 | 1 | 21 | 32 |
| 109 | 1 | 21 | 35 |
| 110 | 1 | 21 | 36 |
| 111 | 1 | 21 | 38 |
| 112 | 1 | 21 | 39 |
| 113 | 1 | 21 | 40 |
| 114 | 1 | 21 | 41 |
| 115 | 1 | 21 | 42 |
| 116 | 1 | 21 | 43 |
| 117 | 1 | 21 | 44 |
| 118 | 1 | 21 | 45 |
| 119 | 1 | 21 | 46 |
| 120 | 1 | 21 | 47 |
| 121 | 1 | 21 | 48 |
| 122 | 1 | 21 | 49 |
| 123 | 1 | 21 | 50 |
| 124 | 1 | 21 | 51 |
| 125 | 1 | 22 | 31 |
| 126 | 1 | 22 | 32 |
| 127 | 1 | 22 | 34 |
| 128 | 1 | 22 | 35 |
| 129 | 1 | 22 | 36 |
| 130 | 1 | 22 | 37 |
| 131 | 1 | 22 | 38 |
| 132 | 1 | 22 | 39 |
| 133 | 1 | 22 | 40 |
| 134 | 1 | 22 | 41 |
| 135 | 1 | 22 | 42 |
| 136 | 1 | 22 | 43 |
| 137 | 1 | 22 | 44 |
| 138 | 1 | 22 | 45 |
| 139 | 1 | 22 | 46 |
| 140 | 1 | 22 | 47 |
| 141 | 1 | 22 | 48 |
| 142 | 1 | 22 | 49 |
| 143 | 1 | 22 | 50 |
| 144 | 1 | 22 | 51 |
| 145 | 1 | 23 | 31 |
| 146 | 1 | 23 | 32 |
| 147 | 1 | 23 | 35 |
| 148 | 1 | 23 | 36 |
| 149 | 1 | 23 | 38 |
| 150 | 1 | 23 | 39 |
| 151 | 1 | 23 | 40 |
| 152 | 1 | 23 | 41 |
| 153 | 1 | 23 | 42 |
| 154 | 1 | 23 | 43 |
| 155 | 1 | 23 | 44 |
| 156 | 1 | 23 | 45 |
| 157 | 1 | 23 | 46 |
| 158 | 1 | 23 | 47 |
| 159 | 1 | 23 | 48 |
| 160 | 1 | 23 | 49 |
| 161 | 1 | 23 | 50 |
| 162 | 1 | 23 | 51 |
| 163 | 1 | 24 | 31 |
| 164 | 1 | 24 | 32 |
| 165 | 1 | 24 | 33 |
| 166 | 1 | 24 | 34 |
| 167 | 1 | 24 | 35 |
| 168 | 1 | 24 | 36 |
| 169 | 1 | 24 | 37 |
| 170 | 1 | 24 | 38 |
| 171 | 1 | 24 | 39 |
| 172 | 1 | 24 | 40 |
| 173 | 1 | 24 | 41 |
| 174 | 1 | 24 | 42 |
| 175 | 1 | 24 | 43 |
| 176 | 1 | 24 | 44 |
| 177 | 1 | 24 | 45 |
| 178 | 1 | 24 | 46 |
| 179 | 1 | 24 | 47 |
| 180 | 1 | 24 | 48 |
| 181 | 1 | 24 | 49 |
| 182 | 1 | 24 | 50 |
| 183 | 1 | 24 | 51 |
| 184 | 1 | 25 | 31 |
| 185 | 1 | 25 | 32 |
| 186 | 1 | 25 | 33 |
| 187 | 1 | 25 | 34 |
| 188 | 1 | 25 | 35 |
| 189 | 1 | 25 | 36 |
| 190 | 1 | 25 | 37 |
| 191 | 1 | 25 | 38 |
| 192 | 1 | 25 | 39 |
| 193 | 1 | 25 | 40 |
| 194 | 1 | 25 | 41 |
| 195 | 1 | 25 | 42 |
| 196 | 1 | 25 | 43 |
| 197 | 1 | 25 | 44 |
| 198 | 1 | 25 | 45 |
| 199 | 1 | 25 | 46 |
| 200 | 1 | 25 | 47 |
| 201 | 1 | 25 | 48 |
| 202 | 1 | 25 | 49 |
| 203 | 1 | 25 | 50 |
| 204 | 1 | 25 | 51 |
| 205 | 1 | 26 | 31 |
| 206 | 1 | 26 | 32 |
| 207 | 1 | 26 | 33 |
| 208 | 1 | 26 | 34 |
| 209 | 1 | 26 | 35 |
| 210 | 1 | 26 | 36 |
| 211 | 1 | 26 | 37 |
| 212 | 1 | 26 | 38 |
| 213 | 1 | 26 | 39 |
| 214 | 1 | 26 | 40 |
| 215 | 1 | 26 | 41 |
| 216 | 1 | 26 | 42 |
| 217 | 1 | 26 | 43 |
| 218 | 1 | 26 | 44 |
| 219 | 1 | 26 | 45 |
| 220 | 1 | 26 | 46 |
| 221 | 1 | 26 | 47 |
| 222 | 1 | 26 | 48 |
| 223 | 1 | 26 | 49 |
| 224 | 1 | 26 | 50 |
| 225 | 1 | 26 | 51 |
| 226 | 1 | 27 | 31 |
| 227 | 1 | 27 | 32 |
| 228 | 1 | 27 | 33 |
| 229 | 1 | 27 | 34 |
| 230 | 1 | 27 | 35 |
| 231 | 1 | 27 | 36 |
| 232 | 1 | 27 | 37 |
| 233 | 1 | 27 | 38 |
| 234 | 1 | 27 | 39 |
| 235 | 1 | 27 | 40 |
| 236 | 1 | 27 | 41 |
| 237 | 1 | 27 | 42 |
| 238 | 1 | 27 | 43 |
| 239 | 1 | 27 | 44 |
| 240 | 1 | 27 | 45 |
| 241 | 1 | 27 | 46 |
| 242 | 1 | 27 | 47 |
| 243 | 1 | 27 | 48 |
| 244 | 1 | 27 | 49 |
| 245 | 1 | 27 | 50 |
| 246 | 1 | 27 | 51 |
| 247 | 1 | 28 | 31 |
| 248 | 1 | 28 | 32 |
| 249 | 1 | 28 | 33 |
| 250 | 1 | 28 | 34 |
| 251 | 1 | 28 | 35 |
| 252 | 1 | 28 | 36 |
| 253 | 1 | 28 | 37 |
| 254 | 1 | 28 | 38 |
| 255 | 1 | 28 | 39 |
| 256 | 1 | 28 | 40 |
| 257 | 1 | 28 | 41 |
| 258 | 1 | 28 | 42 |
| 259 | 1 | 28 | 43 |
| 260 | 1 | 28 | 44 |
| 261 | 1 | 28 | 45 |
| 262 | 1 | 28 | 46 |
| 263 | 1 | 28 | 47 |
| 264 | 1 | 28 | 48 |
| 265 | 1 | 28 | 49 |
| 266 | 1 | 28 | 50 |
| 267 | 1 | 28 | 51 |
| 268 | 1 | 29 | 31 |
| 269 | 1 | 29 | 32 |
| 270 | 1 | 29 | 33 |
| 271 | 1 | 29 | 34 |
| 272 | 1 | 29 | 35 |
| 273 | 1 | 29 | 36 |
| 274 | 1 | 29 | 37 |
| 275 | 1 | 29 | 38 |
| 276 | 1 | 29 | 39 |
| 277 | 1 | 29 | 40 |
| 278 | 1 | 29 | 41 |
| 279 | 1 | 29 | 42 |
| 280 | 1 | 29 | 43 |
| 281 | 1 | 29 | 44 |
| 282 | 1 | 29 | 45 |
| 283 | 1 | 29 | 46 |
| 284 | 1 | 29 | 47 |
| 285 | 1 | 29 | 48 |
| 286 | 1 | 29 | 49 |
| 287 | 1 | 29 | 50 |
| 288 | 1 | 29 | 51 |
| 289 | 1 | 30 | 31 |
| 290 | 1 | 30 | 32 |
| 291 | 1 | 30 | 33 |
| 292 | 1 | 30 | 34 |
| 293 | 1 | 30 | 35 |
| 294 | 1 | 30 | 36 |
| 295 | 1 | 30 | 37 |
| 296 | 1 | 30 | 38 |
| 297 | 1 | 30 | 39 |
| 298 | 1 | 30 | 40 |
| 299 | 1 | 30 | 41 |
| 300 | 1 | 30 | 42 |
| 301 | 1 | 30 | 43 |
| 302 | 1 | 30 | 44 |
| 303 | 1 | 30 | 45 |
| 304 | 1 | 30 | 46 |
| 305 | 1 | 30 | 47 |
| 306 | 1 | 30 | 48 |
| 307 | 1 | 30 | 49 |
| 308 | 1 | 30 | 50 |
| 309 | 1 | 30 | 51 |
| 310 | 1 | 31 | 31 |
| 311 | 1 | 31 | 32 |
| 312 | 1 | 31 | 33 |
| 313 | 1 | 31 | 34 |
| 314 | 1 | 31 | 35 |
| 315 | 1 | 31 | 36 |
| 316 | 1 | 31 | 37 |
| 317 | 1 | 31 | 38 |
| 318 | 1 | 31 | 39 |
| 319 | 1 | 31 | 40 |
| 320 | 1 | 31 | 41 |
| 321 | 1 | 31 | 42 |
| 322 | 1 | 31 | 43 |
| 323 | 1 | 31 | 44 |
| 324 | 1 | 31 | 45 |
| 325 | 1 | 31 | 46 |
| 326 | 1 | 31 | 47 |
| 327 | 1 | 31 | 48 |
| 328 | 1 | 31 | 49 |
| 329 | 1 | 31 | 50 |
| 330 | 1 | 31 | 51 |
| 331 | 1 | 32 | 32 |
| 332 | 1 | 32 | 33 |
| 333 | 1 | 32 | 34 |
| 334 | 1 | 32 | 35 |
| 335 | 1 | 32 | 36 |
| 336 | 1 | 32 | 37 |
| 337 | 1 | 32 | 38 |
| 338 | 1 | 32 | 39 |
| 339 | 1 | 32 | 40 |
| 340 | 1 | 32 | 41 |
| 341 | 1 | 32 | 42 |
| 342 | 1 | 32 | 43 |
| 343 | 1 | 32 | 44 |
| 344 | 1 | 32 | 45 |
| 345 | 1 | 32 | 46 |
| 346 | 1 | 32 | 47 |
| 347 | 1 | 32 | 48 |
| 348 | 1 | 32 | 49 |
| 349 | 1 | 32 | 50 |
| 350 | 1 | 32 | 51 |
| 351 | 1 | 33 | 33 |
| 352 | 1 | 33 | 35 |
| 353 | 1 | 33 | 36 |
| 354 | 1 | 33 | 37 |
| 355 | 1 | 33 | 38 |
| 356 | 1 | 33 | 39 |
| 357 | 1 | 33 | 40 |
| 358 | 1 | 33 | 41 |
| 359 | 1 | 33 | 42 |
| 360 | 1 | 33 | 43 |
| 361 | 1 | 33 | 44 |
| 362 | 1 | 33 | 45 |
| 363 | 1 | 33 | 46 |
| 364 | 1 | 33 | 47 |
| 365 | 1 | 33 | 48 |
| 366 | 1 | 33 | 49 |
| 367 | 1 | 33 | 50 |
| 368 | 1 | 33 | 51 |
| 369 | 1 | 34 | 34 |
| 370 | 1 | 34 | 35 |
| 371 | 1 | 34 | 36 |
| 372 | 1 | 34 | 37 |
| 373 | 1 | 34 | 38 |
| 374 | 1 | 34 | 39 |
| 375 | 1 | 34 | 40 |
| 376 | 1 | 34 | 41 |
| 377 | 1 | 34 | 42 |
| 378 | 1 | 34 | 43 |
| 379 | 1 | 34 | 44 |
| 380 | 1 | 34 | 45 |
| 381 | 1 | 34 | 46 |
| 382 | 1 | 34 | 47 |
| 383 | 1 | 34 | 48 |
| 384 | 1 | 34 | 49 |
| 385 | 1 | 34 | 50 |
| 386 | 1 | 34 | 51 |
| 387 | 1 | 35 | 35 |
| 388 | 1 | 35 | 36 |
| 389 | 1 | 35 | 37 |
| 390 | 1 | 35 | 38 |
| 391 | 1 | 35 | 39 |
| 392 | 1 | 35 | 40 |
| 393 | 1 | 35 | 41 |
| 394 | 1 | 35 | 42 |
| 395 | 1 | 35 | 43 |
| 396 | 1 | 35 | 44 |
| 397 | 1 | 35 | 45 |
| 398 | 1 | 35 | 46 |
| 399 | 1 | 35 | 47 |
| 400 | 1 | 35 | 48 |
| 401 | 1 | 35 | 49 |
| 402 | 1 | 35 | 50 |
| 403 | 1 | 35 | 51 |
| 404 | 1 | 36 | 36 |
| 405 | 1 | 36 | 37 |
| 406 | 1 | 36 | 38 |
| 407 | 1 | 36 | 39 |
| 408 | 1 | 36 | 40 |
| 409 | 1 | 36 | 41 |
| 410 | 1 | 36 | 42 |
| 411 | 1 | 36 | 43 |
| 412 | 1 | 36 | 44 |
| 413 | 1 | 36 | 45 |
| 414 | 1 | 36 | 46 |
| 415 | 1 | 36 | 47 |
| 416 | 1 | 36 | 48 |
| 417 | 1 | 36 | 49 |
| 418 | 1 | 36 | 50 |
| 419 | 1 | 36 | 51 |
| 420 | 1 | 37 | 37 |
| 421 | 1 | 37 | 38 |
| 422 | 1 | 37 | 39 |
| 423 | 1 | 37 | 40 |
| 424 | 1 | 37 | 41 |
| 425 | 1 | 37 | 42 |
| 426 | 1 | 37 | 43 |
| 427 | 1 | 37 | 44 |
| 428 | 1 | 37 | 45 |
| 429 | 1 | 37 | 46 |
| 430 | 1 | 37 | 47 |
| 431 | 1 | 37 | 48 |
| 432 | 1 | 37 | 49 |
| 433 | 1 | 37 | 50 |
| 434 | 1 | 37 | 51 |
| 435 | 1 | 38 | 38 |
| 436 | 1 | 38 | 39 |
| 437 | 1 | 38 | 40 |
| 438 | 1 | 38 | 42 |
| 439 | 1 | 38 | 43 |
| 440 | 1 | 38 | 44 |
| 441 | 1 | 38 | 45 |
| 442 | 1 | 38 | 46 |
| 443 | 1 | 38 | 47 |
| 444 | 1 | 38 | 48 |
| 445 | 1 | 38 | 49 |
| 446 | 1 | 38 | 50 |
| 447 | 1 | 38 | 51 |
| 448 | 1 | 39 | 39 |
| 449 | 1 | 39 | 40 |
| 450 | 1 | 39 | 41 |
| 451 | 1 | 39 | 42 |
| 452 | 1 | 39 | 43 |
| 453 | 1 | 39 | 44 |
| 454 | 1 | 39 | 45 |
| 455 | 1 | 39 | 46 |
| 456 | 1 | 39 | 47 |
| 457 | 1 | 39 | 48 |
| 458 | 1 | 39 | 49 |
| 459 | 1 | 39 | 50 |
| 460 | 1 | 39 | 51 |
| 461 | 1 | 40 | 40 |
| 462 | 1 | 40 | 41 |
| 463 | 1 | 40 | 42 |
| 464 | 1 | 40 | 43 |
| 465 | 1 | 40 | 44 |
| 466 | 1 | 40 | 45 |
| 467 | 1 | 40 | 46 |
| 468 | 1 | 40 | 47 |
| 469 | 1 | 40 | 48 |
| 470 | 1 | 40 | 49 |
| 471 | 1 | 40 | 50 |
| 472 | 1 | 40 | 51 |
| 473 | 1 | 41 | 41 |
| 474 | 1 | 41 | 42 |
| 475 | 1 | 41 | 43 |
| 476 | 1 | 41 | 44 |
| 477 | 1 | 41 | 45 |
| 478 | 1 | 41 | 46 |
| 479 | 1 | 41 | 47 |
| 480 | 1 | 41 | 48 |
| 481 | 1 | 41 | 49 |
| 482 | 1 | 41 | 50 |
| 483 | 1 | 41 | 51 |
| 484 | 1 | 42 | 42 |
| 485 | 1 | 42 | 43 |
| 486 | 1 | 42 | 44 |
| 487 | 1 | 42 | 45 |
| 488 | 1 | 42 | 46 |
| 489 | 1 | 42 | 47 |
| 490 | 1 | 42 | 48 |
| 491 | 1 | 42 | 49 |
| 492 | 1 | 42 | 50 |
| 493 | 1 | 42 | 51 |
| 494 | 1 | 43 | 43 |
| 495 | 1 | 43 | 44 |
| 496 | 1 | 43 | 45 |
| 497 | 1 | 43 | 46 |
| 498 | 1 | 43 | 47 |
| 499 | 1 | 43 | 48 |
| 500 | 1 | 43 | 49 |
| 501 | 1 | 43 | 50 |
| 502 | 1 | 43 | 51 |
| 503 | 1 | 44 | 44 |
| 504 | 1 | 44 | 45 |
| 505 | 1 | 44 | 46 |
| 506 | 1 | 44 | 47 |
| 507 | 1 | 44 | 48 |
| 508 | 1 | 44 | 49 |
| 509 | 1 | 44 | 50 |
| 510 | 1 | 44 | 51 |
| 511 | 1 | 45 | 45 |
| 512 | 1 | 45 | 46 |
| 513 | 1 | 45 | 47 |
| 514 | 1 | 45 | 48 |
| 515 | 1 | 45 | 49 |
| 516 | 1 | 45 | 50 |
| 517 | 1 | 45 | 51 |
| 518 | 1 | 46 | 46 |
| 519 | 1 | 46 | 47 |
| 520 | 1 | 46 | 48 |
| 521 | 1 | 46 | 49 |
| 522 | 1 | 46 | 50 |
| 523 | 1 | 46 | 51 |
| 524 | 1 | 47 | 47 |
| 525 | 1 | 47 | 48 |
| 526 | 1 | 47 | 49 |
| 527 | 1 | 47 | 50 |
| 528 | 1 | 47 | 51 |
| 529 | 1 | 48 | 48 |
| 530 | 1 | 48 | 49 |
| 531 | 1 | 48 | 50 |
| 532 | 1 | 48 | 51 |
| 533 | 1 | 49 | 49 |
| 534 | 1 | 49 | 50 |
| 535 | 1 | 49 | 51 |
| 536 | 1 | 50 | 50 |
| 537 | 1 | 50 | 51 |
| 538 | 1 | 51 | 51 |
| 539 | 12 | 21 | 33 |
| 540 | 12 | 21 | 34 |
| 541 | 12 | 21 | 35 |
| 542 | 12 | 21 | 36 |
| 543 | 12 | 21 | 37 |
| 544 | 12 | 21 | 38 |
| 545 | 12 | 21 | 39 |
| 546 | 12 | 21 | 40 |
| 547 | 12 | 21 | 41 |
| 548 | 12 | 21 | 42 |
| 549 | 12 | 21 | 43 |
| 550 | 12 | 21 | 44 |
| 551 | 12 | 21 | 45 |
| 552 | 12 | 21 | 46 |
| 553 | 12 | 21 | 47 |
| 554 | 12 | 21 | 48 |
| 555 | 12 | 21 | 49 |
| 556 | 12 | 21 | 50 |
| 557 | 12 | 21 | 51 |
| 558 | 12 | 21 | 52 |
| 559 | 12 | 22 | 33 |
| 560 | 12 | 22 | 34 |
| 561 | 12 | 22 | 35 |
| 562 | 12 | 22 | 36 |
| 563 | 12 | 22 | 37 |
| 564 | 12 | 22 | 38 |
| 565 | 12 | 22 | 39 |
| 566 | 12 | 22 | 40 |
| 567 | 12 | 22 | 41 |
| 568 | 12 | 22 | 42 |
| 569 | 12 | 22 | 43 |
| 570 | 12 | 22 | 44 |
| 571 | 12 | 22 | 45 |
| 572 | 12 | 22 | 46 |
| 573 | 12 | 22 | 47 |
| 574 | 12 | 22 | 48 |
| 575 | 12 | 22 | 49 |
| 576 | 12 | 22 | 50 |
| 577 | 12 | 22 | 51 |
| 578 | 12 | 22 | 52 |
| 579 | 1 | 21 | 52 |
| 580 | 1 | 22 | 52 |

The amine compound according to one or more embodiments represented by Formula 1 includes 1′:2′,1″:4″,1′″-quarterphenyl moiety, and, in particular, has characteristics in that the 1′:2′,1″:4″,1′″-quarterphenyl moiety is bonded to a nitrogen atom of the amine group at a specific position. In some embodiments, the amine compound according to one or more embodiments may include at least one, as a substituent, selected from among a substituted or unsubstituted naphthylphenyl group, a substituted or unsubstituted dibenzofuranyl group, a substituted or unsubstituted dibenzothiophenyl group, and a substituted or unsubstituted carbazolyl group. The amine compound according to one or more embodiments may have excellent or suitable stability and high charge transport ability by introduction of such a substituent and specification of a substitution position. Therefore, the amine compound according to one or more embodiments may have improved efficiency and lifespan. In some embodiments, the light-emitting element ED containing the amine compound according to one or more embodiments may have improved luminous efficiency and lifespan.

Referring to FIG. 3 to FIG. 6 again, the light-emitting element ED according to one or more embodiments of the disclosure will be described.

As described previously, the hole transport region HTR contains the herein-described amine compound according to one or more embodiments of the disclosure. For example, the hole transport region HTR contains the amine compound represented by Formula 1.

When the hole transport region HTR has a multi-layered structure having a plurality of layers, any one layer among the plurality of layers may contain the amine compound represented by Formula 1. For example, the hole transport region HTR includes a hole injection layer HIL arranged on the first electrode EL1, and a hole transport layer HTL arranged on the hole injection layer HIL, and the hole transport layer HTL may include (e.g., contain) the amine compound represented by Formula 1. However, one or more embodiments of the disclosure is not limited thereto, and for example, the hole injection layer HIL may include (e.g., contain) the amine compound represented by Formula 1.

The hole transport region HTR may include (e.g., contain) one or two, or more amine compounds represented by Formula 1. For example, the hole transport region HTR may include (e.g., contain) at least one among (e.g., selected from among) the compounds present in Compound Group 1, described previously.

The hole transport region HTR may further include a compound represented by Formula H-1:

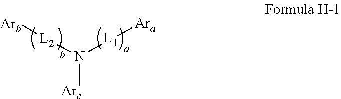

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, if (e.g., when) a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Arₐ and Ar_{b} may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar_{c} may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. In one or more embodiments, the compound represented by Formula H-1 may be a diamine compound in which at least one among Arₐ to Ar_{c} includes the amine group as a substituent. In some embodiments, the compound represented by Formula H-1 may be a carbazole-based compound including a substituted or unsubstituted carbazole group in at least one of Arₐ or Ar_{b}, or a fluorene-based compound including a substituted or unsubstituted fluorene group in at least one of Arₐ or Ar_{b}.

The compound represented by Formula H-1 may be represented by any one among (e.g., selected from among) the compounds in Compound Group H. However, the compounds listed in Compound Group H are examples, and the compounds represented by Formula H-1 are not limited to those represented by Compound Group H:

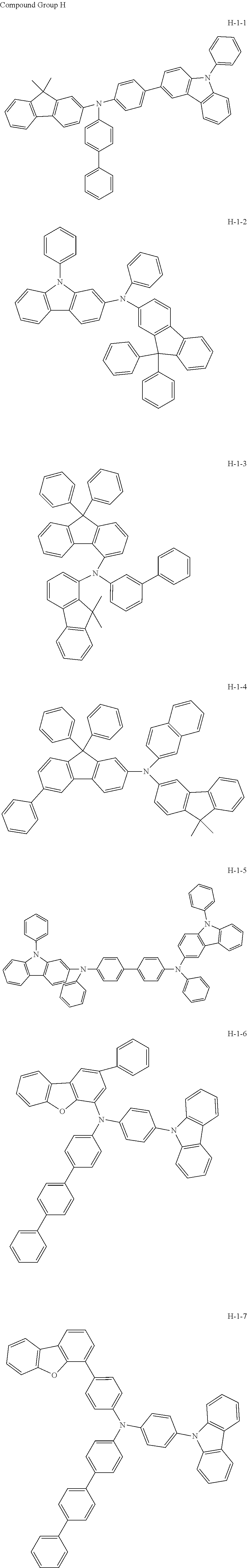

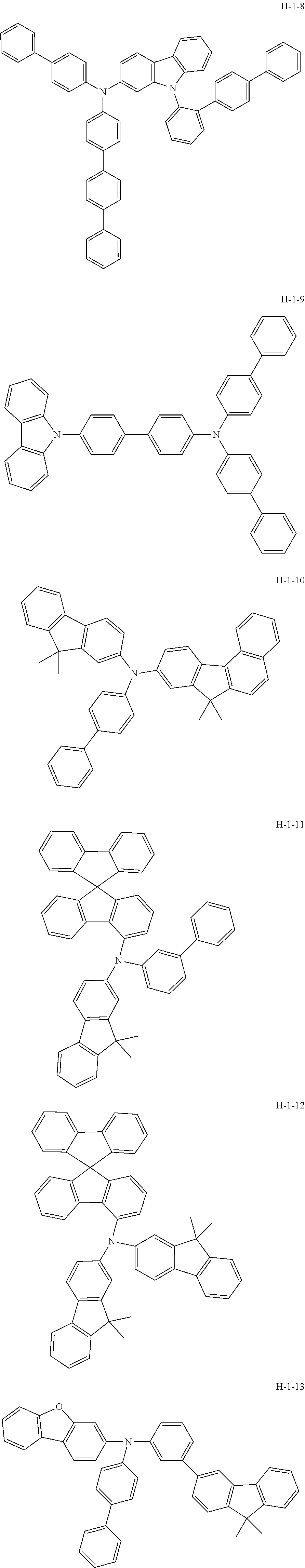

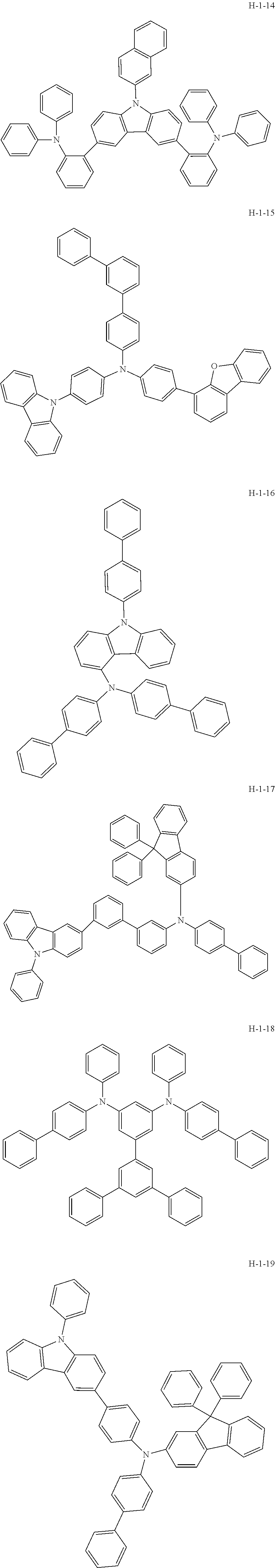

Besides, the hole transport region HTR may further include a suitable hole transport material.

For example, the hole transport region HTR may include a phthalocyanine compound such as copper phthalocyanine; N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium[tetrakis(pentafluorophenyl)borate], dipyrazino[2,3-f:2′,3′-h]quinoxaline-2,3,6,7,10,11-hexacarbonitrile (HATCN), and/or the like.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the herein-described compounds of the hole transport region in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, if (e.g., when) the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the herein-described ranges, satisfactory hole transport properties may be achieved without a substantial increase in driving voltage.

The hole transport region HTR may further include a charge generating material to increase conductivity in addition to the herein-described materials. The charge generating material may be dispersed uniformly (e.g., substantially uniformly) or non-uniformly (e.g., substantially non-uniformly) in the hole transport region HTR. The charge generating material may be, for example, a p-dopant. The p-dopant may include at least one of a halogenated metal compound, a quinone derivative, a metal oxide, or a cyano group-containing compound, but one or more embodiments of the disclosure is not limited thereto. For example, the p-dopant may include a metal halide compound such as CuI or RbI, a quinone derivative such as tetracyanoquinodimethane (TCNQ) or 2,3,5,6-tetrafluoro-7,7′8,8-tetracyanoquinodimethane (F4-TCNQ), a metal oxide such as tungsten oxide or molybdenum oxide, a cyano group-containing compound such as dipyrazino[2,3-f:2′,3′-h]quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP 9), and/or the like, but one or more embodiments of the disclosure is not limited thereto.

As described herein, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the light-emitting layer EML and may thus increase light emission efficiency. A material that may be included in the hole transport region HTR may be used as a material to be included in the buffer layer. The electron blocking layer EBL is a layer that serves to prevent or reduce the electron injection from the electron transport region ETR to the hole transport region HTR.

The light-emitting layer EML is provided on the hole transport region HTR. The light-emitting layer EML may have a thickness of, for example, about 100 Å to about 1,000 Å or about 100 Å to about 300 Å. The light-emitting layer EML may have a single layer formed of a single material, a single layer formed of multiple different materials, or a multi-layered structure having a plurality of layers formed of multiple different materials.

In the light-emitting element ED of one or more embodiments, the light-emitting layer EML may be to emit blue light. The light-emitting element ED of one or more embodiments may include the amine compound of one or more embodiments in a hole transport region HTR and may show high efficiency and long-life characteristics in a blue emission region. However, one or more embodiments of the disclosure is not limited thereto.

In the light-emitting element ED of one or more embodiments, the light-emitting layer EML may include anthracene derivatives, pyrene derivatives, fluoranthene derivatives, chrysene derivatives, dihydrobenzanthracene derivatives, and/or triphenylene derivatives. For example, the light-emitting layer EML may include anthracene derivatives and/or pyrene derivatives.

In the light-emitting elements ED of embodiments, shown in FIG. 3 to FIG. 6, the light-emitting layer EML may include a host and a dopant, and the light-emitting layer EML may include a compound represented by Formula E-1. The compound represented by Formula E-1 may be used as a fluorescence host material.

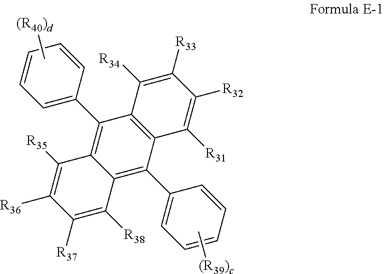

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In one or more embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

Formula E-1 may be represented by any one among Compound E1 to Compound E19.

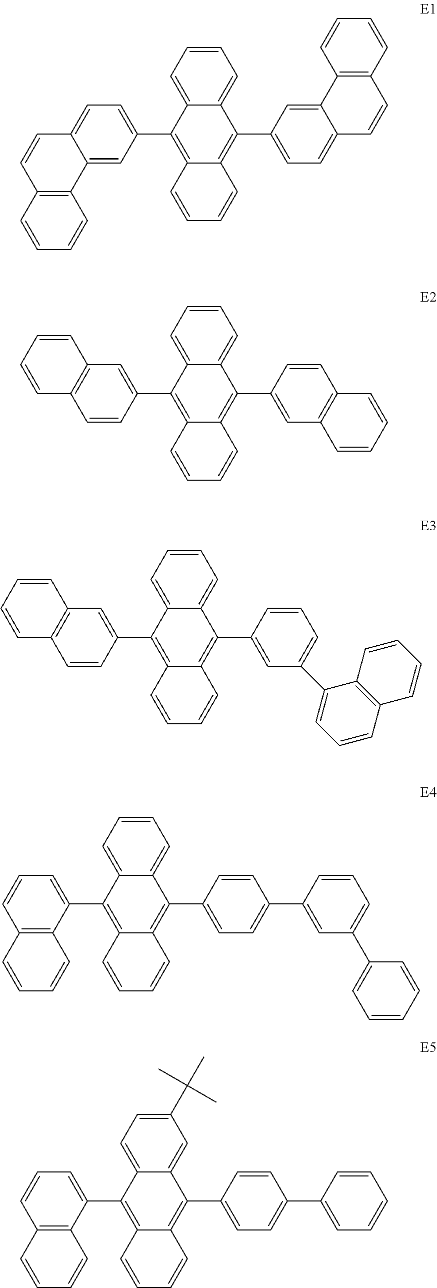

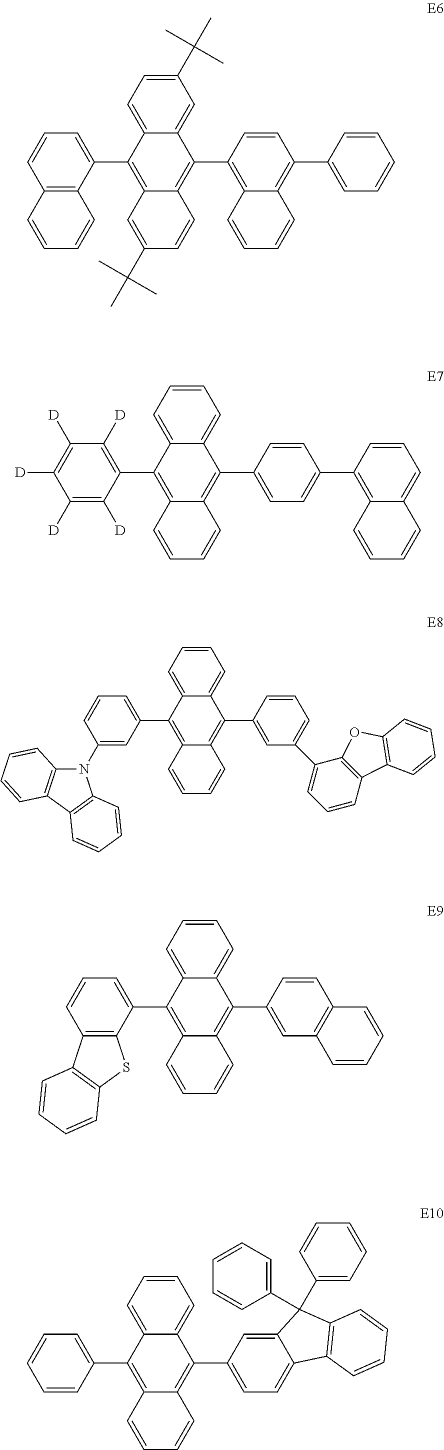

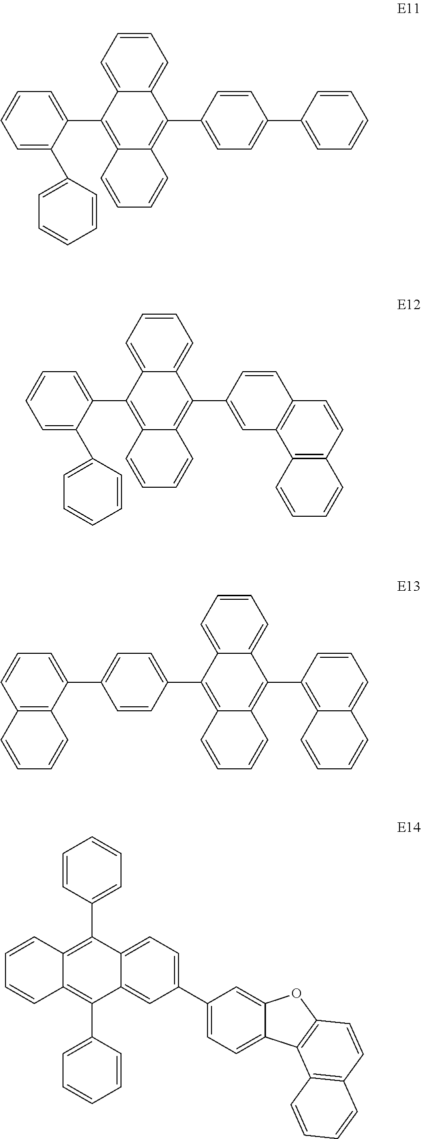

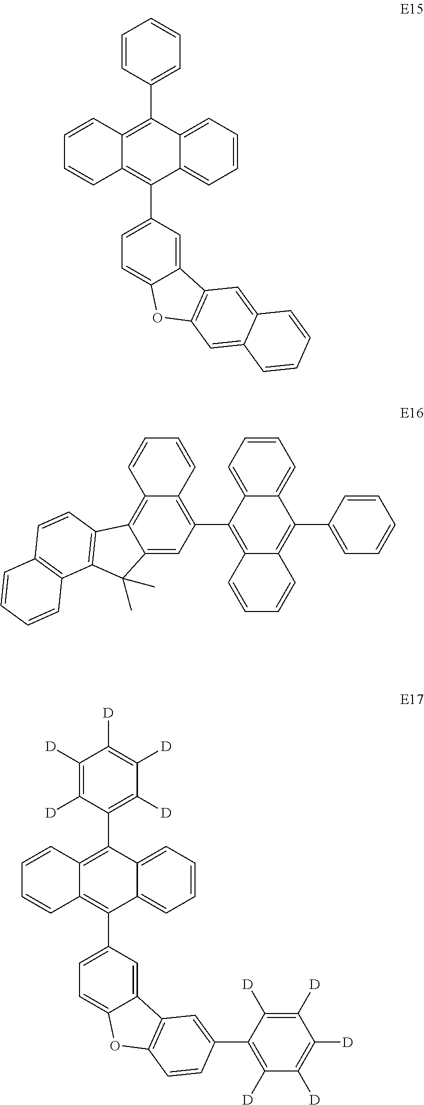

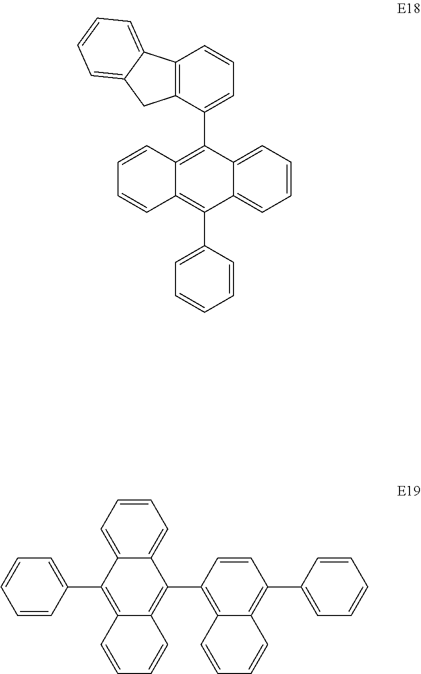

In one or more embodiments, the light-emitting layer EML may include a compound represented by Formula E-2a or Formula E-2b. The compound represented by Formula E-2a or Formula E-2b may be used as a phosphorescence host material.

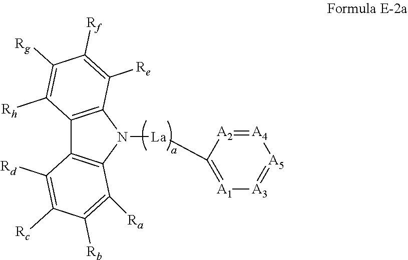

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if “a” is an integer of 2 or more, multiple La may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A₅ may each independently be N or CRi. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Rₐ to Rᵢ may be combined with an adjacent group to form a hydrocarbon ring or a heterocycle including N, O, S, and/or the like as a ring-forming atom.

In one or more embodiments, in Formula E-2a, two or three selected from among A₁ to A₅ may be N, and the remainder may be CRᵢ.

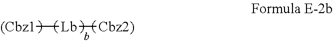

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any (e.g., at least) one selected from among the compounds in Compound Group E-2. However, the compounds listed in Compound Group E-2 are only illustrations, and the compound represented by Formula E-2a or Formula E-2b is not limited to the compounds represented in Compound Group E-2.

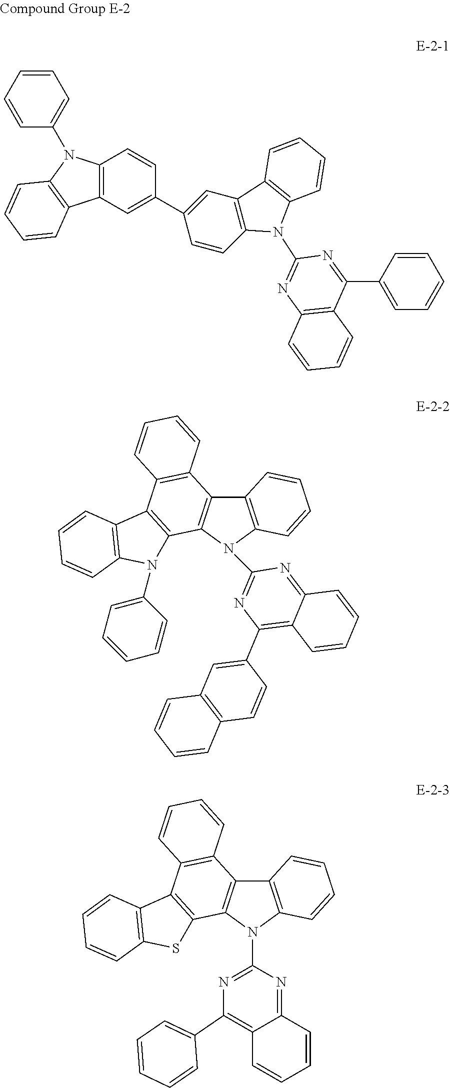 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

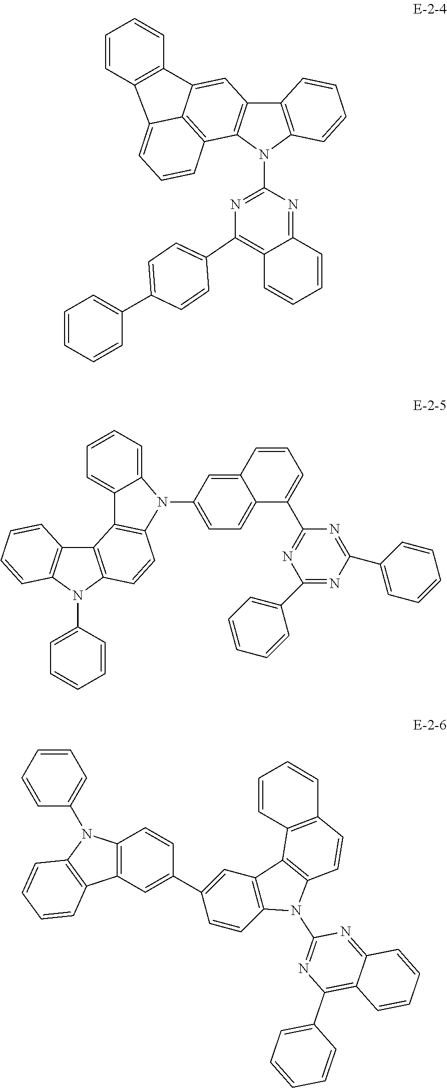 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

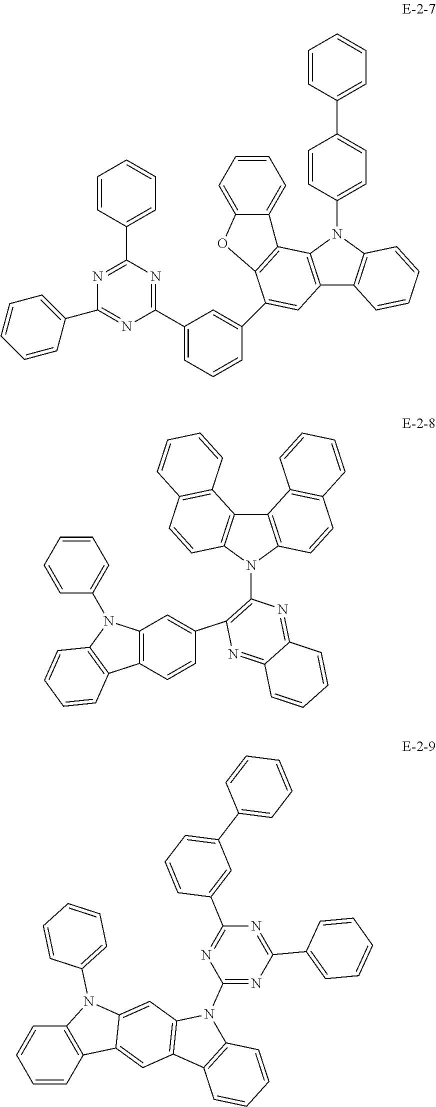 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

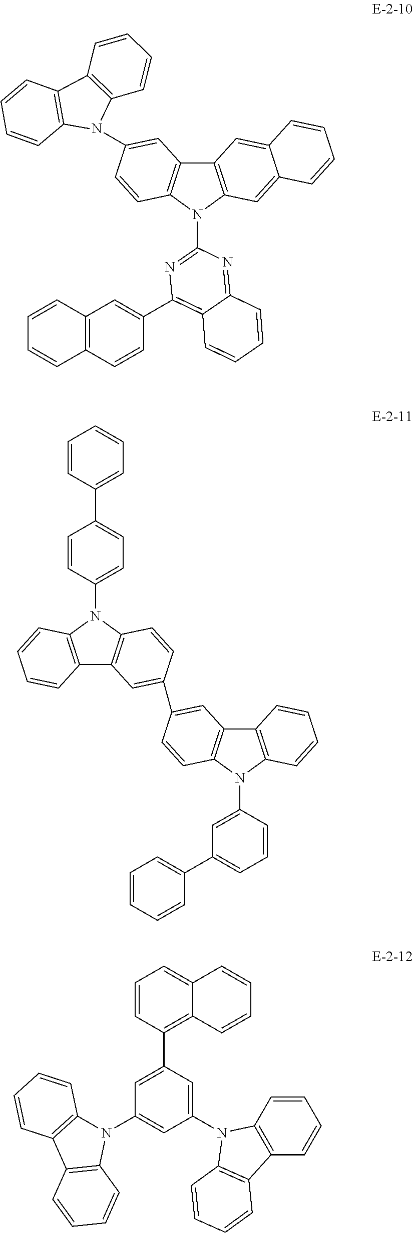

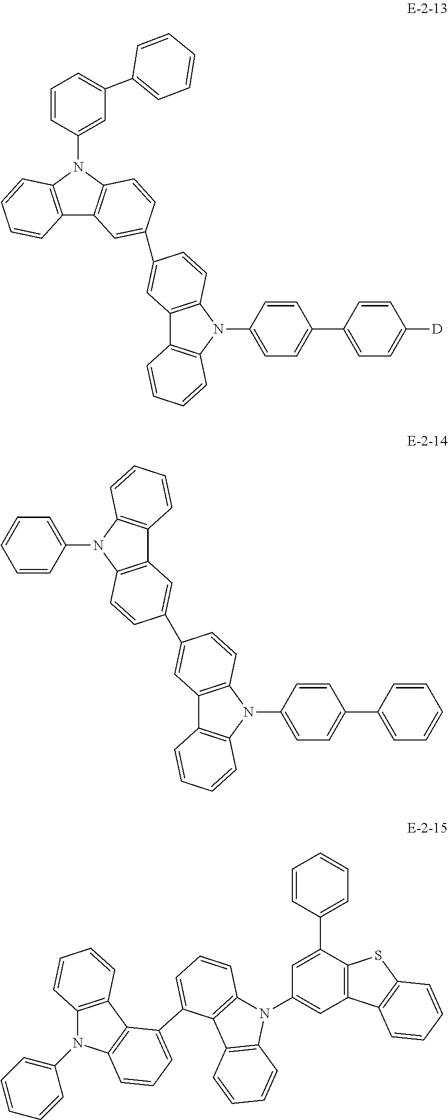 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

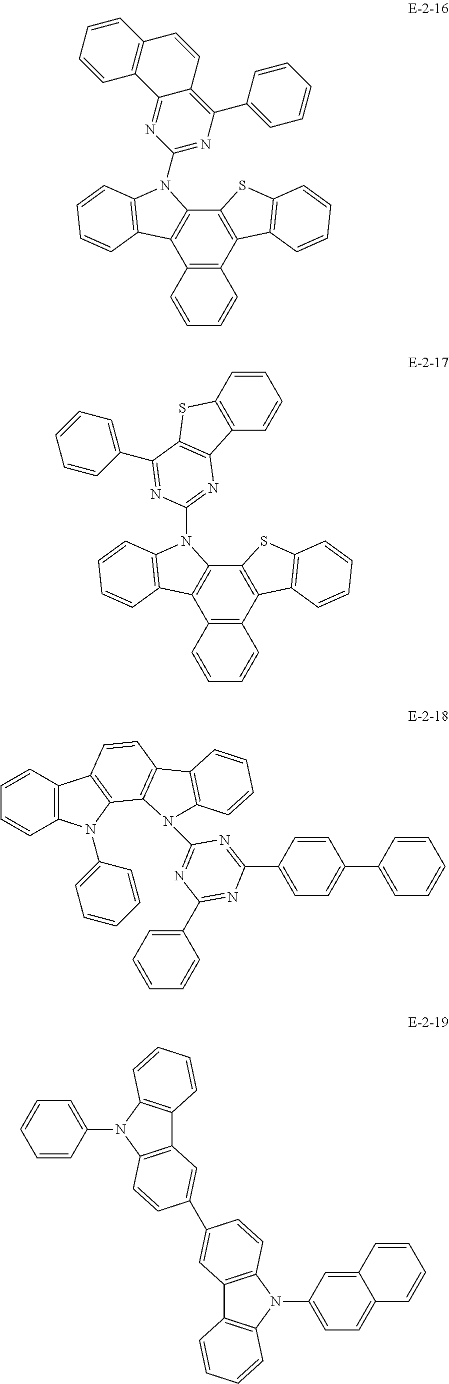 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

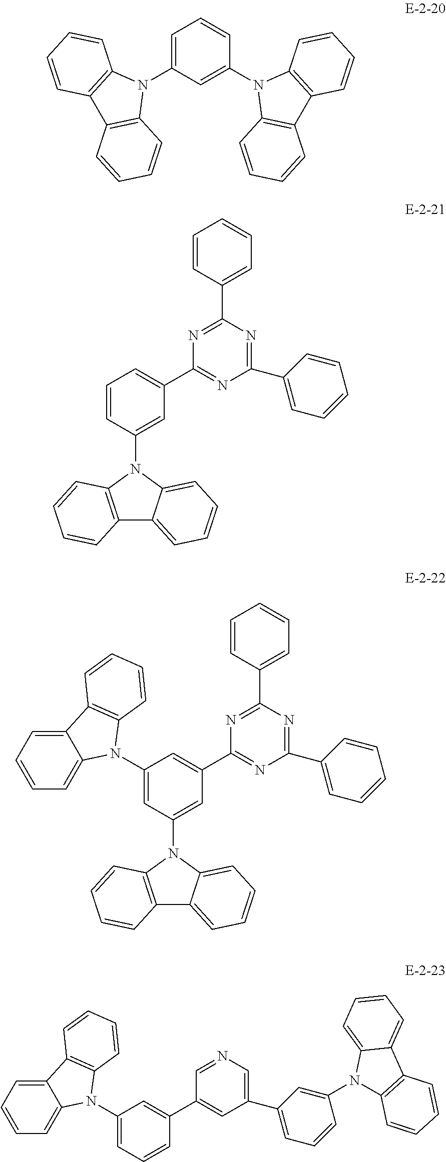 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

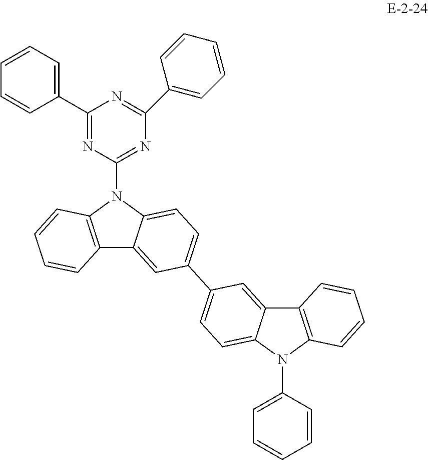 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The light-emitting layer EML may further include a common material well-suitable in the art as a host material. For example, the light-emitting layer EML may include as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, one or more embodiments of the disclosure is not limited thereto. For example, tris(8-hydroxyquinolinato)aluminum (Alq3), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP 1), 1,4-bis(triphenylsilyl)benzene (UGH2), hexaphenylcyclotrisiloxane (DPSiO₃), octaphenylcyclotetra siloxane (DPSiO₄), and/or the like may be used as the host material.

The light-emitting layer EML may include a compound represented by Formula M-a or Formula M-b. The compound represented by Formula M-a or Formula M-b may be used as a phosphorescence dopant material. In some embodiments, in one or more embodiments, the compound represented by Formula M-a or Formula M-b may be used as an auxiliary dopant material.

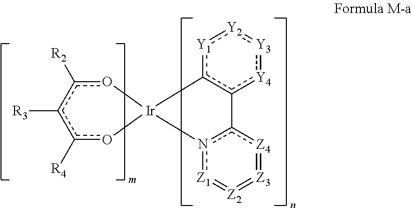

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” may be 0 or 1, and “n” may be 2 or 3. In Formula M-a, if “m” is 0, “n” is 3, and if “m” is 1, “n” is 2.

The compound represented by Formula M-a may be used as a phosphorescence dopant.

The compound represented by Formula M-a may be represented by any (e.g., at least) one selected from among Compounds M-a1 to M-a25. However, Compounds M-a1 to M-a25 are illustrations, and the compound represented by Formula M-a is not limited to the compounds represented by Compounds M-a1 to M-a25.

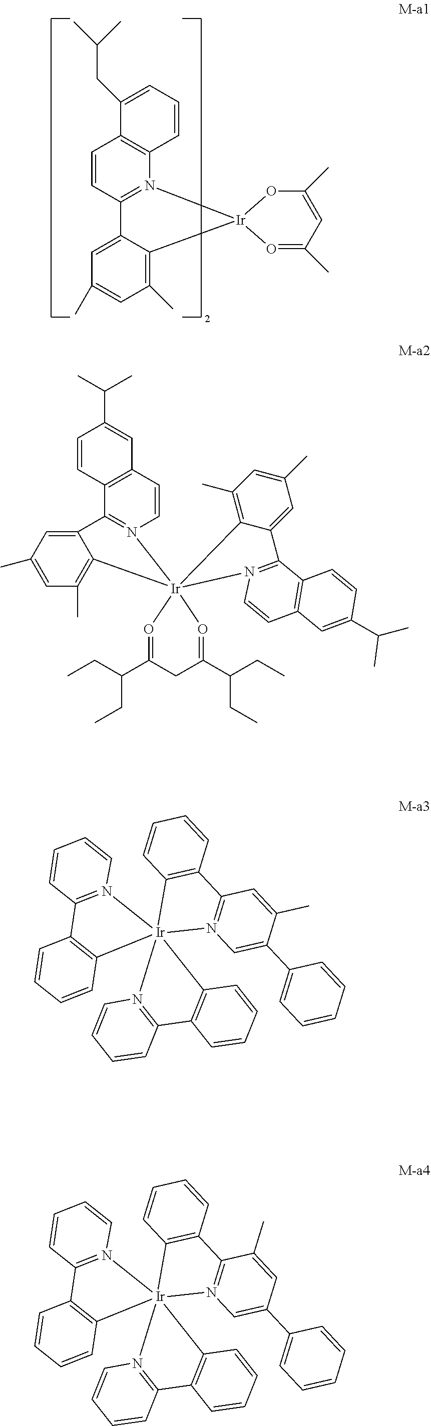

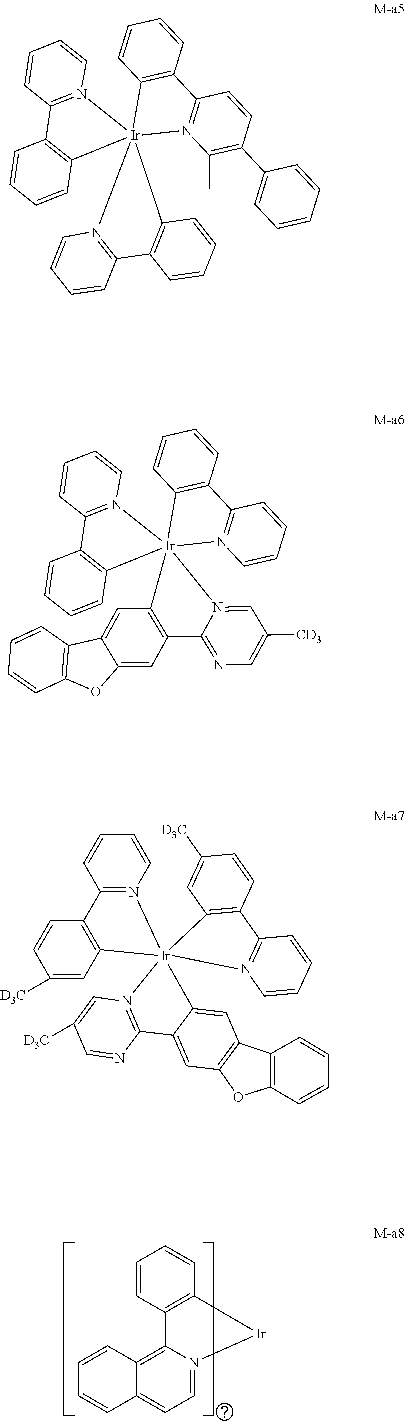

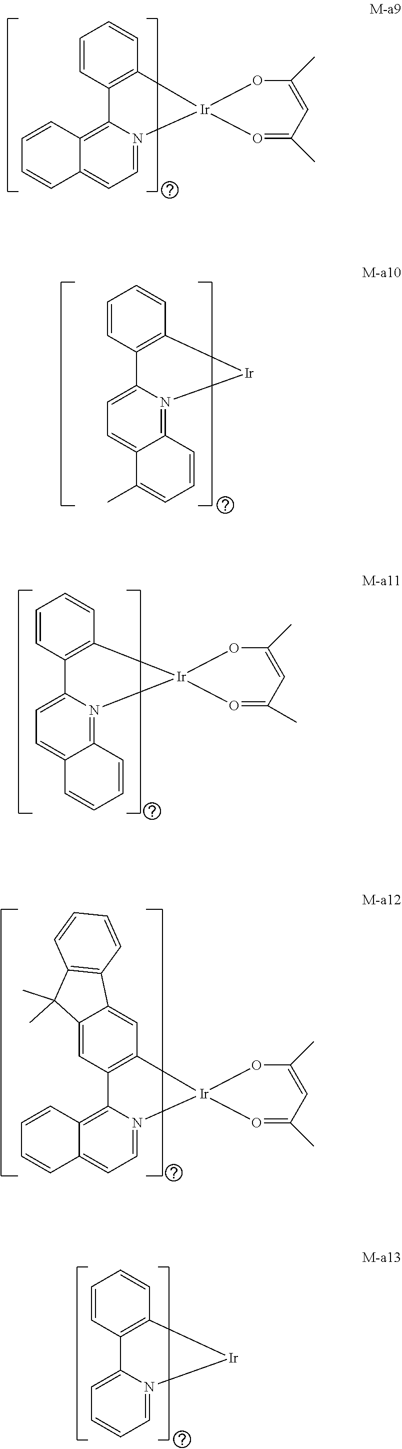

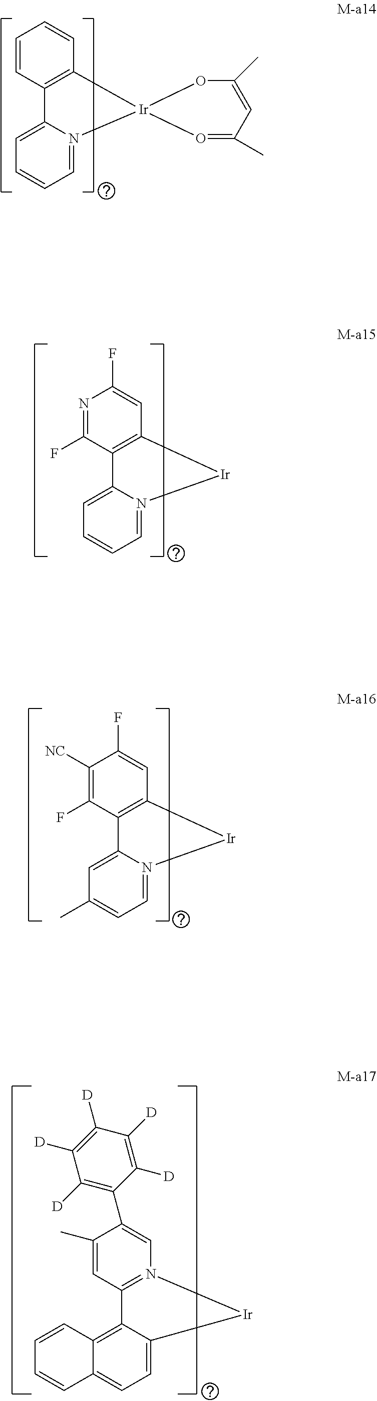

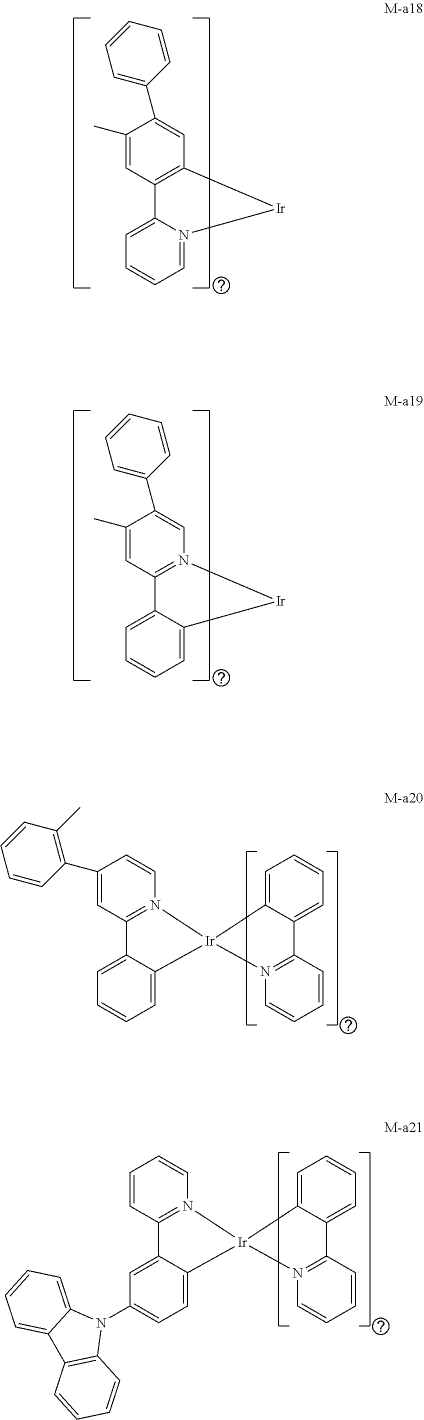

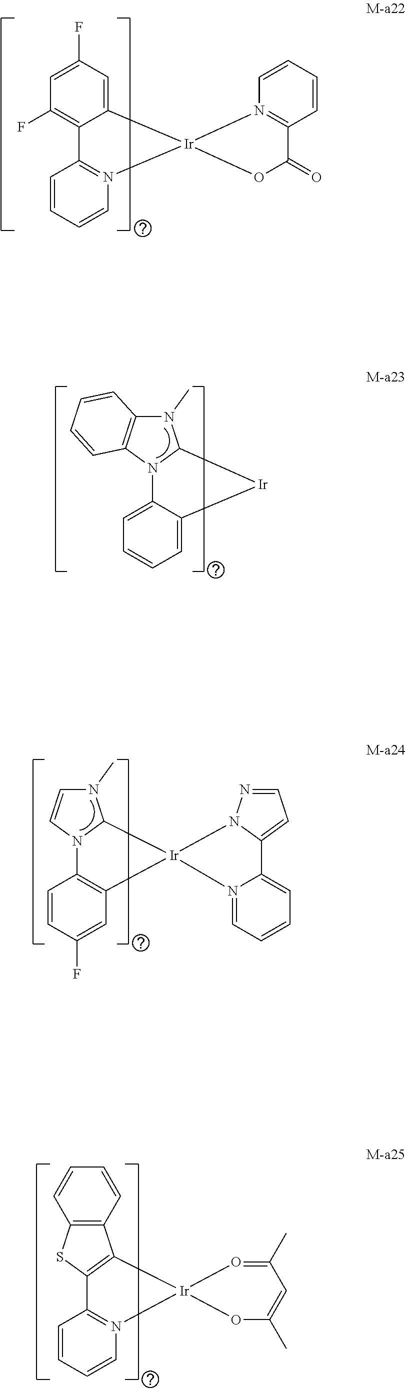

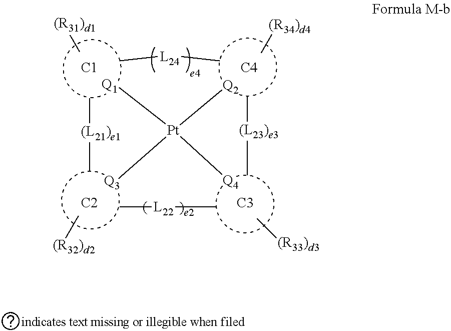

In Formula M-b, Q₁ to Q₄ may each independently be C or N, C₁ to C₄ may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

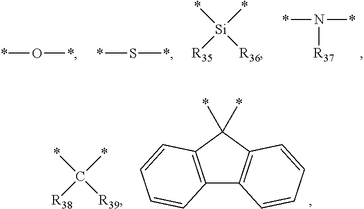

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and d1 to d4 may each independently be an integer of 0 to 4.

The compound represented by Formula M-b may be used as a blue phosphorescence dopant or a green phosphorescence dopant. In some embodiments, the compound represented by Formula M-b may be an auxiliary dopant in one or more embodiments and may be further included in the light-emitting layer EML.

The compound represented by Formula M-b may be represented by any one among Compound M-b-1 to Compound M-b-11. However, the compounds are illustrations, and the compound represented by Formula M-b is not limited to Compound M-b-1 to Compound M-b-11.

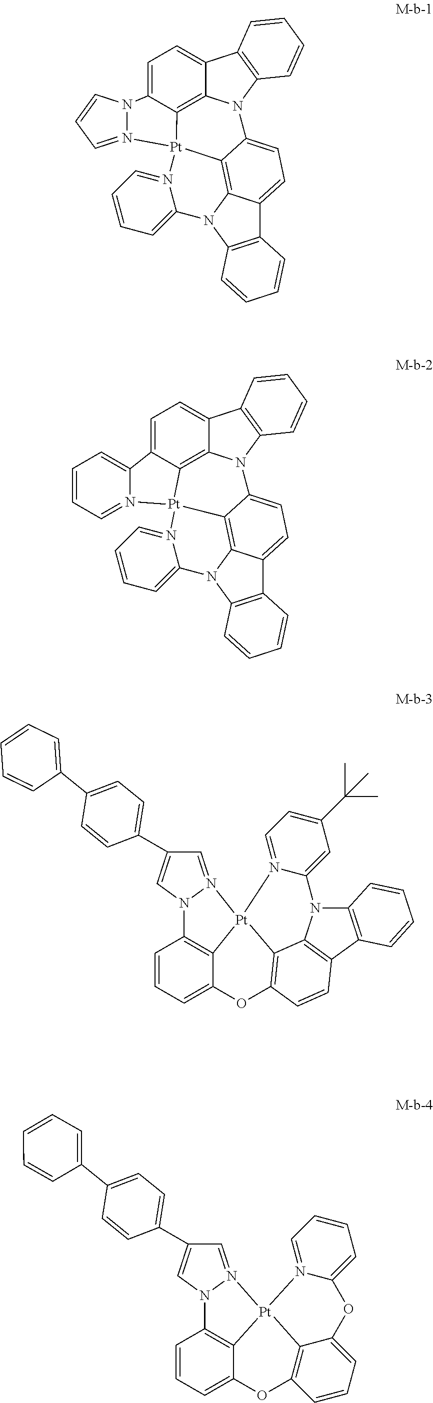

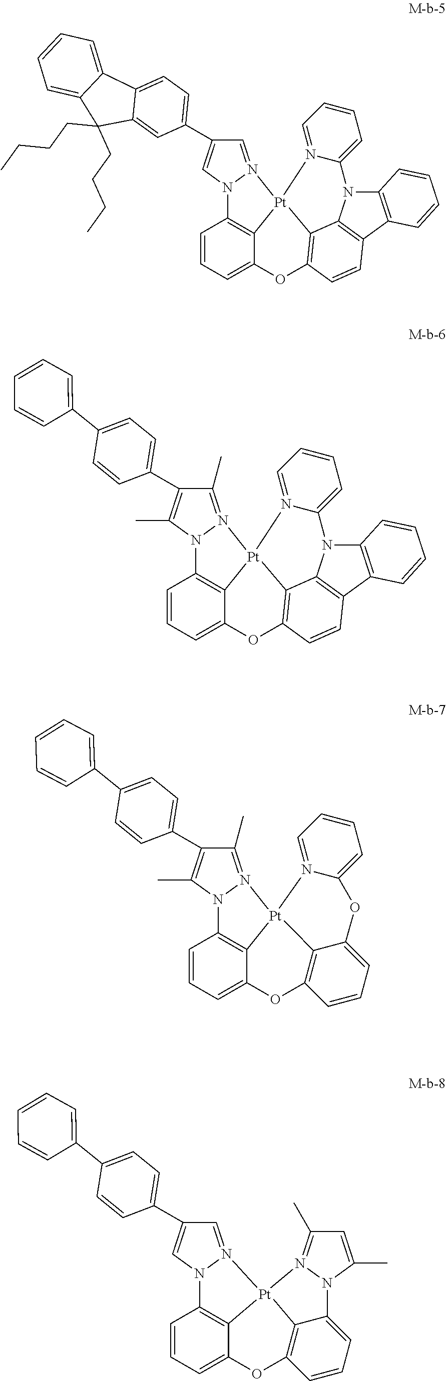

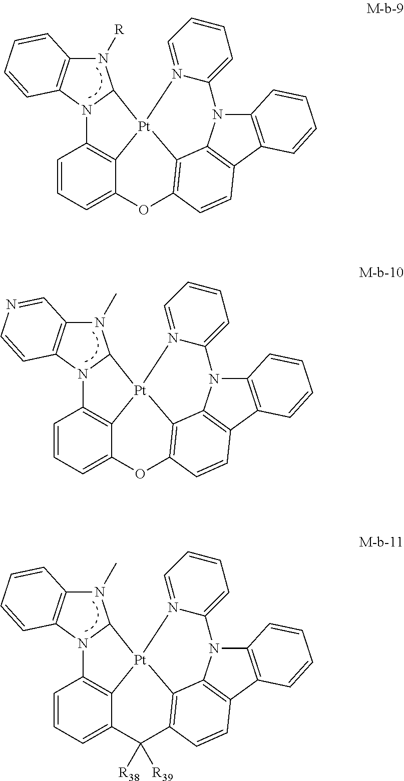

In the preceding compounds, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The light-emitting layer EML may include (e.g., contain) a first compound represented by any one among Formulas F-a to F-c, a second compound represented by Formula HT-1, a third compound represented by Formula ET-1, and a fourth compound represented by Formula D-1.

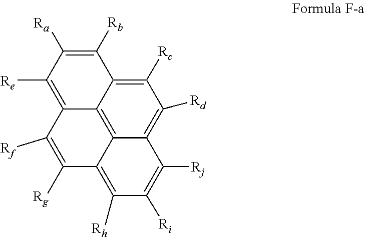

In Formula F-a, two selected from among Rₐ to Rⱼ may each independently be substituted with

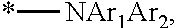

The remainder not substituted with

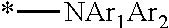

among Rₐ to Rⱼ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In

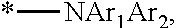

Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one among Ar₁ and Ar₂ may be a heteroaryl group including O or S as a ring-forming atom.

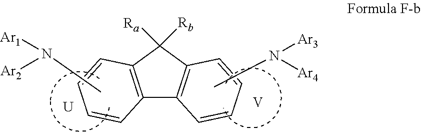

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring.

In Formula F-b, Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, if the number of U or V is 1, one ring forms a fused ring at the designated part by U or V, and if the number of U or V is 0, a ring is not present at the designated part by U or V. for example, if the number of U is 0, and the number of V is 1, or if the number of U is 1, and the number of V is 0, a fused ring having the fluorene core of Formula F-b may be a ring compound with four rings. In some embodiments, if the number of both (e.g., simultaneously) U and V is 0, the fused ring of Formula F-b may be a ring compound with three rings. In some embodiments, if the number of both (e.g., simultaneously) U and V is 1, a fused ring having the fluorene core of Formula F-b may be a ring compound with five rings.

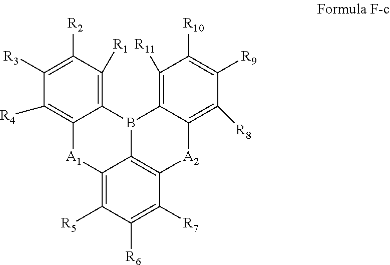

In Formula F-c, A₁ and A₂ may each independently be O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A₁ and A₂ may each independently be combined with the substituents of an adjacent ring to form a fused ring. For example, if A₁ and A₂ may each independently be NRₘ, A₁ may be combined with R₄ or R₅ to form a ring. In some embodiments, A₂ may be combined with R₇ or R₈ to form a ring.

In one or more embodiments, the second compound may be used as a hole transporting host material of the light-emitting layer EML.

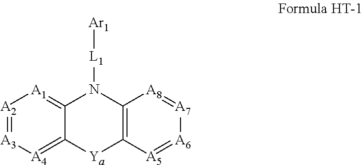

In Formula HT-1, A1 to A5 may each independently be N or CR₅₁. For example, all M₁ to M₈ may be CR₅₁. In one or more embodiments, any one among A1 to A5 may be N, and the remainder may be CR₅₁.

In Formula HT-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, L₁ may be a direct linkage, a substituted or unsubstituted phenylene group, a substituted or unsubstituted divalent biphenyl group, a substituted or unsubstituted divalent carbazole group, and/or the like, but one or more embodiments of the disclosure is not limited thereto.

In Formula HT-1, Yₐ may be a direct linkage, CR₅₂R₅₃, or SiR₅₄R₅₅. For example, it may refer to that two benzene rings connected with the nitrogen atom of Formula HT-1 may be connected via a direct linkage,

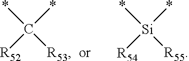

In Formula HT-1, if Yₐ is a direct linkage, the substituent represented by Formula HT-1 may include a carbazole moiety.

In Formula HT-1, Ar₁ may be a substituted or unsubstituted aryl group of 6 to 30 ring-forming carbon atoms, or a substituted or unsubstituted heteroaryl group of 2 to 30 ring-forming carbon atoms. For example, Ar₁ may be a substituted or unsubstituted carbazole group, a substituted or unsubstituted dibenzofuran group, a substituted or unsubstituted dibenzothiophene group, a substituted or unsubstituted biphenyl group, and/or the like, but one or more embodiments of the disclosure is not limited thereto.

In Formula HT-1, R₅₁ to R₅₅ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, each of R₅₁ to R₅₅ may be combined with an adjacent group to form a ring. For example, R₅₁ to R₅₅ may each independently be a hydrogen atom or a deuterium atom. R₅₁ to R₅₅ may each independently be an unsubstituted methyl group or an unsubstituted phenyl group.

In one or more embodiments, the second compound represented by Formula HT-1 may be represented by any one among (e.g., selected from among) the compounds represented in Compound Group 2. A light-emitting layer EML may include at least one among (e.g., selected from among) the compounds represented in Compound Group 2 as a hole transport host material.

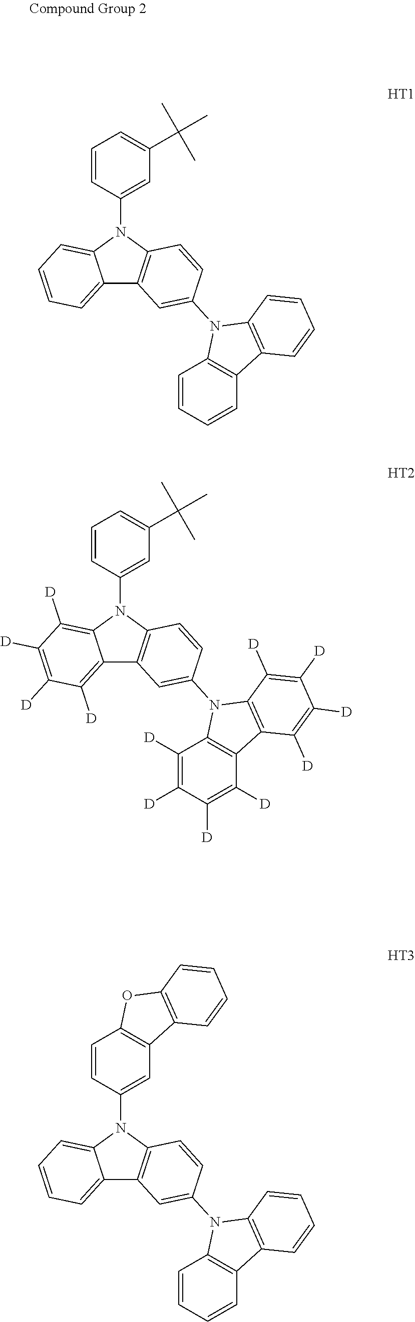 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

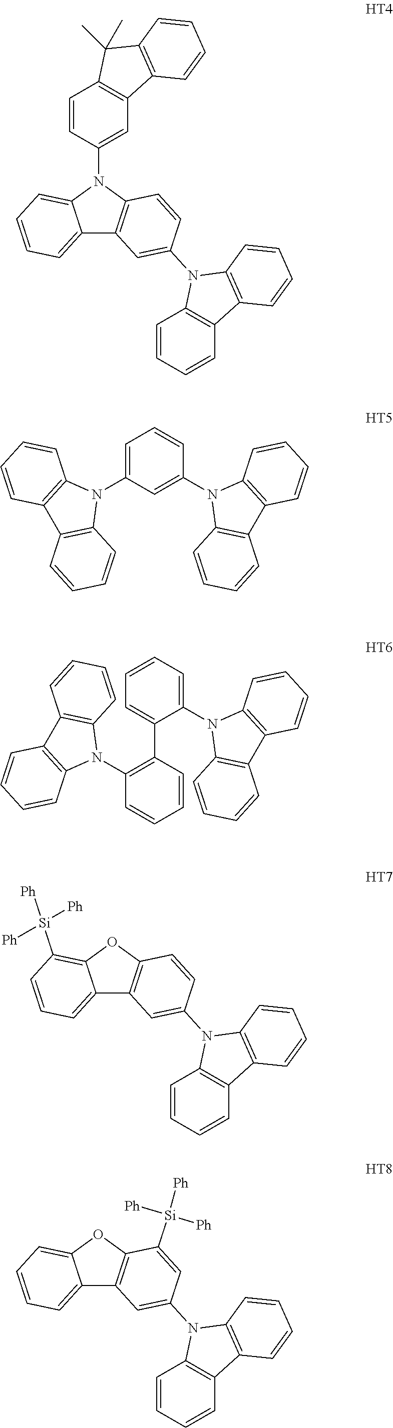 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

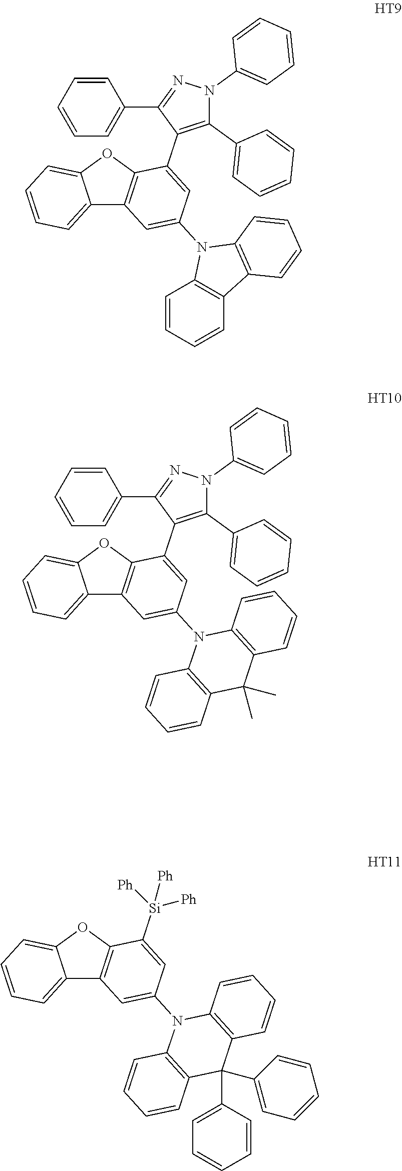 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

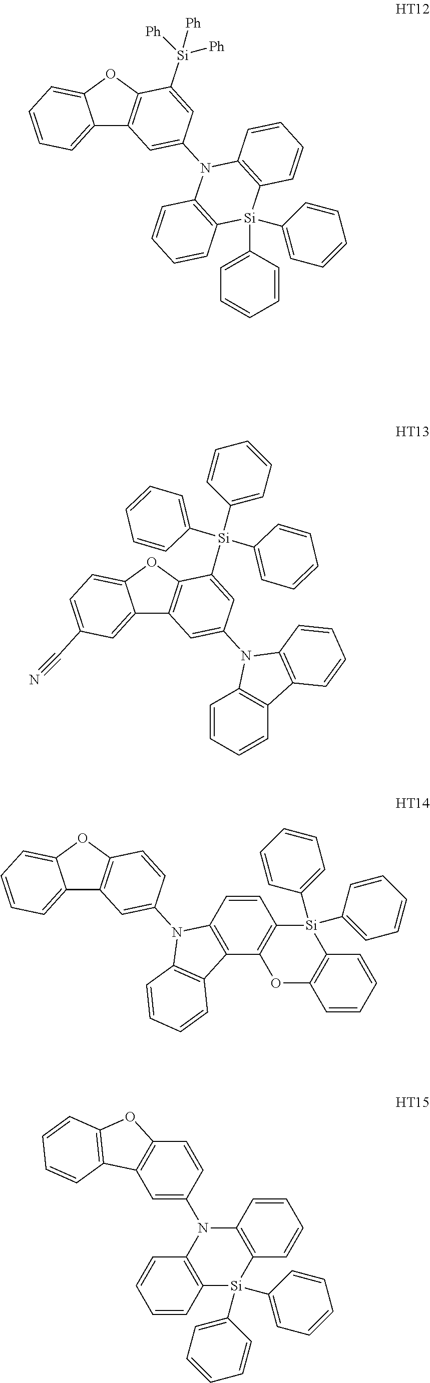 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1]

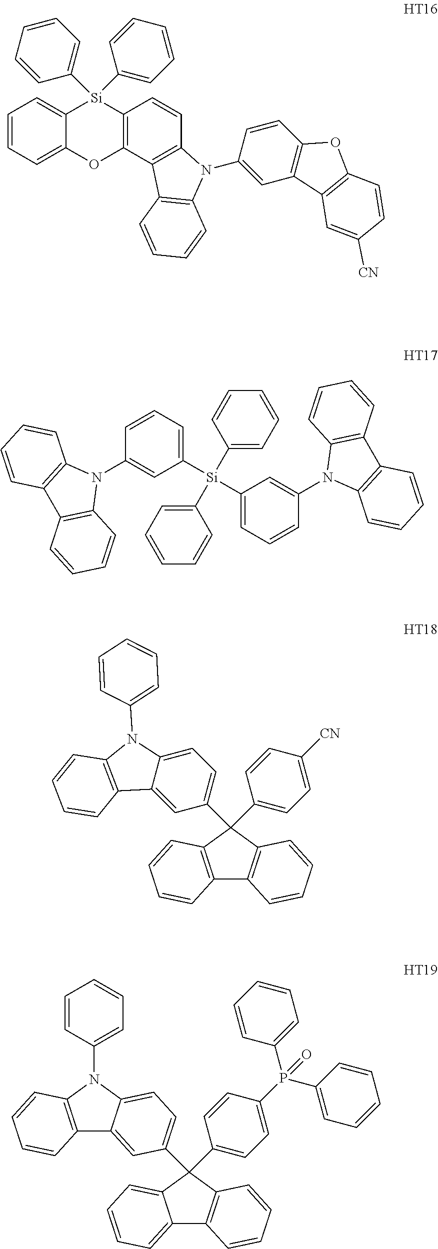 [SMILES: N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1]

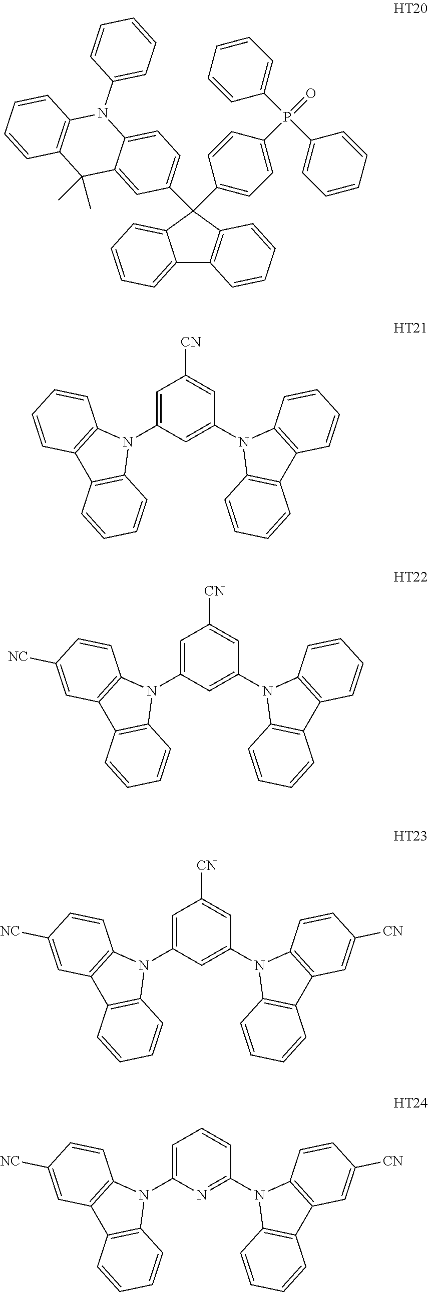 [SMILES: CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1]

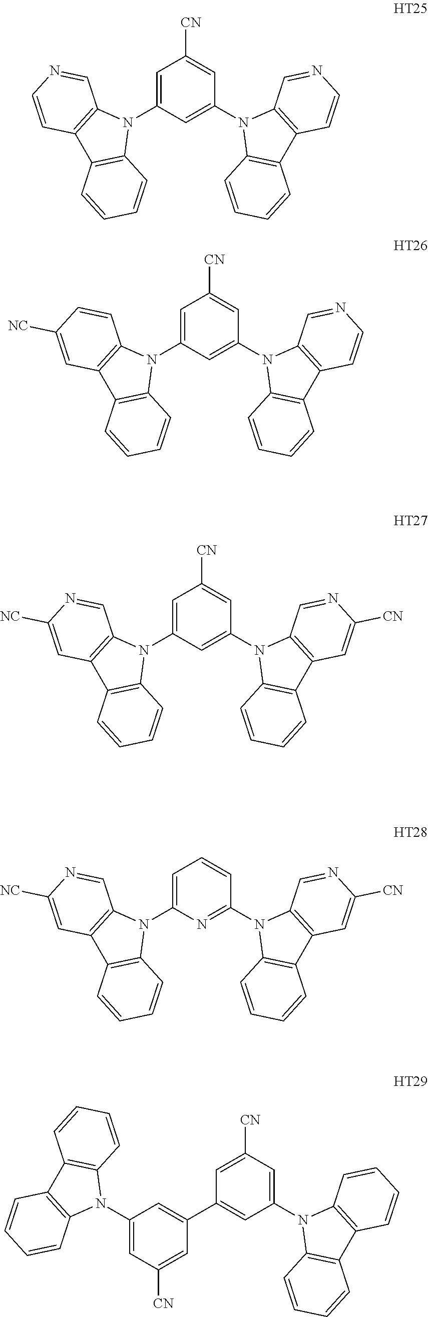 [SMILES: N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1]

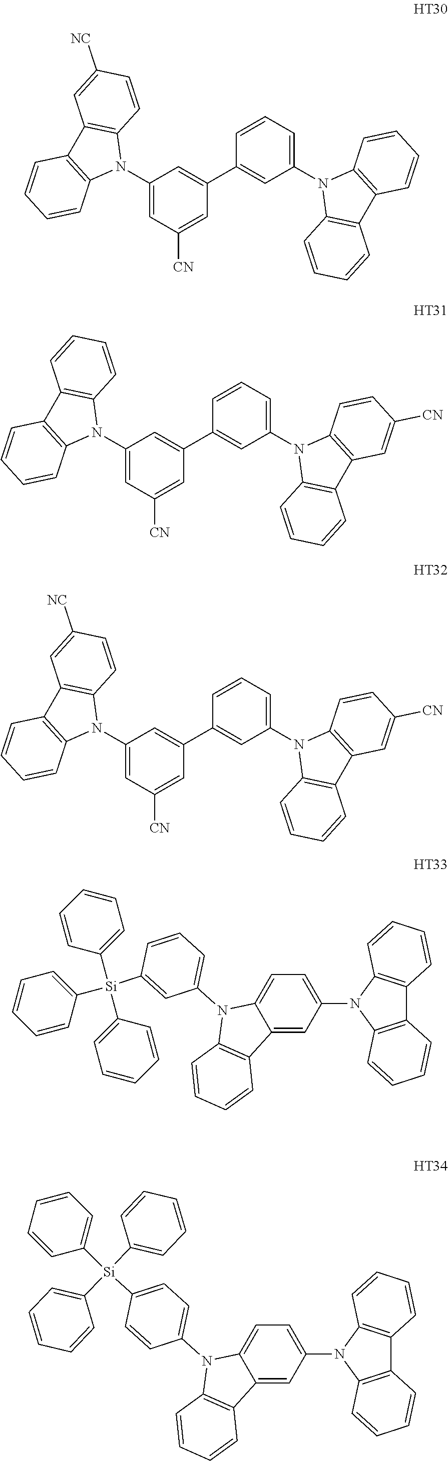 [SMILES: N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1 ; c1ccc([Si](c2ccccc2)(c2ccccc2)c2cccc(-n3c4ccccc4c4cc(-n5c6ccccc6c6ccccc65)ccc43)c2)cc1 ; c1ccc([Si](c2ccccc2)(c2ccccc2)c2ccc(-n3c4ccccc4c4cc(-n5c6ccccc6c6ccccc65)ccc43)cc2)cc1]

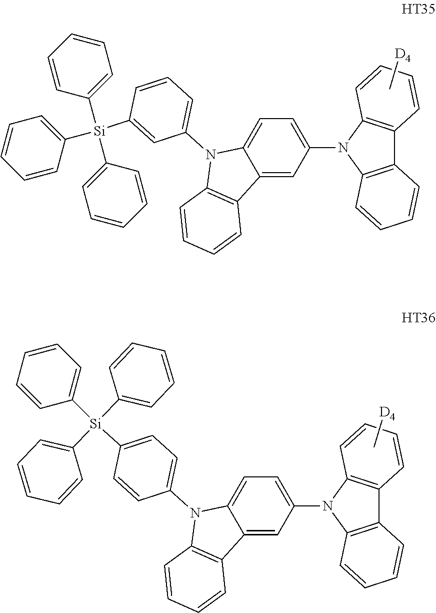

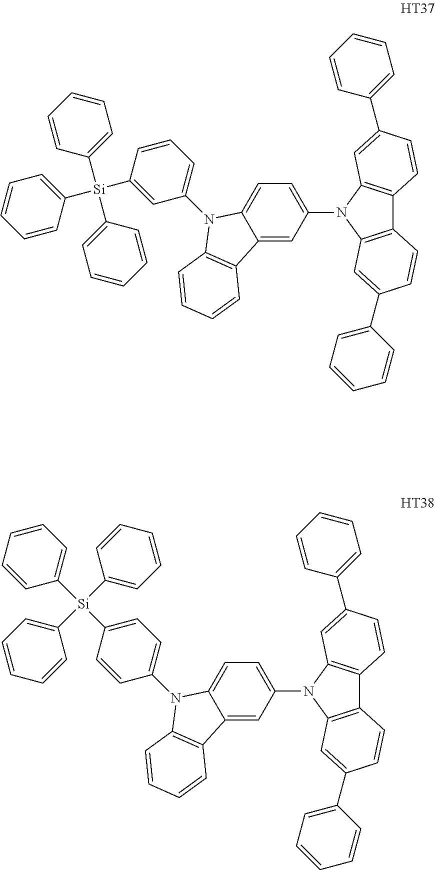 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1]

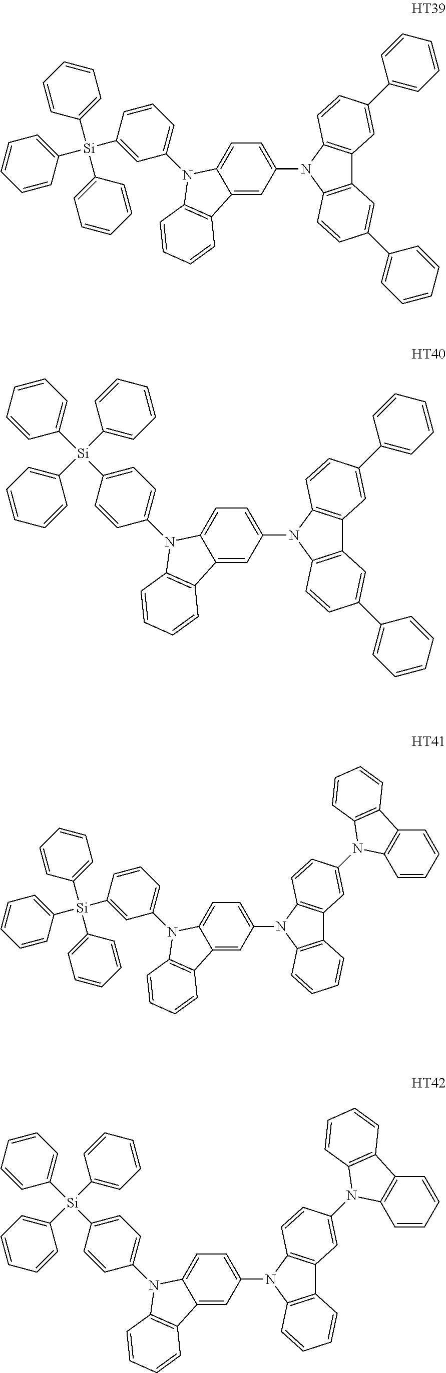 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

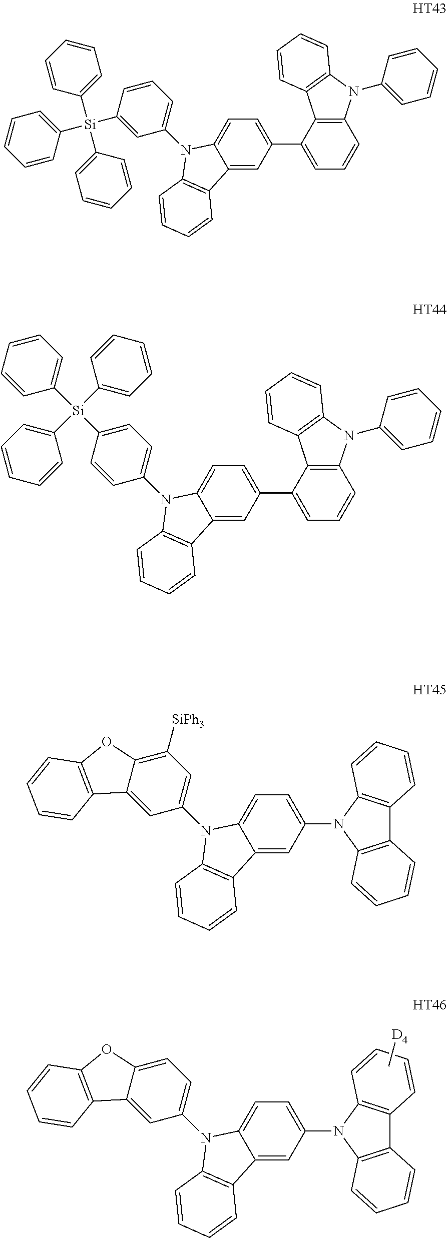

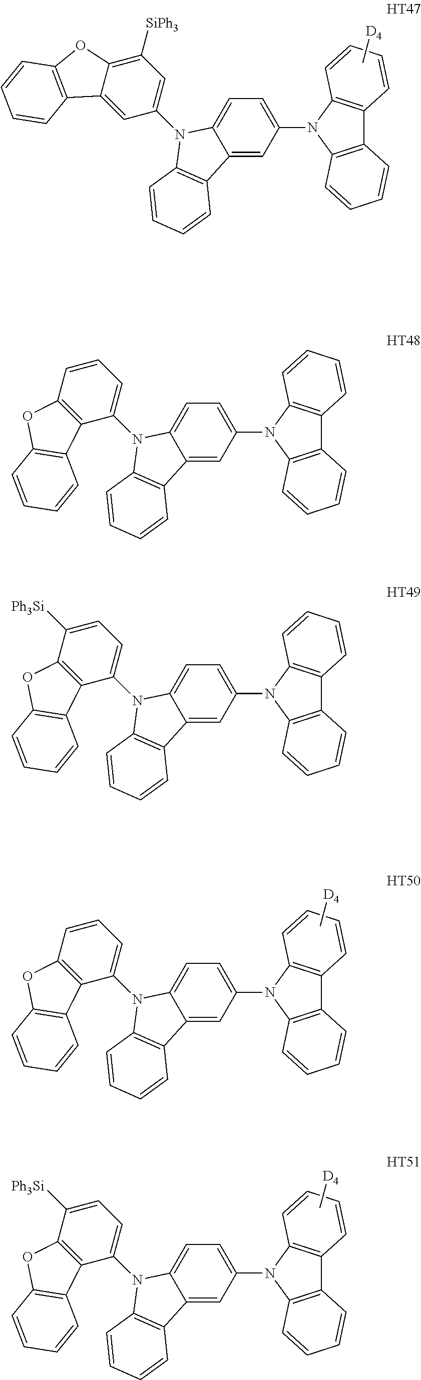

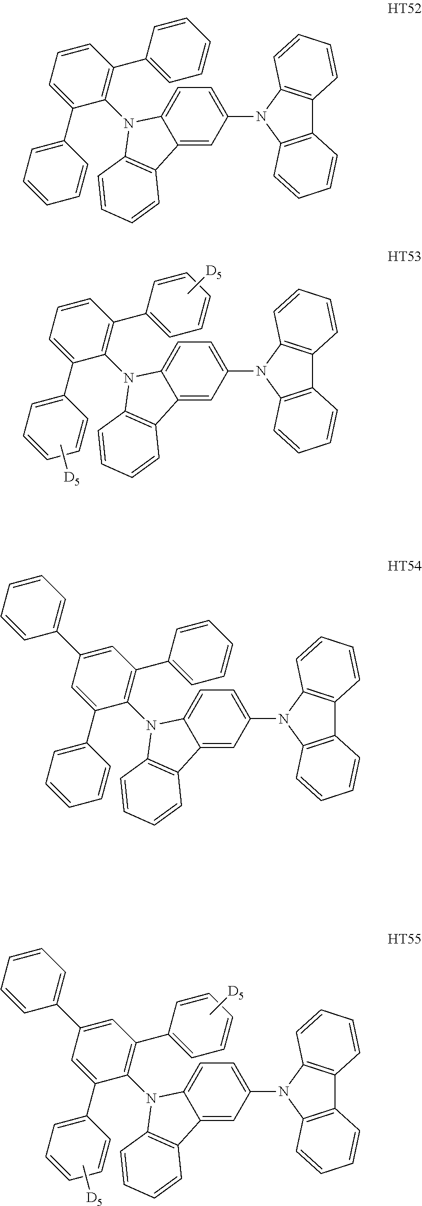

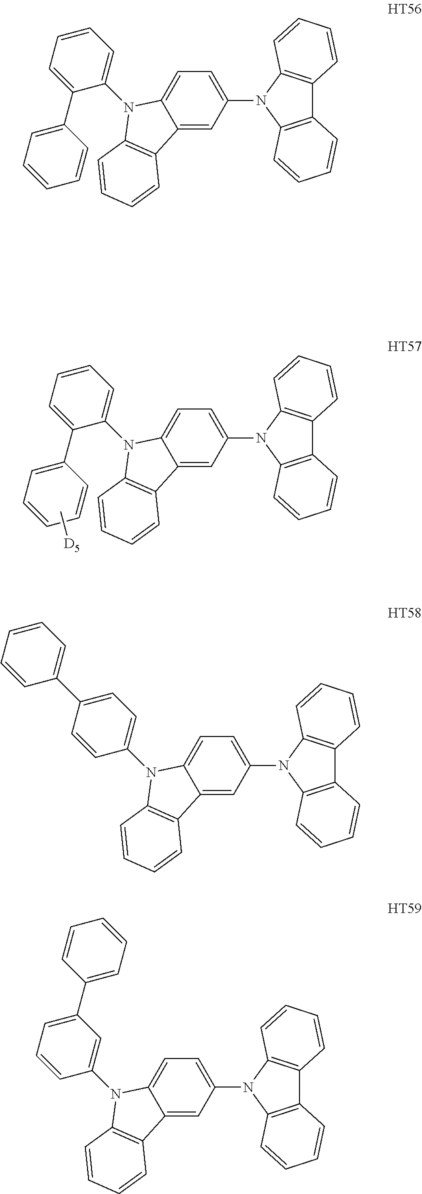

In the compounds suggested in Compound Group 2, “D” refers to a deuterium atom, and “Ph” may refer to a substituted or unsubstituted phenyl group. For example, in the particular compounds suggested in Compound Group 2, “Ph” may be an unsubstituted phenyl group.

In one or more embodiments, the light-emitting layer EML may contain the third compound represented by Formula ET-1. For example, the third compound may be used as an electron transporting host material of the light-emitting layer EML.

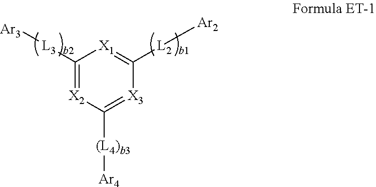

In Formula ET-1, at least one among X₁ to X₃ may be N, and the remainder may be CR₅₆. For example, at least one among X₁ to X₃ may be N, and the remainder two may each independently be CR₅₆. In this case, the third compound represented by Formula ET-1 may include a pyridine moiety. In one or more embodiments, at least two among (e.g., selected from among) X₁ to X₃ may be N, and the remainder may be CR₅₆. In this case, the third compound represented by Formula ET-1 may include a pyrimidine moiety. In one or more embodiments, X₁ to X₃ may be all N. In this case, the third compound represented by Formula ET-1 may include a triazine moiety.

In Formula ET-1, R₅₆ may be a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

In Formula ET-1, b1 to b3 may each independently be an integer of 0 to 10.

In Formula ET-1, Ar₂ to Ar₄ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Ar₂ to Ar₄ may be substituted or unsubstituted phenyl groups or substituted or unsubstituted carbazole groups.

In Formula ET-1, L₂ to L₄ may each independently be a direct linkage, a substituted or unsubstituted arylene group of 6 to 30 ring-forming carbon atoms, or a substituted or unsubstituted heteroarylene group of 2 to 30 ring-forming carbon atoms.

In one or more embodiments, if each of b1 to b3 is an integer of 2 or more, L₂ to L₄ may each independently be a substituted or unsubstituted arylene group of 6 to 30 ring-forming carbon atoms, or a substituted or unsubstituted heteroarylene group of 2 to 30 ring-forming carbon atoms.

In one or more embodiments, the third compound may be represented by any one among (e.g., selected from among) the compounds in Compound Group 3. The light-emitting element ED of one or more embodiments may include any or at least one among (e.g., selected from among) the compounds in Compound Group 3.

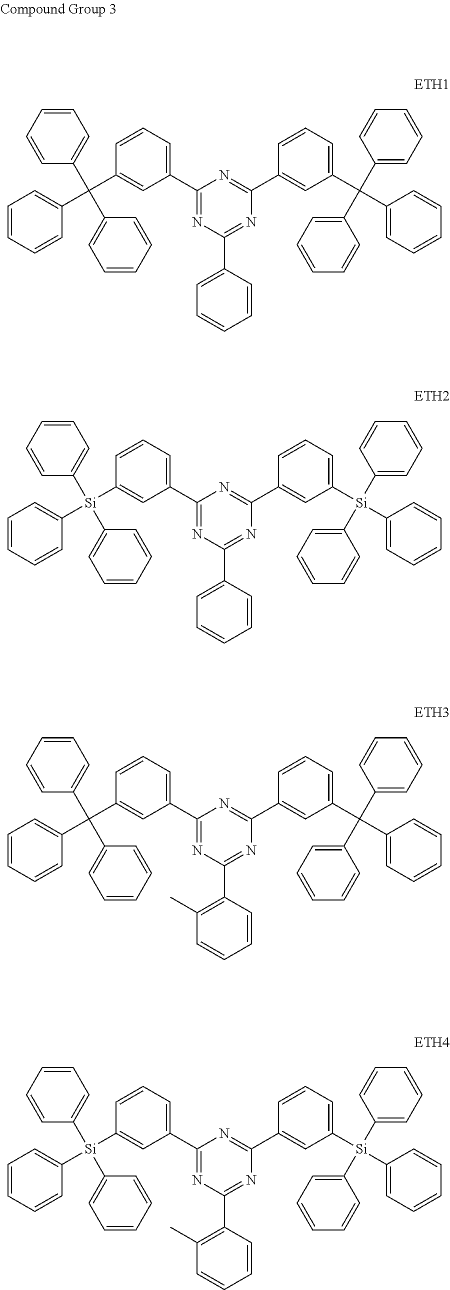 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

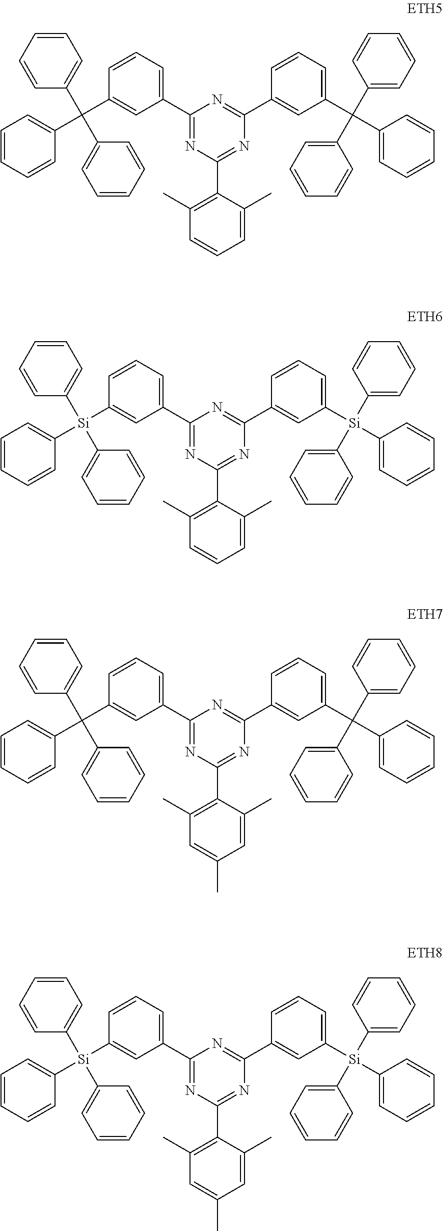 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

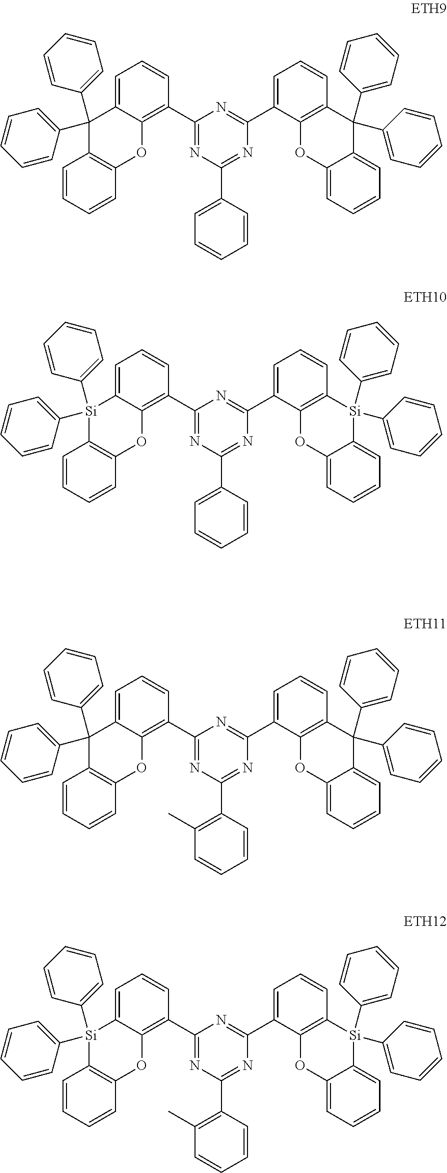 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

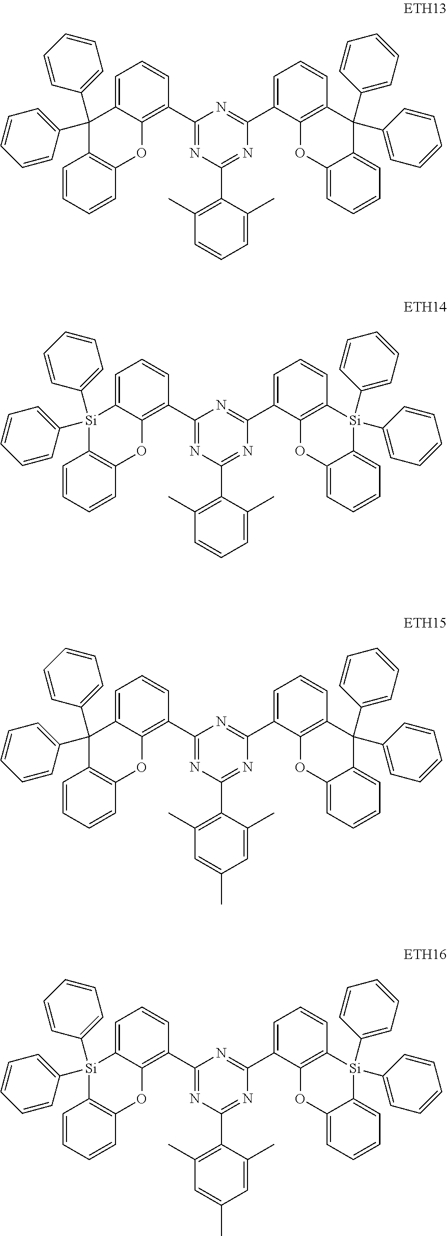 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

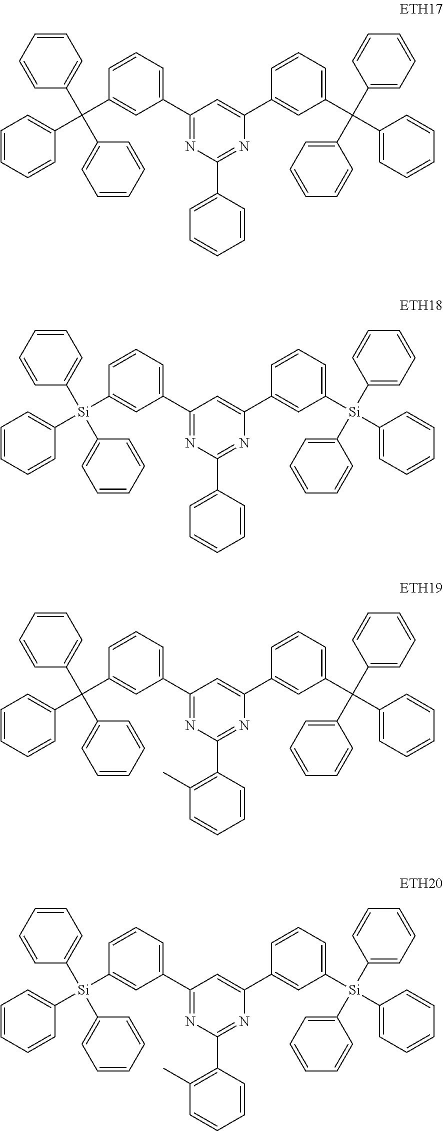 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

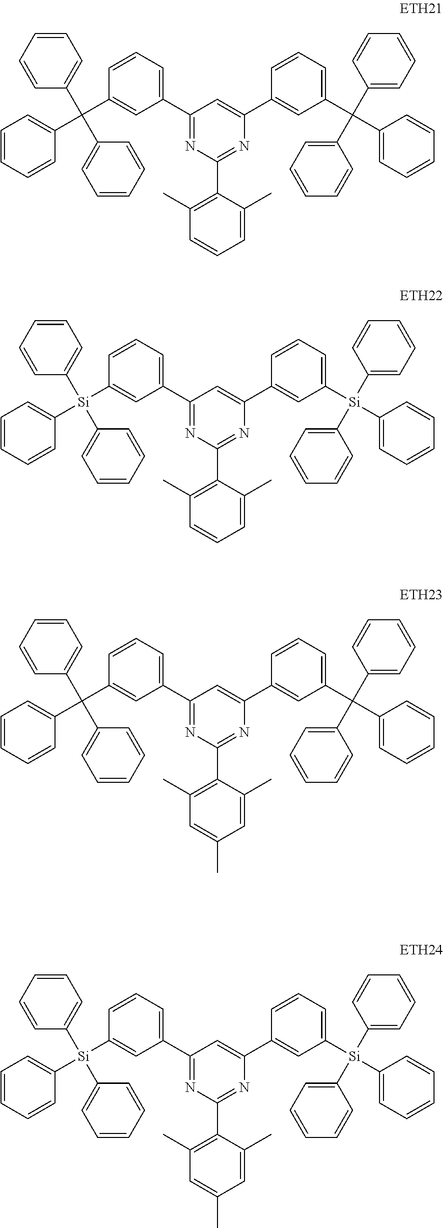 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

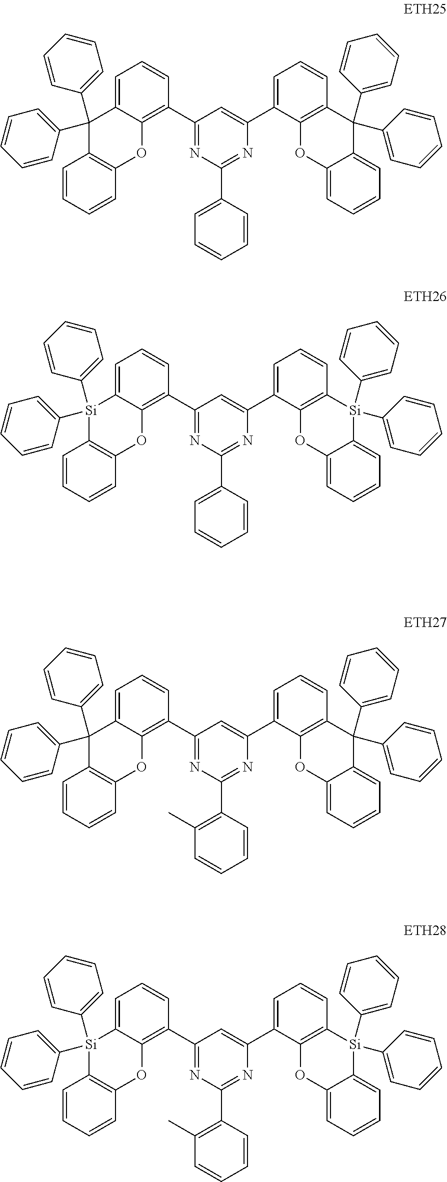 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

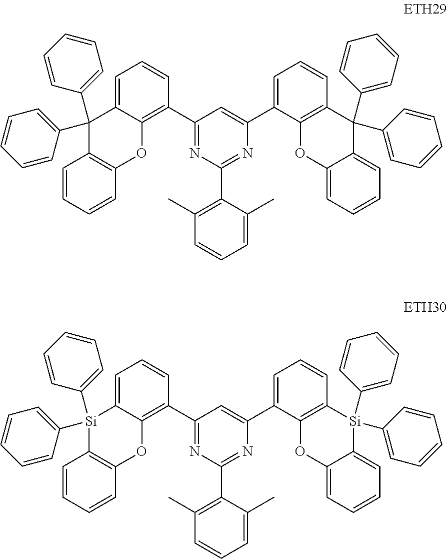 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

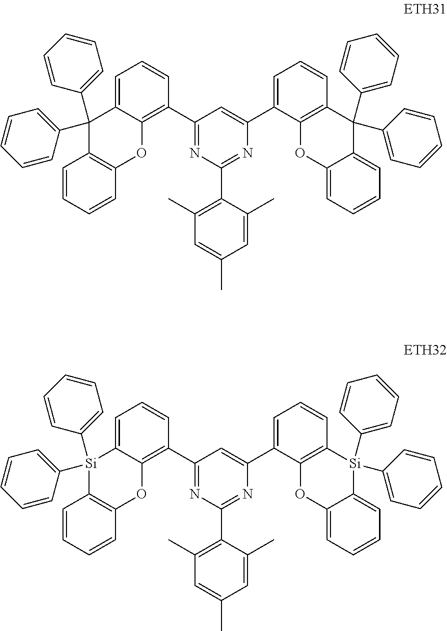 [SMILES: Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

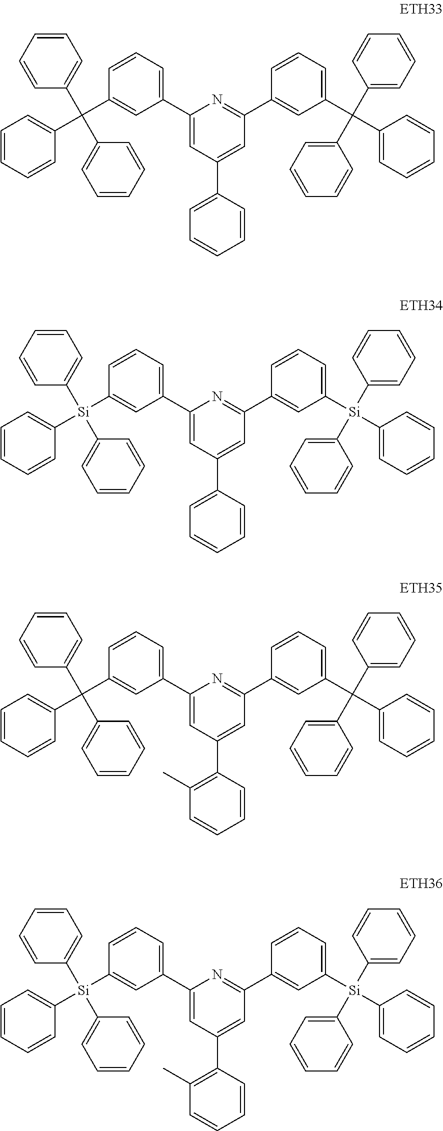 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

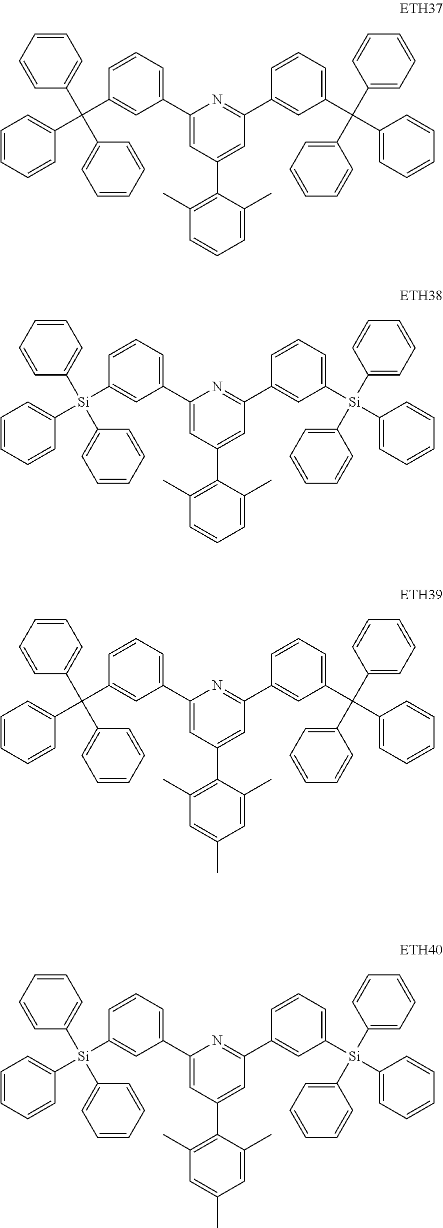 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

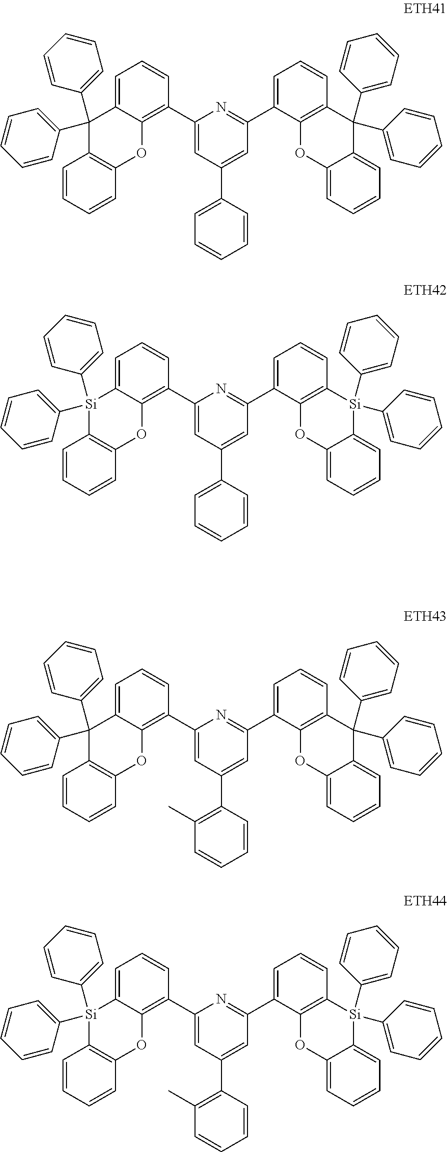 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

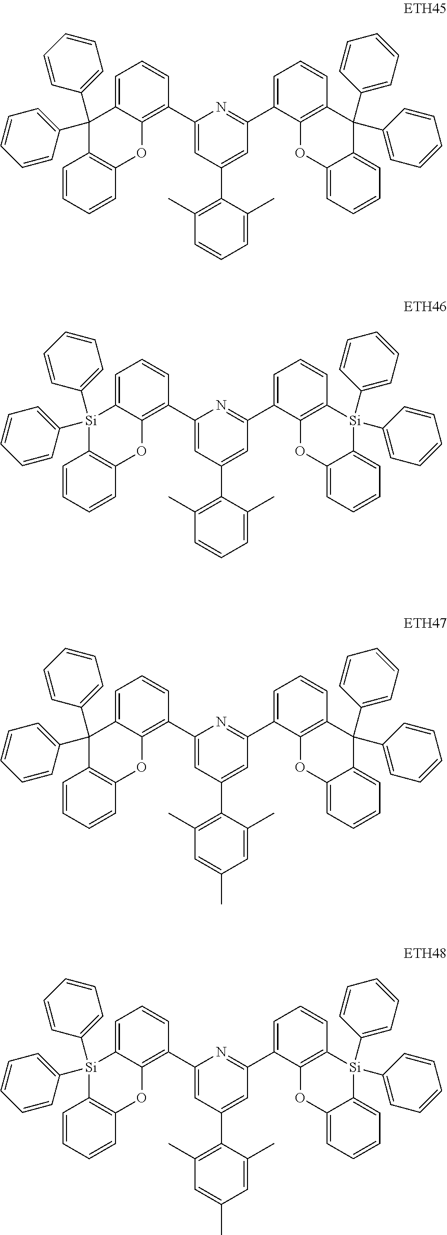 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

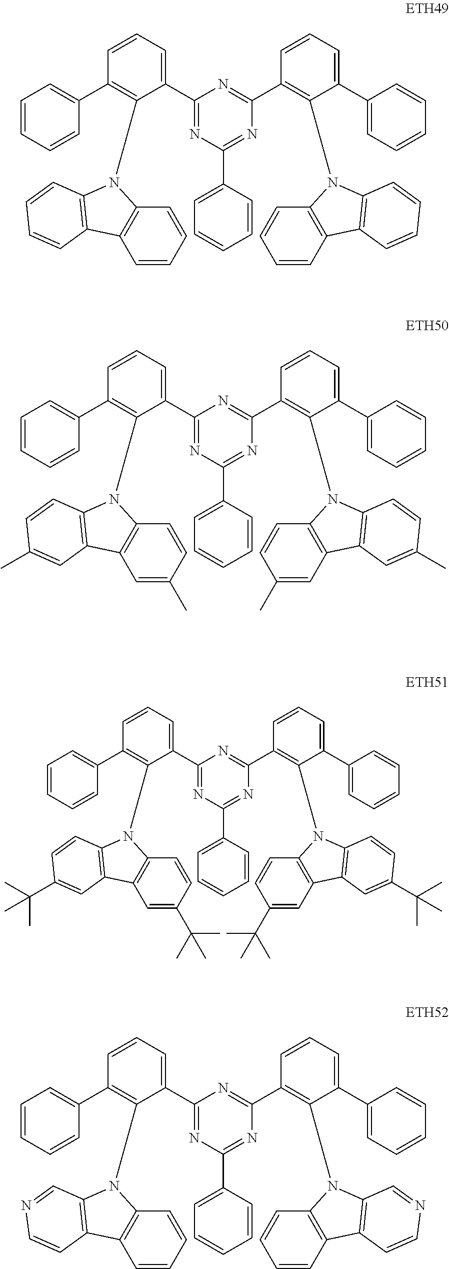

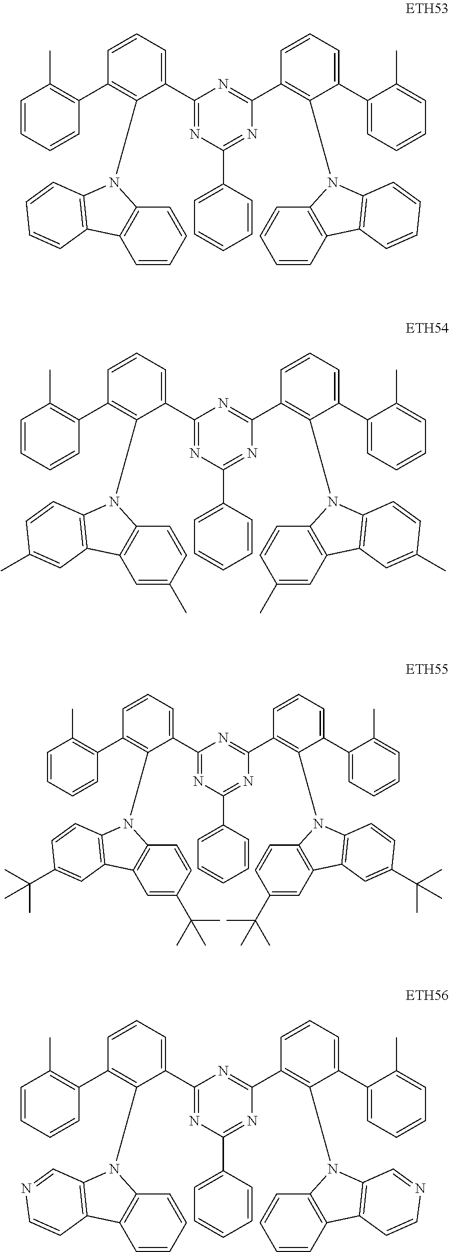

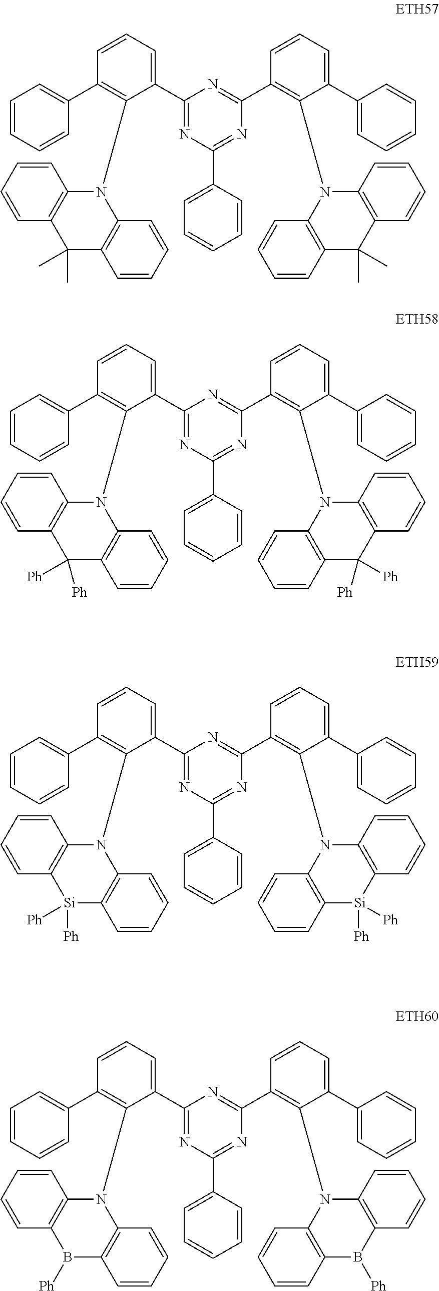

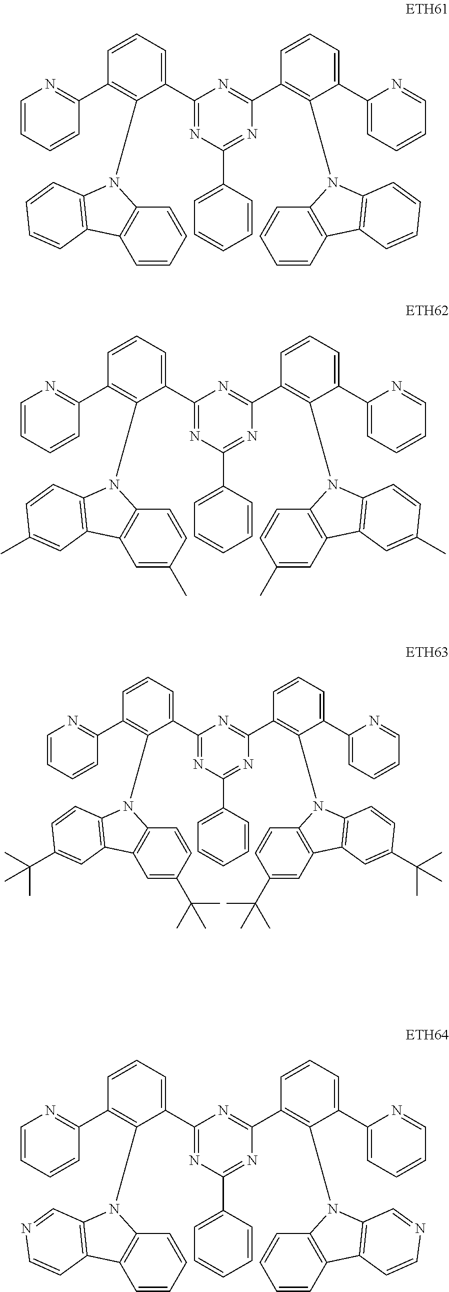

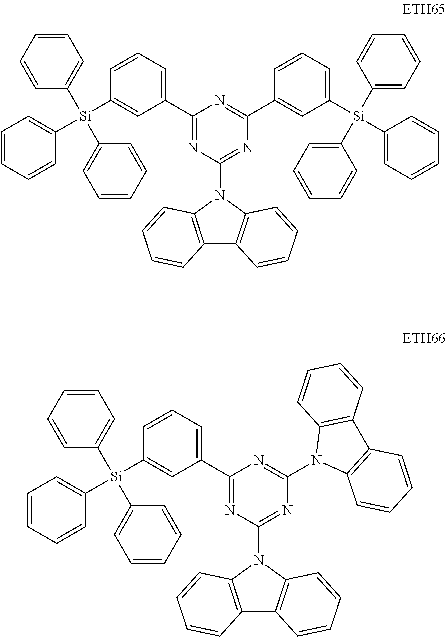 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

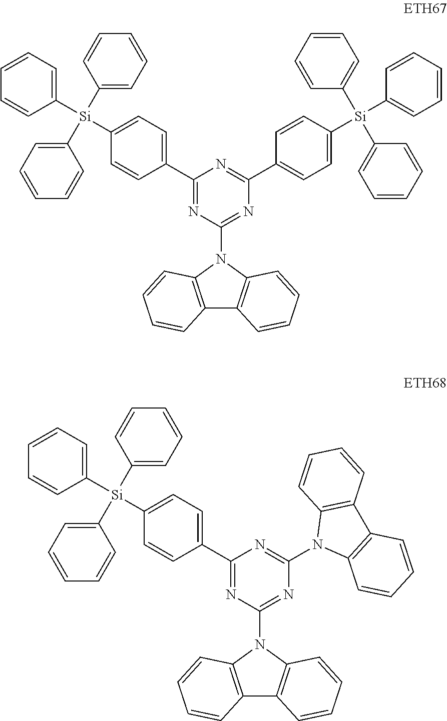 [SMILES: c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

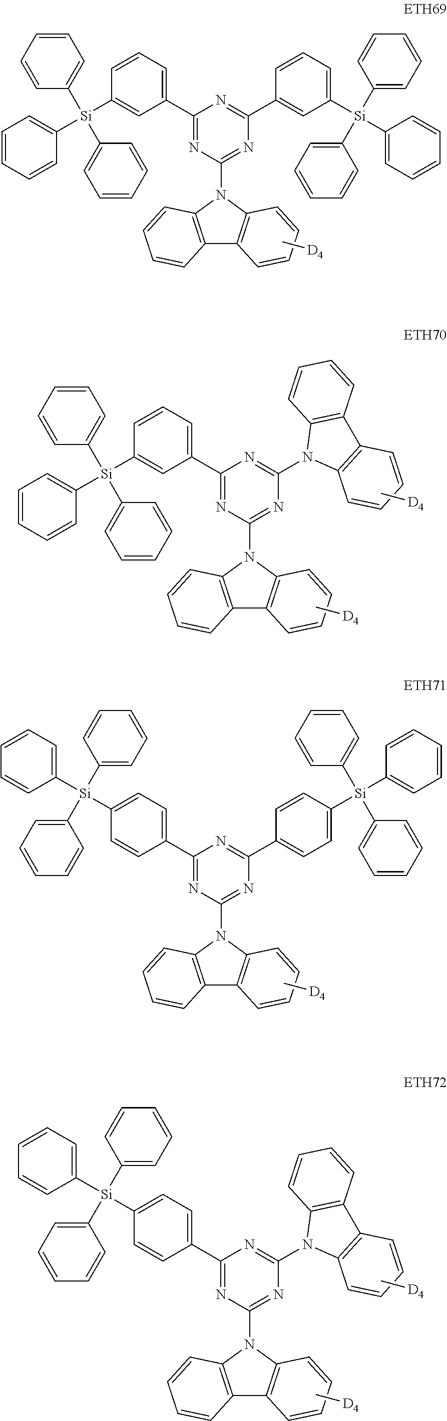

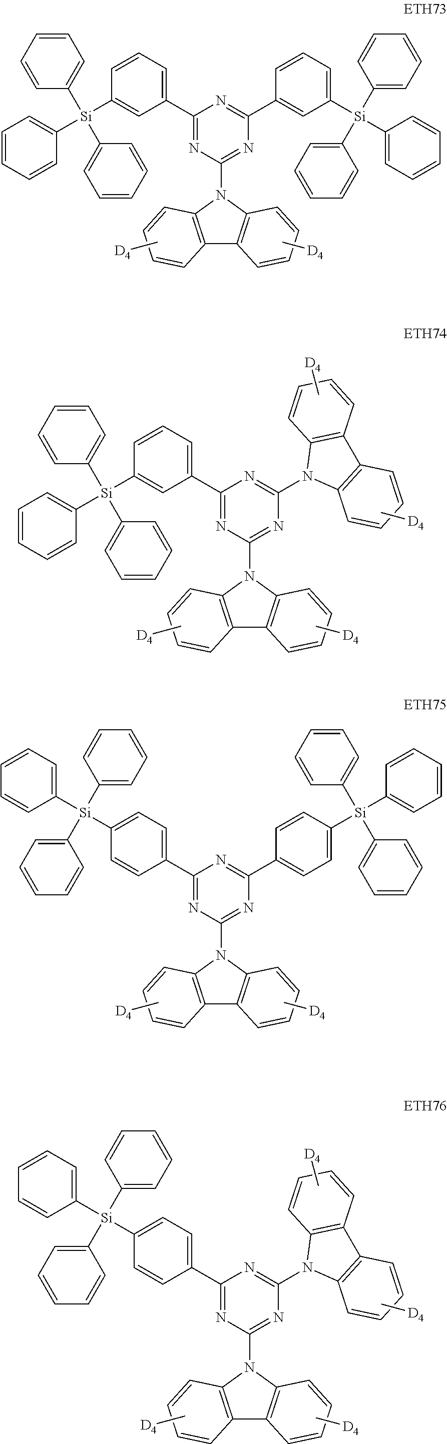

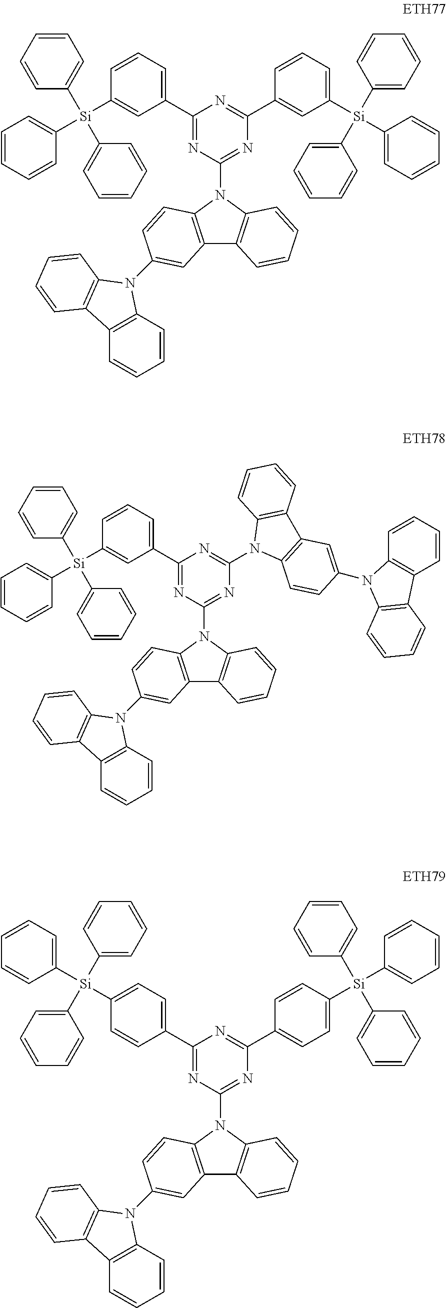 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

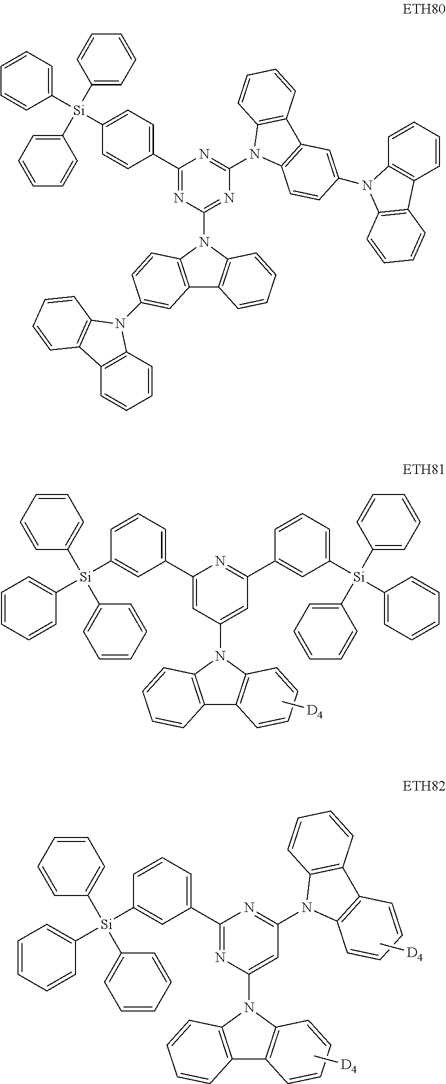

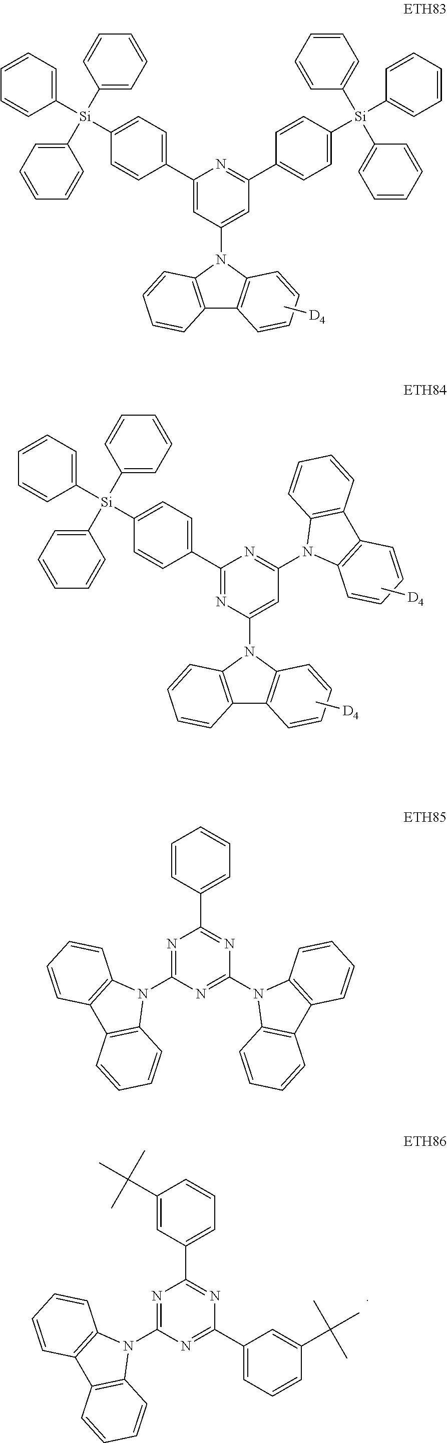

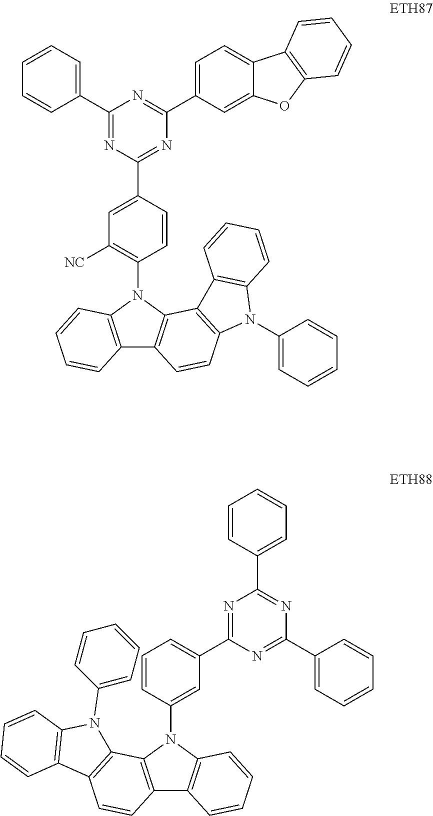 [SMILES: N#Cc1cc(-c2nc(-c3ccccc3)nc(-c3ccc4c(c3)oc3ccccc34)n2)ccc1-n1c2ccccc2c2ccc3c(c4ccccc4n3-c3ccccc3)c21 ; c1ccc(-c2nc(-c3ccccc3)nc(-c3cccc(-n4c5ccccc5c5ccc6c7ccccc7n(-c7ccccc7)c6c54)c3)n2)cc1]

In the particular compounds suggested in Compound Group 3, “D” refers to a deuterium atom, and “Ph” refers to an unsubstituted phenyl group.

The light-emitting layer EML may include the second compound and the third compound, and the second compound and the third compound may form exciplex. In the light-emitting layer EML, exciplex may be formed by a hole transport host and an electron transport host. In this case, the triplet energy of the exciplex formed by the hole transport host and the electron transport host may correspond to a difference between the lowest unoccupied molecular orbital (LUMO) energy level of the electron transport host and the highest occupied molecular orbital (HOMO) energy level of the hole transport host.

For example, the absolute value of the triplet energy level (T1) of the exciplex formed by the hole transport host and the electron transport host may be about 2.4 electron volt (eV) to about 3.0 eV. In some embodiments, the triplet energy of the exciplex may be a smaller value than the energy gap of each host material. The exciplex may have a triplet energy of about 3.0 eV or less, that is the energy gap between the hole transport host and the electron transport host.

In one or more embodiments, the light-emitting layer EML may include a fourth compound in addition to the first compound to the third compound. The fourth compound may be used as a phosphorescence sensitizer of a light-emitting layer EML. Because energy may transfer from the fourth compound to the first compound, light emission may arise.

For example, the light-emitting layer EML may include an organometallic complex which contains platinum (Pt) as a center metal atom and contain ligands bonded to the center metal atom, as the fourth compound. In the light-emitting element ED according to one or more embodiments, the light-emitting layer EML may contain a compound represented by Formula D-1 as the fourth compound.

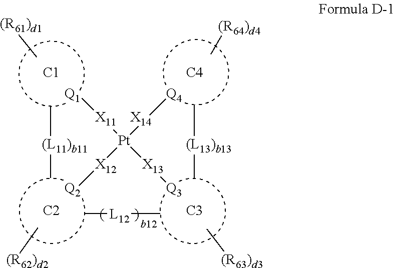

In Formula D-1, Q₁ to Q₄ may each independently be C or N.

In Formula D-1, C1 to C4 may each independently be a substituted or unsubstituted hydrocarbon ring of 5 to 30 ring-forming carbon atoms, or a substituted or unsubstituted heterocycle of 2 to 30 ring-forming carbon atoms.

In Formula D-1, X₁₁ to X₁₄ may each independently be a direct linkage, or

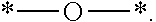

For example, any one of the X₁₁ to X₁₄ may be

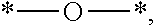

and the remainder may be a direct linkage.

In Formula D-1, L₁₁ to L₁₃ may each independently be a direct linkage,

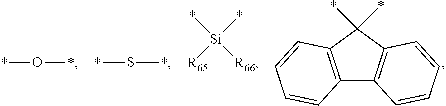

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃, “ [symbol] ” refers to a part connected with C1 to C4.

In Formula D-1, b11 to b13 may each independently be 0 or 1. If b11 is 0, C1 and C2 may be unconnected. If b12 is 0, C2 and C3 may be unconnected. If b13 is 0, C3 and C4 may be unconnected.

In Formula D-1, R₆₁ to R₆₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, each of R₆₁ to R₆₆ may be combined with an adjacent group to form a ring. R₆₁ to R₆₆ may each independently be a substituted or unsubstituted methyl group, or a substituted or unsubstituted t-butyl group.

In Formula D-1, d1 to d4 may each independently be an integer of 0 to 4. In Formula D-1, if d1 to d4 are 0, the fourth compound may be unsubstituted with R₆₁ to R₆₄, respectively. A case where d1 to d4 are 4, and R₆₁ to R₆₄ are hydrogen atoms, may be the same as a case where d1 to d4 are 0. If d1 to d4 are integers of 2 or more, each of multiple R₆₁ to R₆₄ may be all the same, or at least one among multiple R₆₁ to R₆₄ may be different.

In Formula D-1, C1 to C4 may each independently be a substituted or unsubstituted hydrocarbon ring or a substituted or unsubstituted heterocycle, represented by any one among C-1 to C-5.

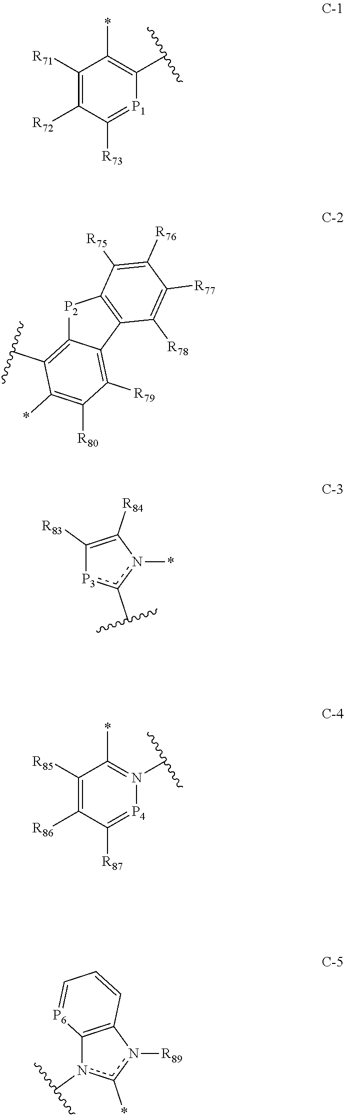

In C-1 to C-5, P₁ may be [symbol] or CR₇₄, P₂ may be [symbol] or NR₈₁, P₃ may be [symbol] or NR₈₂, and P₄ may be [symbol] or CR₈₈, P₆ may be [symbol] or CR₉₀. R₇₁ to R₉₀ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In some embodiments, in C-1 to C-5,

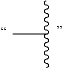

is a part connected with a central metal atom of Pt, and “ [symbol] ” corresponds to a part connected with an adjacent ring group (C1 to C4) or linker (L₁₁ to L₁₃).

The light-emitting layer EML of one or more embodiments may include the first compound that is a fused polycyclic compound, and at least one among the second to fourth compounds. For example, the light-emitting layer EML may include the first compound, the second compound and the third compound. In the light-emitting layer EML, the second compound and the third compound may form exciplex, and in the exciplex, energy transfer to the first compound may arise, and light emission may arise.

In some embodiments, the light-emitting layer EML may include the first compound, the second compound, the third compound and the fourth compound. In the light-emitting layer EML, the second compound and the third compound may form exciplex, and in the exciplex, energy transfer to the fourth compound and the first compound may arise, and light emission may arise. In one or more embodiments, the fourth compound may be a sensitizer. In the light-emitting element ED of one or more embodiments, the fourth compound included in the light-emitting layer EML may act as a sensitizer and may play the role of transferring energy from a host to the first compound that is a light-emitting dopant. For example, the fourth compound that plays the role of an auxiliary dopant may accelerate energy transfer to the first compound that is a light emitting dopant and increase the light emitting ratio of the first compound. Accordingly, the emission efficiency of the light-emitting layer EML of one or more embodiments may be improved. In some embodiments, if the energy transfer to the first compound increases, excitons formed in the light-emitting layer EML may not be accumulated but rapidly emit light, and the deterioration of a device may be reduced. Accordingly, the lifetime of the light-emitting element ED of one or more embodiments may increase.

The light-emitting element ED of one or more embodiments includes all of the first compound, the second compound, the third compound and the fourth compound, and the light-emitting layer EML may include the combination of two host materials and two dopant materials. In the light-emitting element ED of one or more embodiments, the light-emitting layer EML may include the second compound and the third compound, which are two different hosts, the first compound which emits delayed fluorescence, and the fourth compound including an organometallic complex, concurrently (e.g., simultaneously), and may show excellent or suitable emission efficiency properties.

In one or more embodiments, the fourth compound represented by Formula D-1 may be represented by at least or any one among (e.g., selected from among) the compounds represented in Compound Group 4. The light-emitting layer EML may include at least one among (e.g., selected from among) the compounds represented in Compound Group 4 as a sensitizer material.

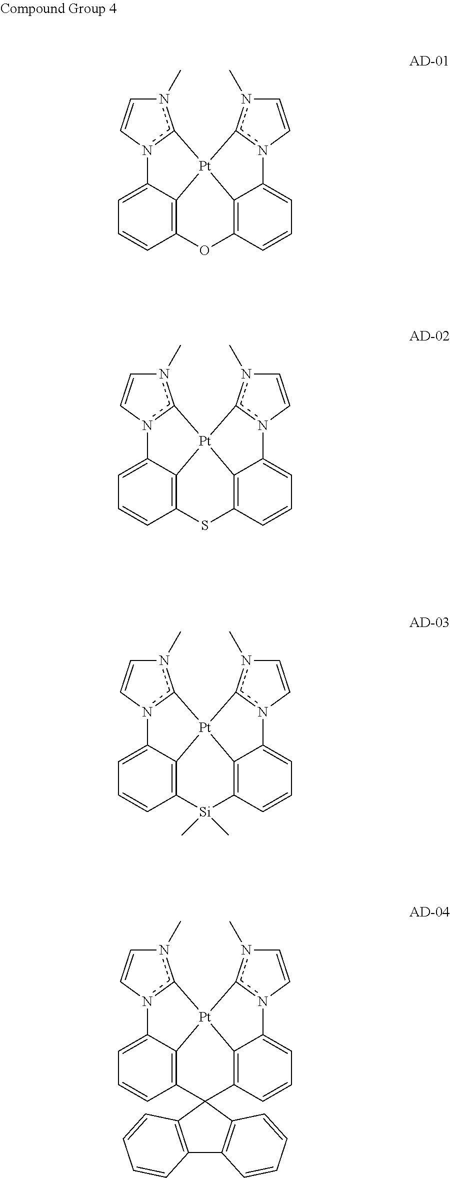

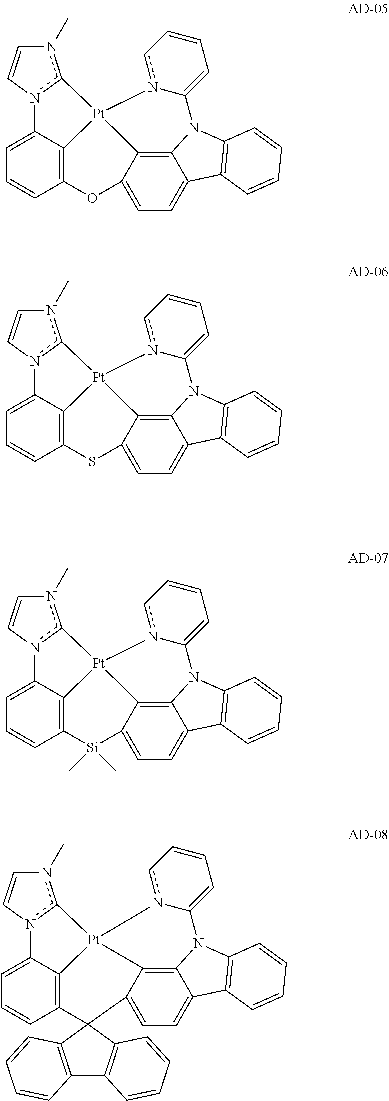

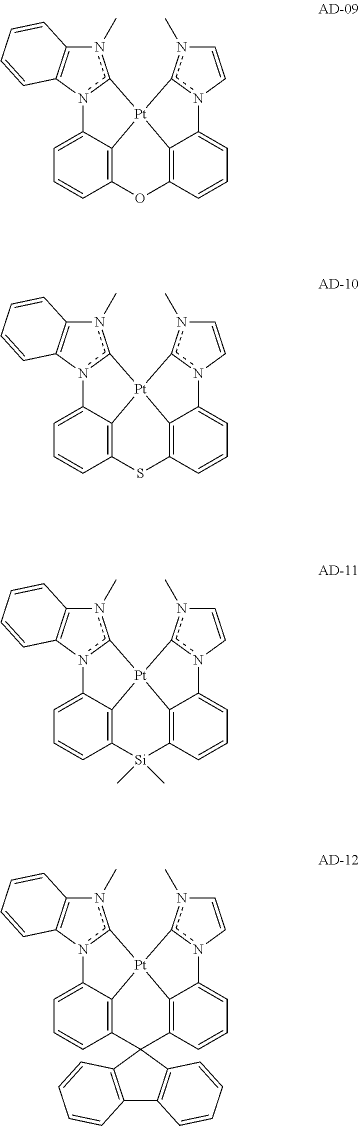

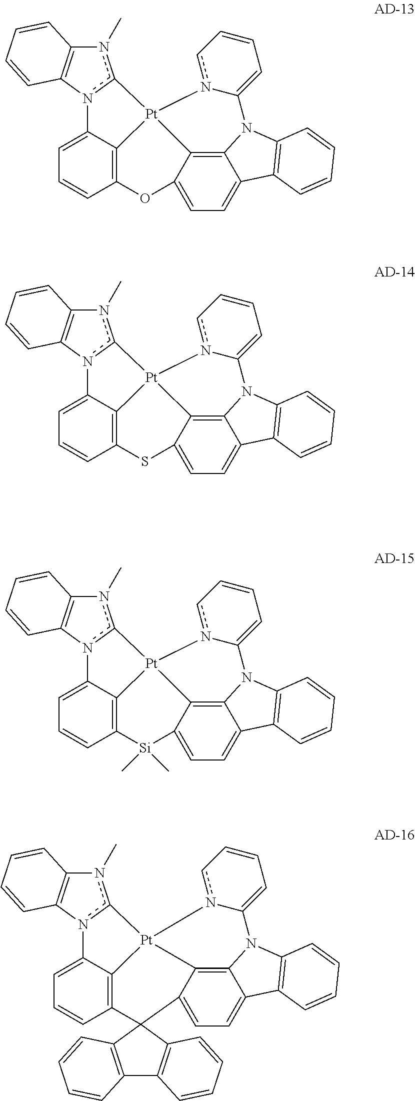

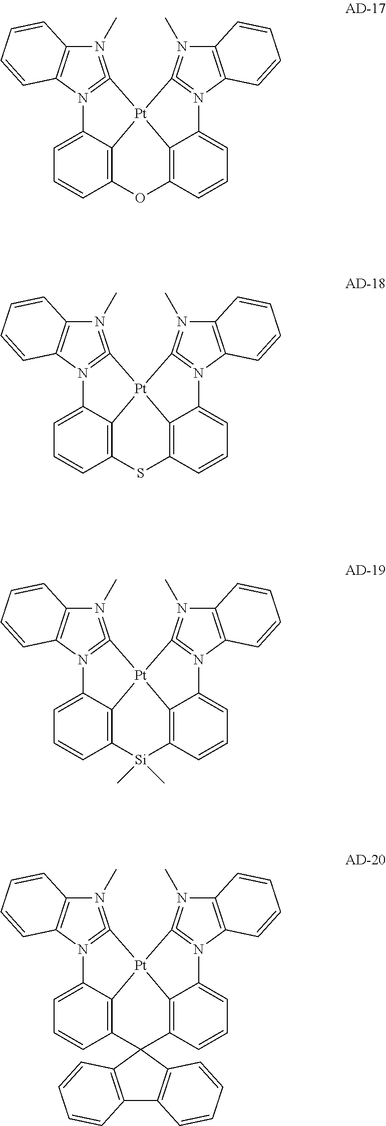

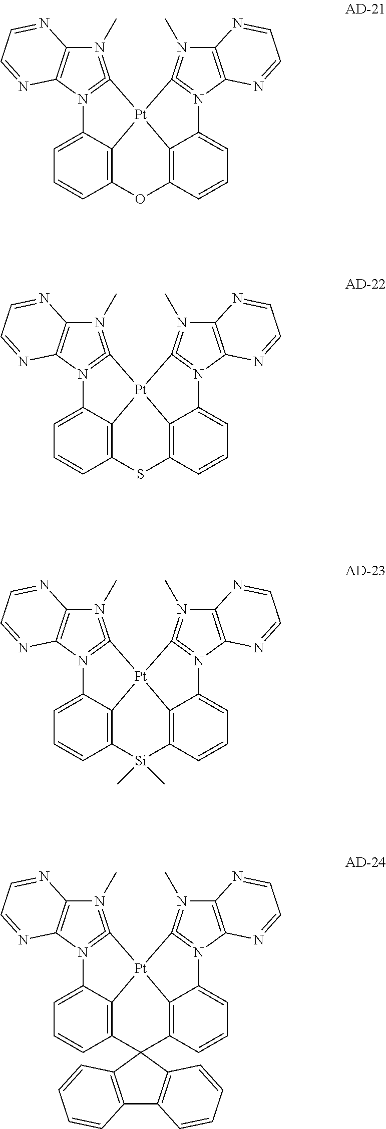

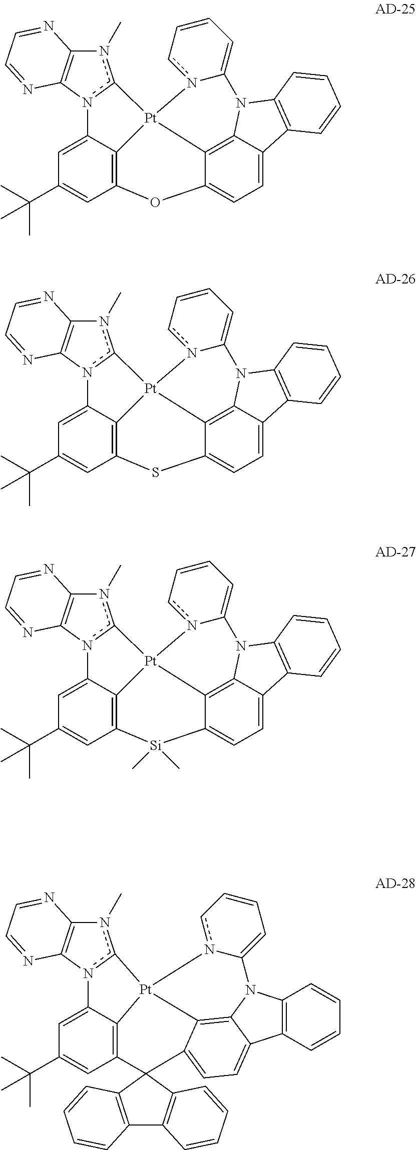

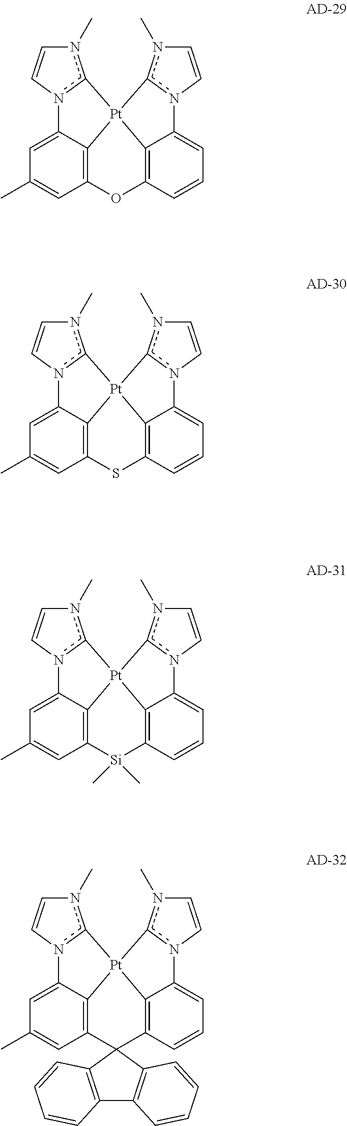

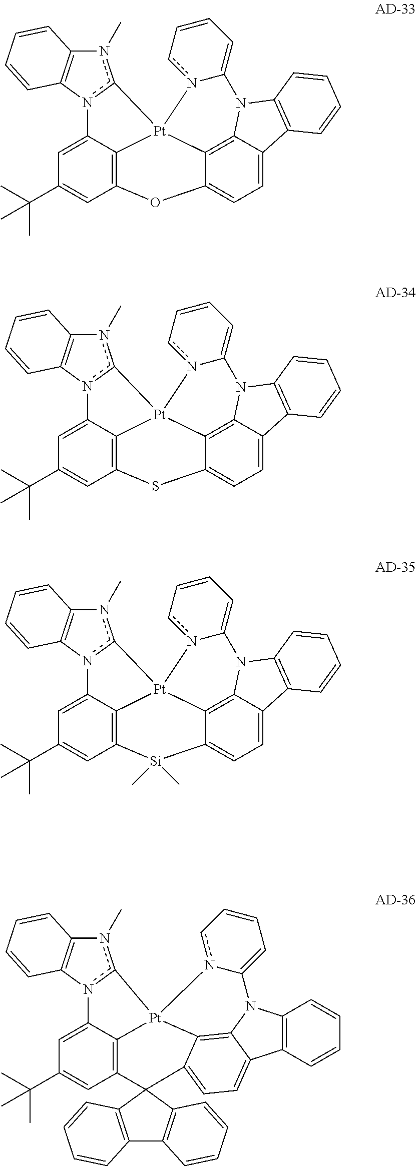

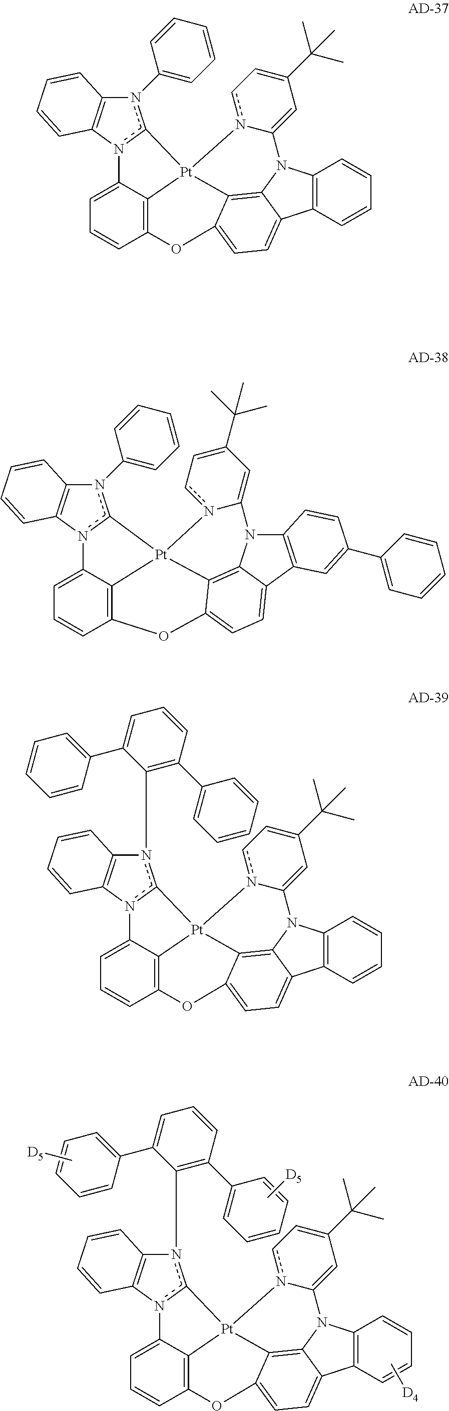

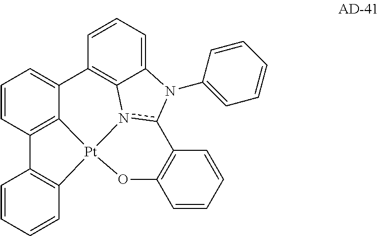

In the particular compounds suggested in Compound Group 4, “D” refers to a deuterium atom.

In the light-emitting element ED of one or more embodiments, if the light-emitting layer EML includes all of the first compound, the second compound, the third compound, and the fourth compound, the amount of the first compound may be about 0.1 wt % to about 5 wt % based on the total weight of the first compound, the second compound, the third compound, and the fourth compound. However, one or more embodiments of the disclosure is not limited thereto. If the amount of the first compound satisfies the herein-described ratio, energy transfer from the second compound and the third compound to the first compound may increase, and accordingly, the emission efficiency and device lifetime may increase.

In the light-emitting layer EML, the total amount of the second compound and the third compound may be the remaining amount excluding the amount of the first compound and the fourth compound. For example, the total amount of the second compound and the third compound may be about 65 wt % to about 95 wt % based on the total weight of the first compound, the second compound, the third compound, and the fourth compound.

In the total amount of the second compound and the third compound, the weight ratio of the second compound and the third compound may be about 3:7 to about 7:3.

If the total amount of the second compound and the third compound satisfies the herein-described ratio, charge balance properties in the light-emitting layer EML may be improved, and emission efficiency and device lifetime may be improved. If the total amount of the second compound and the third compound deviates from the herein-described ratio range, charge balance in the light-emitting layer EML may be broken, emission efficiency may be degraded, and the device may be easily deteriorated.

If the light-emitting layer EML includes the fourth compound, the amount of the fourth compound may be about 4 wt % to 30 wt % based on the total weight of the first compound, the second compound, the third compound and the fourth compound in the light-emitting layer EML. However, one or more embodiments of the disclosure is not limited thereto. If the amount of the fourth compound satisfies the herein-described amount, energy transfer from a host to the first compound that is a light emitting dopant may increase, and emission ratio may be improved. Accordingly, the emission efficiency of the light-emitting layer EML may be improved. If the amount ratio of the first compound, the second compound, the third compound and the fourth compound, included in the light-emitting layer EML satisfies the herein-described amount ratio, excellent or suitable emission efficiency and long lifetime may be achieved.

In one or more embodiments, the light-emitting layer EML may include as a suitable dopant material, styryl derivatives (for example, 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino) pyrene), and/or the like.

The light-emitting layer EML may include a suitable phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) and/or thulium (Tm). For example, iridium (III) bis(4,6-difluorophenylpyridinato-N,C2′) picolinate (FIrpic), bis(2,4-difluorophenylpyridinato)-tetrakis(1-pyrazolyl)borate iridium (III) (Fir6), and/or platinum octaethyl porphyrin (PtOEP) may be used as the phosphorescence dopant. However, one or more embodiments of the disclosure is not limited thereto.

In one or more embodiments, the light-emitting layer EML may include a hole transport host and an electron transport host. In some embodiments, the light-emitting layer EML may include an auxiliary dopant and a light emitting dopant. In one or more embodiments, the auxiliary dopant may include a phosphorescence dopant material or a thermally activated delayed fluorescence dopant. For example, in one or more embodiments, the light-emitting layer EML may include a hole transport host, an electron transport host, an auxiliary dopant, and a light emitting dopant.

The light-emitting layer may include a quantum dot.

In the description, the quantum dot refers to the crystal of a semiconductor compound. The quantum dot may be to emit light in one or more suitable emission wavelengths according to the size of the crystal. The quantum dot may be to emit light in one or more suitable emission wavelengths by controlling the element ratio in the quantum dot compound.

The diameter of the quantum dot may be, for example, about 1 nanometer (nm) to about 10 nm. In the present disclosure, when quantum dot, quantum dots, or quantum dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dot may be synthesized by a chemical bath deposition, a metal organic chemical vapor deposition, a molecular beam epitaxy or a similar process therewith.

The chemical bath deposition is a method of mixing an organic solvent and a precursor material and then, growing a quantum dot particle crystal. During growing the crystal, the organic solvent may naturally play the role of a dispersant which is coordinated on the surface of the quantum dot crystal and may control the growth of the crystal. Accordingly, the chemical bath deposition is more advantageous if (e.g., when) compared to a vapor deposition method including a metal organic chemical vapor deposition (MOCVD) and molecular beam epitaxy (MBE), and the growth of the quantum dot particle may be controlled or selected through a low-cost process.

The light-emitting layer EML may include a quantum dot material. The core of the quantum dot may be selected from among a II-VI group compound, a III-VI group compound, a I-III-VI group compound, a III-V group compound, a III-II-V group compound, a IV-VI group compound, a IV group element, a IV group compound, and/or one or more (e.g., any suitable) combinations thereof.

The Group II-VI compound may be selected from among the group consisting of a binary compound selected from among the group consisting of CdSe, CdTe, CdS, ZnS, ZnSe, ZnTe, ZnO, HgS, HgSe, HgTe, MgSe, MgS, and/or a (e.g., any suitable) mixture thereof; a ternary compound selected from among the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or a (e.g., any suitable) mixture thereof; and a quaternary compound selected from among the group consisting of HgZnTeS, CdZnSeS, CdZnSeTe, CdZnSTe, CdHgSeS, CdHgSeTe, CdHgSTe, HgZnSeS, HgZnSeTe, and/or a (e.g., any suitable) mixture thereof. In one or more embodiments, the Group II-VI compound may further include a Group I metal and/or a Group IV element. The Group I-II-VI compound may be selected from among CuSnS or CuZnS, and the Group II-IV-VI compound may be selected from among ZnSnS and/or the like. The Group I-II-IV-VI compound may be selected from among quaternary compounds selected from among the group consisting of Cu₂ZnSnS₂, Cu₂ZnSnS₄, Cu₂ZnSnSe₄, Ag₂ZnSnS₂, and/or a (e.g., any suitable) mixture thereof.

The III-VI group compound may include a binary compound such as In₂S₃, and In₂Se₃, a ternary compound such as InGaS₃, and InGaSe₃, or arbitrary combinations thereof.

The I-III-VI group compound may be selected from among a ternary compound selected from among the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The III-V group compound may be selected from among the group consisting of a binary compound selected from among the group consisting of GaN, GaP, GaAs, GaSb, AlN, AlP, AlAs, AlSb, InN, InP, InAs, InSb, and mixtures thereof, a ternary compound selected from among the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from among the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In one or more embodiments, the III-V group compound may further include a II group metal. For example, InZnP, and/or the like may be selected as a III-II-V group compound.

The Group IV-VI compound may be selected from among the group consisting of a binary compound selected from among the group consisting of SnS, SnSe, SnTe, PbS, PbSe, PbTe, and/or a (e.g., any suitable) mixture thereof, a ternary compound selected from among the group consisting of SnSeS, SnSeTe, SnSTe, PbSeS, PbSeTe, PbSTe, SnPbS, SnPbSe, SnPbTe, and/or a (e.g., any suitable) mixture thereof, and a quaternary compound selected from among the group consisting of SnPbSSe, SnPbSeTe, SnPbSTe, and/or a (e.g., any suitable) mixture thereof.

The Group II-IV-V compound may be selected from among a ternary compound selected from among the group consisting of ZnSnP, ZnSnP₂, ZnSnAS₂, ZnGeP₂, ZnGeAs₂, CdSnP₂, and CdGeP₂ and/or a (e.g., any suitable) mixture thereof.

The Group IV element may be selected from among the group consisting of Si, Ge, and/or a (e.g., any suitable) mixture thereof. The Group IV compound may be a binary compound selected from among the group consisting of SiC, SiGe, and/or a (e.g., any suitable) mixture thereof.

Each element included in the multi-element compound such as the binary compound, ternary compound, and quaternary compound may be present in particles at a substantially uniform concentration or a non-substantially uniform concentration. For example, the preceding Formulas indicate the types (kinds) of elements included in a compound, and element ratios in the compound may be different. For example, AgInGaS₂ may indicate AgInₓGa₁₋ₓS₂ (x is a real number between 0 and 1).

In this case, the binary compound, the ternary compound or the quaternary compound may be present at substantially uniform concentration in a particle or may be present at a partially different concentration distribution state in substantially the same particle. In some embodiments, a core/shell structure in which one quantum dot wraps another quantum dot may be possible. The interface of the core and the shell may have a concentration gradient in which the concentration of an element present in the shell is decreased toward the center.

In one or more embodiments, the quantum dot may have the herein-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, and/or one or more (e.g., any suitable) combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but one or more embodiments of the present disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but one or more embodiments of the present disclosure is not limited thereto.

The quantum dot may have a full width of half maximum (FWHM) of emission wavelength spectrum of about 45 nm or less, about 40 nm or less, more, about 30 nm or less. Within this range, color purity or color reproducibility may be improved. In some embodiments, light emitted via such quantum dot is emitted in all directions, and light view angle properties may be improved.

In some embodiments, the shape of the quantum dot may be generally used shapes in the art, without specific limitation. More particularly, the shape of spherical, pyramidal, multi-arm, or cubic nanoparticle, nanotube, nanowire, nanofiber, nanoplate particle, and/or the like. may be used.

As the size of the quantum dot or the ratio of elements in the quantum dot compound is regulated, the energy band gap may be accordingly controlled or selected to obtain light of one or more suitable wavelengths from the quantum dot light-emitting layer. Therefore, by using the quantum dots as described herein (using quantum dots of different sizes or having different element ratios in the quantum dot compound), a light-emitting element emitting light of one or more suitable wavelengths may be obtained. For example, the size of the quantum dots or the ratio of elements in the quantum dot compound may be regulated to emit red, green, and/or blue light. In some embodiments, the quantum dots may be configured to emit white light by combining light of one or more suitable colors.

In the light-emitting elements ED of embodiments, as shown in FIG. 3 to FIG. 6, the electron transport region ETR is provided on the light-emitting layer EML. The electron transport region ETR may include at least one of an electron blocking layer HBL, an electron transport layer ETL or an electron injection layer EIL. However, one or more embodiments of the disclosure is not limited thereto.

The electron transport region ETR may have a single layer formed using a single material, a single layer formed using multiple different materials, or a multilayer structure having multiple layers formed using multiple different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using multiple different materials, or a structure stacked from the light-emitting layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1.

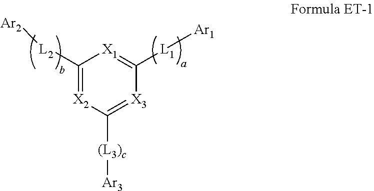

In Formula ET-1, at least one among X₁ to X₃ is N, and the remainder are CRₐ. Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, “a” to “c” may each independently be an integer of 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if “a” to “c” are integers of 2 or more, Li to L₃ may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, one or more embodiments of the present disclosure is not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-yl)phenyl)-9,10-dinaphthylanthracene, 1,3,5-tri (1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalen-2-yl)anthracene (ADN), 1,3-bis[3,5-di(pyridin-3-yl)phenyl]benzene (BmPyPhB), and/or CNNPTRZ(4′-(4-(4-(4,6-diphenyl-1,3,5-triazin-2-yl)phenyl)naphthalen-1-yl)-[1,1′-biphenyl]-4-carbonitrile) and mixtures thereof, without limitation.

The electron transport region ETR may include at least one among (e.g., selected from among) compounds ET1 to ET36.

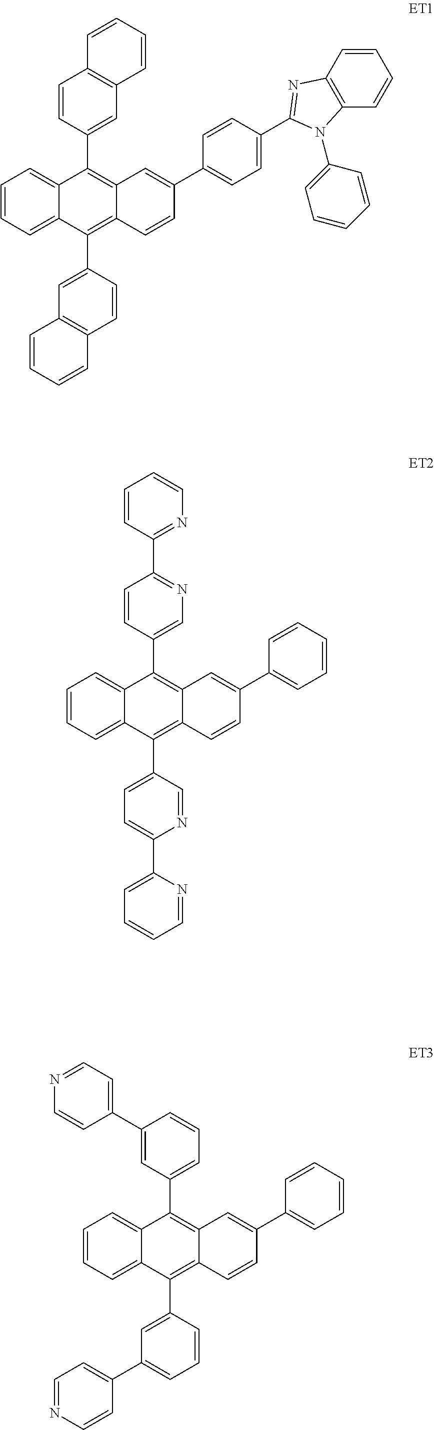 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

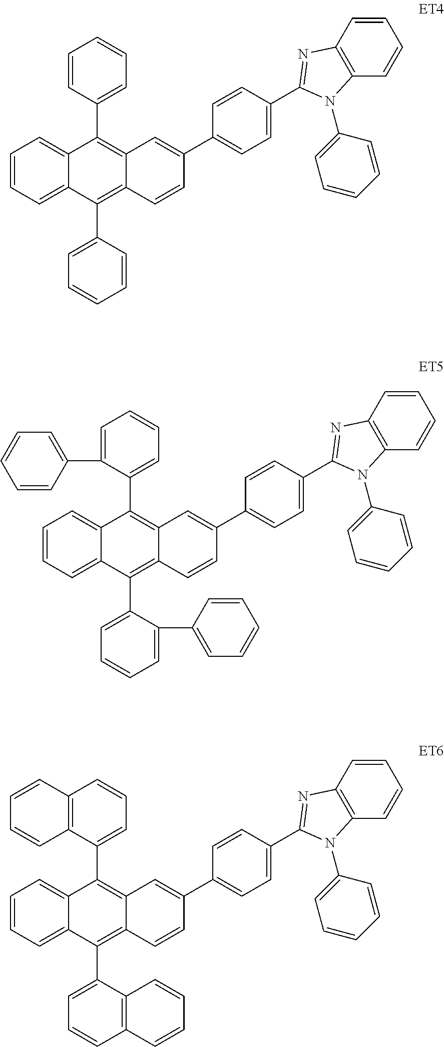 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

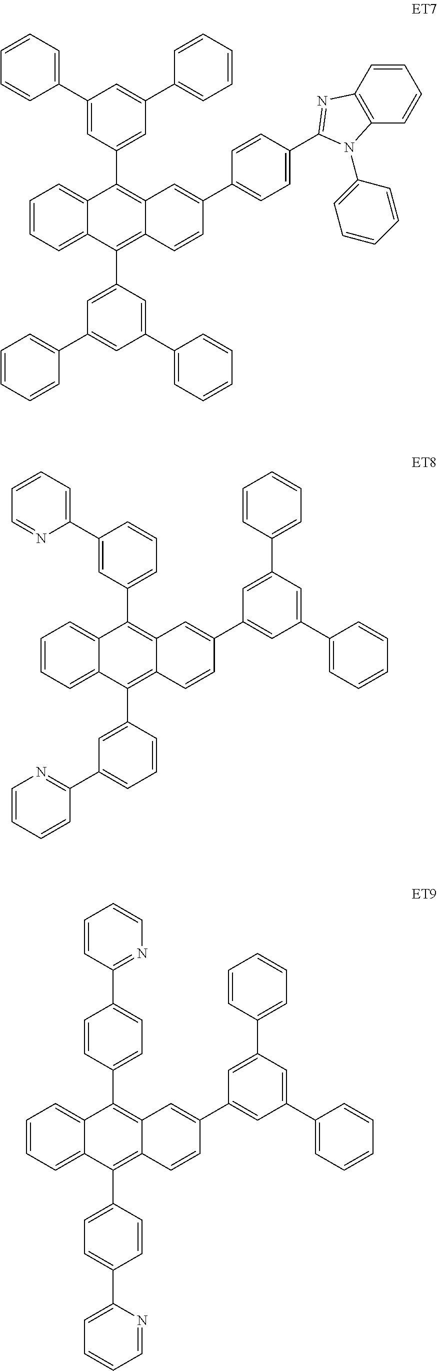 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

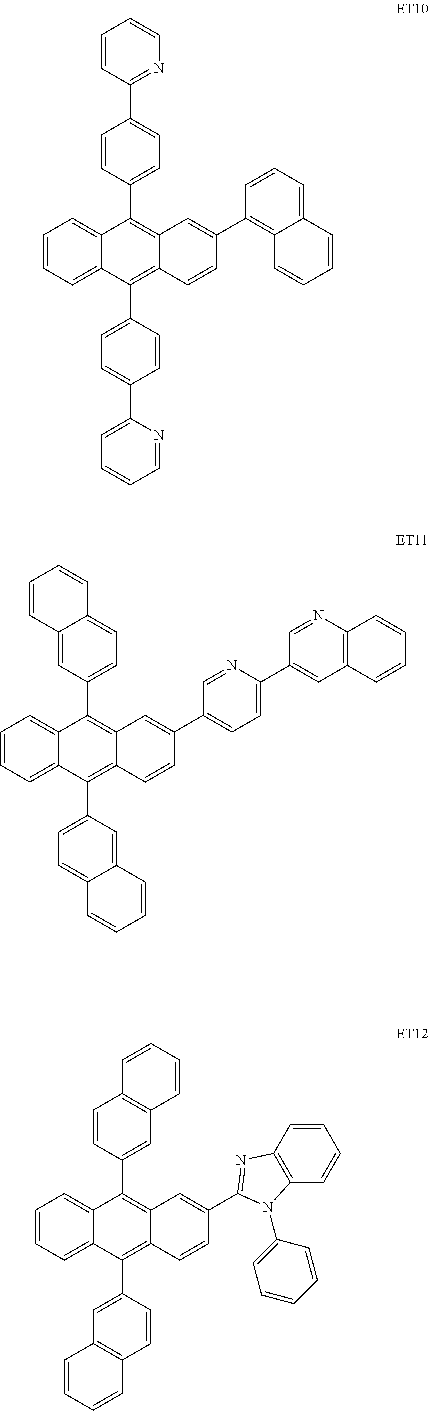 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

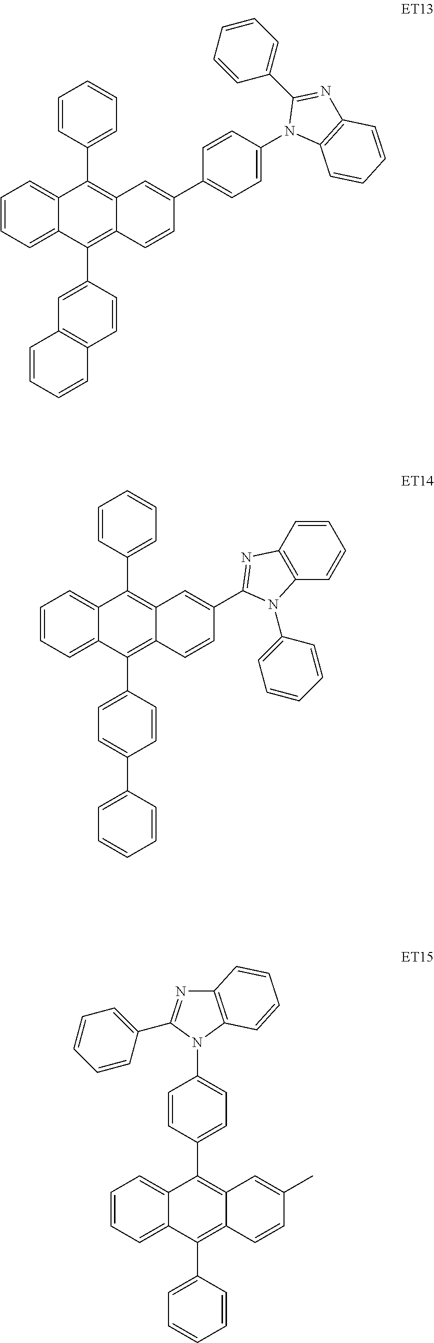 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

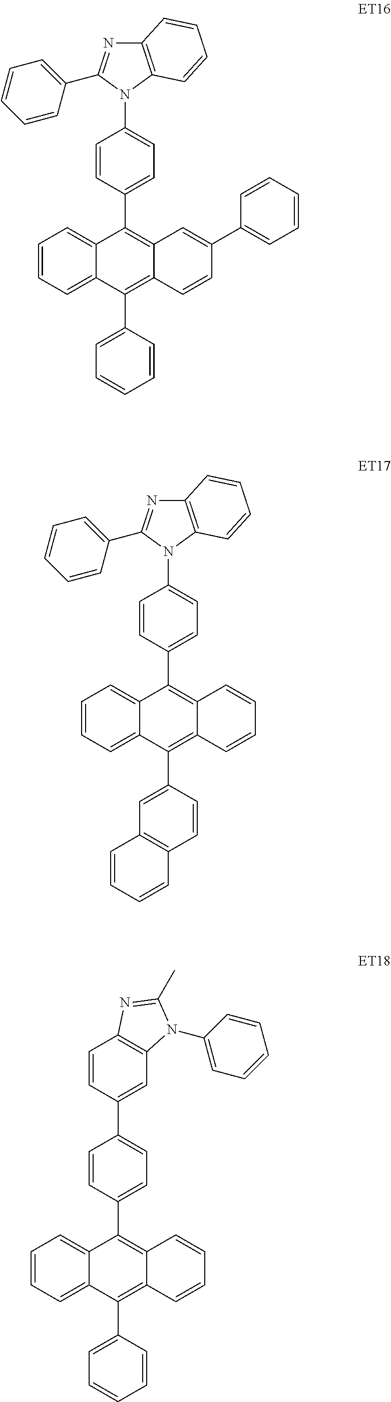 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

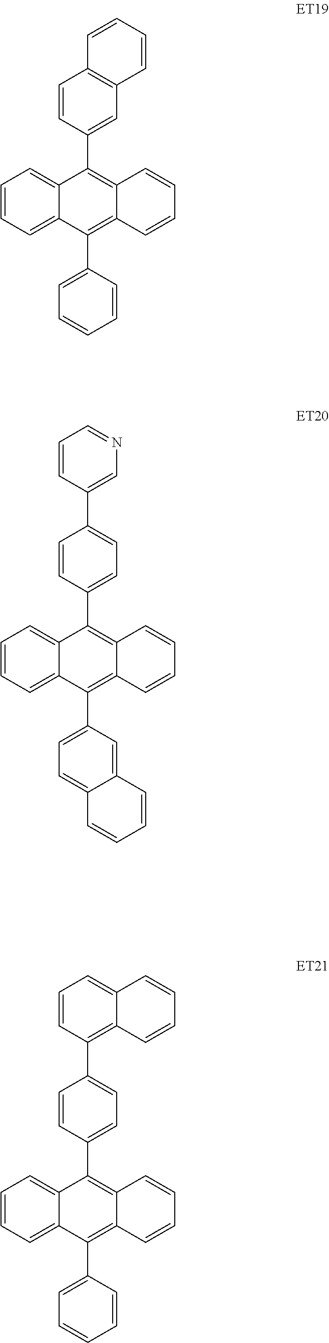

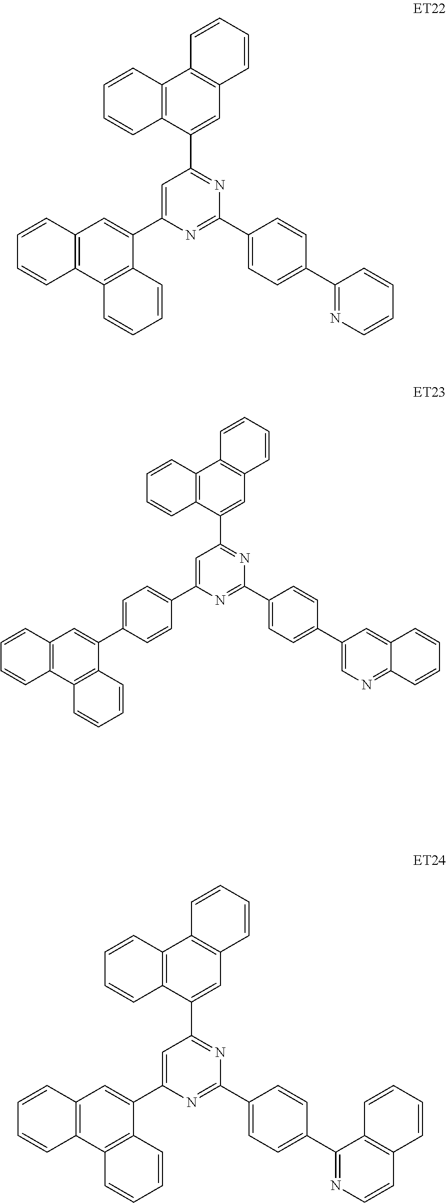 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

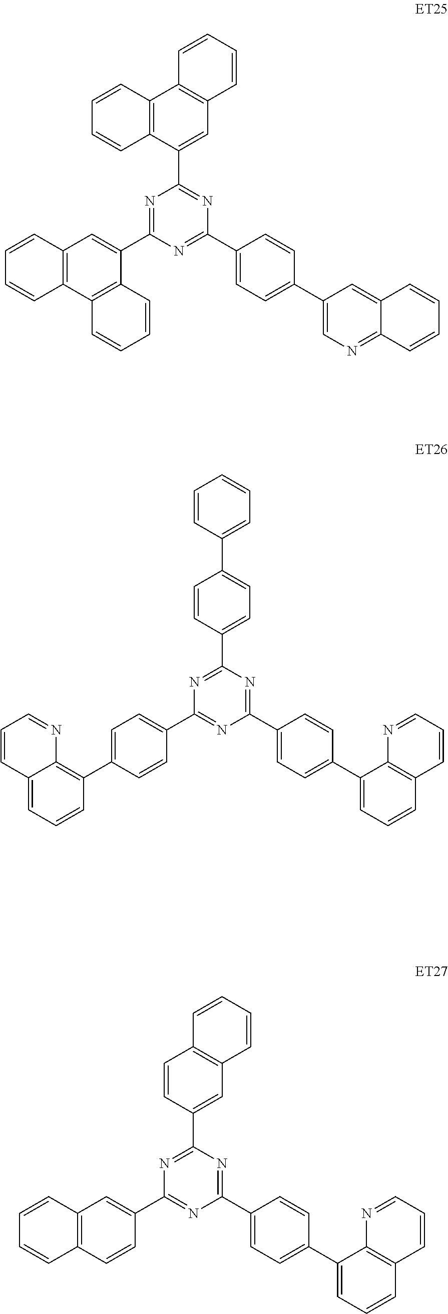 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

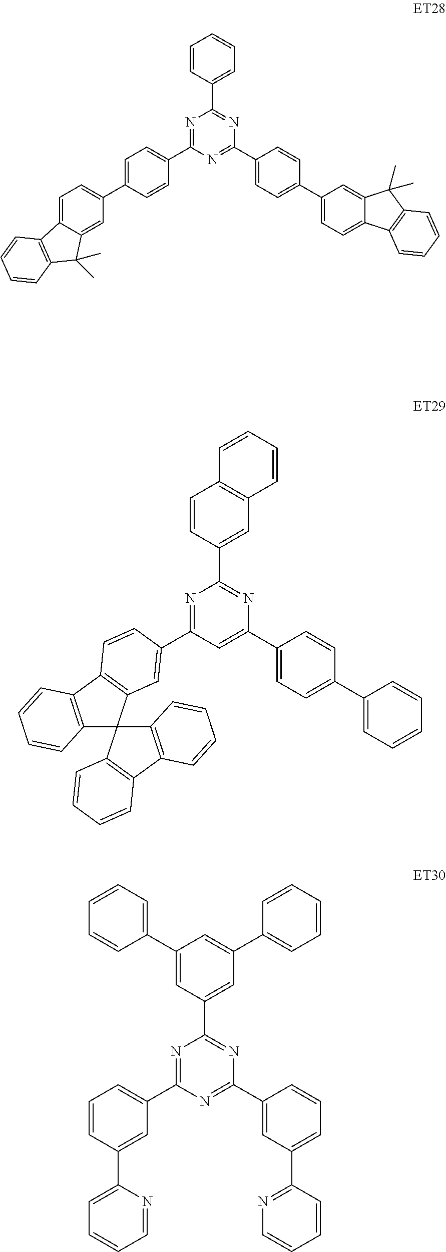 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

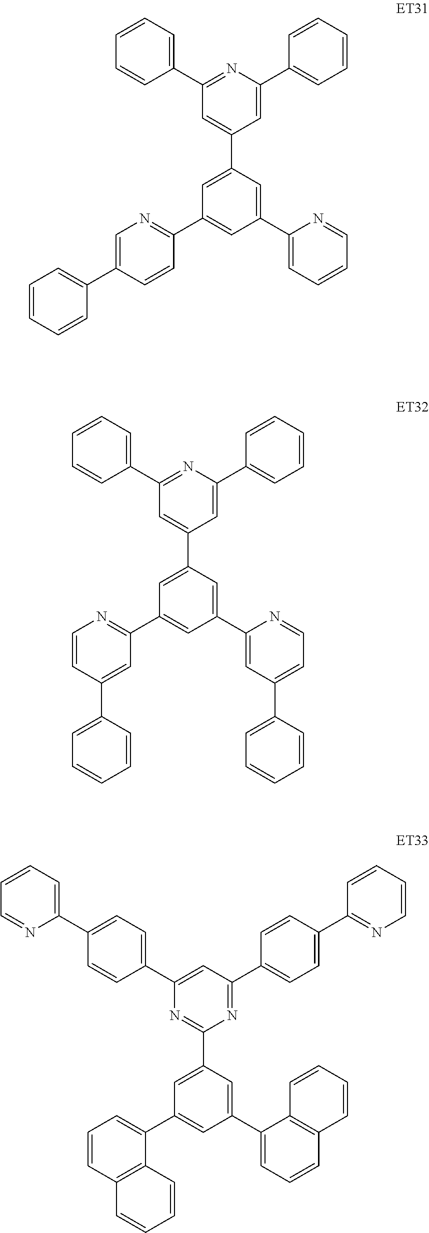 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

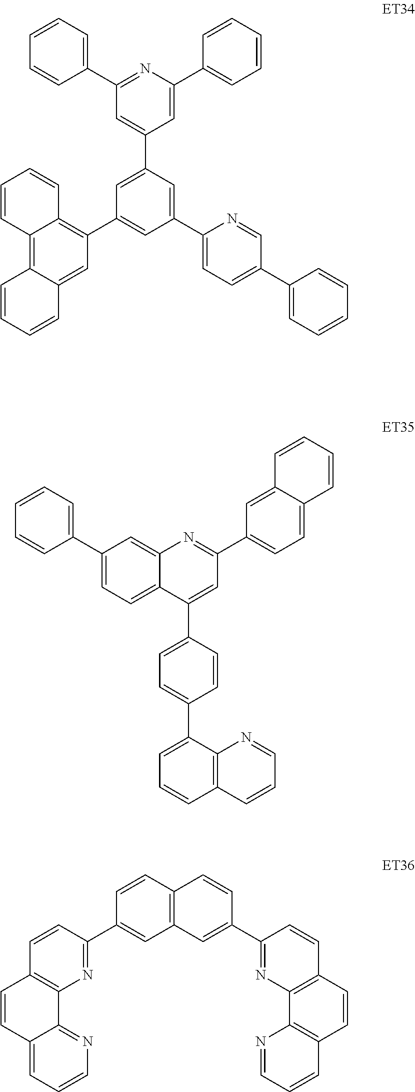 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI and/or KI, a lanthanide metal such as Yb, or a co-depositing material of the metal halide and the lanthanide metal. For example, the electron transport region ETR may include KI:Yb, RbI:Yb, LiF:Yb, and/or the like, as the co-depositing material. In one or more embodiments, the electron transport region ETR may use a metal oxide such as Li₂O and BaO, or 8-hydroxy-lithium quinolate (Liq). However, one or more embodiments of the disclosure is not limited thereto. The electron transport region ETR also may be formed using a mixture material of an electron transport material and an insulating organo metal salt. The organo metal salt may be a material having an energy band gap of about 4 eV or more. for example, the organo metal salt may include, for example, metal acetates, metal benzoates, metal acetoacetates, metal acetylacetonates, and/or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, one or more embodiments of the disclosure is not limited thereto.

The electron transport region ETR may include the compounds of the electron transport region in at least one among an electron injection layer EIL, an electron transport layer ETL, and a hole blocking layer HBL.

If the electron transport region ETR includes the electron transport layer ETL, the thickness of the electron transport layer ETL may be from about 100 Å to about 1,000 Å, for example, from about 150 Å to about 500 Å. If the thickness of the electron transport layer ETL satisfies the herein-described range, satisfactory electron transport properties may be obtained without substantial increase of a driving voltage. If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies the herein described range, satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but one or more embodiments of the disclosure is not limited thereto. For example, if the first electrode EL1 is an anode, the second cathode EL2 may be a cathode, and if the first electrode EL1 is a cathode, the second electrode EL2 may be an anode. The second electrode EL2 may include at least one selected among Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, or oxides thereof.

The second electrode EL2 may be a transmissive electrode, a transflective electrode or a reflective electrode. If the second electrode EL2 is the transmissive electrode, the second electrode EL2 may include a transparent metal oxide, for example, ITO, IZO, ZnO, ITZO, and/or the like.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, and/or W, compounds including thereof, or mixtures thereof (for example, AgMg, AgYb, or MgAg). In one or more embodiments, the second electrode EL2 may have a multilayered structure including a reflective layer or a transflective layer formed using the herein-described materials and a transparent conductive layer formed using ITO, IZO, ZnO, ITZO, and/or the like. For example, the second electrode EL2 may include the aforementioned metal materials, combinations of two or more metal materials selected from among the aforementioned metal materials, or oxides of the aforementioned metal materials.

In some embodiments, the second electrode EL2 may be connected with an auxiliary electrode. If the second electrode EL2 is connected with the auxiliary electrode, the resistance of the second electrode EL2 may decrease.

In one or more embodiments, on the second electrode EL2 in the light-emitting element ED of one or more embodiments, a capping layer CPL may be further arranged. The capping layer CPL may include a multilayer or a single layer.

In one or more embodiments, the capping layer CPL may be an organic layer or an inorganic layer. For example, if the capping layer CPL includes an inorganic material, the inorganic material may include an alkali metal compound such as LiF, an alkaline earth metal compound such as SiON, SiNₓ, SiO_{y}, and/or the like.

For example, if the capping layer CPL includes an organic material, the organic material may include 2,2′-dimethyl-N,N′-di-[(1-naphthyl)-N,N′-diphenyl]-1,1′-biphenyl-4,4′-diamine (α-NPD), NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol sol-9-yl)triphenylamine (TCTA), and/or the like, or may include an epoxy resin, or acrylate such as methacrylate. In some embodiments, a capping layer CPL may include at least one among (e.g., selected from among) compounds P1 to P5, but one or more embodiments of the disclosure is not limited thereto.

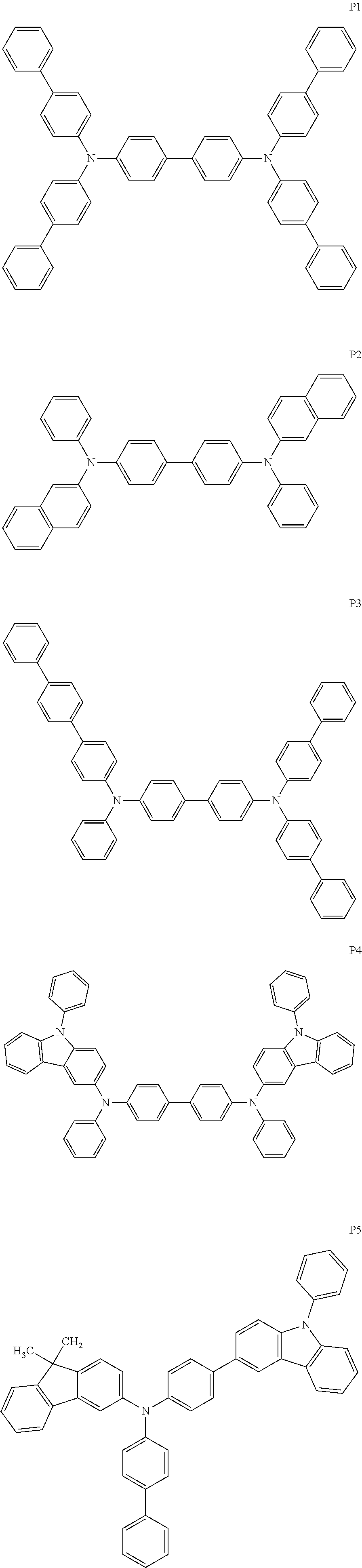 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be at least about 1.6 (e.g., or more). for example, the refractive index of the capping layer CPL with respect to light in a wavelength range of about 550 nm to about 660 nm may be at least about 1.6 (e.g., or more).

FIG. 7 to FIG. 10 are cross-sectional views on display apparatuses according to one or more embodiments, respectively. In the explanation on the display apparatuses of embodiments referring to FIG. 7 to FIG. 10, the overlapping parts with the explanation on FIG. 1 to FIG. 6 will not be explained again, and the different features will be explained chiefly.

Referring to FIG. 7, the display apparatus DD-a according to one or more embodiments may include a display panel DP including a display device layer DP-ED, a light controlling layer CCL arranged on the display panel DP and a color filter layer CFL.

In one or more embodiments shown in FIG. 7, the display panel DP includes a base layer BS, a circuit layer DP-CL provided on the base layer BS and a display device layer DP-ED, and the display device layer DP-ED may include a light-emitting device ED.

The light-emitting device ED may include a first electrode EL1, a hole transport region HTR arranged on the first electrode EL1, a light-emitting layer EML arranged on the hole transport region HTR, an electron transport region ETR arranged on the light-emitting layer EML, and a second electrode EL2 arranged on the electron transport region ETR. In one or more embodiments, the same structures as the light emitting devices of FIG. 3 to FIG. 6 may be applied to the structure of the light-emitting device ED shown in FIG. 7.

The hole transport region HTR of the light-emitting element ED included in the display device DD-a according to one or more embodiments may include the amine compound of one or more embodiments described herein.

Referring to FIG. 7, the light-emitting layer EML may be arranged in an opening part OH defined in a pixel definition layer PDL. For example, the light-emitting layer EML divided by the pixel definition layer PDL and correspondingly provided to each of luminous areas PXA-R, PXA-G and PXA-B may be to emit light in substantially the same wavelength region. In the display apparatus DD-a of one or more embodiments, the light-emitting layer EML may be to emit blue light. In one or more embodiments, different from the drawings, in one or more embodiments, the light-emitting layer EML may be provided as a common layer for all luminous areas PXA-R, PXA-G and PXA-B.

The light controlling layer CCL may be arranged on the display panel DP. The light controlling layer CCL may include a light converter. The light converter may be a quantum dot or a phosphor. The light converter may transform the wavelength of light provided and then emit. For example, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be arranged between the separated light controlling parts CCP1, CCP2 and CCP3, but one or more embodiments of the disclosure is not limited thereto. In FIG. 7, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 converting first color light provided from the light-emitting element ED into second color light, a second light controlling part CCP2 including a second quantum dot QD2 converting first color light into third color light, and a third light controlling part CCP3 transmitting first color light.

In one or more embodiments, the first light controlling part CCP1 may provide red light which is the second color light, and the second light controlling part CCP2 may provide green light which is the third color light. The third color controlling part CCP3 may be to transmit and provide blue light which is the first color light provided from the light-emitting element ED. For example, the first quantum dot QD1 may be a red quantum dot, and the second quantum dot QD2 may be a green quantum dot. On the quantum dots QD1 and QD2, the same contents as those described herein may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a quantum dot but include the scatterer SP.

The scatterer SP may be an inorganic particle. For example, the scatterer SP may include at least one among TiO₂, ZnO, Al₂O₃, SiO₂, and hollow silica. The scatterer SP may include at least one among TiO₂, ZnO, Al₂O₃, SiO₂, and hollow silica, or may be a mixture of two or more materials selected among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP. In one or more embodiments, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3.

The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may be acrylic resins, urethane-based resins, silicone-based resins, epoxy-based resins, and/or the like. The base resins BR1, BR2 and BR3 may be transparent resins. In one or more embodiments, the first base resin BR1, the second base resin BR2 and the third base resin BR3 may be the same or different from each other.

The light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may block the exposure of the light controlling parts CCP1, CCP2 and CCP3 to humidity/oxygen. In one or more embodiments, the barrier layer BFL1 may cover the light controlling parts CCP1, CCP2 and CCP3. In some embodiments, a color filter layer CFL, which will be explained later, may include a barrier layer BFL2 arranged on the light controlling parts CCP1, CCP2 and CCP3.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and silicon oxynitride or a metal thin film securing light transmittance. In one or more embodiments, the barrier layers BFL1 and BFL2 may further include an organic layer. The barrier layers BFL1 and BFL2 may be composed of a single layer of multiple layers.

In the display apparatus DD-a of one or more embodiments, the color filter layer CFL may be arranged on the light controlling layer CCL. For example, the color filter layer CFL may be arranged directly on the light controlling layer CCL. In this case, the barrier layer BFL2 may not be provided.

The color filter layer CFL may include filters CF1, CF2 and CF3. Each of the first to third filters CF1, CF2 and CF3 may be arranged corresponding to a red luminous area PXA-R, a green luminous area PXA-G, and a blue luminous area PXA-B, respectively.

The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment or dye. The first filter CF1 may include a red pigment or dye, the second filter CF2 may include a green pigment or dye, and the third filter CF3 may include a blue pigment or dye.

In one or more embodiments, one or more embodiments of the disclosure is not limited thereto, and the third filter CF3 may not include (e.g., may exclude) a (e.g., any) pigment or dye. The third filter CF3 may include a polymer photosensitive resin and not include a pigment or dye. The third filter CF3 may be transparent. The third filter CF3 may be formed using a transparent photosensitive resin.

In some embodiments, in one or more embodiments, the first filter CF1 and the second filter CF2 may be yellow filters. The first filter CF1 and the second filter CF2 may be provided in one body without distinction. The first to third filters CF1, CF2 and CF3 may be arranged corresponding to the red luminous area PXA-R, the green luminous area PXA-G and the blue luminous area PXA-B, respectively.

In one or more embodiments, though not shown, the color filter layer CFL may further include a light blocking part. The color filter layer CFL may include a light blocking part arranged at the boundaries to overlap with adjacent filters CF1, CF2 and CF3. The light blocking part may be a black matrix. The light blocking part may be formed by including an organic light blocking material or an inorganic light blocking material, including a black pigment or black dye. The light blocking part may divide the boundaries among adjacent filters CF1, CF2 and CF3. In some embodiments, in one or more embodiments, the light blocking part may be formed as a blue filter.

On the color filter layer CFL, a base substrate BL may be arranged. The base substrate BL may be a member providing a base surface on which the color filter layer CFL, the light controlling layer CCL, and/or the like are arranged. The base substrate BL may be a glass substrate, a metal substrate, a plastic substrate, and/or the like. However, one or more embodiments of the disclosure is not limited thereto, and the base substrate BL may be an inorganic layer, an organic layer or a composite material layer. In some embodiments, different from the drawing, the base substrate BL may not be provided in one or more embodiments.

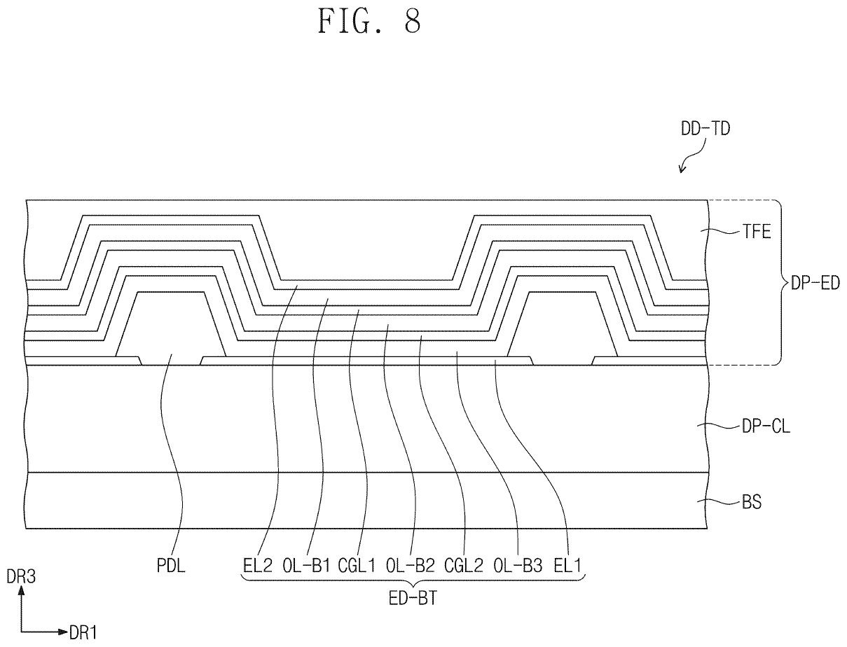

FIG. 8 is a cross-sectional view showing a part of the display device according to one or more embodiments. In a display device DD-TD of one or more embodiments, the light-emitting element ED-BT may include multiple light emitting structures OL-B1, OL-B2 and OL-B3. The light-emitting element ED-BT may include oppositely arranged first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include the light-emitting layer EML (FIG. 7), and a hole transport region HTR and an electron transport region ETR, arranged with the light-emitting layer (FIG. 7) therebetween.

For example, the light-emitting element ED-BT included in the display device DD-TD of one or more embodiments may be a light-emitting element of a tandem structure including multiple light-emitting layers.

In one or more embodiments shown in FIG. 8, light emitted from the light emitting structures OL-B1, OL-B2 and OL-B3 may be all blue light. However, one or more embodiments of the disclosure is not limited thereto, and the wavelength regions of light emitted from the light emitting structures OL-B1, OL-B2 and OL-B3 may be different from each other. For example, the light-emitting element ED-BT including the multiple light emitting structures OL-B1, OL-B2 and OL-B3 emitting light in different wavelength regions may be to emit white light.

Between neighboring light emitting structures OL-B1, OL-B2 and OL-B3, charge generating layers CGL1 and CGL2 may be arranged. The charge generating layers CGL1 and CGL2 may include a p-type (kind) charge generating layer and/or an n-type (kind) charge generating layer.

The herein-described amine compound according to one or more embodiments may be contained in at least one among emission structures OL-B1, OL-B2, and OL-B3 included in the display device DD-TD according to one or more embodiments.

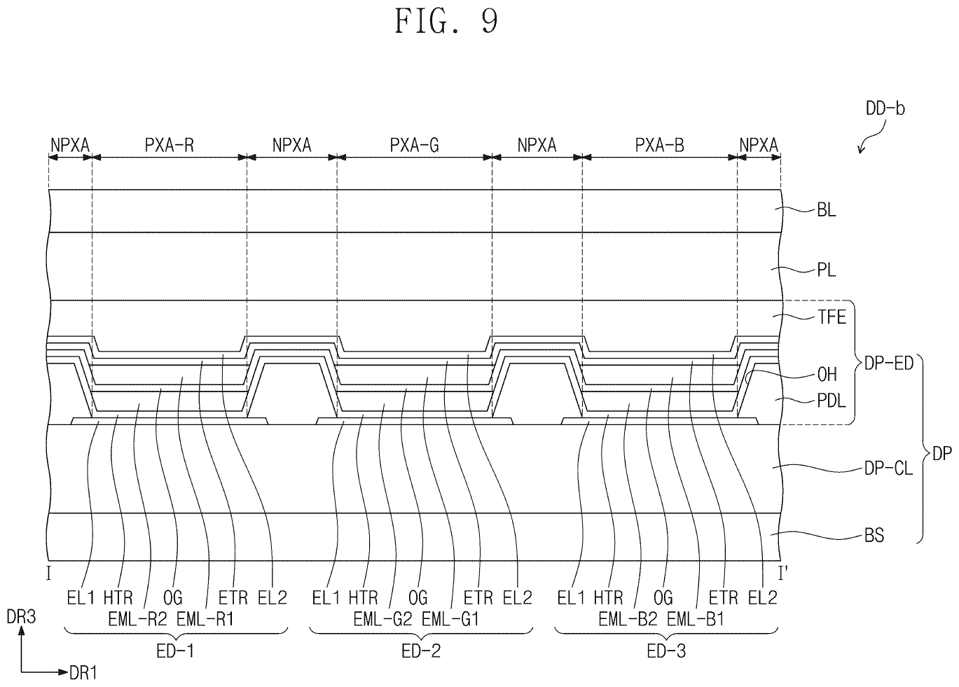

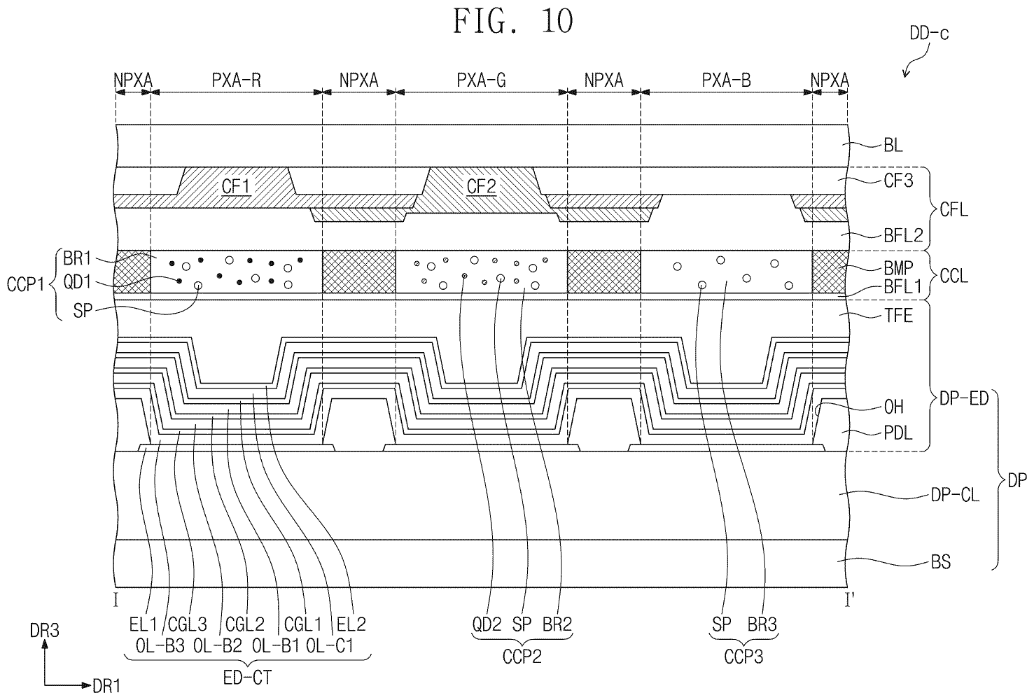

Referring to FIG. 9, a display device DD-b according to one or more embodiments may include light-emitting elements ED-1, ED-2 and ED-3, formed by stacking two light-emitting layers. Compared to the display device DD of one or more embodiments, shown in FIG. 2, one or more embodiments shown in FIG. 9 is different in that first to third light-emitting elements ED-1, ED-2 and ED-3 include two light-emitting layers stacked in a thickness direction, each. In the first to third light-emitting elements ED-1, ED-2 and ED-3, two light-emitting layers may be to emit light in substantially the same wavelength region.

The first light-emitting element ED-1 may include a first red light-emitting layer EML-R1 and a second red light-emitting layer EML-R2. The second light-emitting element ED-2 may include a first green light-emitting layer EML-G1 and a second green light-emitting layer EML-G2. In some embodiments, the third light-emitting element ED-3 may include a first blue light-emitting layer EML-B1 and a second blue light-emitting layer EML-B2. Between the first red light-emitting layer EML-R1 and the second red light-emitting layer EML-R2, between the first green light-emitting layer EML-G1 and the second green light-emitting layer EML-G2, and between the first blue light-emitting layer EML-B1 and the second blue light-emitting layer EML-B2, an emission auxiliary part OG may be arranged.

The emission auxiliary part OG may include a single layer or a multilayer. The emission auxiliary part OG may include a charge generating layer. More particularly, the emission auxiliary part OG may include an electron transport region, a charge generating layer, and a hole transport region stacked in order. The emission auxiliary part OG may be provided as a common layer in all of the first to third light-emitting elements ED-1, ED-2 and ED-3. However, one or more embodiments of the disclosure is not limited thereto, and the emission auxiliary part OG may be patterned and provided in an opening part OH defined in a pixel definition layer PDL.

The first red light-emitting layer EML-R1, the first green light-emitting layer EML-G1 and the first blue light-emitting layer EML-B1 may be arranged between the electron transport region ETR and the emission auxiliary part OG. The second red light-emitting layer EML-R2, the second green light-emitting layer EML-G2 and the second blue light-emitting layer EML-B2 may be arranged between the emission auxiliary part OG and the hole transport region HTR.

For example, the first light-emitting element ED-1 may include a first electrode EL1, a hole transport region HTR, a second red light-emitting layer EML-R2, an emission auxiliary part OG, a first red light-emitting layer EML-R1, an electron transport region ETR, and a second electrode EL2, stacked in order. The second light-emitting element ED-2 may include a first electrode EL1, a hole transport region HTR, a second green light-emitting layer EML-G2, an emission auxiliary part OG, a first green light-emitting layer EML-G1, an electron transport region ETR, and a second electrode EL2, stacked in order. The third light-emitting element ED-3 may include a first electrode EL1, a hole transport region HTR, a second blue light-emitting layer EML-B2, an emission auxiliary part OG, a first blue light-emitting layer EML-B1, an electron transport region ETR, and a second electrode EL2, stacked in order.

In one or more embodiments, an optical auxiliary layer PL may be arranged on a display device layer DP-ED. The optical auxiliary layer PL may include a polarization layer. The optical auxiliary layer PL may be arranged on a display panel DP and may control reflected light at the display panel DP by external light. Different from the drawings, the optical auxiliary layer PL may not be provided from the display apparatus according to one or more embodiments.

Different from FIG. 8 and FIG. 9, a display apparatus DD-c in FIG. 10 is shown to include four light emitting structures OL-B1, OL-B2, OL-B3 and OL-C1. A light-emitting element ED-CT may include oppositely arranged first electrode EL1 and second electrode EL2, and first to fourth light emitting structures OL-B1, OL-B2, OL-B3 and OL-C1 stacked in order in a thickness direction between the first electrode EL1 and the second electrode EL2. The third light emitting structures OL-B3, the second light emitting structures OL-B2, the first light emitting structures OL-B1, and the fourth light emitting structures OL-C1 are stacked in order in a thickness direction. Between the first to fourth light emitting structures OL-B1, OL-B2, OL-B3 and OL-C1, charge generating layers CGL1, CGL2 and CGL3 may be arranged. For example, A first charge generating layer CGL1 is arranged between the first light emitting structures OL-B1 and the fourth light emitting structures OL-C1. A second charge generating layer CGL2 is arranged between the first light emitting structures OL-B1 and the second light emitting structures OL-B2. A third charge generating layer CGL3 is arranged between the second light emitting structures OL-B2 and the third light emitting structures OL-B3.

Among the four light emitting structures, the first to third light emitting structures OL-B1, OL-B2 and OL-B3 emit blue light, and the fourth light emitting structure OL-C1 may be to emit green light. However, one or more embodiments of the disclosure is not limited thereto, and the first to fourth light emitting structures OL-B1, OL-B2, OL-B3 and OL-C1 may be to emit different wavelengths of light.

Charge generating layers CGL1, CGL2 and CGL3 arranged among neighboring light emitting structures OL-B1, OL-B2, OL-B3 and OL-C1 may include a p-type (kind) charge generating layer and/or an n-type (kind) charge generating layer.

The herein-described amine compound according to one or more embodiments may be contained in at least one among emission structures OL-B1, OL-B2, OL-B3, and OL-C1 included in the display device DD-c according to one or more embodiments.

The light-emitting element ED according to one or more embodiments of the disclosure contains the herein-described amine compound according to one or more embodiments in at least one functional layer arranged between the first electrode EL1 and the second electrode EL2, and thus may exhibit characteristics of improved luminous efficiency and lifespan. The light-emitting element ED according to one or more embodiments may contain the herein-described amine compound according to one or more embodiments in at least one among a hole transport region HTR, a light-emitting layer EML, and an electron transport region ETR arranged between the first electrode EL1 and the second electrode EL2, or may contain in a capping layer CPL.

For example, the amine compound according to one or more embodiments may be contained in the hole transport region HTR of the light-emitting element ED according to one or more embodiments, and the light-emitting element according to one or more embodiments may exhibit characteristics of high efficiency and long lifespan.

The herein-described amine compound according to one or more embodiments includes a first core, and a second and third substituents, and thus stability of materials may increase, and hole transporting properties may be improved. Therefore, the light-emitting element according to one or more embodiments containing the amine compound according to one or more embodiments may have increased lifespan and efficiency. In some embodiments, the light-emitting element according to one or more embodiments may exhibit characteristics of increased efficiency and lifespan by containing the amine compound according to one or more embodiments in the hole transport layer.

In one or more embodiments, the electronic device or an electronic apparatus may include a display device including multiple light-emitting elements and a control part controlling the display device. The electronic device or electronic apparatus of one or more embodiments may be an electronic device or electronic apparatus activated according to electrical signals. The electronic device or electronic apparatus may include display devices of one or more suitable embodiments. For example, the electronic device or electronic apparatus may include televisions, monitors, large-size display devices such as outside billboards, personal computers, laptop computers, personal digital terminals, display devices for automobiles, game consoles, portable electronic devices, medium- and small-size display devices such as cameras.

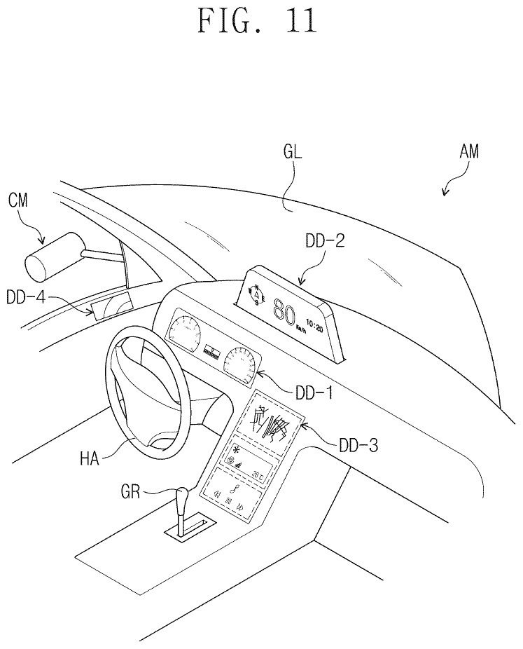

FIG. 11 is a diagram showing an automobile AM in which first to fourth display devices DD-1, DD-2, DD-3 and DD-4 are arranged. At least one among (e.g., selected from among) the first to fourth display devices DD-1, DD-2, DD-3 and DD-4 may include the same configurations of the display devices DD, DD-TD, DD-a, DD-b and DD-c of embodiments, explained referring to FIGS. 1, 2, and 7 to 10.

In FIG. 11, a vehicle is shown as an automobile AM, but this is an illustration, and the first to fourth display devices DD-1, DD-2, DD-3 and DD-4 may be arranged on other transport refers to such as bicycles, motorcycles, trains, ships and airplanes. In some embodiments, at least one among (e.g., selected from among) the first to fourth display devices DD-1, DD-2, DD-3 and DD-4 including the same configurations of the display devices DD, DD-TD, DD-a, DD-b and DD-c may be introduced in personal computers, laptop computers, personal digital terminals, game consoles, portable electronic devices, televisions, monitors, external billboards, and/or the like. In some embodiments, these are suggested as examples, and the display device may be introduced in other electronic devices as long as not deviated from the disclosure.

At least one among (e.g., selected from among) the first to fourth display devices DD-1, DD-2, DD-3, and DD-4 may include the light-emitting element ED of one or more embodiments as described with reference to FIGS. 3 to 6. The light-emitting element ED of one or more embodiments may include a fused polycyclic compound of one or more embodiments. At least one among (e.g., selected from among) the first to fourth display devices DD-1, DD-2, DD-3, and DD-4 may include the light-emitting element ED including the fused polycyclic compound of one or more embodiments, thereby improving a display service life.

Referring to FIG. 11, an automobile AM may include a steering wheel HA for the operation of the automobile AM and a gear GR. In some embodiments, the automobile AM may include a front window GL arranged to face a driver.

A first display device DD-1 may be arranged in a first region overlapping with the steering wheel HA. For example, the first display device DD-1 may be a digital cluster displaying the first information of the automobile AM. The first information may include a first graduation showing the running speed of the automobile AM, a second graduation showing the number of revolution of an engine (i.e., revolutions per minute (RPM)), and images showing a fuel state. First graduation and second graduation may be represented by digital images.

A second display device DD-2 may be arranged in a second region opposite to (e.g., facing) a driver's seat and overlapping with the front window GL. The driver's seat may be a seat where the steering wheel HA is arranged. For example, the second display device DD-2 may be a head up display (HUD) showing the second information of the automobile AM. The second display device DD-2 may be optically clear. The second information may include digital numbers showing the running speed of the automobile AM and may further include information including the current time. Different from the drawing, the second information of the second display device DD-2 may be projected and displayed on the front window GL.

A third display device DD-3 may be arranged in a third region adjacent to the gear GR. For example, the third display device DD-3 may be a center information display (CID) for an automobile, arranged between a driver's seat and a passenger seat and showing third information. The passenger seat may be a seat separated from the driver's seat with the gear GR therebetween. The third information may include information on road conditions (for example, navigation information), on playing music or radio, on playing a dynamic image (or image), on the temperature in the automobile AM, and/or the like.

A fourth display device DD-4 may be arranged in a fourth region separated from the steering wheel HA and the gear GR and adjacent to the side of the automobile AM. For example, the fourth display device DD-4 may be a digital wing mirror displaying fourth information. The fourth display device DD-4 may display the external image of the automobile AM, taken by a camera module CM arranged at the outside of the automobile AM. The fourth information may include the external image of the automobile AM.

The herein-described first to fourth information is for illustration, and the first to fourth display devices DD-1, DD-2, DD-3 and DD-4 may further display information on the inside and outside of the automobile. The first to fourth information may include different information from each other. However, one or more embodiments of the disclosure is not limited thereto, and a portion of the first to fourth information may include the same information.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light-emitting element, the display device, the electronic device, a device of manufacturing thereof, and/or any other relevant devices or components according to embodiments of the present disclosure described herein may be implemented utilizing any suitable hardware, firmware (e.g., an application-specific integrated circuit), software, or a combination of software, firmware, and hardware. For example, the one or more suitable components of the light-emitting element, and the display and/or electronic device may be formed on one integrated circuit (IC) chip or on separate IC chips. Further, the one or more suitable components of the light-emitting element and the display and/or electronic device may be implemented on a flexible printed circuit film, a tape carrier package (TCP), a printed circuit board (PCB), or formed on one substrate. Further, the one or more suitable components of the light-emitting element and the display and/or electronic device may be a process or thread, running on one or more processors, in one or more computing devices, executing computer program instructions and interacting with other system components for performing the one or more suitable functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of one or more suitable computing devices may be combined or integrated into a single computing device, or the functionality of a particular computing device may be distributed across one or more other computing devices without departing from the scope of the embodiments of the present disclosure.

Hereinafter, with reference to examples and comparative examples, an amine compound according to one or more embodiments of the disclosure and a light-emitting element according to one or more embodiments will be described in more detail. In some embodiments, examples to be described in more detail are provided to aid in understanding the disclosure and are merely illustrative, and a range of the disclosure is not limited to thereto.

### EXAMPLES

### 1. Synthesis of Amine Compound

First, synthetic methods of amine compounds according to one or more embodiments of the present disclosure will be described in more detail. For example, the synthetic methods of Compounds 50, 52, 67, 79, 94, 123, 143, 154, 352, 368, and 405 will be described. In some embodiments, the synthetic method of the amine compound to be described hereinafter is merely one or more example embodiments, and the synthetic method of the amine compound is not limited to one or more embodiments.

The amine compound of the present disclosure may be synthesized by a following reaction scheme.

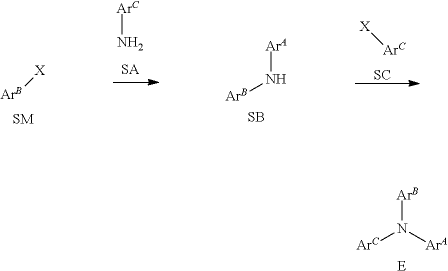

Referring to the preceding reaction scheme, a second amine intermediate SB may be obtained by reacting a starting material SM (aryl halide) with a first amine intermediate SA, and Compound E may be obtained by reacting the second amine intermediate SB with an aryl halide SC. When (e.g., if) it is possible to obtain an already suitable compound separately, some steps in the reaction scheme may not be provided. In this synthesis, the synthesis of Intermediate SB¹ to Intermediate SB⁶, Intermediate SB⁸, and Intermediate SB⁹ will not be provided because they are the suitable compounds, and the synthesis of Intermediate SB⁷, Intermediate SC¹ to Intermediate SC⁴ will be described. Ar^{A} to Ar^{C} in the reaction scheme are not necessarily consistent with Ar^{A} to Ar^{C} of specific example compounds suggested in the Compound Combination Table 1, and X refers to any halogen atom. The more specific synthesis method is described in more detail as follows.

Structures of Intermediate SM¹ to Intermediate SM³, Intermediate SB¹ to Intermediate SB¹⁰, Intermediate SC¹ to Intermediate SC⁴, Intermediates BA¹ to BA³, SA¹, and SA² used in the following synthesis examples are disclosed.

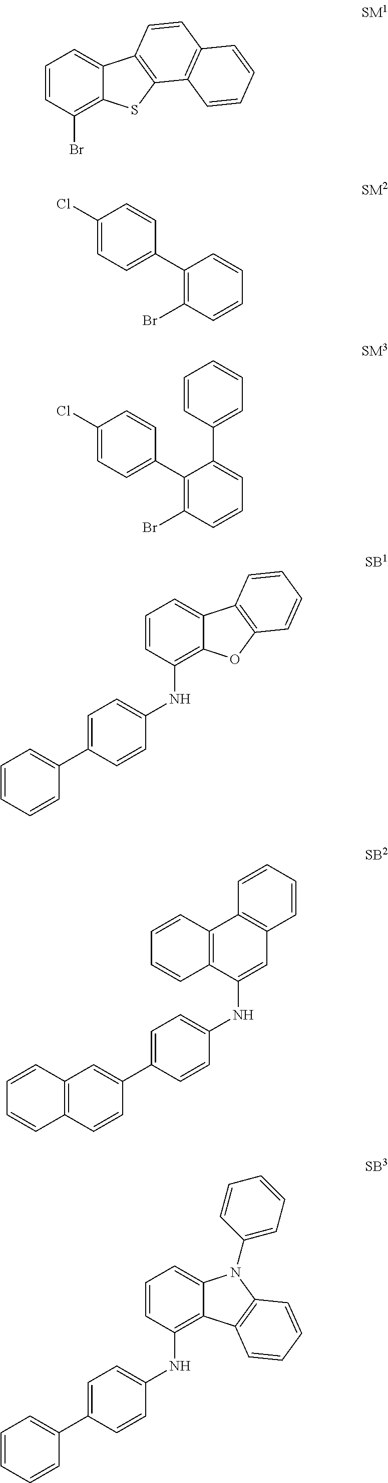

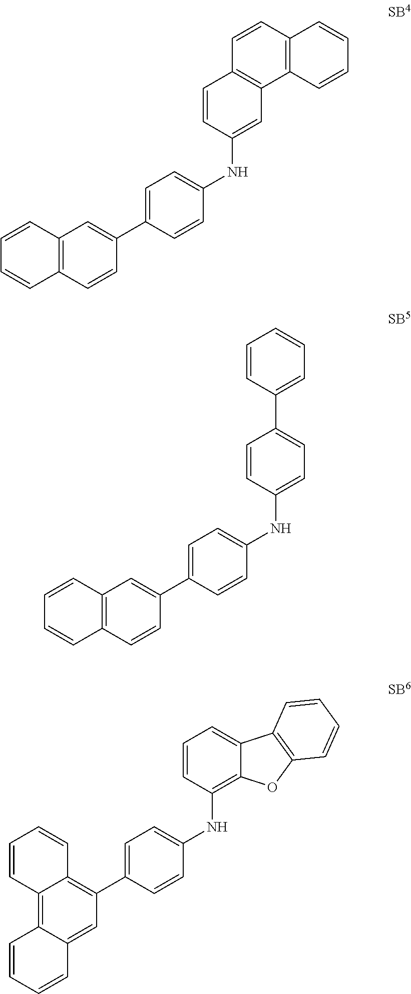 [SMILES: c1ccc2cc(-c3ccc(Nc4ccc5ccc6ccccc6c5c4)cc3)ccc2c1 ; c1ccc(-c2ccc(Nc3ccc(-c4ccc5ccccc5c4)cc3)cc2)cc1 ; c1ccc2c(c1)cc(-c1ccc(Nc3cccc4c3oc3ccccc34)cc1)c1ccccc12]

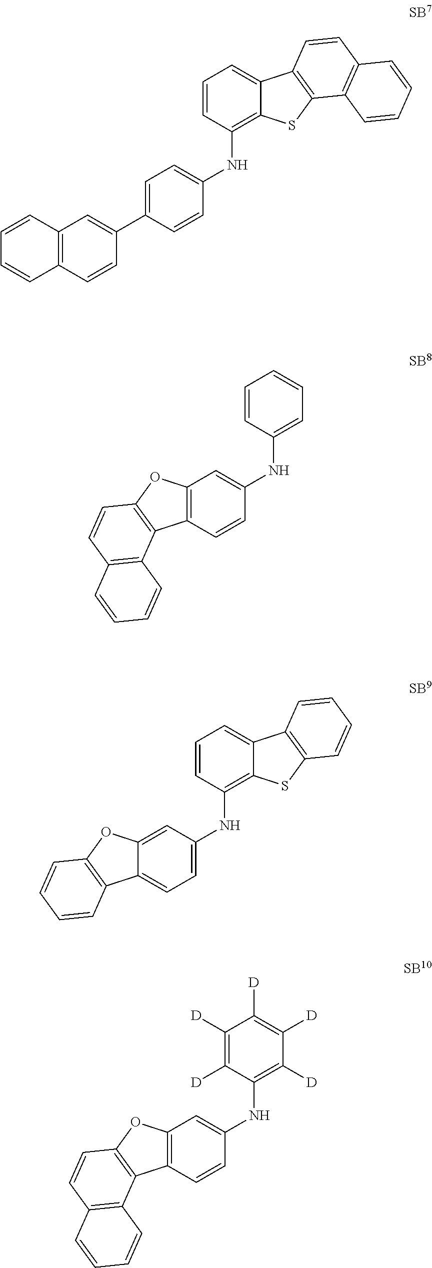 [SMILES: c1ccc2cc(-c3ccc(Nc4cccc5c4sc4c6ccccc6ccc54)cc3)ccc2c1 ; c1ccc(Nc2ccc3c(c2)oc2ccc4ccccc4c23)cc1 ; c1ccc2c(c1)oc1cc(Nc3cccc4c3sc3ccccc34)ccc12 ; [2H]c1c([2H])c([2H])c(Nc2ccc3c(c2)oc2ccc4ccccc4c23)c([2H])c1[2H]]

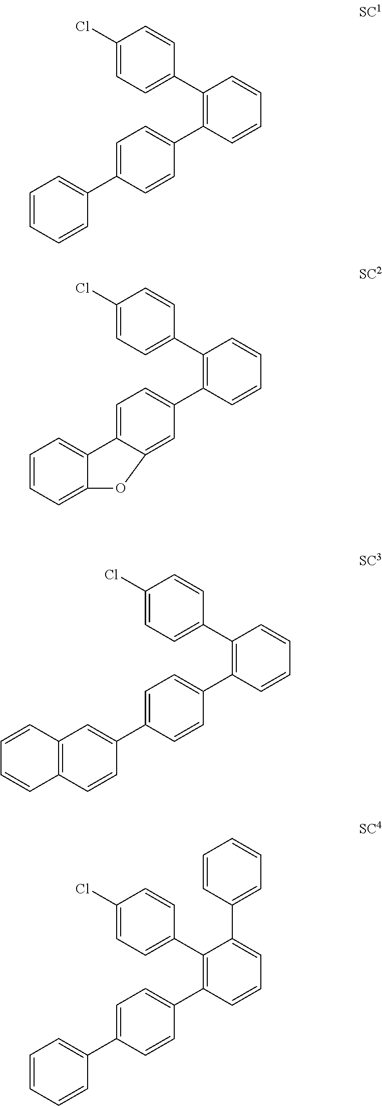 [SMILES: Clc1ccc(-c2ccccc2-c2ccc(-c3ccccc3)cc2)cc1 ; Clc1ccc(-c2ccccc2-c2ccc3c(c2)oc2ccccc23)cc1 ; Clc1ccc(-c2ccccc2-c2ccc(-c3ccc4ccccc4c3)cc2)cc1 ; Clc1ccc(-c2c(-c3ccccc3)cccc2-c2ccc(-c3ccccc3)cc2)cc1]

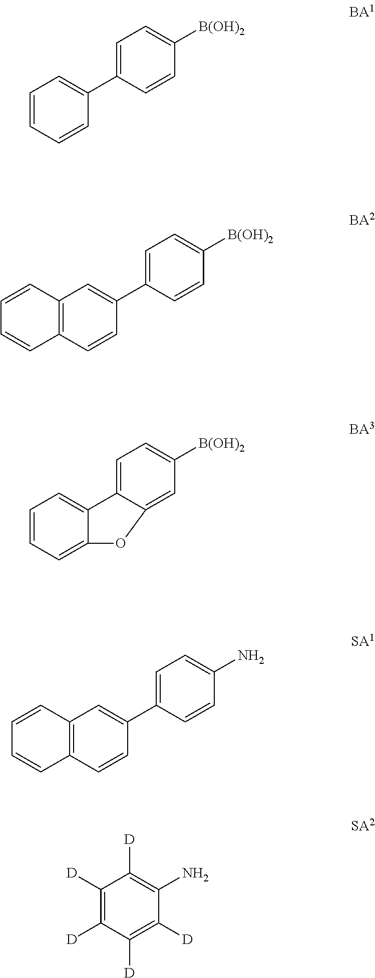 [SMILES: OB(O)c1ccc(-c2ccccc2)cc1 ; OB(O)c1ccc(-c2ccc3ccccc3c2)cc1 ; OB(O)c1ccc2c(c1)oc1ccccc12 ; Nc1ccc(-c2ccc3ccccc3c2)cc1 ; [2H]c1c([2H])c([2H])c(N)c([2H])c1[2H]]

### 1. Synthesis of Intermediate

### (Synthesis of Intermediate SB⁷)

Intermediate SB⁷ may be synthesized by the method of Reaction Scheme 1.

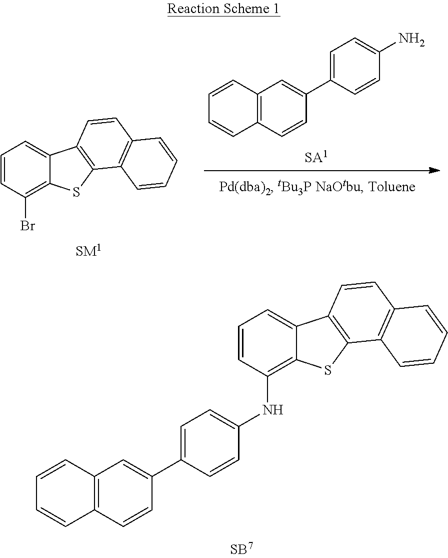 [SMILES: Brc1cccc2c1sc1c3ccccc3ccc21 ; Nc1ccc(-c2ccc3ccccc3c2)cc1 ; c1ccc2cc(-c3ccc(Nc4cccc5c4sc4c6ccccc6ccc54)cc3)ccc2c1]

A mixture of benzonaphthothiophene SM¹ (14.2 g, 45.6 mmol), arylamine SA¹ (10.0 g, 45.6 mmol), bis(dibenzylideneacetone) palladium (0) (Pd (dba) 2) (262 mg, 0.456 mmol), tri-tert-butylphosphine (^{t}Bu₃P) (2.0 M in toluene, 0.910 mL, 1.82 mmol), sodium tert-butoxide (NaO ^{t}Bu) (6.57 g, 68.4 mmol), and toluene (200 mL) was degassed, and then stirred for about 4 hours in an argon atmosphere while heating at about 60° C. The reaction mixture was cooled, and then diluted with tetrahydrofuran (THF), filtered, and concentrated to obtain a residue. The corresponding residue was crystallized with THF and hexane, and then washed with ethanol to obtain SB⁷ (7.64 g, yield of 37%).

### (Synthesis of Intermediate SB¹⁰)

Intermediate SB¹⁰ was synthesized in substantially the same manner as the synthesis of Intermediate SB⁷ by the Reaction Scheme 1. However, Benzonaphthofuran SM³ was used in place of Benzonaphthothiophene SM¹, and Arylamine SA² was used in place of Arylamine SA¹ to obtain Intermediate SB¹⁰ (yield of 86%)

### (Synthesis of Intermediate SC¹)

Intermediate SC¹ may be synthesized by the method of Reaction Scheme 2.

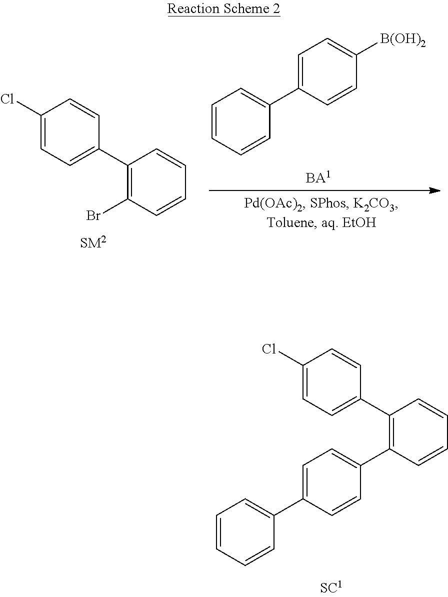

After degassing of a mixture of SM² (11.4 g, 42.6 mmol), BA¹ (9.29 g, 46.9 mmol, palladium acetate (Pd(OAc)₂) (669 mg, 2.98 mmol), SP hos (2.45 g, 5.96 mmol), potassium carbonate (K₂CO₃) (16.5 g, 119 mmol), toluene (120 mL), and aqueous ethanol (aq. ethanol) (EtOH/H₂O=1/2, 60 mL), the mixture was stirred for about 5 hours while heating at about 60° C. in an argon atmosphere. The reaction solution was filtered, and then extracted with toluene and concentrated. The obtained residue was crystallized with toluene-ethanol to obtain Intermediate SC¹ (7.69 g, 53%).

### (Synthesis of Intermediate SC²)

Intermediate SC² was synthesized by the Reaction Scheme 2. Intermediate SC² was synthesized in substantially the same manner as the synthesis of Intermediate SC¹ except that Intermediate BA³ was used in place of Intermediate BA¹. (yield of 55%)

### (Synthesis of Intermediate SC³)

Intermediate SC³ was synthesized by the Reaction Scheme 2. Intermediate SC³ was synthesized in substantially the same manner as the synthesis of Intermediate SC¹ except that Intermediate BA² was used in place of Intermediate BA¹. (yield of 50%)

### (Synthesis of Intermediate SC⁴)

Intermediate SC⁴ was synthesized by the Reaction Scheme 2. Intermediate SC⁴ was synthesized in substantially the same manner as the synthesis of Intermediate SC¹ except that Intermediate SM³ was used in place of Intermediate SM². (yield of 42%)

### 2. Synthesis of Example Compound

Example Compounds 50, 52, 67, 79, 94, 123, 143, 154, 352, 368, and 405 may be synthesized by the method of Reaction Scheme 3. In Reaction Scheme 3, E1 to E11 respectively correspond to Example Compounds 50, 52, 67, 79, 94, 123, 143, 154, 352, 368, and 405.

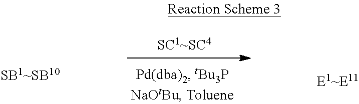

### (Synthesis of Compound 368)

A mixture of Intermediate SB⁷ (7.50 g, 16.6 mmol), Intermediate SC¹ (5.66 g, 16.6 mmol), bis(dibenzylideneacetone) palladium (0) (Pd (dba) 2) (95.5 mg, 0.170 mmol), tri-tert-butylphosphine (^{t}Bu₃P) (2.0 M in toluene, 0.330 mL, 0.660 mmol), sodium tri-tert-butoxide (NaO ^{t}Bu) (2.39 g, 24.9 mmol), and toluene (100 mL) was degassed, and then was stirred for about 10 hours while heating in an argon atmosphere at about 105° C. After cooling, toluene and tetrahydrofuran (THF) were added, the obtained was dissolved and filtered, and recrystallized with THE to obtain Compound 368 (9.32 g, 74%). The formation of Compound 368 was identified by fast atom bombardment mass spectroscopy (FAB-MS) measurement (m/z=755.3).

### (Synthesis of Compound 67)

Compound 67 was synthesized by the method of Reaction Scheme 3. Compound 67 (yield of 78%) was obtained in substantially the same manner as the synthesis of Compound 368 except that Intermediate SB¹ was used in place of Intermediate SB⁷. The formation of Compound 67 was identified by FAB-MS measurement (m/z=639.3).

### (Synthesis of Compound 352)

Compound 352 was synthesized by the method of Reaction Scheme 3. Compound 352 (yield of 74%) was obtained in substantially the same manner as the synthesis of Compound 368 except that Intermediate SB² was used in place of Intermediate SB⁷. The formation of Compound 352 was identified by FAB-MS measurement (m/z=699.3).

### (Synthesis of Compound 154)

Compound 154 was synthesized by the method of Reaction Scheme 3. Compound 154 (yield of 65%) was obtained in substantially the same manner as the synthesis of Compound 368 except that Intermediate SB³ was used in place of Intermediate SB⁷. The formation of Compound 154 was identified by FAB-MS measurement (m/z=714.3).

### (Synthesis of Compound 79)

Compound 79 was synthesized by a method of Reaction Scheme 3. Compound 79 (yield of 76%) was obtained in substantially the same manner as the synthesis of Compound 368 except that Intermediate SB⁴ was used in place of Intermediate SB⁷. The formation of Compound 79 was identified by FAB-MS measurement (m/z=713.3).

### (Synthesis of Compound 50)

Compound 50 was synthesized by a method of Reaction Scheme 3. Compound 50 (yield of 81%) was obtained in substantially the same manner as the synthesis of Compound 368 except that Intermediate SB⁵ was used in place of Intermediate SB⁷ Intermediate SC³ was used in place of Intermediate SC¹. The formation of Compound 50 was identified by FAB-MS measurement (m/z=725.3).

### (Synthesis of Compound 405)

Compound 405 was synthesized by a method of Reaction Scheme 3. Compound 405 (yield of 76%) was obtained in substantially the same manner as the synthesis of Compound 368 except that Intermediate SB⁶ was used in place of Intermediate SB⁷. The formation of Compound 405 was identified by FAB-MS measurement (m/z=739.3).

### (Synthesis of Compound 52)

Compound 52 was synthesized by a method of Reaction Scheme 3. Compound 52 (yield of 62%) was obtained in substantially the same manner as the synthesis of Compound 368 except that Intermediate SC⁴ was used in place of Intermediate SC¹ and Intermediate SB⁵ was used in place of Intermediate SB⁷. The formation of Compound 52 was identified by FAB-MS measurement (m/z=751.3).

### (Synthesis of Compound 123)

Compound 123 was synthesized by a method of Reaction Scheme 3. Compound 123 (yield of 70%) was obtained in substantially the same manner as the synthesis of Compound 368 except that Intermediate SB⁸ was used in place of Intermediate SB⁷. The formation of Compound 123 was identified by FAB-MS measurement (m/z=613.2).

### (Synthesis of Compound 94)

Compound 94 was synthesized by a method of Reaction Scheme 3. Compound 94 (yield of 72%) was obtained in substantially the same manner as the synthesis of Compound 368 except that Intermediate SB⁹ was used in place of Intermediate SB⁷ and Intermediate SC³ was used in place of Intermediate SC¹. The formation of Compound 94 was identified by FAB-MS measurement (m/z=719.2).

### (Synthesis of Compound 143)

Compound 143 was synthesized by a method of Reaction Scheme 3. Compound 143 (yield of 68%) was obtained in substantially the same manner as the synthesis of Compound 368 except that Intermediate SB¹⁰ was used in place of Intermediate SB⁷. The formation of Compound 143 was identified by FAB-MS measurement (m/z=618.3).

### 2. Manufacture and Evaluation of Light-Emitting Element

A light-emitting element according to one or more embodiments, containing an amine compound according to one or more embodiments in a hole transport layer was manufactured by a following method. The light-emitting elements according to Example 1 to Example 11 were manufactured using, as a material in the hole transport layer, Amine Compounds 50, 52, 67, 79, 94, 123, 143, 154, 352, 368, and 405, which were the herein-described example compounds. The light-emitting elements according to Comparative Example 1 to Comparative Example 8 correspond to the light-emitting elements manufactured respectively using Comparative Example Compound C1 to Comparative Example Compound C8 as a material in the hole transport layer.

### Example Compounds

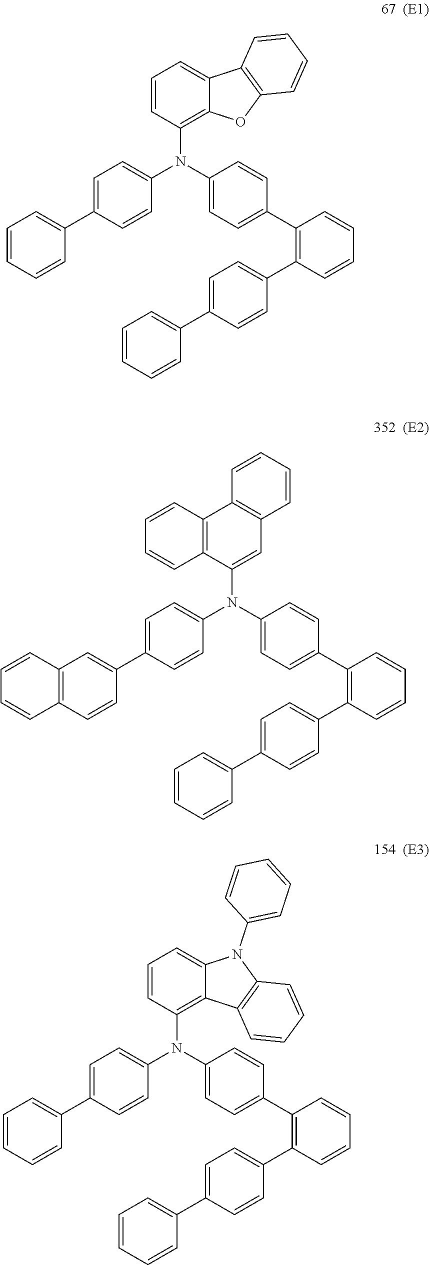 [SMILES: c1ccc(-c2ccc(-c3ccccc3-c3ccc(N(c4ccc(-c5ccccc5)cc4)c4cccc5c4oc4ccccc45)cc3)cc2)cc1 ; c1ccc(-c2ccc(-c3ccccc3-c3ccc(N(c4ccc(-c5ccc6ccccc6c5)cc4)c4cc5ccccc5c5ccccc45)cc3)cc2)cc1 ; c1ccc(-c2ccc(-c3ccccc3-c3ccc(N(c4ccc(-c5ccccc5)cc4)c4cccc5c4c4ccccc4n5-c4ccccc4)cc3)cc2)cc1]

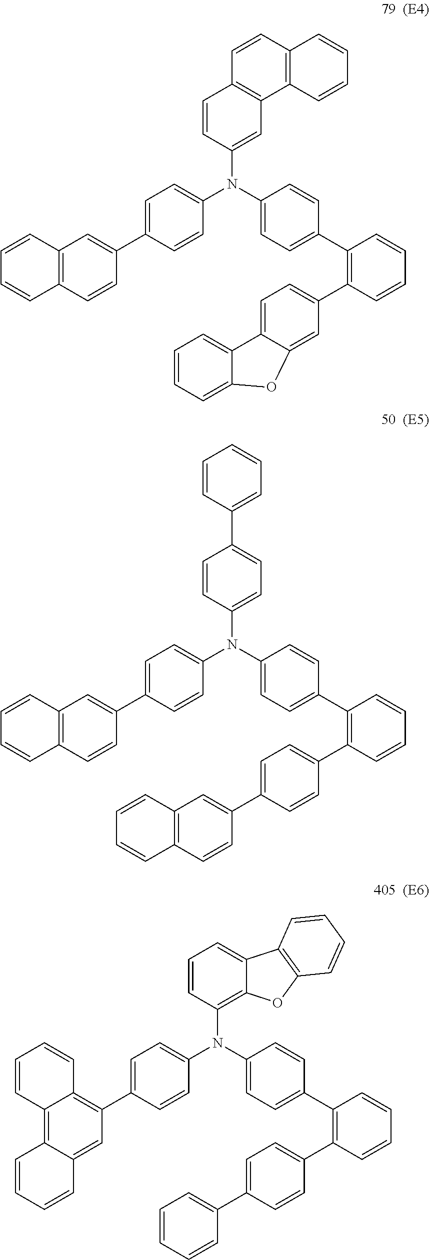 [SMILES: c1ccc(-c2ccc3c(c2)oc2ccccc23)c(-c2ccc(N(c3ccc(-c4ccc5ccccc5c4)cc3)c3ccc4ccc5ccccc5c4c3)cc2)c1 ; c1ccc(-c2ccc(N(c3ccc(-c4ccc5ccccc5c4)cc3)c3ccc(-c4ccccc4-c4ccc(-c5ccc6ccccc6c5)cc4)cc3)cc2)cc1 ; c1ccc(-c2ccc(-c3ccccc3-c3ccc(N(c4ccc(-c5cc6ccccc6c6ccccc56)cc4)c4cccc5c4oc4ccccc45)cc3)cc2)cc1]

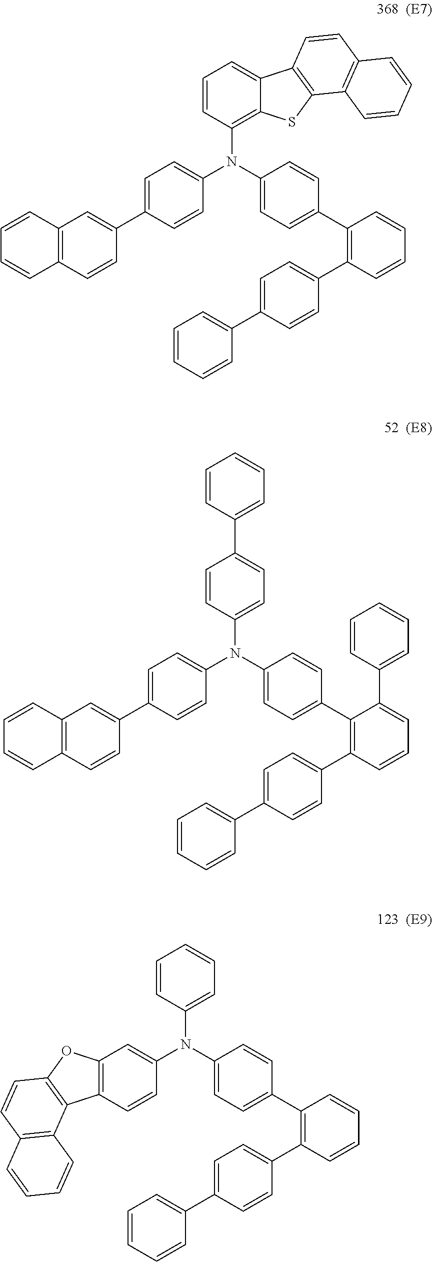 [SMILES: c1ccc(-c2ccc(-c3ccccc3-c3ccc(N(c4ccc(-c5ccc6ccccc6c5)cc4)c4cccc5c4sc4c6ccccc6ccc54)cc3)cc2)cc1 ; c1ccc(-c2ccc(-c3cccc(-c4ccccc4)c3-c3ccc(N(c4ccc(-c5ccccc5)cc4)c4ccc(-c5ccc6ccccc6c5)cc4)cc3)cc2)cc1 ; c1ccc(-c2ccc(-c3ccccc3-c3ccc(N(c4ccccc4)c4ccc5c(c4)oc4ccc6ccccc6c45)cc3)cc2)cc1]

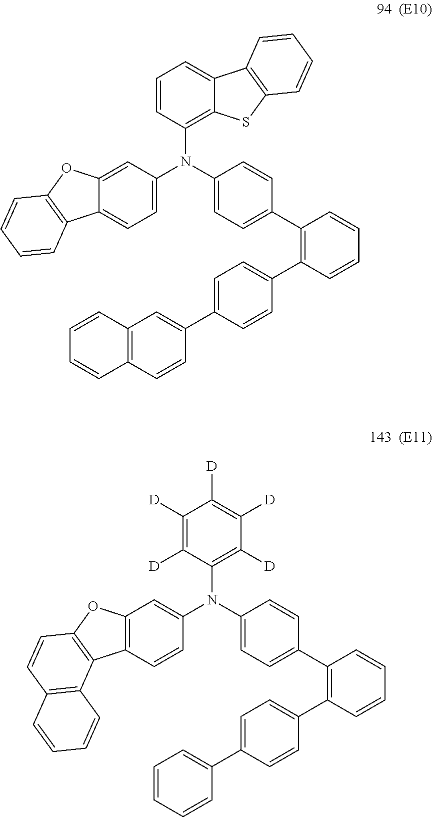 [SMILES: c1ccc(-c2ccc(N(c3ccc4c(c3)oc3ccccc34)c3cccc4c3sc3ccccc34)cc2)c(-c2ccc(-c3ccc4ccccc4c3)cc2)c1 ; [2H]c1c([2H])c([2H])c(N(c2ccc(-c3ccccc3-c3ccc(-c4ccccc4)cc3)cc2)c2ccc3c(c2)oc2ccc4ccccc4c23)c([2H])c1[2H]]

### Comparative Example Compounds

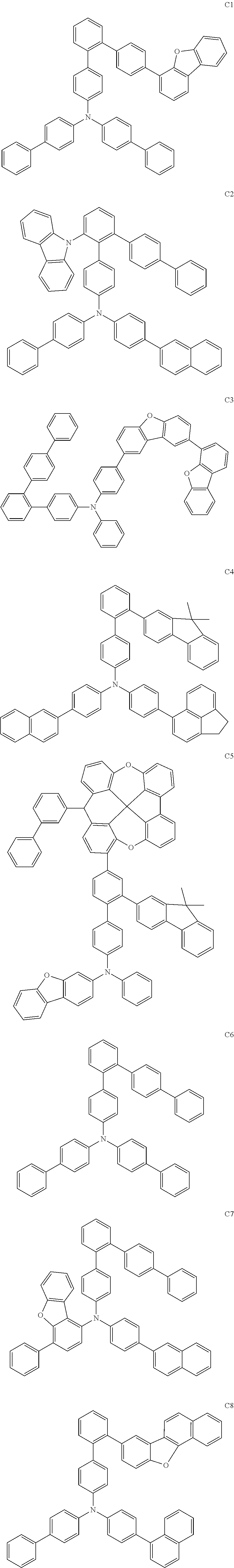

A glass substrate of 15 ohm per square centimeter (Ω/cm²) (1500 angstrom (Å)) ITO by Corning was cut in a size of about 50 millimeter (mm)×50 mm×0.7 mm, washed with isopropyl alcohol and ultrapure water, and cleansed using ultrasonic waves for about 5 minutes. Then, the glass substrate was irradiated to UV for about 30 minutes and then treated with ozone. Thereafter, 4,4′,4″-tris {N,-(2-naphthyl)-N-phenylamino}-triphenylamine (2-TNATA) was vacuum deposited to a thickness of about 600 Å to form a hole injection layer, and then an example compound or a comparative example compound was vacuum deposited to a thickness of about 300 Å to form a hole transport layer.

9,10-di(naphthalen-2-yl)anthracene (AND), which is a blue fluorescent host, and 2,5,8,11-tetra-t-butylperylene (TBP), which is a fluorescent dopant, were co-deposited in a ratio (e.g., amount) of about 97:3 to form a light-emitting layer having a thickness of about 250 Å on the hole transport layer.

An electron transport layer having a thickness of about 250 Å was formed with tris(8-hydroxyquinolino)aluminum (Alq₃) on the light-emitting layer, and then LiF was deposited to about 10 Å to from an electron injection layer. A second electrode having a thickness of about 1000 Å was formed on the electron injection layer with aluminum (Al).

For example, the compounds of each functional group used in the manufacture of the light-emitting element are as follows.

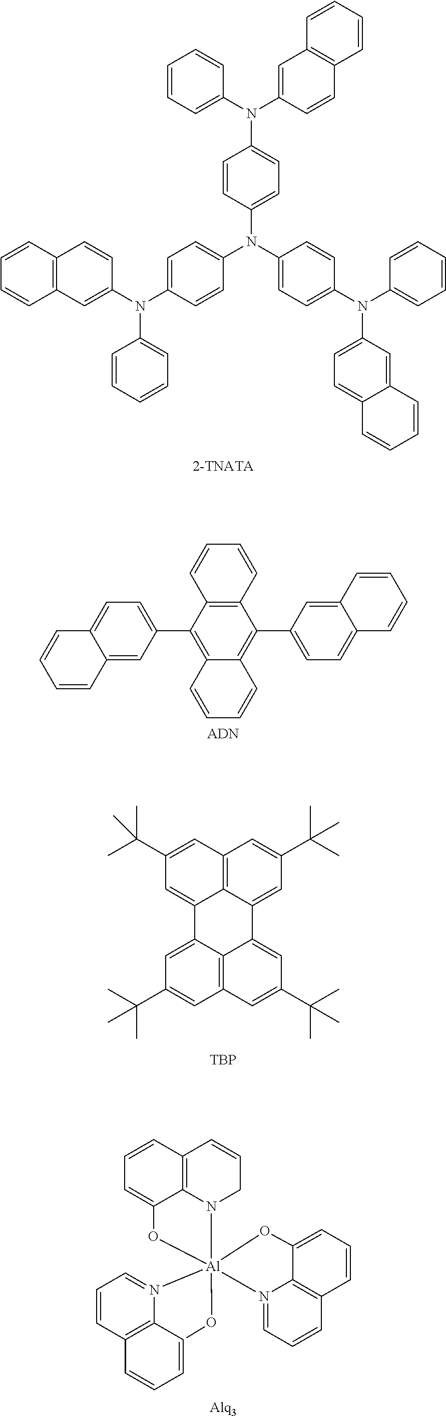

### (Evaluation of Light-Emitting Element)

Light-emitting elements according to Example 1 to Example 11, and Comparative Example 1 to Comparative Example 8 were evaluated and the evaluation results were listed in Table 1. In Table 1, luminous efficiency and lifespan of each manufactured light-emitting element are shown.

In evaluation results of properties of each light-emitting elements according to examples and comparative examples, which are listed in Table 1, the luminous efficiency refers to an efficiency value at a current density of 10 milliampere per square centimeter (mA/cm²), and the lifespan of the element refers to a luminous half-lifespan at a current density of 10 mA/cm². A decrease in purity was expressed as % of difference in purity between the material before and after deposition.

Evaluations of element current density and luminous efficiency were performed using a source meter of 2400 Series, which is a product made by Keithley Instruments, a colorimeter of CS-200, which is a product made by Konica Minolta Co., Ltd., and LabVIEW8.2, which is a PC software for measurement made by National Instruments Japan Co., Ltd. in a dark room.

In some embodiments, the luminous efficiency and element lifespan values of Comparative Example 1 are set to 100% and the relative values of luminous efficiency and element lifespan of each light-emitting element are calculated and listed.

TABLE 1

| | | | Life- | Decrease in |
|---|---|---|---|---|
| | Hole transport | Efficiency | span | purity |
| | material | [%] | [%] | [%] |
| Example 1 | Compound 67 (E1) | 115 | 130 | 0.0 |
| Example 2 | Compound 352 (E2) | 125 | 115 | 0.0 |
| Example 3 | Compound 154 (E3) | 120 | 115 | 0.0 |
| Example 4 | Compound 79 (E4) | 120 | 120 | 0.0 |
| Example 5 | Compound 50 (E5) | 115 | 120 | 0.0 |
| Example 6 | Compound 405 (E6) | 125 | 115 | 0.0 |
| Example 7 | Compound 368 (E7) | 120 | 125 | 0.0 |
| Example 8 | Compound 52 (E8) | 115 | 115 | 0.0 |
| Example 9 | Compound 123 (E9) | 115 | 120 | 0.0 |
| Example 10 | Compound 94 (E10) | 120 | 120 | 0.0 |
| Example 11 | Compound 143 (E11) | 115 | 125 | 0.0 |
| Comparative | Comparative Example | 100 | 100 | 0.0 |
| Example 1 | Compound C1 | | | |
| Comparative | Comparative Example | 105 | 90 | 0.2 |
| Example 2 | Compound C2 | | | |
| Comparative | Comparative Example | 90 | 70 | 0.2 |
| Example 3 | Compound C3 | | | |
| Comparative | Comparative Example | 95 | 60 | 0.4 |
| Example 4 | Compound C4 | | | |
| Comparative | Comparative Example | 95 | 50 | 0.4 |
| Example 5 | Compound C5 | | | |
| Comparative | Comparative Example | 97 | 100 | 0.0 |
| Example 6 | Compound C6 | | | |
| Comparative | Comparative Example | 102 | 75 | 0.2 |
| Example 7 | Compound C7 | | | |
| Comparative | Comparative Example | 97 | 95 | 0.0 |
| Example 8 | Compound C8 | | | |

Referring to Table 1, it may be seen that the light-emitting elements according to examples using the amine compound according to one or more embodiments of the disclosure as a material for the hole transport layer exhibit high luminous efficiency and long element lifespan, compared to the light-emitting elements according to comparative examples. It may be seen that when the comparative example compounds, compared to the example compounds, are applied to the light-emitting elements, the light-emitting elements have reduced luminous efficiency and lifespan. For example, referring to Table 1, it may be seen that the light-emitting elements using the amine compound according to one or more embodiments exhibit improved element characteristics in luminous efficiency and element lifespan, compared to the light-emitting elements according to comparative examples.

The amine compound according to one or more embodiments includes a 1,1′:2′,1″:4″,1′″-quarterphenyl moiety, and may have characteristics in that the 1,1′:2′,1″:4″,1″-quarterphenyl moiety is bonded to a nitrogen atom of the amine group at a specific position. In some embodiments, the amine compound according to one or more embodiments may include at least one selected from among a substituted or unsubstituted naphthylphenyl group, a substituted or unsubstituted dibenzofuranyl group, a substituted or unsubstituted dibenzothiophenyl group, and a substituted or unsubstituted carbazolyl group, as a substituent (e.g., bonded to a nitrogen atom of the amine group at a specific position). The amine compound according to one or more embodiments may have excellent or suitable stability and high charge transporting ability by introducing such a substituent and specification of a substitution position. Therefore, the amine compound according to one or more embodiments may have improved efficiency and lifespan. In some embodiments, the light-emitting element according to one or more embodiments containing the amine compound according to one or more embodiments may have improved luminous efficiency and lifespan.

The light-emitting elements according to Comparative Examples 1 and 6 have reduced luminous efficiency and element lifespan. Each of Comparative Example Compounds C1 and C6 respectively contained in the light-emitting elements according to Comparative Examples 1 and 6 does not include (e.g., bonded to a nitrogen atom of the amine group at a specific position), at least one substituent selected from among a substituted or unsubstituted naphthylphenyl group, a substituted or unsubstituted dibenzofuranyl group, a substituted or unsubstituted dibenzothiophenyl group, and a substituted or unsubstituted carbazolyl group, which differs from example compounds. Example compounds may exhibit characteristics of high molecular stability and improved molecular orientation by including the specific substituent. Therefore, it can be thought that the light-emitting elements according to examples exhibit element characteristics of long lifespan and high efficiency, compared to the light-emitting elements according to Comparative Examples 1 and 6.

The light-emitting elements according to Comparative Example 2 has reduced luminous efficiency and element lifespan, compared to the light-emitting elements according to examples. Comparative Example Compound C2 contained in the light-emitting element according to Comparative Example 2 corresponds a case where a carbazolyl group is substituted at R⁵ position in Formula 1 of the present disclosure. The substitution position of R⁵ exists at a bent position of a quarterphenyl moiety, and when a substituent having a larger volume than a phenyl group such as a carbazolyl group exists at the corresponding position, an excessive distortion in the bent portion of the quarterphenyl moiety is caused, and thus molecular stability may be reduced. Therefore, it can be confirmed that the light-emitting element according to Comparative Example 2 containing Comparative Example Compound C2 has reduced luminous efficiency and element lifespan, compared to the light-emitting elements according to examples.

The light-emitting elements according to Comparative Example 3 has reduced luminous efficiency and element lifespan, compared to the light-emitting elements according to examples. Comparative Example Compound C3 contained in the light-emitting element according to Comparative Example 3 includes two consecutively bonded heteroaryl groups, which differs from example compounds. Because Comparative Example Compound C3 includes two consecutively bonded heteroaryl groups, stability of the whole molecule is reduced. Therefore, it is thought that the light-emitting element according to Comparative Example 3 has reduced luminous efficiency and lifespan.

The light-emitting elements according to Comparative Example 4 has reduced luminous efficiency and element lifespan, compared to the light-emitting elements according to examples. Comparative Example Compound C4 contained in the light-emitting element according to Comparative Example 4 corresponds a compound which includes acenaphthene in a molecule. The acenaphthene having a fused structure, in which a cycloalkyl ring is fused to an aryl ring, is thermally and chemically unstable due to a cycloalkyl skeleton that includes two sp³ carbons, thereby tending to easily decompose during deposition. Therefore, the light-emitting element according to Comparative Example 4 containing Comparative Example Compound C4 has reduced luminous efficiency and lifespan, compared to the light-emitting elements according to examples.

The light-emitting elements according to Comparative Example 5 has reduced luminous efficiency and element lifespan, compared to the light-emitting elements according to examples. As Comparative Example Compound 5, when a heteroaryl group having 26 or more carbons is included as a substituent linked to a quaterphenyl moiety, a deposition temperature increases, and thus a lifespan thereof may be reduced due to decomposition of the compound. Therefore, it is thought that the light-emitting element according to Comparative Example 5 containing Comparative Example Compound C5 has deteriorated element characteristics, compared to the light-emitting elements according to examples.

The light-emitting elements according to Comparative Example 7 has reduced luminous efficiency and element lifespan, compared to the light-emitting elements according to examples. Comparative Example Compound C7 contained in the light-emitting element according to Comparative Example 7 corresponds to a case where Ar¹ or Ar² of the present disclosure is a 4-phenyl-dibenzofuran-1-yl group. In a case of a compound in which Ar¹ or Ar² is a 4-phenyl-dibenzofuran-1-yl group, resonance stabilization of a dibenzofuran ring is relatively small, and a first carbon and a fourth carbon of the dibenzofuran ring is respectively bonded to a nitrogen atom and a phenyl group of amine group, and thus thermal stability of the whole molecule is reduced due to distortion of the molecule. It is thought that the light-emitting element according to Comparative Example 7 has element reduced characteristics according to these characteristics of Comparative Example Compound C7, compared to the light-emitting elements according to examples.

The light-emitting element according to Comparative Example 8 has reduced luminous efficiency and element lifespan, compared to the light-emitting elements according to examples. In Comparative Example Compound C8 contained in the light-emitting element according to Comparative Example 8, a 1,1′:2′,1″:3″,1′″-quaterphenylyl group is in place of a portion of an essential 1,1′:2′,1″:4″,1″-quaterphenylyl group in the disclosure, and a 4″,1″-(para) connection structure has disappeared, becoming a 3″,1′″-(meta) connection structure. Although the corresponding part, which is the 4″,1″-connection part, rotates along a connection axis, no significant change in the structure occurs. However, significant changes are caused in the structure with the 3″,1′″-connection by rotation along a connection axis, reducing the robustness of the corresponding group. Therefore, it is thought that the light-emitting element has reduced efficiency because charge transporting properties and/or the like are affected.

The light-emitting element according to one or more embodiments may exhibit characteristics of high efficiency and long lifespan by including an amine compound according to one or more embodiments.

The light-emitting element may exhibit characteristics of high efficiency and long lifespan when the amine compound according to one or more embodiments applied to the light-emitting element.

The display device according to one or more embodiments may exhibit excellent or suitable display quality.

Again, referring to Table 1, it is evident that light-emitting elements using the amine compound from one or more embodiments exhibit higher luminous efficiency and longer lifespan compared to those using comparative example compounds. The amine compound includes a 1,1′:2′,1″:4″,1″-quarterphenyl moiety bonded to a nitrogen atom of the amine group at a specific position, and may also include other substituents like naphthylphenyl, dibenzofuranyl, dibenzothiophenyl, and/or carbazolyl groups. These compounds provide excellent stability and high charge transporting ability, leading to improved efficiency and lifespan of the light-emitting elements.

Comparative examples show reduced luminous efficiency and lifespan due to the absence of these specific substituents or due to structural issues like excessive distortion or instability. For instance, compounds with acenaphthene or large heteroaryl groups exhibit thermal and chemical instability, leading to decomposition during deposition. Overall, the light-emitting elements according to the embodiments demonstrate superior performance in terms of efficiency and lifespan.

In the context of the present application and unless otherwise defined, the terms “use,” “using,” and “used” may be considered synonymous with the terms “utilize,” “utilizing,” and “utilized,” respectively.

A person of ordinary skill in the art, in view of the present disclosure in its entirety, would appreciate that each suitable feature of the one or more suitable embodiments of the present disclosure may be combined or combined with each other, partially or entirely, and may be technically interlocked and operated in one or more suitable ways, and each embodiment may be implemented independently of each other or in conjunction with each other in any suitable manner unless otherwise stated or implied.

Hitherto, although one or more embodiments of present disclosure have been described with reference to example embodiments, it is understood that present disclosure should not be limited to these embodiments, but one or more suitable changes and modifications can be made by one ordinary skilled in the art within the spirit and scope of present disclosure as hereinafter claimed. Therefore, the technical scope of the present disclosure is not intended to be limited to the contents set forth in the detailed description of the specification, but is intended to be defined by the appended claims, and equivalents thereof.

What is claimed is:

1. A light-emitting element comprising:
a first electrode;
a second electrode on the first electrode; and
at least one functional layer between the first electrode and the second electrode and comprising an amine compound, the amine compound represented by Formula 1: 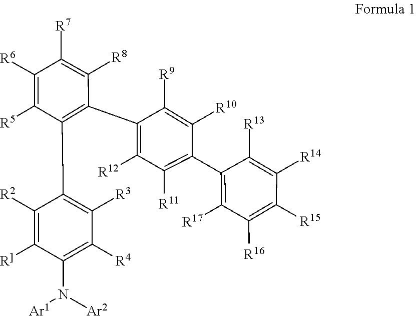
wherein, in Formula 1,
R¹ to R¹⁷ are each independently a hydrogen atom, a deuterium atom, a substituted or unsubstituted oxy group, a substituted or unsubstituted thio group, a substituted or unsubstituted amine group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 25 ring-forming carbons, and/or bonded to an adjacent group to form a ring,
wherein
a case where R⁵ is a substituted or unsubstituted carbazolyl group is excluded,
Ar¹ and Ar² are each independently a substituted or unsubstituted aryl group having 6 to 30 ring-forming carbons, or a substituted or unsubstituted heteroaryl group having 2 to 30 ring-forming carbons, and
wherein
at least one selected from among Ar¹ and Ar² comprises a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group,
when each of Ar¹ and Ar² comprises a substituted group, then a substituent of the substituted group is to bond to an adjacent group to form a ring,
a case where at least one selected from among Ar¹ and Ar² is a 4-phenyl-dibenzofuran-1-yl group is excluded,
a case where each Ar¹ and Ar² comprises a heteroaryl group substituted with a heteroaryl group is excluded,
a case where the amine compound comprises at least one selected from among a chlorine atom and acenaphthene is excluded, and
at least one hydrogen atom of the amine compound is substituted with a deuterium atom.

2. The light-emitting element of claim 1,
wherein the at least one functional layer comprises:
a light-emitting layer;
a hole transport region between the first electrode and the light-emitting layer; and
an electron transport region between the light-emitting layer and the second electrode, and
the hole transport region includes the amine compound.

3. The light-emitting element of claim 2,
wherein the hole transport region comprises:
a hole injection layer on the first electrode; and
a hole transport layer on the hole injection layer, and
the hole transport layer includes the amine compound.

4. The light-emitting element of claim 1,
wherein at least one selected from among Ar¹ and Ar² is represented by any one selected from among Formula 2-1 to Formula 2-3: 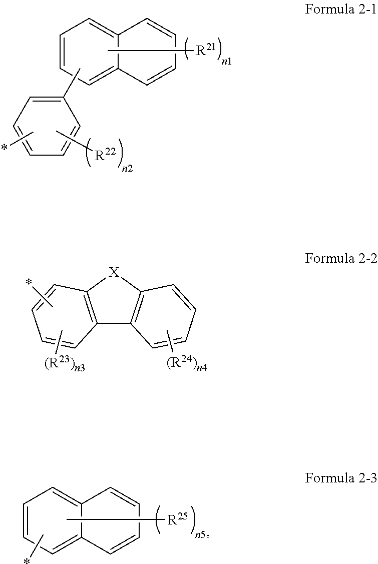
wherein, in Formula 2-1 to Formula 2-3,
X is O, S, or NR²⁶,
R²¹, R²², R²⁵, and R²⁶ are each independently a hydrogen atom, a deuterium atom, a halogen atom, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or bonded to an adjacent group to form a ring,
R²³ and R²⁴ are each independently a hydrogen atom, a deuterium atom, a halogen atom, a substituted or unsubstituted alkyl group having 1 to 20 carbons, a substituted or unsubstituted alkenyl group having 2 to 20 carbons, or a substituted or unsubstituted aryl group having 6 to 30 ring-forming carbons, and/or bonded to an adjacent group to form a ring,
n1 is an integer of 0 to 7,
n2 and n4 are each independently an integer of 0 to 4,
n3 is an integer of 0 to 3,
n5 is an integer of 0 to 7, and [symbol]
is a position to which Formula 1 is connected.

5. The light-emitting element of claim 4, wherein:
one selected from among Ar¹ and Ar² is represented by any one selected from among Formula 2-1 to Formula 2-3; and
the other, selected from among Ar¹ and Ar², is a substituted or unsubstituted phenyl group, a substituted or unsubstituted biphenylyl group, a substituted or unsubstituted terphenylyl group, a substituted or unsubstituted quarterphenylyl group, a substituted or unsubstituted naphthyl group, a substituted or unsubstituted naphthylphenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a substituted or unsubstituted phenanthrenyl group.

6. The light-emitting element of claim 1, wherein the amine compound is represented by Formula 3: 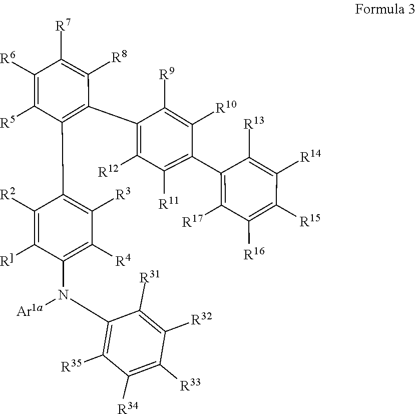
wherein, in Formula 3,
R³¹ to R³⁵ are each independently a hydrogen atom, a deuterium atom, a halogen atom, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, a substituted or unsubstituted heteroaryl group having 2 to 30 ring-forming carbons, and/or bonded to an adjacent group to form a ring,
wherein
a case where at least one selected from among R³¹ to R³⁵ is a heteroaryl group substituted with a heteroaryl group is excluded, and
Ar^{1a} comprises a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group, and when Ar^{1a} comprises a substituted naphthyl group, a substituted naphthylphenyl group, a substituted dibenzofuranyl group, a substituted dibenzothiophenyl group, or a substituted carbazolyl group, then the substituent is to bond to an adjacent group to form a ring, and
wherein
a case where Ar^{1a} comprises a heteroaryl group substituted with a heteroaryl group is excluded,
a case where Ar^{1a} is represented by Formula 3-a is excluded: 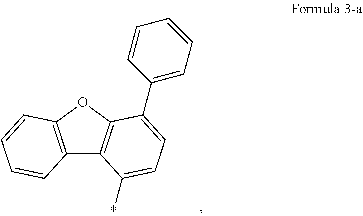 [SMILES: *c1ccc(-c2ccccc2)c2oc3ccccc3c12]
and
R¹ to R¹⁷ are as defined in Formula 1.

7. The light-emitting element of claim 6, wherein
R³¹ to R³⁵ are each independently a hydrogen atom, a deuterium atom, a substituted or unsubstituted phenyl group, a substituted or unsubstituted naphthyl group, or a substituted or unsubstituted phenanthrenyl group, or
wherein at least one pair of groups, selected from among R³¹ to R³⁵, is bonded together to form a ring.

8. The light-emitting element of claim 1, wherein the amine compound is represented by any one selected from among Formula 4-1 to Formula 4-3: 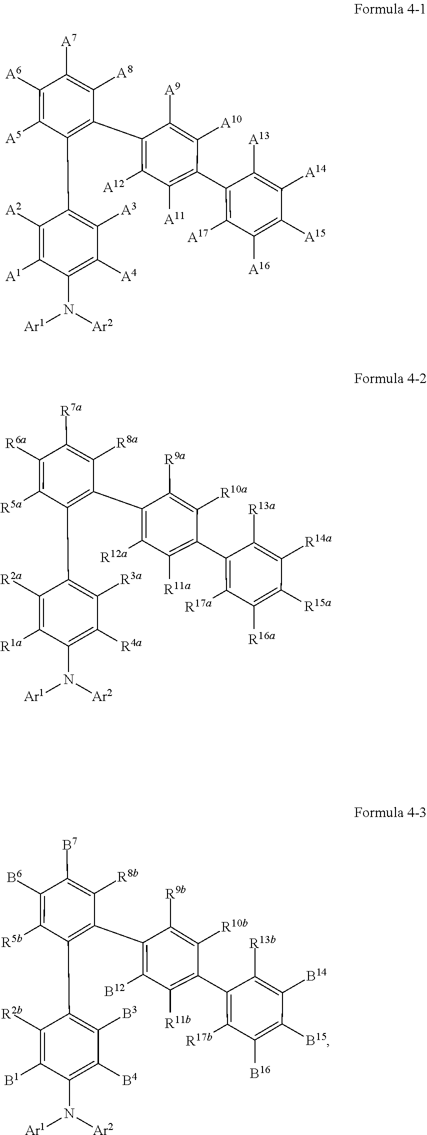
wherein,
in Formula 4-1,
A¹ to A¹⁷ are each independently a hydrogen atom, a deuterium atom, or a substituted or unsubstituted aryl group having 6 to 30 ring-forming carbons,
in Formula 4-2,
R^{1a} to R^{17a} are each independently a hydrogen atom, a deuterium atom, a substituted or unsubstituted oxy group, a substituted or unsubstituted thio group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or bonded to an adjacent group to form a ring, and
at least one pair of groups, selected from among R^{1a} to R^{17a}, are bonded together to form an aromatic hydrocarbon ring,
in Formula 4-3,
B¹, B³, B⁴, B⁶, B⁷, B¹², B¹⁴, B¹⁵, and B¹⁶ are each independently a hydrogen atom or a deuterium atom,
R^{2b}, R^{5b}, R^{8b}, R^{9b}, R^{10b}, R^{11b}, R^{13b}, and R^{17b} are each independently a hydrogen atom, a deuterium atom, a substituted or unsubstituted oxy group, a substituted or unsubstituted thio group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or bonded to an adjacent group to form a ring, and
at least one pair selected from among R^{2b} and R^{5b}, R^{8b} and R^{9b}, R^{10b} and R^{13b}, and R^{11b} and R^{17b} are bonded to each other to form a ring, and
in Formula 4-1 to 4-3,
Ar¹ and Ar² are as defined in Formula 1.

9. The light-emitting element of claim 1,
wherein the amine compound is represented by Formula 5, and satisfies any one selected from among combinations present in Compound Combination Table 1: 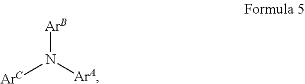
and
wherein, in Formula 5,
Ar^{A} is selected from among the group consisting of Substituent 1 to Substituent 12 in Substituent Group A,
Ar^{B} is selected from among the group consisting of Substituent 1 to Substituent 12, and Substituent 21 to Substituent 52 in Substituent Group A, and
Ar^{C} is selected from among the group consisting of Substituent 2 to Substituent 7, Substituent 9 to Substituent 11, and Substituent 31 to Substituent 52 in Substituent Group A: 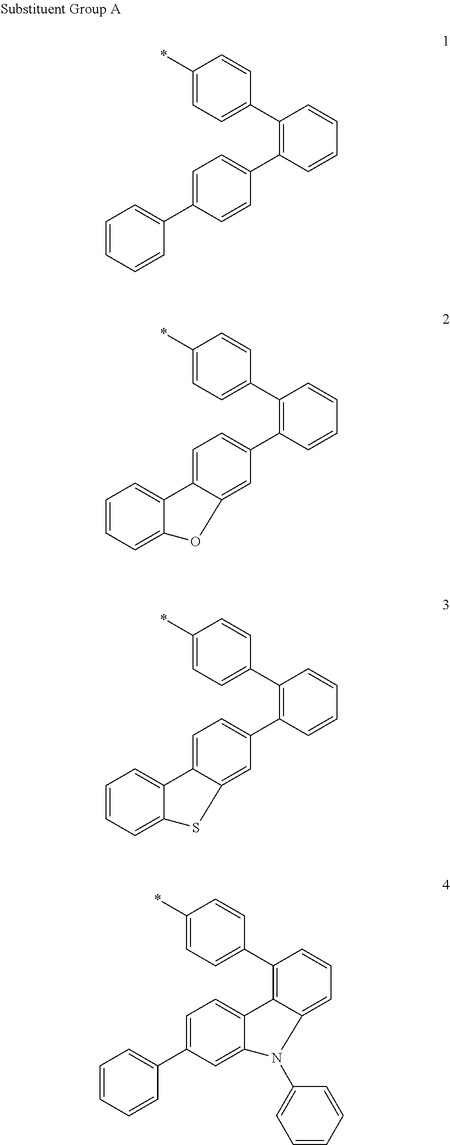 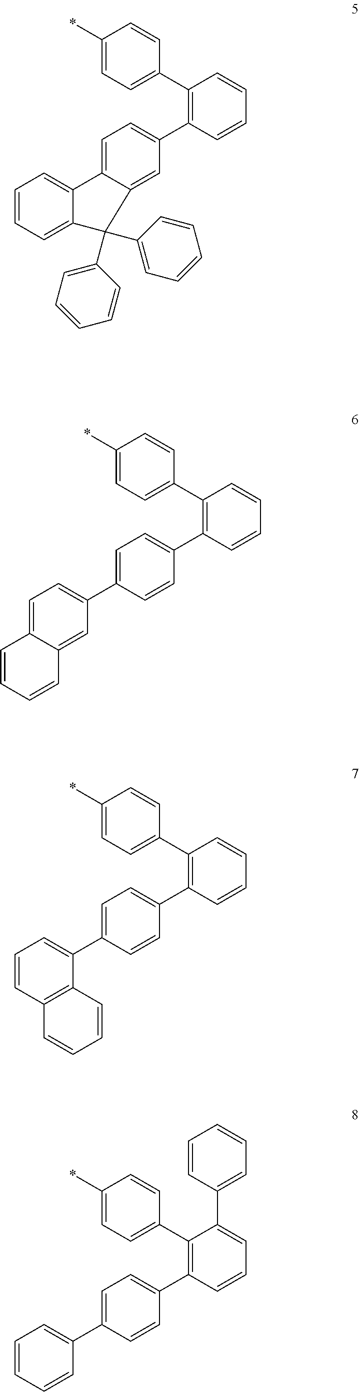 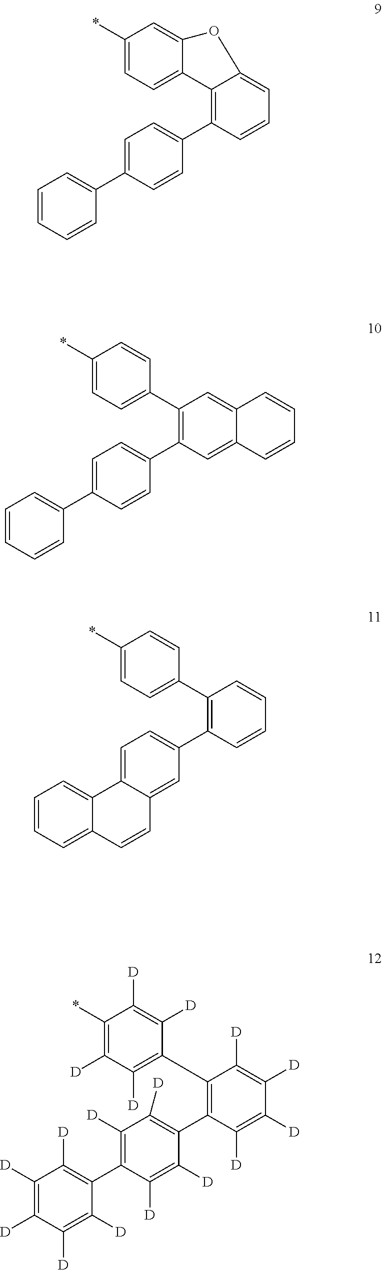 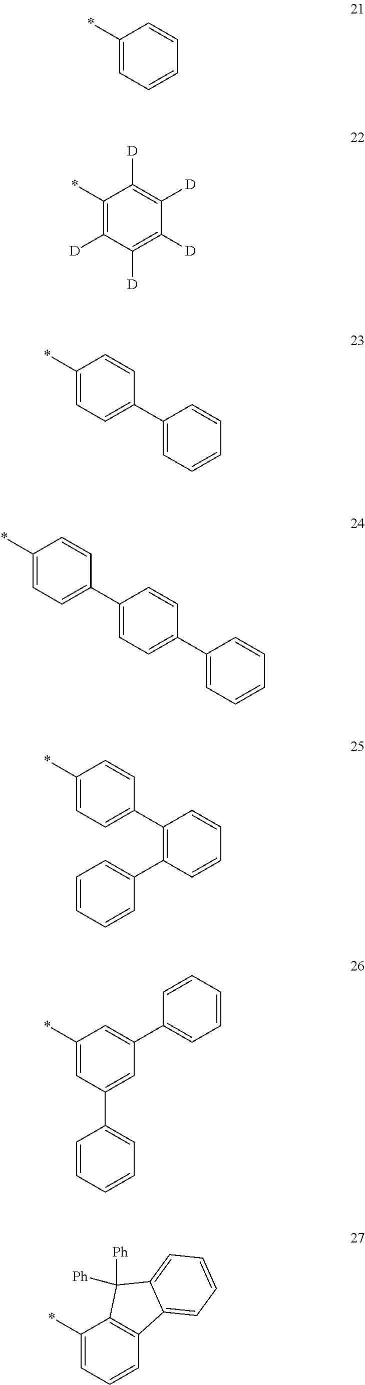 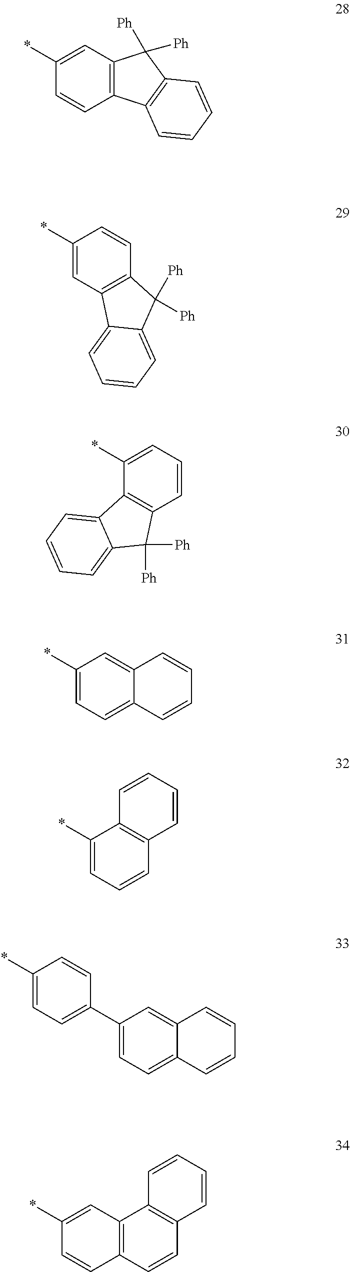 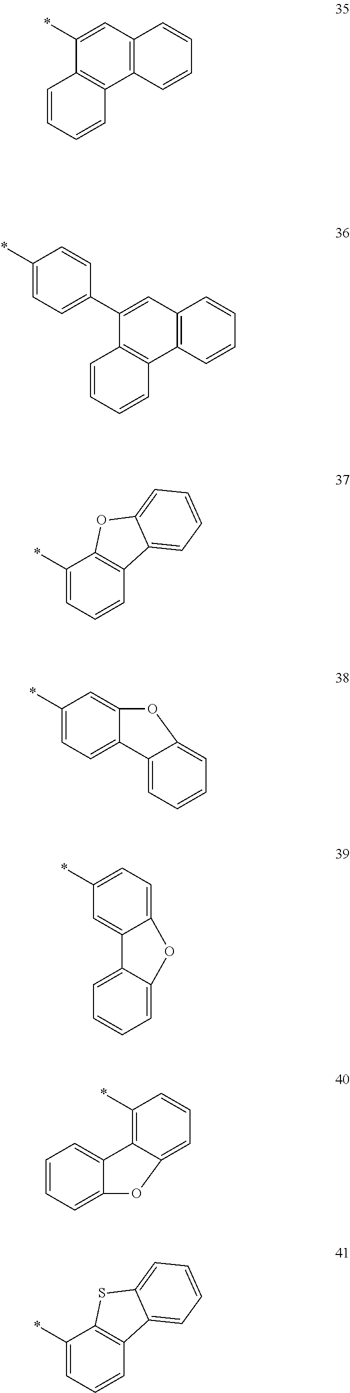 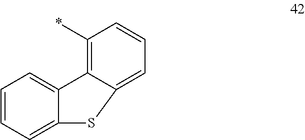 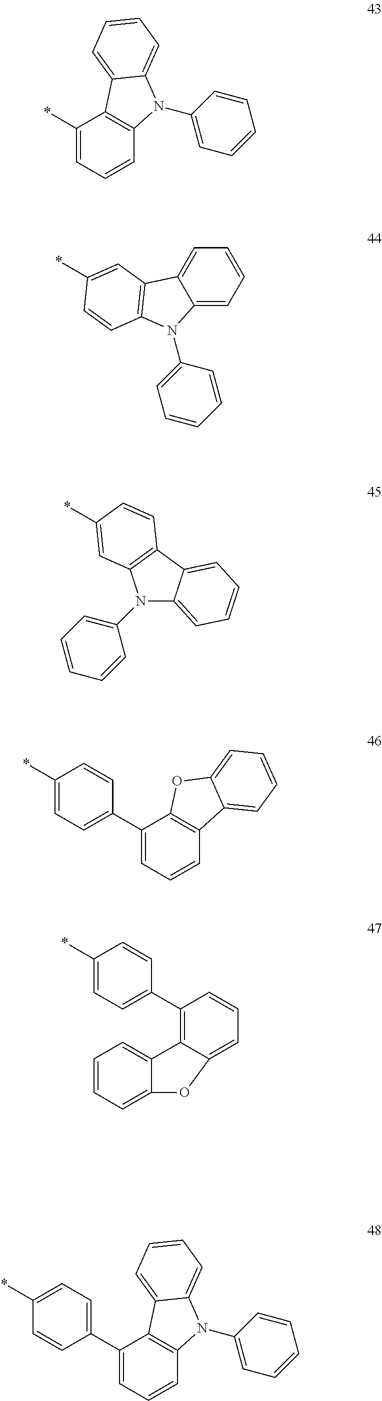 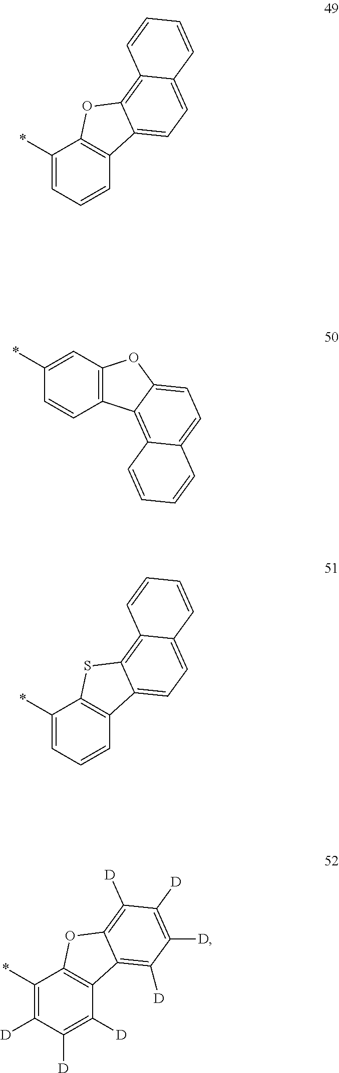
wherein, [symbol] is a position to which Formula 1 is connected,
Compound Combination Table 1

| No | Ar^{A} | Ar^{B} | Ar^{C} |
|---|---|---|---|
| 1 | 1 | 21 | 33 |
| 2 | 2 | 21 | 33 |
| 3 | 3 | 21 | 33 |
| 4 | 4 | 21 | 33 |
| 5 | 5 | 21 | 33 |
| 6 | 6 | 21 | 33 |
| 7 | 7 | 21 | 33 |
| 8 | 8 | 21 | 33 |
| 9 | 9 | 21 | 33 |
| 10 | 10 | 21 | 33 |
| 11 | 11 | 21 | 33 |
| 12 | 1 | 21 | 34 |
| 13 | 2 | 21 | 34 |
| 14 | 3 | 21 | 34 |
| 15 | 4 | 21 | 34 |
| 16 | 5 | 21 | 34 |
| 17 | 6 | 21 | 34 |
| 18 | 7 | 21 | 34 |
| 19 | 8 | 21 | 34 |
| 20 | 9 | 21 | 34 |
| 21 | 10 | 21 | 34 |
| 22 | 11 | 21 | 34 |
| 23 | 1 | 21 | 37 |
| 24 | 2 | 21 | 37 |
| 25 | 3 | 21 | 37 |
| 26 | 4 | 21 | 37 |
| 27 | 5 | 21 | 37 |
| 28 | 6 | 21 | 37 |
| 29 | 7 | 21 | 37 |
| 30 | 8 | 21 | 37 |
| 31 | 9 | 21 | 37 |
| 32 | 10 | 21 | 37 |
| 33 | 11 | 21 | 37 |
| 34 | 1 | 22 | 33 |
| 35 | 2 | 22 | 33 |
| 36 | 3 | 22 | 33 |
| 37 | 4 | 22 | 33 |
| 38 | 5 | 22 | 33 |
| 39 | 6 | 22 | 33 |
| 40 | 7 | 22 | 33 |
| 41 | 8 | 22 | 33 |
| 42 | 9 | 22 | 33 |
| 43 | 10 | 22 | 33 |
| 44 | 11 | 22 | 33 |
| 45 | 1 | 23 | 33 |
| 46 | 2 | 23 | 33 |
| 47 | 3 | 23 | 33 |
| 48 | 4 | 23 | 33 |
| 49 | 5 | 23 | 33 |
| 50 | 6 | 23 | 33 |
| 51 | 7 | 23 | 33 |
| 52 | 8 | 23 | 33 |
| 53 | 9 | 23 | 33 |
| 54 | 10 | 23 | 33 |
| 55 | 11 | 23 | 33 |
| 56 | 1 | 23 | 34 |
| 57 | 2 | 23 | 34 |
| 58 | 3 | 23 | 34 |
| 59 | 4 | 23 | 34 |
| 60 | 5 | 23 | 34 |
| 61 | 6 | 23 | 34 |
| 62 | 7 | 23 | 34 |
| 63 | 8 | 23 | 34 |
| 64 | 9 | 23 | 34 |
| 65 | 10 | 23 | 34 |
| 66 | 11 | 23 | 34 |
| 67 | 1 | 23 | 37 |
| 68 | 2 | 23 | 37 |
| 69 | 3 | 23 | 37 |
| 70 | 4 | 23 | 37 |
| 71 | 5 | 23 | 37 |
| 72 | 6 | 23 | 37 |
| 73 | 7 | 23 | 37 |
| 74 | 8 | 23 | 37 |
| 75 | 9 | 23 | 37 |
| 76 | 10 | 23 | 37 |
| 77 | 11 | 23 | 37 |
| 78 | 1 | 33 | 34 |
| 79 | 2 | 33 | 34 |
| 80 | 3 | 33 | 34 |
| 81 | 4 | 33 | 34 |
| 82 | 5 | 33 | 34 |
| 83 | 6 | 33 | 34 |
| 84 | 7 | 33 | 34 |
| 85 | 8 | 33 | 34 |
| 86 | 9 | 33 | 34 |
| 87 | 10 | 33 | 34 |
| 88 | 11 | 33 | 34 |
| 89 | 1 | 38 | 41 |
| 90 | 2 | 38 | 41 |
| 91 | 3 | 38 | 41 |
| 92 | 4 | 38 | 41 |
| 93 | 5 | 38 | 41 |
| 94 | 6 | 38 | 41 |
| 95 | 7 | 38 | 41 |
| 96 | 8 | 38 | 41 |
| 97 | 9 | 38 | 41 |
| 98 | 10 | 38 | 41 |
| 99 | 11 | 38 | 41 |
| 100 | 1 | 1 | 31 |
| 101 | 1 | 1 | 33 |
| 102 | 1 | 1 | 38 |
| 103 | 1 | 2 | 21 |
| 104 | 1 | 6 | 21 |
| 105 | 6 | 6 | 21 |
| 106 | 6 | 6 | 6 |
| 107 | 1 | 21 | 31 |
| 108 | 1 | 21 | 32 |
| 109 | 1 | 21 | 35 |
| 110 | 1 | 21 | 36 |
| 111 | 1 | 21 | 38 |
| 112 | 1 | 21 | 39 |
| 113 | 1 | 21 | 40 |
| 114 | 1 | 21 | 41 |
| 115 | 1 | 21 | 42 |
| 116 | 1 | 21 | 43 |
| 117 | 1 | 21 | 44 |
| 118 | 1 | 21 | 45 |
| 119 | 1 | 21 | 46 |
| 120 | 1 | 21 | 47 |
| 121 | 1 | 21 | 48 |
| 122 | 1 | 21 | 49 |
| 123 | 1 | 21 | 50 |
| 124 | 1 | 21 | 51 |
| 125 | 1 | 22 | 31 |
| 126 | 1 | 22 | 32 |
| 127 | 1 | 22 | 34 |
| 128 | 1 | 22 | 35 |
| 129 | 1 | 22 | 36 |
| 130 | 1 | 22 | 37 |
| 131 | 1 | 22 | 38 |
| 132 | 1 | 22 | 39 |
| 133 | 1 | 22 | 40 |
| 134 | 1 | 22 | 41 |
| 135 | 1 | 22 | 42 |
| 136 | 1 | 22 | 43 |
| 137 | 1 | 22 | 44 |
| 138 | 1 | 22 | 45 |
| 139 | 1 | 22 | 46 |
| 140 | 1 | 22 | 47 |
| 141 | 1 | 22 | 48 |
| 142 | 1 | 22 | 49 |
| 143 | 1 | 22 | 50 |
| 144 | 1 | 22 | 51 |
| 145 | 1 | 23 | 31 |
| 146 | 1 | 23 | 32 |
| 147 | 1 | 23 | 35 |
| 148 | 1 | 23 | 36 |
| 149 | 1 | 23 | 38 |
| 150 | 1 | 23 | 39 |
| 151 | 1 | 23 | 40 |
| 152 | 1 | 23 | 41 |
| 153 | 1 | 23 | 42 |
| 154 | 1 | 23 | 43 |
| 155 | 1 | 23 | 44 |
| 156 | 1 | 23 | 45 |
| 157 | 1 | 23 | 46 |
| 158 | 1 | 23 | 47 |
| 159 | 1 | 23 | 48 |
| 160 | 1 | 23 | 49 |
| 161 | 1 | 23 | 50 |
| 162 | 1 | 23 | 51 |
| 163 | 1 | 24 | 31 |
| 164 | 1 | 24 | 32 |
| 165 | 1 | 24 | 33 |
| 166 | 1 | 24 | 34 |
| 167 | 1 | 24 | 35 |
| 168 | 1 | 24 | 36 |
| 169 | 1 | 24 | 37 |
| 170 | 1 | 24 | 38 |
| 171 | 1 | 24 | 39 |
| 172 | 1 | 24 | 40 |
| 173 | 1 | 24 | 41 |
| 174 | 1 | 24 | 42 |
| 175 | 1 | 24 | 43 |
| 176 | 1 | 24 | 44 |
| 177 | 1 | 24 | 45 |
| 178 | 1 | 24 | 46 |
| 179 | 1 | 24 | 47 |
| 180 | 1 | 24 | 48 |
| 181 | 1 | 24 | 49 |
| 182 | 1 | 24 | 50 |
| 183 | 1 | 24 | 51 |
| 184 | 1 | 25 | 31 |
| 185 | 1 | 25 | 32 |
| 186 | 1 | 25 | 33 |
| 187 | 1 | 25 | 34 |
| 188 | 1 | 25 | 35 |
| 189 | 1 | 25 | 36 |
| 190 | 1 | 25 | 37 |
| 191 | 1 | 25 | 38 |
| 192 | 1 | 25 | 39 |
| 193 | 1 | 25 | 40 |
| 194 | 1 | 25 | 41 |
| 195 | 1 | 25 | 42 |
| 196 | 1 | 25 | 43 |
| 197 | 1 | 25 | 44 |
| 198 | 1 | 25 | 45 |
| 199 | 1 | 25 | 46 |
| 200 | 1 | 25 | 47 |
| 201 | 1 | 25 | 48 |
| 202 | 1 | 25 | 49 |
| 203 | 1 | 25 | 50 |
| 204 | 1 | 25 | 51 |
| 205 | 1 | 26 | 31 |
| 206 | 1 | 26 | 32 |
| 207 | 1 | 26 | 33 |
| 208 | 1 | 26 | 34 |
| 209 | 1 | 26 | 35 |
| 210 | 1 | 26 | 36 |
| 211 | 1 | 26 | 37 |
| 212 | 1 | 26 | 38 |
| 213 | 1 | 26 | 39 |
| 214 | 1 | 26 | 40 |
| 215 | 1 | 26 | 41 |
| 216 | 1 | 26 | 42 |
| 217 | 1 | 26 | 43 |
| 218 | 1 | 26 | 44 |
| 219 | 1 | 26 | 45 |
| 220 | 1 | 26 | 46 |
| 221 | 1 | 26 | 47 |
| 222 | 1 | 26 | 48 |
| 223 | 1 | 26 | 49 |
| 224 | 1 | 26 | 50 |
| 225 | 1 | 26 | 51 |
| 226 | 1 | 27 | 31 |
| 227 | 1 | 27 | 32 |
| 228 | 1 | 27 | 33 |
| 229 | 1 | 27 | 34 |
| 230 | 1 | 27 | 35 |
| 231 | 1 | 27 | 36 |
| 232 | 1 | 27 | 37 |
| 233 | 1 | 27 | 38 |
| 234 | 1 | 27 | 39 |
| 235 | 1 | 27 | 40 |
| 236 | 1 | 27 | 41 |
| 237 | 1 | 27 | 42 |
| 238 | 1 | 27 | 43 |
| 239 | 1 | 27 | 44 |
| 240 | 1 | 27 | 45 |
| 241 | 1 | 27 | 46 |
| 242 | 1 | 27 | 47 |
| 243 | 1 | 27 | 48 |
| 244 | 1 | 27 | 49 |
| 245 | 1 | 27 | 50 |
| 246 | 1 | 27 | 51 |
| 247 | 1 | 28 | 31 |
| 248 | 1 | 28 | 32 |
| 249 | 1 | 28 | 33 |
| 250 | 1 | 28 | 34 |
| 251 | 1 | 28 | 35 |
| 252 | 1 | 28 | 36 |
| 253 | 1 | 28 | 37 |
| 254 | 1 | 28 | 38 |
| 255 | 1 | 28 | 39 |
| 256 | 1 | 28 | 40 |
| 257 | 1 | 28 | 41 |
| 258 | 1 | 28 | 42 |
| 259 | 1 | 28 | 43 |
| 260 | 1 | 28 | 44 |
| 261 | 1 | 28 | 45 |
| 262 | 1 | 28 | 46 |
| 263 | 1 | 28 | 47 |
| 264 | 1 | 28 | 48 |
| 265 | 1 | 28 | 49 |
| 266 | 1 | 28 | 50 |
| 267 | 1 | 28 | 51 |
| 268 | 1 | 29 | 31 |
| 269 | 1 | 29 | 32 |
| 270 | 1 | 29 | 33 |
| 271 | 1 | 29 | 34 |
| 272 | 1 | 29 | 35 |
| 273 | 1 | 29 | 36 |
| 274 | 1 | 29 | 37 |
| 275 | 1 | 29 | 38 |
| 276 | 1 | 29 | 39 |
| 277 | 1 | 29 | 40 |
| 278 | 1 | 29 | 41 |
| 279 | 1 | 29 | 42 |
| 280 | 1 | 29 | 43 |
| 281 | 1 | 29 | 44 |
| 282 | 1 | 29 | 45 |
| 283 | 1 | 29 | 46 |
| 284 | 1 | 29 | 47 |
| 285 | 1 | 29 | 48 |
| 286 | 1 | 29 | 49 |
| 287 | 1 | 29 | 50 |
| 288 | 1 | 29 | 51 |
| 289 | 1 | 30 | 31 |
| 290 | 1 | 30 | 32 |
| 291 | 1 | 30 | 33 |
| 292 | 1 | 30 | 34 |
| 293 | 1 | 30 | 35 |
| 294 | 1 | 30 | 36 |
| 295 | 1 | 30 | 37 |
| 296 | 1 | 30 | 38 |
| 297 | 1 | 30 | 39 |
| 298 | 1 | 30 | 40 |
| 299 | 1 | 30 | 41 |
| 300 | 1 | 30 | 42 |
| 301 | 1 | 30 | 43 |
| 302 | 1 | 30 | 44 |
| 303 | 1 | 30 | 45 |
| 304 | 1 | 30 | 46 |
| 305 | 1 | 30 | 47 |
| 306 | 1 | 30 | 48 |
| 307 | 1 | 30 | 49 |
| 308 | 1 | 30 | 50 |
| 309 | 1 | 30 | 51 |
| 310 | 1 | 31 | 31 |
| 311 | 1 | 31 | 32 |
| 312 | 1 | 31 | 33 |
| 313 | 1 | 31 | 34 |
| 314 | 1 | 31 | 35 |
| 315 | 1 | 31 | 36 |
| 316 | 1 | 31 | 37 |
| 317 | 1 | 31 | 38 |
| 318 | 1 | 31 | 39 |
| 319 | 1 | 31 | 40 |
| 320 | 1 | 31 | 41 |
| 321 | 1 | 31 | 42 |
| 322 | 1 | 31 | 43 |
| 323 | 1 | 31 | 44 |
| 324 | 1 | 31 | 45 |
| 325 | 1 | 31 | 46 |
| 326 | 1 | 31 | 47 |
| 327 | 1 | 31 | 48 |
| 328 | 1 | 31 | 49 |
| 329 | 1 | 31 | 50 |
| 330 | 1 | 31 | 51 |
| 331 | 1 | 32 | 32 |
| 332 | 1 | 32 | 33 |
| 333 | 1 | 32 | 34 |
| 334 | 1 | 32 | 35 |
| 335 | 1 | 32 | 36 |
| 336 | 1 | 32 | 37 |
| 337 | 1 | 32 | 38 |
| 338 | 1 | 32 | 39 |
| 339 | 1 | 32 | 40 |
| 340 | 1 | 32 | 41 |
| 341 | 1 | 32 | 42 |
| 342 | 1 | 32 | 43 |
| 343 | 1 | 32 | 44 |
| 344 | 1 | 32 | 45 |
| 345 | 1 | 32 | 46 |
| 346 | 1 | 32 | 47 |
| 347 | 1 | 32 | 48 |
| 348 | 1 | 32 | 49 |
| 349 | 1 | 32 | 50 |
| 350 | 1 | 32 | 51 |
| 351 | 1 | 33 | 33 |
| 352 | 1 | 33 | 35 |
| 353 | 1 | 33 | 36 |
| 354 | 1 | 33 | 37 |
| 355 | 1 | 33 | 38 |
| 356 | 1 | 33 | 39 |
| 357 | 1 | 33 | 40 |
| 358 | 1 | 33 | 41 |
| 359 | 1 | 33 | 42 |
| 360 | 1 | 33 | 43 |
| 361 | 1 | 33 | 44 |
| 362 | 1 | 33 | 45 |
| 363 | 1 | 33 | 46 |
| 364 | 1 | 33 | 47 |
| 365 | 1 | 33 | 48 |
| 366 | 1 | 33 | 49 |
| 367 | 1 | 33 | 50 |
| 368 | 1 | 33 | 51 |
| 369 | 1 | 34 | 34 |
| 370 | 1 | 34 | 35 |
| 371 | 1 | 34 | 36 |
| 372 | 1 | 34 | 37 |
| 373 | 1 | 34 | 38 |
| 374 | 1 | 34 | 39 |
| 375 | 1 | 34 | 40 |
| 376 | 1 | 34 | 41 |
| 377 | 1 | 34 | 42 |
| 378 | 1 | 34 | 43 |
| 379 | 1 | 34 | 44 |
| 380 | 1 | 34 | 45 |
| 381 | 1 | 34 | 46 |
| 382 | 1 | 34 | 47 |
| 383 | 1 | 34 | 48 |
| 384 | 1 | 34 | 49 |
| 385 | 1 | 34 | 50 |
| 386 | 1 | 34 | 51 |
| 387 | 1 | 35 | 35 |
| 388 | 1 | 35 | 36 |
| 389 | 1 | 35 | 37 |
| 390 | 1 | 35 | 38 |
| 391 | 1 | 35 | 39 |
| 392 | 1 | 35 | 40 |
| 393 | 1 | 35 | 41 |
| 394 | 1 | 35 | 42 |
| 395 | 1 | 35 | 43 |
| 396 | 1 | 35 | 44 |
| 397 | 1 | 35 | 45 |
| 398 | 1 | 35 | 46 |
| 399 | 1 | 35 | 47 |
| 400 | 1 | 35 | 48 |
| 401 | 1 | 35 | 49 |
| 402 | 1 | 35 | 50 |
| 403 | 1 | 35 | 51 |
| 404 | 1 | 36 | 36 |
| 405 | 1 | 36 | 37 |
| 406 | 1 | 36 | 38 |
| 407 | 1 | 36 | 39 |
| 408 | 1 | 36 | 40 |
| 409 | 1 | 36 | 41 |
| 410 | 1 | 36 | 42 |
| 411 | 1 | 36 | 43 |
| 412 | 1 | 36 | 44 |
| 413 | 1 | 36 | 45 |
| 414 | 1 | 36 | 46 |
| 415 | 1 | 36 | 47 |
| 416 | 1 | 36 | 48 |
| 417 | 1 | 36 | 49 |
| 418 | 1 | 36 | 50 |
| 419 | 1 | 36 | 51 |
| 420 | 1 | 37 | 37 |
| 421 | 1 | 37 | 38 |
| 422 | 1 | 37 | 39 |
| 423 | 1 | 37 | 40 |
| 424 | 1 | 37 | 41 |
| 425 | 1 | 37 | 42 |
| 426 | 1 | 37 | 43 |
| 427 | 1 | 37 | 44 |
| 428 | 1 | 37 | 45 |
| 429 | 1 | 37 | 46 |
| 430 | 1 | 37 | 47 |
| 431 | 1 | 37 | 48 |
| 432 | 1 | 37 | 49 |
| 433 | 1 | 37 | 50 |
| 434 | 1 | 37 | 51 |
| 435 | 1 | 38 | 38 |
| 436 | 1 | 38 | 39 |
| 437 | 1 | 38 | 40 |
| 438 | 1 | 38 | 42 |
| 439 | 1 | 38 | 43 |
| 440 | 1 | 38 | 44 |
| 441 | 1 | 38 | 45 |
| 442 | 1 | 38 | 46 |
| 443 | 1 | 38 | 47 |
| 444 | 1 | 38 | 48 |
| 445 | 1 | 38 | 49 |
| 446 | 1 | 38 | 50 |
| 447 | 1 | 38 | 51 |
| 448 | 1 | 39 | 39 |
| 449 | 1 | 39 | 40 |
| 450 | 1 | 39 | 41 |
| 451 | 1 | 39 | 42 |
| 452 | 1 | 39 | 43 |
| 453 | 1 | 39 | 44 |
| 454 | 1 | 39 | 45 |
| 455 | 1 | 39 | 46 |
| 456 | 1 | 39 | 47 |
| 457 | 1 | 39 | 48 |
| 458 | 1 | 39 | 49 |
| 459 | 1 | 39 | 50 |
| 460 | 1 | 39 | 51 |
| 461 | 1 | 40 | 40 |
| 462 | 1 | 40 | 41 |
| 463 | 1 | 40 | 42 |
| 464 | 1 | 40 | 43 |
| 465 | 1 | 40 | 44 |
| 466 | 1 | 40 | 45 |
| 467 | 1 | 40 | 46 |
| 468 | 1 | 40 | 47 |
| 469 | 1 | 40 | 48 |
| 470 | 1 | 40 | 49 |
| 471 | 1 | 40 | 50 |
| 472 | 1 | 40 | 51 |
| 473 | 1 | 41 | 41 |
| 474 | 1 | 41 | 42 |
| 475 | 1 | 41 | 43 |
| 476 | 1 | 41 | 44 |
| 477 | 1 | 41 | 45 |
| 478 | 1 | 41 | 46 |
| 479 | 1 | 41 | 47 |
| 480 | 1 | 41 | 48 |
| 481 | 1 | 41 | 49 |
| 482 | 1 | 41 | 50 |
| 483 | 1 | 41 | 51 |
| 484 | 1 | 42 | 42 |
| 485 | 1 | 42 | 43 |
| 486 | 1 | 42 | 44 |
| 487 | 1 | 42 | 45 |
| 488 | 1 | 42 | 46 |
| 489 | 1 | 42 | 47 |
| 490 | 1 | 42 | 48 |
| 491 | 1 | 42 | 49 |
| 492 | 1 | 42 | 50 |
| 493 | 1 | 42 | 51 |
| 494 | 1 | 43 | 43 |
| 495 | 1 | 43 | 44 |
| 496 | 1 | 43 | 45 |
| 497 | 1 | 43 | 46 |
| 498 | 1 | 43 | 47 |
| 499 | 1 | 43 | 48 |
| 500 | 1 | 43 | 49 |
| 501 | 1 | 43 | 50 |
| 502 | 1 | 43 | 51 |
| 503 | 1 | 44 | 44 |
| 504 | 1 | 44 | 45 |
| 505 | 1 | 44 | 46 |
| 506 | 1 | 44 | 47 |
| 507 | 1 | 44 | 48 |
| 508 | 1 | 44 | 49 |
| 509 | 1 | 44 | 50 |
| 510 | 1 | 44 | 51 |
| 511 | 1 | 45 | 45 |
| 512 | 1 | 45 | 46 |
| 513 | 1 | 45 | 47 |
| 514 | 1 | 45 | 48 |
| 515 | 1 | 45 | 49 |
| 516 | 1 | 45 | 50 |
| 517 | 1 | 45 | 51 |
| 518 | 1 | 46 | 46 |
| 519 | 1 | 46 | 47 |
| 520 | 1 | 46 | 48 |
| 521 | 1 | 46 | 49 |
| 522 | 1 | 46 | 50 |
| 523 | 1 | 46 | 51 |
| 524 | 1 | 47 | 47 |
| 525 | 1 | 47 | 48 |
| 526 | 1 | 47 | 49 |
| 527 | 1 | 47 | 50 |
| 528 | 1 | 47 | 51 |
| 529 | 1 | 48 | 48 |
| 530 | 1 | 48 | 49 |
| 531 | 1 | 48 | 50 |
| 532 | 1 | 48 | 51 |
| 533 | 1 | 49 | 49 |
| 534 | 1 | 49 | 50 |
| 535 | 1 | 49 | 51 |
| 536 | 1 | 50 | 50 |
| 537 | 1 | 50 | 51 |
| 538 | 1 | 51 | 51 |
| 539 | 12 | 21 | 33 |
| 540 | 12 | 21 | 34 |
| 541 | 12 | 21 | 35 |
| 542 | 12 | 21 | 36 |
| 543 | 12 | 21 | 37 |
| 544 | 12 | 21 | 38 |
| 545 | 12 | 21 | 39 |
| 546 | 12 | 21 | 40 |
| 547 | 12 | 21 | 41 |
| 548 | 12 | 21 | 42 |
| 549 | 12 | 21 | 43 |
| 550 | 12 | 21 | 44 |
| 551 | 12 | 21 | 45 |
| 552 | 12 | 21 | 46 |
| 553 | 12 | 21 | 47 |
| 554 | 12 | 21 | 48 |
| 555 | 12 | 21 | 49 |
| 556 | 12 | 21 | 50 |
| 557 | 12 | 21 | 51 |
| 558 | 12 | 21 | 52 |
| 559 | 12 | 22 | 33 |
| 560 | 12 | 22 | 34 |
| 561 | 12 | 22 | 35 |
| 562 | 12 | 22 | 36 |
| 563 | 12 | 22 | 37 |
| 564 | 12 | 22 | 38 |
| 565 | 12 | 22 | 39 |
| 566 | 12 | 22 | 40 |
| 567 | 12 | 22 | 41 |
| 568 | 12 | 22 | 42 |
| 569 | 12 | 22 | 43 |
| 570 | 12 | 22 | 44 |
| 571 | 12 | 22 | 45 |
| 572 | 12 | 22 | 46 |
| 573 | 12 | 22 | 47 |
| 574 | 12 | 22 | 48 |
| 575 | 12 | 22 | 49 |
| 576 | 12 | 22 | 50 |
| 577 | 12 | 22 | 51 |
| 578 | 12 | 22 | 52 |
| 579 | 1 | 21 | 52 |
| 580 | 1 | 22 | 52 |

10. An electronic device comprising:
a base layer;
a circuit layer on the base layer; and
a display element layer on the circuit layer and comprising a light-emitting element,
wherein the light-emitting element comprises a first electrode, a second electrode on the first electrode, and at least one functional layer between the first electrode and the second electrode and comprising an amine compound, the amine compound represented by Formula 1: 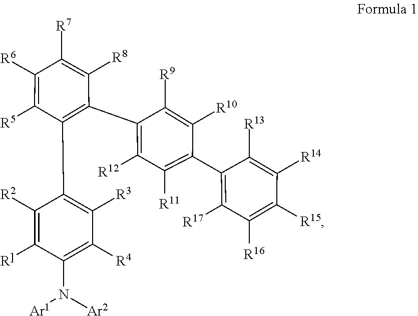
wherein, in Formula 1,
R¹ to R¹⁷ are each independently a hydrogen atom, a deuterium atom, a substituted or unsubstituted oxy group, a substituted or unsubstituted thio group, a substituted or unsubstituted amine group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, a substituted or unsubstituted heteroaryl group having 2 to 25 ring-forming carbons, and/or bonded to an adjacent group to form a ring,
wherein
a case where R⁵ is a substituted or unsubstituted carbazolyl group is excluded,
Ar¹ and Ar² are each independently a substituted or unsubstituted aryl group having 6 to 30 ring-forming carbons, or a substituted or unsubstituted heteroaryl group having 2 to 30 ring-forming carbons, and
wherein
at least one selected from among Ar¹ and Ar² comprises a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group,
when each of Ar¹ and Ar² comprises a substituted group, then a substituent of the substituted group is to bond to an adjacent group to form a ring,
a case where at least one selected from among Ar¹ and Ar² is a 4-phenyl-dibenzofuran-1-yl group is excluded,
a case where each Ar¹ and Ar² comprises a heteroaryl group substituted with a heteroaryl group is excluded,
a case where the amine compound comprises at least one selected from among a chlorine atom and acenaphthene is excluded, and
at least one hydrogen atom of the amine compound is substituted with a deuterium atom.

11. The electronic device of claim 10,
wherein the light-emitting element further comprises a capping layer on the second electrode, and
the capping layer has a refractive index of at least about 1.6 for light in a wavelength range of about 550 nanometer (nm) to about 660 nm.

12. The electronic device of claim 10, further comprising an optical control layer on the display element layer and comprising a quantum dot,
wherein the light-emitting element is configured to emit a first color light, and
the optical control layer comprises:
a first optical control part comprising a first quantum dot configured to convert the first color light into a second color light, the second color light having a wavelength region that is longer than a wavelength region of the first color light;
a second optical control part comprising a second quantum dot configured to convert the first color light into third color light, the third color light having a wavelength region that is longer than a wavelength region of the first color light and a wavelength region of the second color light; and
a third optical control part configured to transmit the first color light.

13. The electronic device of claim 12, further comprising a color filter layer on the optical control layer,
wherein the color filter layer comprises:
a first filter configured to transmit the second color light;
a second filter configured to transmit the third color light; and
a third filter configured to transmit the first color light.

14. An amine compound represented by Formula 1: 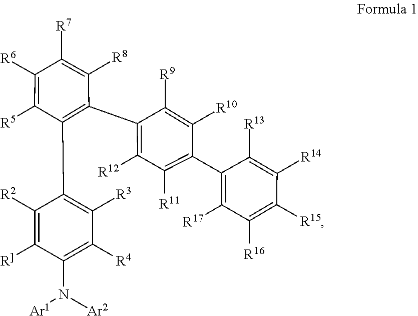
wherein, in Formula 1,
R¹ to R¹⁷ are each independently a hydrogen atom, a deuterium atom, a substituted or unsubstituted oxy group, a substituted or unsubstituted thio group, a substituted or unsubstituted amine group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 25 ring-forming carbons, and/or bonded to an adjacent group to form a ring,
wherein
a case where R⁵ is a substituted or unsubstituted carbazolyl group is excluded,
Ar¹ and Ar² are each independently a substituted or unsubstituted aryl group having 6 to 30 ring-forming carbons, or a substituted or unsubstituted heteroaryl group having 2 to 30 ring-forming carbons, and
wherein
at least one selected from among Ar¹ and Ar² comprises a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group,
when each of Ar¹ and Ar² comprises a substituted group, then a substituent of the substituted group is to bond to an adjacent group to form a ring,
a case where at least one selected from among Ar¹ and Ar² is a 4-phenyl-dibenzofuran-1-yl group is excluded,
a case where each Ar¹ and Ar² comprises a heteroaryl group substituted with a heteroaryl group is excluded,
a case where the amine compound comprises at least one selected from among a chlorine atom and acenaphthene is excluded, and
at least one hydrogen atom of the amine compound is substituted with a deuterium atom.

15. The amine compound of claim 14, wherein at least one selected from among Ar¹ and Ar² is represented by any one selected from among Formula 2-1 to 2-3: 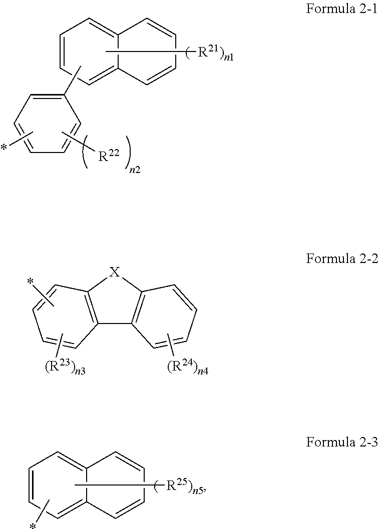
wherein, in Formula 2-1 to Formula 2-3,
X is O, S, or NR²⁶,
R²¹, R²², R²⁵, and R²⁶ are each independently a hydrogen atom, a deuterium atom, a halogen atom, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or bonded to an adjacent group to form a ring,
R²³ and R²⁴ are each independently a hydrogen atom, a deuterium atom, a halogen atom, a substituted or unsubstituted alkyl group having 1 to 20 carbons, a substituted or unsubstituted alkenyl group having 2 to 20 carbons, or a substituted or unsubstituted aryl group having 6 to 30 ring-forming carbons, and/or bonded to an adjacent group to form a ring,
n1 is an integer of 0 to 7,
n2 and n4 are each independently an integer of 0 to 4,
n3 is an integer of 0 to 3,
n5 is an integer of 0 to 7, and [symbol]
is a position to which Formula 1 is connected.

16. The amine compound of claim 15,
wherein one selected from among Ar¹ and Ar² is represented by any one selected from among Formula 2-1 to Formula 2-3, and
the other, selected from among Ar¹ and Ar², is a substituted or unsubstituted phenyl group, a substituted or unsubstituted biphenylyl group, a substituted or unsubstituted terphenylyl group, a substituted or unsubstituted quarterphenylyl group, a substituted or unsubstituted naphthyl group, a substituted or unsubstituted naphthylphenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a substituted or unsubstituted phenanthrenyl group.

17. The amine compound of claim 14, wherein the amine compound is represented by Formula 3: 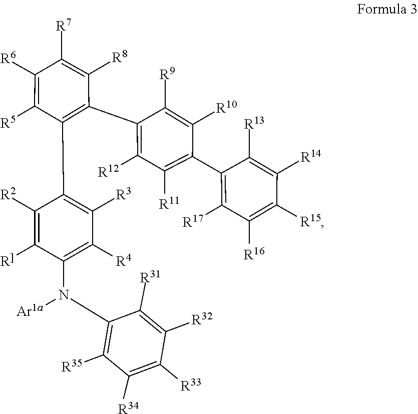
wherein, in Formula 3,
R³¹ to R³⁵ are each independently a hydrogen atom, a deuterium atom, a halogen atom, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or bonded to an adjacent group to form a ring,
wherein
a case where at least one selected from among R³¹ to R³⁵ is a heteroaryl group substituted with a heteroaryl group is excluded, and
Ar^{1a} comprises a substituted or unsubstituted naphthyl group, a substituted or unsubstituted naphthylphenyl group, a substituted or unsubstituted dibenzofuranyl group, a substituted or unsubstituted dibenzothiophenyl group, or a substituted or unsubstituted carbazolyl group, and when Ar^{1a} comprises a substituted naphthyl group, a substituted naphthylphenyl group, a substituted dibenzofuranyl group, a substituted dibenzothiophenyl group, or a substituted carbazolyl group, then the substituent is to bond to an adjacent group to form a ring, and
wherein
a case where Ar^{1a} comprises a heteroaryl group substituted with a heteroaryl group is excluded,
a case where Ar^{1a} is represented by Formula 3-a is excluded 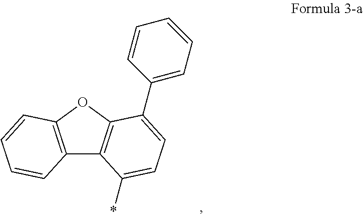 [SMILES: *c1ccc(-c2ccccc2)c2oc3ccccc3c12]
and
R¹ to R¹⁷ are as defined in Formula 1.

18. The amine compound of claim 17,
wherein R³¹ to R³⁵ are each independently a hydrogen atom, a deuterium atom, a substituted or unsubstituted phenyl group, a substituted or unsubstituted naphthyl group, or a substituted or unsubstituted phenanthrenyl group, or
wherein at least one pair of groups, selected from among R³¹ to R³⁵, is bonded together to form a ring.

19. The amine compound of claim 14, wherein the amine compound is represented by any one selected from among Formula 4-1 to Formula 4-3: 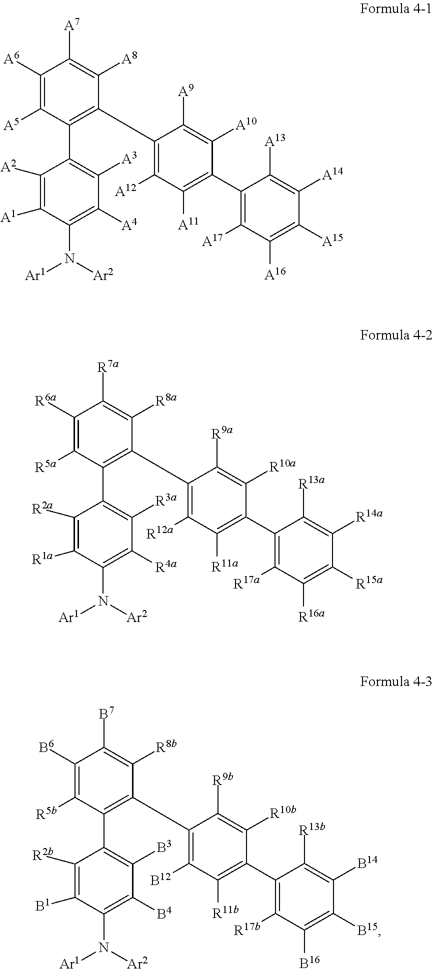
and
wherein,
in Formula 4-1,
A¹ to A¹⁷ are each independently a hydrogen atom, a deuterium atom, or a substituted or unsubstituted aryl group having 6 to 30 ring-forming carbons,
in Formula 4-2,
R^{1a} to R^{17a} are each independently a hydrogen atom, a deuterium atom, a substituted or unsubstituted oxy group, a substituted or unsubstituted thio group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or bonded to an adjacent group to form a ring, and
at least one pair of groups, selected from among R^{1a} to R^{17a}, are bonded together to form an aromatic hydrocarbon ring,
in Formula 4-3,
B¹, B³, B⁴, B⁶, B⁷, B¹², B¹⁴, B¹⁵, and B¹⁶ are each independently a hydrogen atom, or deuterium atom,
R^{2b}, R^{5b}, R^{8b}, R^{9b}, R^{10b}, R^{11b}, R^{13b}, and R^{17b} are each independently a hydrogen atom, a deuterium atom, a substituted or unsubstituted oxy group, a substituted or unsubstituted thio group, a substituted or unsubstituted alkyl group having 1 to 20 carbons, a substituted or unsubstituted alkenyl group having 2 to 20 carbons, a substituted or unsubstituted aryl group having 6 to 30 ring-forming carbons, or a substituted or unsubstituted heteroaryl group having 2 to 30 ring-forming carbons, and/or bonded to an adjacent group to form a ring, and
at least one pair among R^{2b} and R^{5b}, R^{8b} and R^{9b}, R^{10b} and R^{13b}, and R^{11b} and R^{17b} are bonded to each other to form a ring, and
in Formula 4-1 to 4-3,
Ar¹ and Ar² are as defined in Formula 1.

20. The amine compound of claim 14, where the amine compound is represented by Formula 5, and satisfies any one selected from among combinations present in Compound Combination Table 1: 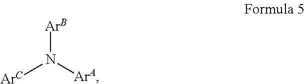
and
wherein, in Formula 5,
Ar^{A} is selected from among the group consisting of Substituent 1 to Substituent 12 in Substituent Group A,
Ar^{B} is selected from among the group consisting of Substituent 1 to Substituent 12, and Substituent 21 to Substituent 52 in Substituent Group A, and
Ar^{C} is selected from among the group consisting of Substituent 2 to Substituent 7, Substituent 9 to Substituent 11, and Substituent 31 to Substituent 52 in Substituent Group A: 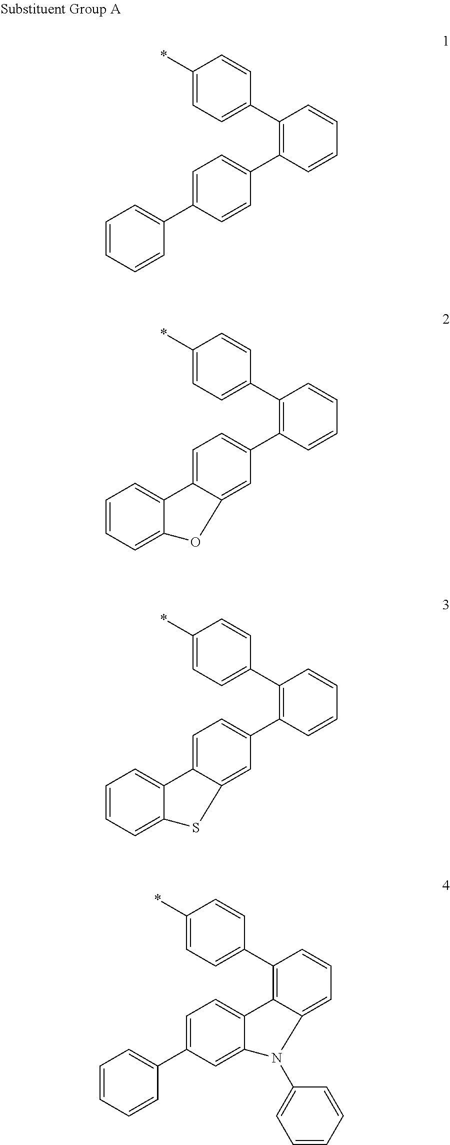 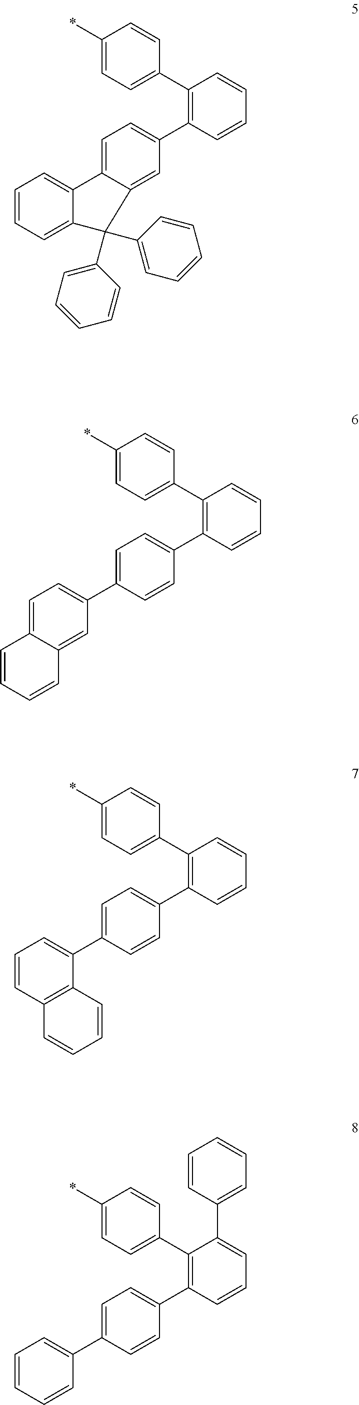 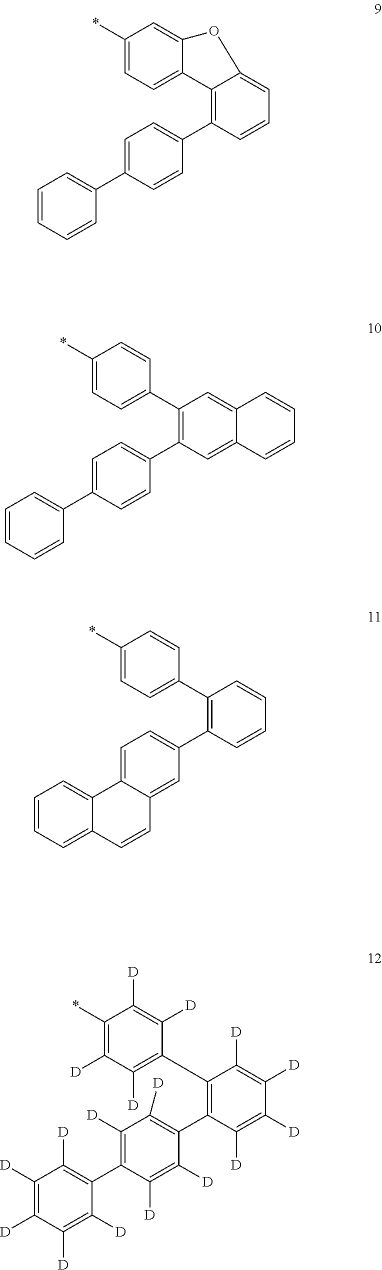 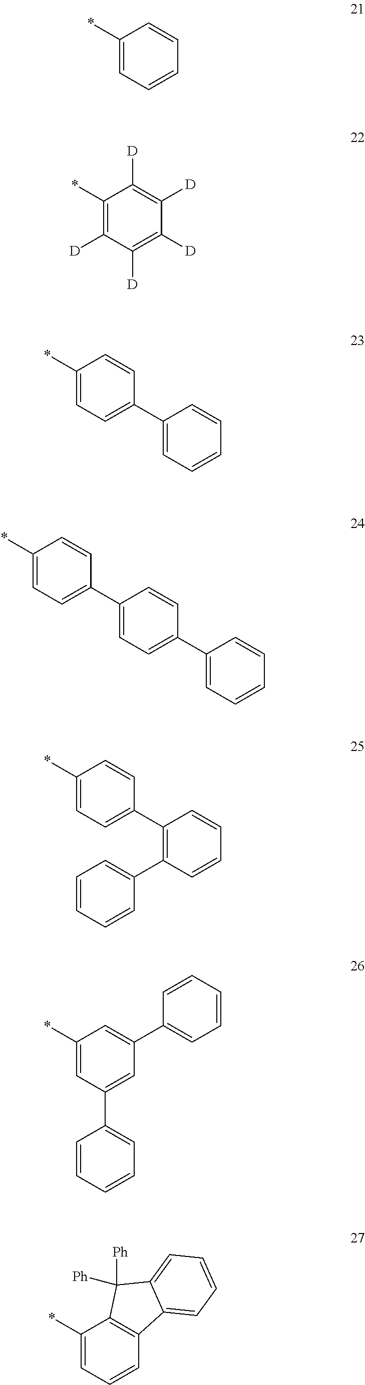 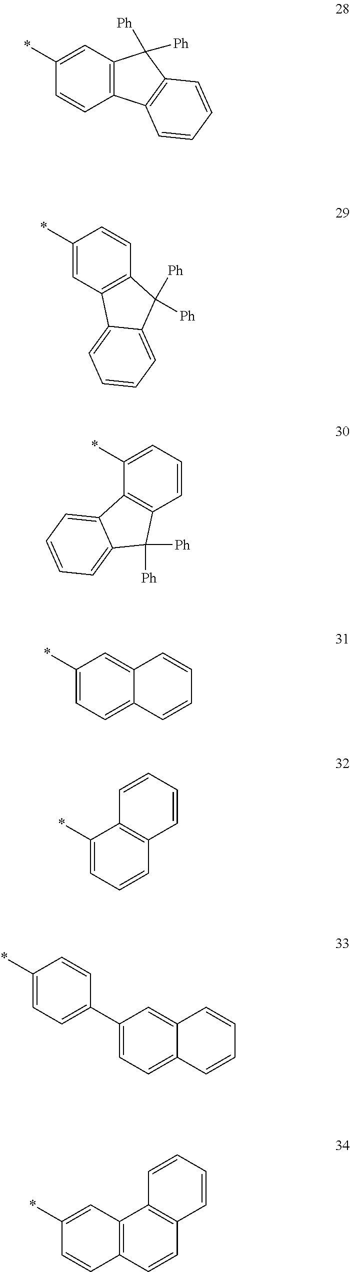 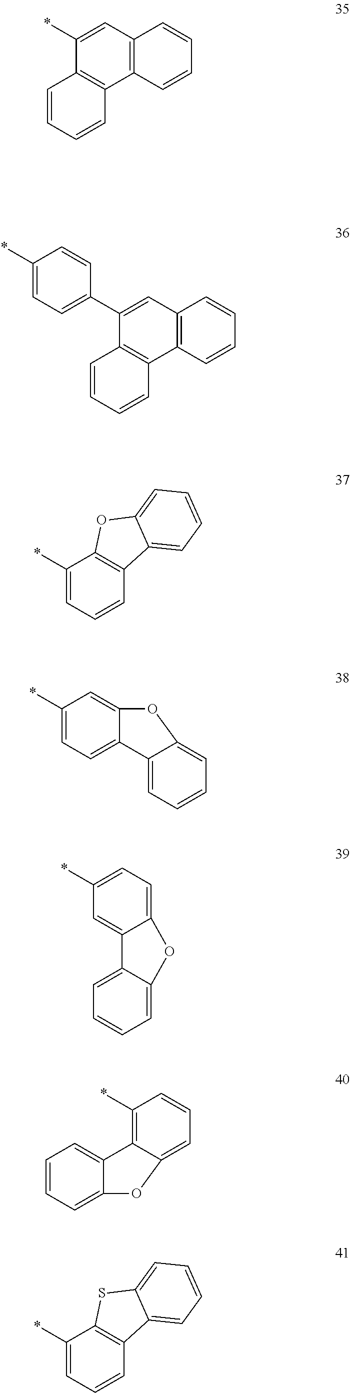 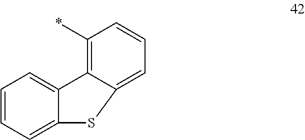 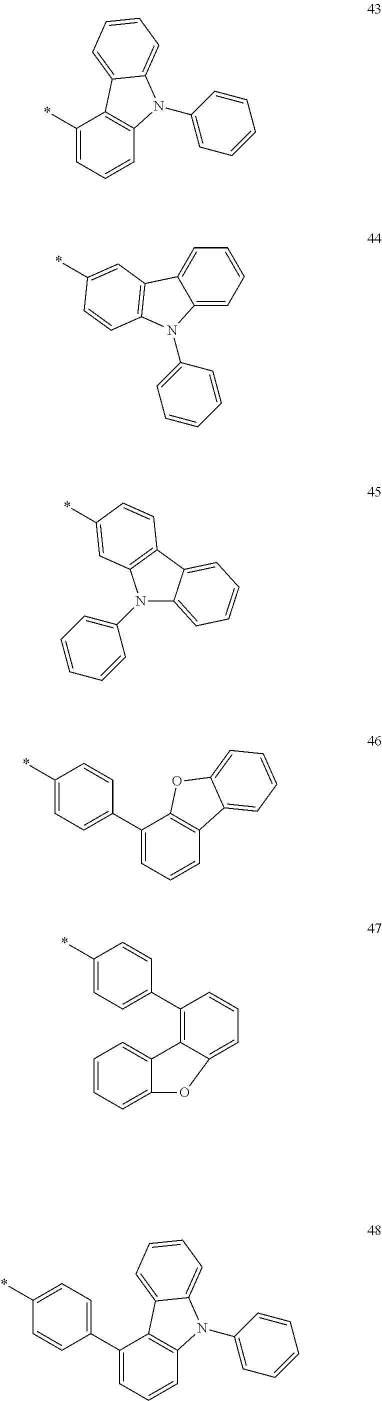 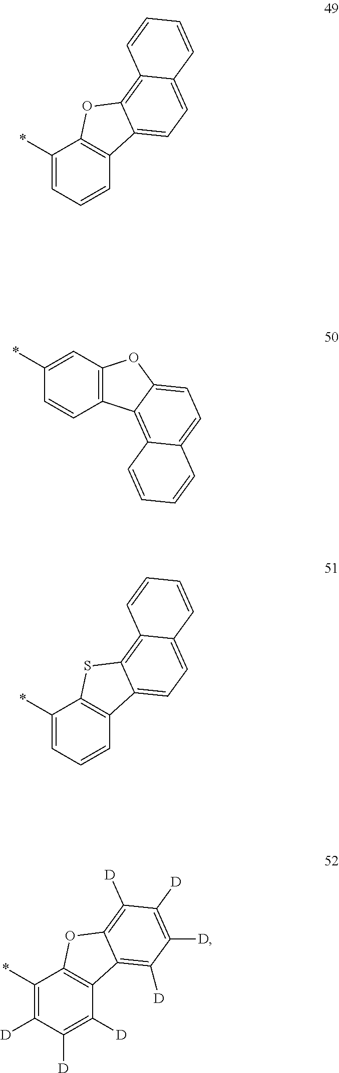
wherein [symbol] is a position to which Formula 1 is connected,
Compound Combination Table 1

| No. | Ar^{A} | Ar^{B} | Ar^{C} |
|---|---|---|---|
| 1 | 1 | 21 | 33 |
| 2 | 2 | 21 | 33 |
| 3 | 3 | 21 | 33 |
| 4 | 4 | 21 | 33 |
| 5 | 5 | 21 | 33 |
| 6 | 6 | 21 | 33 |
| 7 | 7 | 21 | 33 |
| 8 | 8 | 21 | 33 |
| 9 | 9 | 21 | 33 |
| 10 | 10 | 21 | 33 |
| 11 | 11 | 21 | 33 |
| 12 | 1 | 21 | 34 |
| 13 | 2 | 21 | 34 |
| 14 | 3 | 21 | 34 |
| 15 | 4 | 21 | 34 |
| 16 | 5 | 21 | 34 |
| 17 | 6 | 21 | 34 |
| 18 | 7 | 21 | 34 |
| 19 | 8 | 21 | 34 |
| 20 | 9 | 21 | 34 |
| 21 | 10 | 21 | 34 |
| 22 | 11 | 21 | 34 |
| 23 | 1 | 21 | 37 |
| 24 | 2 | 21 | 37 |
| 25 | 3 | 21 | 37 |
| 26 | 4 | 21 | 37 |
| 27 | 5 | 21 | 37 |
| 28 | 6 | 21 | 37 |
| 29 | 7 | 21 | 37 |
| 30 | 8 | 21 | 37 |
| 31 | 9 | 21 | 37 |
| 32 | 10 | 21 | 37 |
| 33 | 11 | 21 | 37 |
| 34 | 1 | 22 | 33 |
| 35 | 2 | 22 | 33 |
| 36 | 3 | 22 | 33 |
| 37 | 4 | 22 | 33 |
| 38 | 5 | 22 | 33 |
| 39 | 6 | 22 | 33 |
| 40 | 7 | 22 | 33 |
| 41 | 8 | 22 | 33 |
| 42 | 9 | 22 | 33 |
| 43 | 10 | 22 | 33 |
| 44 | 11 | 22 | 33 |
| 45 | 1 | 23 | 33 |
| 46 | 2 | 23 | 33 |
| 47 | 3 | 23 | 33 |
| 48 | 4 | 23 | 33 |
| 49 | 5 | 23 | 33 |
| 50 | 6 | 23 | 33 |
| 51 | 7 | 23 | 33 |
| 52 | 8 | 23 | 33 |
| 53 | 9 | 23 | 33 |
| 54 | 10 | 23 | 33 |
| 55 | 11 | 23 | 33 |
| 56 | 1 | 23 | 34 |
| 57 | 2 | 23 | 34 |
| 58 | 3 | 23 | 34 |
| 59 | 4 | 23 | 34 |
| 60 | 5 | 23 | 34 |
| 61 | 6 | 23 | 34 |
| 62 | 7 | 23 | 34 |
| 63 | 8 | 23 | 34 |
| 64 | 9 | 23 | 34 |
| 65 | 10 | 23 | 34 |
| 66 | 11 | 23 | 34 |
| 67 | 1 | 23 | 37 |
| 68 | 2 | 23 | 37 |
| 69 | 3 | 23 | 37 |
| 70 | 4 | 23 | 37 |
| 71 | 5 | 23 | 37 |
| 72 | 6 | 23 | 37 |
| 73 | 7 | 23 | 37 |
| 74 | 8 | 23 | 37 |
| 75 | 9 | 23 | 37 |
| 76 | 10 | 23 | 37 |
| 77 | 11 | 23 | 37 |
| 78 | 1 | 33 | 34 |
| 79 | 2 | 33 | 34 |
| 80 | 3 | 33 | 34 |
| 81 | 4 | 33 | 34 |
| 82 | 5 | 33 | 34 |
| 83 | 6 | 33 | 34 |
| 84 | 7 | 33 | 34 |
| 85 | 8 | 33 | 34 |
| 86 | 9 | 33 | 34 |
| 87 | 10 | 33 | 34 |
| 88 | 11 | 33 | 34 |
| 89 | 1 | 38 | 41 |
| 90 | 2 | 38 | 41 |
| 91 | 3 | 38 | 41 |
| 92 | 4 | 38 | 41 |
| 93 | 5 | 38 | 41 |
| 94 | 6 | 38 | 41 |
| 95 | 7 | 38 | 41 |
| 96 | 8 | 38 | 41 |
| 97 | 9 | 38 | 41 |
| 98 | 10 | 38 | 41 |
| 99 | 11 | 38 | 41 |
| 100 | 1 | 1 | 31 |
| 101 | 1 | 1 | 33 |
| 102 | 1 | 1 | 38 |
| 103 | 1 | 2 | 21 |
| 104 | 1 | 6 | 21 |
| 105 | 6 | 6 | 21 |
| 106 | 6 | 6 | 6 |
| 107 | 1 | 21 | 31 |
| 108 | 1 | 21 | 32 |
| 109 | 1 | 21 | 35 |
| 110 | 1 | 21 | 36 |
| 111 | 1 | 21 | 38 |
| 112 | 1 | 21 | 39 |
| 113 | 1 | 21 | 40 |
| 114 | 1 | 21 | 41 |
| 115 | 1 | 21 | 42 |
| 116 | 1 | 21 | 43 |
| 117 | 1 | 21 | 14 |
| 118 | 1 | 21 | 45 |
| 119 | 1 | 21 | 46 |
| 120 | 1 | 21 | 47 |
| 121 | 1 | 21 | 48 |
| 122 | 1 | 21 | 49 |
| 123 | 1 | 21 | 50 |
| 124 | 1 | 21 | 51 |
| 125 | 1 | 22 | 31 |
| 126 | 1 | 22 | 32 |
| 127 | 1 | 22 | 34 |
| 128 | 1 | 22 | 35 |
| 129 | 1 | 22 | 36 |
| 130 | 1 | 22 | 37 |
| 131 | 1 | 22 | 38 |
| 132 | 1 | 22 | 39 |
| 133 | 1 | 22 | 40 |
| 134 | 1 | 22 | 41 |
| 135 | 1 | 22 | 42 |
| 136 | 1 | 22 | 43 |
| 137 | 1 | 22 | 44 |
| 138 | 1 | 22 | 45 |
| 139 | 1 | 22 | 46 |
| 140 | 1 | 22 | 47 |
| 141 | 1 | 22 | 48 |
| 142 | 1 | 22 | 49 |
| 143 | 1 | 22 | 50 |
| 144 | 1 | 22 | 51 |
| 145 | 1 | 23 | 31 |
| 146 | 1 | 23 | 32 |
| 147 | 1 | 23 | 35 |
| 148 | 1 | 23 | 36 |
| 149 | 1 | 23 | 38 |
| 150 | 1 | 23 | 39 |
| 151 | 1 | 23 | 40 |
| 152 | 1 | 23 | 41 |
| 153 | 1 | 23 | 42 |
| 154 | 1 | 23 | 43 |
| 155 | 1 | 23 | 44 |
| 156 | 1 | 23 | 45 |
| 157 | 1 | 23 | 46 |
| 158 | 1 | 23 | 47 |
| 159 | 1 | 23 | 48 |
| 160 | 1 | 23 | 49 |
| 161 | 1 | 23 | 50 |
| 162 | 1 | 23 | 51 |
| 163 | 1 | 24 | 31 |
| 164 | 1 | 24 | 32 |
| 165 | 1 | 24 | 33 |
| 166 | 1 | 24 | 34 |
| 167 | 1 | 24 | 35 |
| 168 | 1 | 24 | 36 |
| 169 | 1 | 24 | 37 |
| 170 | 1 | 24 | 38 |
| 171 | 1 | 24 | 39 |
| 172 | 1 | 24 | 40 |
| 173 | 1 | 24 | 41 |
| 174 | 1 | 24 | 42 |
| 175 | 1 | 24 | 43 |
| 176 | 1 | 24 | 44 |
| 177 | 1 | 24 | 45 |
| 178 | 1 | 24 | 46 |
| 179 | 1 | 24 | 47 |
| 180 | 1 | 24 | 48 |
| 181 | 1 | 24 | 49 |
| 182 | 1 | 24 | 50 |
| 183 | 1 | 24 | 51 |
| 184 | 1 | 25 | 31 |
| 185 | 1 | 25 | 32 |
| 186 | 1 | 25 | 33 |
| 187 | 1 | 25 | 34 |
| 188 | 1 | 25 | 35 |
| 189 | 1 | 25 | 36 |
| 190 | 1 | 25 | 37 |
| 191 | 1 | 25 | 38 |
| 192 | 1 | 25 | 39 |
| 193 | 1 | 25 | 40 |
| 194 | 1 | 25 | 41 |
| 195 | 1 | 25 | 42 |
| 196 | 1 | 25 | 43 |
| 197 | 1 | 25 | 44 |
| 198 | 1 | 25 | 45 |
| 199 | 1 | 25 | 46 |
| 200 | 1 | 25 | 47 |
| 201 | 1 | 25 | 48 |
| 202 | 1 | 25 | 49 |
| 203 | 1 | 25 | 50 |
| 204 | 1 | 25 | 51 |
| 205 | 1 | 26 | 31 |
| 206 | 1 | 26 | 32 |
| 207 | 1 | 26 | 33 |
| 208 | 1 | 26 | 34 |
| 209 | 1 | 26 | 35 |
| 210 | 1 | 26 | 36 |
| 211 | 1 | 26 | 37 |
| 212 | 1 | 26 | 38 |
| 213 | 1 | 26 | 39 |
| 214 | 1 | 26 | 40 |
| 215 | 1 | 26 | 41 |
| 216 | 1 | 26 | 42 |
| 217 | 1 | 26 | 43 |
| 218 | 1 | 26 | 44 |
| 219 | 1 | 26 | 45 |
| 220 | 1 | 26 | 46 |
| 221 | 1 | 26 | 47 |
| 222 | 1 | 26 | 48 |
| 223 | 1 | 26 | 49 |
| 224 | 1 | 26 | 50 |
| 225 | 1 | 26 | 51 |
| 226 | 1 | 27 | 31 |
| 227 | 1 | 27 | 32 |
| 228 | 1 | 27 | 33 |
| 229 | 1 | 27 | 34 |
| 230 | 1 | 27 | 35 |
| 231 | 1 | 27 | 36 |
| 232 | 1 | 27 | 37 |
| 233 | 1 | 27 | 38 |
| 234 | 1 | 27 | 39 |
| 235 | 1 | 27 | 40 |
| 236 | 1 | 27 | 41 |
| 237 | 1 | 27 | 42 |
| 238 | 1 | 27 | 43 |
| 239 | 1 | 27 | 44 |
| 240 | 1 | 27 | 45 |
| 241 | 1 | 27 | 46 |
| 242 | 1 | 27 | 47 |
| 243 | 1 | 27 | 48 |
| 244 | 1 | 27 | 49 |
| 245 | 1 | 27 | 50 |
| 246 | 1 | 27 | 51 |
| 247 | 1 | 28 | 31 |
| 248 | 1 | 28 | 32 |
| 249 | 1 | 28 | 33 |
| 250 | 1 | 28 | 34 |
| 251 | 1 | 28 | 35 |
| 252 | 1 | 28 | 36 |
| 253 | 1 | 28 | 37 |
| 254 | 1 | 28 | 38 |
| 255 | 1 | 28 | 39 |
| 256 | 1 | 28 | 40 |
| 257 | 1 | 28 | 41 |
| 258 | 1 | 28 | 42 |
| 259 | 1 | 28 | 43 |
| 260 | 1 | 28 | 44 |
| 261 | 1 | 28 | 45 |
| 262 | 1 | 28 | 46 |
| 263 | 1 | 28 | 47 |
| 264 | 1 | 28 | 48 |
| 265 | 1 | 28 | 49 |
| 266 | 1 | 28 | 50 |
| 267 | 1 | 28 | 51 |
| 268 | 1 | 29 | 31 |
| 269 | 1 | 29 | 32 |
| 270 | 1 | 29 | 33 |
| 271 | 1 | 29 | 34 |
| 272 | 1 | 29 | 35 |
| 273 | 1 | 29 | 36 |
| 274 | 1 | 29 | 37 |
| 275 | 1 | 29 | 38 |
| 276 | 1 | 29 | 39 |
| 277 | 1 | 29 | 40 |
| 278 | 1 | 29 | 41 |
| 279 | 1 | 29 | 42 |
| 280 | 1 | 29 | 43 |
| 281 | 1 | 29 | 44 |
| 282 | 1 | 29 | 45 |
| 283 | 1 | 29 | 46 |
| 284 | 1 | 29 | 47 |
| 285 | 1 | 29 | 48 |
| 286 | 1 | 29 | 49 |
| 287 | 1 | 29 | 50 |
| 288 | 1 | 29 | 51 |
| 289 | 1 | 30 | 31 |
| 290 | 1 | 30 | 32 |
| 291 | 1 | 30 | 33 |
| 292 | 1 | 30 | 34 |
| 293 | 1 | 30 | 35 |
| 294 | 1 | 30 | 36 |
| 295 | 1 | 30 | 37 |
| 296 | 1 | 30 | 38 |
| 297 | 1 | 30 | 39 |
| 298 | 1 | 30 | 40 |
| 299 | 1 | 30 | 41 |
| 300 | 1 | 30 | 42 |
| 301 | 1 | 30 | 43 |
| 302 | 1 | 30 | 44 |
| 303 | 1 | 30 | 45 |
| 304 | 1 | 30 | 46 |
| 305 | 1 | 30 | 47 |
| 306 | 1 | 30 | 48 |
| 307 | 1 | 30 | 49 |
| 308 | 1 | 30 | 50 |
| 309 | 1 | 30 | 51 |
| 310 | 1 | 31 | 31 |
| 311 | 1 | 31 | 32 |
| 312 | 1 | 31 | 33 |
| 313 | 1 | 31 | 34 |
| 314 | 1 | 31 | 35 |
| 315 | 1 | 31 | 36 |
| 316 | 1 | 31 | 37 |
| 317 | 1 | 31 | 38 |
| 318 | 1 | 31 | 39 |
| 319 | 1 | 31 | 40 |
| 320 | 1 | 31 | 41 |
| 321 | 1 | 31 | 42 |
| 322 | 1 | 31 | 43 |
| 323 | 1 | 31 | 44 |
| 324 | 1 | 31 | 45 |
| 325 | 1 | 31 | 46 |
| 326 | 1 | 31 | 47 |
| 327 | 1 | 31 | 48 |
| 328 | 1 | 31 | 49 |
| 329 | 1 | 31 | 50 |
| 330 | 1 | 31 | 51 |
| 331 | 1 | 32 | 32 |
| 332 | 1 | 32 | 33 |
| 333 | 1 | 32 | 34 |
| 334 | 1 | 32 | 35 |
| 335 | 1 | 32 | 36 |
| 336 | 1 | 32 | 37 |
| 337 | 1 | 32 | 38 |
| 338 | 1 | 32 | 39 |
| 339 | 1 | 32 | 40 |
| 340 | 1 | 32 | 41 |
| 341 | 1 | 32 | 42 |
| 342 | 1 | 32 | 43 |
| 343 | 1 | 32 | 44 |
| 344 | 1 | 32 | 45 |
| 345 | 1 | 32 | 46 |
| 346 | 1 | 32 | 47 |
| 347 | 1 | 32 | 48 |
| 348 | 1 | 32 | 49 |
| 349 | 1 | 32 | 50 |
| 350 | 1 | 32 | 51 |
| 351 | 1 | 33 | 33 |
| 352 | 1 | 33 | 35 |
| 353 | 1 | 33 | 36 |
| 354 | 1 | 33 | 37 |
| 355 | 1 | 33 | 38 |
| 356 | 1 | 33 | 39 |
| 357 | 1 | 33 | 40 |
| 358 | 1 | 33 | 41 |
| 359 | 1 | 33 | 42 |
| 360 | 1 | 33 | 43 |
| 361 | 1 | 33 | 44 |
| 362 | 1 | 33 | 45 |
| 363 | 1 | 33 | 46 |
| 364 | 1 | 33 | 47 |
| 365 | 1 | 33 | 48 |
| 366 | 1 | 33 | 49 |
| 367 | 1 | 33 | 50 |
| 368 | 1 | 33 | 51 |
| 369 | 1 | 34 | 34 |
| 370 | 1 | 34 | 35 |
| 371 | 1 | 34 | 36 |
| 372 | 1 | 34 | 37 |
| 373 | 1 | 34 | 38 |
| 374 | 1 | 34 | 39 |
| 375 | 1 | 34 | 40 |
| 376 | 1 | 34 | 41 |
| 377 | 1 | 34 | 42 |
| 378 | 1 | 34 | 43 |
| 379 | 1 | 34 | 44 |
| 380 | 1 | 34 | 45 |
| 381 | 1 | 34 | 46 |
| 382 | 1 | 34 | 47 |
| 383 | 1 | 34 | 48 |
| 384 | 1 | 34 | 49 |
| 385 | 1 | 34 | 50 |
| 386 | 1 | 34 | 51 |
| 387 | 1 | 35 | 35 |
| 388 | 1 | 35 | 36 |
| 389 | 1 | 35 | 37 |
| 390 | 1 | 35 | 38 |
| 391 | 1 | 35 | 39 |
| 392 | 1 | 35 | 40 |
| 393 | 1 | 35 | 41 |
| 394 | 1 | 35 | 42 |
| 395 | 1 | 35 | 43 |
| 396 | 1 | 35 | 44 |
| 397 | 1 | 35 | 45 |
| 398 | 1 | 35 | 46 |
| 399 | 1 | 35 | 47 |
| 400 | 1 | 35 | 48 |
| 401 | 1 | 35 | 49 |
| 402 | 1 | 35 | 50 |
| 403 | 1 | 35 | 51 |
| 404 | 1 | 36 | 36 |
| 405 | 1 | 36 | 37 |
| 406 | 1 | 36 | 38 |
| 407 | 1 | 36 | 39 |
| 408 | 1 | 36 | 40 |
| 409 | 1 | 36 | 41 |
| 410 | 1 | 36 | 42 |
| 411 | 1 | 36 | 43 |
| 412 | 1 | 36 | 44 |
| 413 | 1 | 36 | 45 |
| 414 | 1 | 36 | 46 |
| 415 | 1 | 36 | 47 |
| 416 | 1 | 36 | 48 |
| 417 | 1 | 36 | 49 |
| 418 | 1 | 36 | 50 |
| 419 | 1 | 36 | 51 |
| 420 | 1 | 37 | 37 |
| 421 | 1 | 37 | 38 |
| 422 | 1 | 37 | 39 |
| 423 | 1 | 37 | 40 |
| 424 | 1 | 37 | 41 |
| 425 | 1 | 37 | 42 |
| 426 | 1 | 37 | 43 |
| 427 | 1 | 37 | 44 |
| 428 | 1 | 37 | 45 |
| 429 | 1 | 37 | 46 |
| 430 | 1 | 37 | 47 |
| 431 | 1 | 37 | 48 |
| 432 | 1 | 37 | 49 |
| 433 | 1 | 37 | 50 |
| 434 | 1 | 37 | 51 |
| 435 | 1 | 38 | 38 |
| 436 | 1 | 38 | 39 |
| 437 | 1 | 38 | 40 |
| 438 | 1 | 38 | 42 |
| 439 | 1 | 38 | 43 |
| 440 | 1 | 38 | 44 |
| 441 | 1 | 38 | 45 |
| 442 | 1 | 38 | 46 |
| 443 | 1 | 38 | 47 |
| 444 | 1 | 38 | 48 |
| 445 | 1 | 38 | 49 |
| 446 | 1 | 38 | 50 |
| 447 | 1 | 38 | 51 |
| 448 | 1 | 39 | 39 |
| 449 | 1 | 39 | 40 |
| 450 | 1 | 39 | 41 |
| 451 | 1 | 39 | 42 |
| 452 | 1 | 39 | 43 |
| 453 | 1 | 39 | 44 |
| 454 | 1 | 39 | 45 |
| 455 | 1 | 39 | 46 |
| 456 | 1 | 39 | 47 |
| 457 | 1 | 39 | 48 |
| 458 | 1 | 39 | 49 |
| 459 | 1 | 39 | 50 |
| 460 | 1 | 39 | 51 |
| 461 | 1 | 40 | 40 |
| 462 | 1 | 40 | 41 |
| 463 | 1 | 40 | 42 |
| 464 | 1 | 40 | 43 |
| 465 | 1 | 40 | 44 |
| 466 | 1 | 40 | 45 |
| 467 | 1 | 40 | 46 |
| 468 | 1 | 40 | 47 |
| 469 | 1 | 40 | 48 |
| 470 | 1 | 40 | 49 |
| 471 | 1 | 40 | 50 |
| 472 | 1 | 40 | 51 |
| 473 | 1 | 41 | 41 |
| 474 | 1 | 41 | 42 |
| 475 | 1 | 41 | 43 |
| 476 | 1 | 41 | 44 |
| 477 | 1 | 41 | 45 |
| 478 | 1 | 41 | 46 |
| 479 | 1 | 41 | 47 |
| 480 | 1 | 41 | 48 |
| 481 | 1 | 41 | 49 |
| 482 | 1 | 41 | 50 |
| 483 | 1 | 41 | 51 |
| 484 | 1 | 42 | 42 |
| 485 | 1 | 42 | 43 |
| 486 | 1 | 42 | 44 |
| 487 | 1 | 42 | 45 |
| 488 | 1 | 42 | 46 |
| 489 | 1 | 42 | 47 |
| 490 | 1 | 42 | 48 |
| 491 | 1 | 42 | 49 |
| 492 | 1 | 42 | 50 |
| 493 | 1 | 42 | 51 |
| 494 | 1 | 43 | 43 |
| 495 | 1 | 43 | 44 |
| 496 | 1 | 43 | 45 |
| 497 | 1 | 43 | 46 |
| 498 | 1 | 43 | 47 |
| 499 | 1 | 43 | 48 |
| 500 | 1 | 43 | 49 |
| 501 | 1 | 43 | 50 |
| 502 | 1 | 43 | 51 |
| 503 | 1 | 44 | 44 |
| 504 | 1 | 44 | 45 |
| 505 | 1 | 44 | 46 |
| 506 | 1 | 44 | 47 |
| 507 | 1 | 44 | 48 |
| 508 | 1 | 44 | 49 |
| 509 | 1 | 44 | 50 |
| 510 | 1 | 44 | 51 |
| 511 | 1 | 45 | 45 |
| 512 | 1 | 45 | 46 |
| 513 | 1 | 45 | 47 |
| 514 | 1 | 45 | 48 |
| 515 | 1 | 45 | 49 |
| 516 | 1 | 45 | 50 |
| 517 | 1 | 45 | 51 |
| 518 | 1 | 46 | 46 |
| 519 | 1 | 46 | 47 |
| 520 | 1 | 46 | 48 |
| 521 | 1 | 46 | 49 |
| 522 | 1 | 46 | 50 |
| 523 | 1 | 46 | 51 |
| 524 | 1 | 47 | 47 |
| 525 | 1 | 47 | 48 |
| 526 | 1 | 47 | 49 |
| 527 | 1 | 47 | 50 |
| 528 | 1 | 47 | 51 |
| 529 | 1 | 48 | 48 |
| 530 | 1 | 48 | 49 |
| 531 | 1 | 48 | 50 |
| 532 | 1 | 48 | 51 |
| 533 | 1 | 49 | 49 |
| 534 | 1 | 49 | 50 |
| 535 | 1 | 49 | 51 |
| 536 | 1 | 50 | 50 |
| 537 | 1 | 50 | 51 |
| 538 | 1 | 51 | 51 |
| 539 | 12 | 21 | 33 |
| 540 | 12 | 21 | 34 |
| 541 | 12 | 21 | 35 |
| 542 | 12 | 21 | 36 |
| 543 | 12 | 21 | 37 |
| 544 | 12 | 21 | 38 |
| 545 | 12 | 21 | 39 |
| 546 | 12 | 21 | 40 |
| 547 | 12 | 21 | 41 |
| 548 | 12 | 21 | 42 |
| 549 | 12 | 21 | 43 |
| 550 | 12 | 21 | 44 |
| 551 | 12 | 21 | 45 |
| 552 | 12 | 21 | 46 |
| 553 | 12 | 21 | 47 |
| 554 | 12 | 21 | 48 |
| 555 | 12 | 21 | 49 |
| 556 | 12 | 21 | 50 |
| 557 | 12 | 21 | 51 |
| 558 | 12 | 21 | 52 |
| 559 | 12 | 22 | 33 |
| 560 | 12 | 22 | 34 |
| 561 | 12 | 22 | 35 |
| 562 | 12 | 22 | 36 |
| 563 | 12 | 22 | 37 |
| 564 | 12 | 22 | 38 |
| 565 | 12 | 22 | 39 |
| 566 | 12 | 22 | 40 |
| 567 | 12 | 22 | 41 |
| 568 | 12 | 22 | 42 |
| 569 | 12 | 22 | 43 |
| 570 | 12 | 22 | 44 |
| 571 | 12 | 22 | 45 |
| 572 | 12 | 22 | 46 |
| 573 | 12 | 22 | 47 |
| 574 | 12 | 22 | 48 |
| 575 | 12 | 22 | 49 |
| 576 | 12 | 22 | 50 |
| 577 | 12 | 22 | 51 |
| 578 | 12 | 22 | 52 |
| 579 | 1 | 21 | 52 |
| 580 | 1 | 22 | 52 |
